SEQUENCE LISTING (396 sequences)

SEQ ID NO: 1 - protein, 706 aa, synthetic construct
MCGACKENYCLMITFAIFLSLIMLVEVAAAIAGYVGGSGPVRRYQGGPLGVRGGMEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGSGEQKLISEEDLGGPLGMTSGGSGPKNNHTASILDRMQADFKCCGAANYTDWEKIPSMSKNRVPDSCCINVTVGCGINFNEKAIHKEGCVEKIGGWLRKNVVLRDKLVKCEGISLLAQNTSWLLLLLLSLSLLQATDFMSL

SEQ ID NO: 2 - protein, 7 aa, synthetic construct
  VARIANT 1: note = Any amino acid
XLYSTLR

SEQ ID NO: 3 - protein, 6 aa, synthetic construct
PASVRN

SEQ ID NO: 4 - protein, 6 aa, synthetic construct
RPSPFW

SEQ ID NO: 5 - protein, 6 aa, synthetic construct
PAYWRT

SEQ ID NO: 6 - protein, 6 aa, synthetic construct
NRPASW

SEQ ID NO: 7 - protein, 6 aa, synthetic construct
VRAVYS

SEQ ID NO: 8 - protein, 5 aa, synthetic construct
YYLRA

SEQ ID NO: 9 - protein, 6 aa, synthetic construct
PLKITR

SEQ ID NO: 10 - protein, 6 aa, synthetic construct
VSFLQQ

SEQ ID NO: 11 - protein, 6 aa, synthetic construct
TVLNSL

SEQ ID NO: 12 - protein, 6 aa, synthetic construct
NFLRKM

SEQ ID NO: 13 - protein, 6 aa, synthetic construct
NLALKQ

SEQ ID NO: 14 - protein, 6 aa, synthetic construct
LALKMG

SEQ ID NO: 15 - protein, 6 aa, synthetic construct
QQMSLR

SEQ ID NO: 16 - protein, 6 aa, synthetic construct
MLKLQP

SEQ ID NO: 17 - protein, 6 aa, synthetic construct
SWSLIL

SEQ ID NO: 18 - protein, 6 aa, synthetic construct
ALQRLP

SEQ ID NO: 19 - protein, 6 aa, synthetic construct
YYGELL

SEQ ID NO: 20 - protein, 6 aa, synthetic construct
LELRVN

SEQ ID NO: 21 - protein, 7 aa, synthetic construct
SGVNSLR

SEQ ID NO: 22 - protein, 6 aa, synthetic construct
MLLMKQ

SEQ ID NO: 23 - protein, 6 aa, synthetic construct
VTVIAL

SEQ ID NO: 24 - protein, 6 aa, synthetic construct
ALDLKR

SEQ ID NO: 25 - protein, 6 aa, synthetic construct
VGALNK

SEQ ID NO: 26 - protein, 6 aa, synthetic construct
NLVRVR

SEQ ID NO: 27 - protein, 6 aa, synthetic construct
TTSLRL

SEQ ID NO: 28 - protein, 6 aa, synthetic construct
PSRILM

SEQ ID NO: 29 - protein, 6 aa, synthetic construct
PQAVVK

SEQ ID NO: 30 - protein, 20 aa, synthetic construct
EAAAREAAAREAAAREAAAR

SEQ ID NO: 31 - DNA, 60 bp, synthetic construct
  source 1..60: mol_type = other DNA
gaagctgctgcaagagaagctgcagctagggaggctgcagctagggaggctgctgcaaga

SEQ ID NO: 32-33 - skipped (2 entries)

SEQ ID NO: 34 - protein, 6 aa, synthetic construct
GSGSGS

SEQ ID NO: 35 - DNA, 18 bp, synthetic construct
  source 1..18: mol_type = other DNA
ggtagcggcagcggtagc

SEQ ID NO: 36 - protein, 10 aa, synthetic construct
GSGSGSGSGS

SEQ ID NO: 37 - DNA, 30 bp, synthetic construct
  source 1..30: mol_type = other DNA
ggtagcggcagcggtagcggtagcggcagc

SEQ ID NO: 38 - protein, 10 aa, synthetic construct
GENLYFQSGG

SEQ ID NO: 39 - DNA, 30 bp, synthetic construct
  source 1..30: mol_type = other DNA
ggtgaaaatttgtattttcaatctggtggt

SEQ ID NO: 40 - protein, 8 aa, synthetic construct
SACYCELS

SEQ ID NO: 41 - DNA, 24 bp, synthetic construct
  source 1..24: mol_type = other DNA
tccgcttgttactgtgagctttcc

SEQ ID NO: 42 - protein, 6 aa, synthetic construct
GGSGGS

SEQ ID NO: 43 - DNA, 24 bp, synthetic construct
  source 1..24: mol_type = other DNA
ggtggtggttctggtggtggttct

SEQ ID NO: 44 - protein, 10 aa, synthetic construct
GGGGSGGGGS

SEQ ID NO: 45 - DNA, 30 bp, synthetic construct
  source 1..30: mol_type = other DNA
ggtggtggtggttctggtggtggtggttct

SEQ ID NO: 46 - protein, 13 aa, synthetic construct
GGGSGGGGSGGGS

SEQ ID NO: 47 - DNA, 39 bp, synthetic construct
  source 1..39: mol_type = other DNA
ggcggtggctctggaggtggtgggtccggcggtggctct

SEQ ID NO: 48 - protein, 10 aa, synthetic construct
GGGGSGGGGS

SEQ ID NO: 49 - DNA, 30 bp, synthetic construct
  source 1..30: mol_type = other DNA
ggtggcggtggctctggaggtggtgggtcc

SEQ ID NO: 50 - protein, 17 aa, synthetic construct
RPACKIPNDLKQKVMNH

SEQ ID NO: 51 - DNA, 51 bp, synthetic construct
  source 1..51: mol_type = other DNA
cgaccagcctgtaagattccaaatgacctgaagcagaaagttatgaatcac

SEQ ID NO: 52 - protein, 17 aa, synthetic construct
RPACKIPNDLKQKVMNH

SEQ ID NO: 53 - DNA, 45 bp, synthetic construct
  source 1..45: mol_type = other DNA
ggtggaggaggttctggaggcggtggaagtggtggcggaggtagc

SEQ ID NO: 54 - protein, 947 aa, synthetic construct
MSCSVARVGLGLLLLLMGAGLAVQGWFLLQLHWRLGEMVTRLPDGPAGSWEQLIQERRSHEVNPAAGGSGPVRRYQMEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGSGEQKLISEEDLSGGSGSLECTKLCLPQIENVKGTEDSGGSGQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGREDPDAVDIWGQGTMVTVSSGGGGSGGGGSGGGGSDIQMTQSPSSLSASVGDRVTITCRASQGIRSWLAWYQQKPEKARKSLIYAASSLQSGVPSRFSGSGSGTDFTLTISSLQPEDFATYYCQQYNSYPLTFGGGTKVEIKGGGGSGGGGSGGGGSSGTTVLLPLVIFFGLCLLSLLFIGLMYRYGGSGKRGRKKLLYIFKQPFMRPVQTTQEEDG

SEQ ID NO: 55 - DNA, 27 bp, synthetic construct
  source 1..27: mol_type = other DNA
tctggttctggttctggttctggttct

SEQ ID NO: 56 - protein, 12 aa, synthetic construct
SGSGSGSGSGSG

SEQ ID NO: 57 - DNA, 36 bp, synthetic construct
  source 1..36: mol_type = other DNA
tctggttctggttctggttctggttctggttctggt

SEQ ID NO: 58 - protein, 15 aa, synthetic construct
SGSGSGSGSGSGGSG

SEQ ID NO: 59 - DNA, 45 bp, synthetic construct
  source 1..45: mol_type = other DNA
tctggttctggttctggttctggttctggttctggtggttctggt

SEQ ID NO: 60 - protein, 41 aa, synthetic construct
SLLHTSLSPRRRERWAAAAATRASSIPASPQEEEKGGRRRQ

SEQ ID NO: 61 - DNA, 124 bp, synthetic construct
  source 1..124: mol_type = other DNA
agcctcctccataccagcctctccccaagaagaagagaaaggtgggcggcggcggcagctactagagcctcctccataccagcctctccccaagaagaagagaaaggtgggcggcggcggcagc

SEQ ID NO: 62 - protein, 12 aa, synthetic construct
PPPYQPLGGGGS

SEQ ID NO: 63 - DNA, 36 bp, synthetic construct
  source 1..36: mol_type = other DNA
cctcctccataccagcctctcggcggcggcggcagc

SEQ ID NO: 64 - protein, 24 aa, synthetic construct
ALKGGALKGGALKGGALKGGALKG

SEQ ID NO: 65 - DNA, 72 bp, synthetic construct
  source 1..72: mol_type = other DNA
gccttgaaaggtggggctttaaaagggggagccttgaaggggggtgccttgaagggtggggcattgaaaggc

SEQ ID NO: 66 - protein, 128 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGDDDDKGTRGSLEGG

SEQ ID NO: 67 - DNA, 384 bp, synthetic construct
  source 1..384: mol_type = other DNA
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgggttctggttctggtgatgacgatgacaagggtacccggggatccctcgagggtggt

SEQ ID NO: 68 - protein, 8 aa, synthetic construct
GGSGGGSG

SEQ ID NO: 69 - DNA, 24 bp, synthetic construct
  source 1..24: mol_type = other DNA
ggaggctccggcggcggatccggt

SEQ ID NO: 70 - protein, 4 aa, synthetic construct
GGSG

SEQ ID NO: 71 - DNA, 12 bp, synthetic construct
  source 1..12: mol_type = other DNA
ggtggttctggt

SEQ ID NO: 72 - protein, 8 aa, synthetic construct
GGSGGGSG

SEQ ID NO: 73 - DNA, 24 bp, synthetic construct
  source 1..24: mol_type = other DNA
ggtggttctggtggtggttctggt

SEQ ID NO: 74 - protein, 12 aa, synthetic construct
GGSGGGSGGGSG

SEQ ID NO: 75 - DNA, 36 bp, synthetic construct
  source 1..36: mol_type = other DNA
ggtggttctggtggtggttctggtggtggttctggt

SEQ ID NO: 76 - protein, 36 aa, synthetic construct
GGSAGGSGSGSSGGSSGASGTGTAGGTGSGSGTGSG

SEQ ID NO: 77 - DNA, 108 bp, synthetic construct
  source 1..108: mol_type = other DNA
ggtggttctgccggtggctccggttctggctccagcggtggcagctctggtgcgtccggcacgggtactgcgggtggcactggcagcggttccggtactggctctggc

SEQ ID NO: 78 - protein, 39 aa, synthetic construct
SEGGGSGGGSEGGGSEGGGSEGGGSEGGGSEGGGSGGGS

SEQ ID NO: 79 - DNA, 108 bp, synthetic construct
  source 1..108: mol_type = other DNA
ggtggttctggcggcggttctgaaggtggcggctccgaaggcggcggcagcgagggcggtggtagcgaaggtggtggctccgagggtggcggttccggcggcggtagc

SEQ ID NO: 80 - protein, 12 aa, synthetic construct
AAAGGSGGSGGS

SEQ ID NO: 81 - DNA, 36 bp, synthetic construct
  source 1..36: mol_type = other DNA
gcagctgctggtggaagtggaggttctggcgggagc

SEQ ID NO: 82 - protein, 5 aa, synthetic construct
GGGGS

SEQ ID NO: 83 - DNA, 15 bp, synthetic construct
  source 1..15: mol_type = other DNA
ggaggaggaggtagt

SEQ ID NO: 84 - protein, 485 aa, synthetic construct
MTHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGGGGSLVPRGSGGGGSGNSLAKNVLSGGKGNDKLYGSEGADLLDGGEGNDLLKGGYGNDIYRYLSGYGHHIIDDEGGKDDKLSLADIDFRDVAFKREGNDLIMYKAEGNVLSIGHKNGITFKNWFEKESDDLSNHQIEQIFDKDGRVITPDSLKKAFEYQQSNNKVSYVYGHDASTYGSQDNLNPLINEISKIISAAGNFDVKEERSAASLLQLSGNASDFSYGRNSITLTASAHHHHHH

SEQ ID NO: 85 - DNA, 1458 bp, synthetic construct
  source 1..1458: mol_type = other DNA
atgacacacgaacattctgctcagtggctgaataattataaaaaaggttatggttacggtccctacccgctgggaatcaatggaggaatgcactacggtgtggacttctttatgaacattggcacaccggtcaaggcgattagttcaggtaaaatcgtagaagctggttggtcaaactatggcggtgggaatcaaatcggtcttatcgagaatgatggggtgcatcgtcagtggtacatgcacctgagcaaatataatgtgaaagttggcgactatgtaaaggccgggcaaatcattgggtggagcggctctactggttattctacagctccgcatcttcatttccaacgcatggttaatagtttttcaaactctacagcccaagaccctatgccctttttgaagtctgcgggttacgggaaggccggtggaaccgttacccccacgccaaataccggctggaaaacgaataagtatggaacactgtataagtctgaatccgcctcgttcactcccaacacggatattatcacacgcaccactggaccattccgttccatgcctcagtcaggcgttttaaaggcgggtcagacaatccactacgacgaggttatgaagcaggacggacatgtctgggtcgggtacacgggtaactccgggcaacgtatctacttgccggtccgcacgtggaataaaagcaccaatacactgggcgtactttggggcacaattaagggagggggcggatcgctggttcctcgcgggtcaggagggggcgggagcggaaattctcttgctaaaaatgtattatccggtggaaaaggtaatgacaagttgtacggcagtgagggagcagacctgcttgatggcggagaagggaatgatcttctgaaaggtggatatggtaatgatatttatcgttatctttcaggatatggccatcatattattgacgatgaaggggggaaagacgataaactcagtttagctgatatagatttccgggacgttgcctttaagcgagaagggaatgacctcattatgtataaagctgaaggtaatgttctttctattggccacaaaaatggtattacatttaaaaactggtttgaaaaagagtcagatgatctctctaatcatcagatagagcagatttttgataaagacggcagggtaatcacaccagattctcttaaaaaagcatttgaatatcagcagagtaataacaaggtaagttatgtgtatggacatgatgcatcaacttatgggagccaggacaatcttaatccattaattaatgaaatcagcaaaatcatttcagccgcaggtaacttcgatgttaaggaggaaagatctgccgcttctttattgcagttgtccggtaatgccagtgatttttcatatggacggaactcaataactttgacagcatcagcacaccaccaccaccaccactga

SEQ ID NO: 86 - protein, 485 aa, synthetic construct; identical to SEQ ID NO: 84

SEQ ID NO: 87 - DNA, 1496 bp, synthetic construct
  source 1..1496: mol_type = other DNA
taatacgactcactatagggaaagaggagaaatactagatgacacacgaacattctgctcagtggctgaataattataaaaaaggttatggttacggtccctacccgctgggaatcaatggaggaatgcactacggtgtggacttctttatgaacattggcacaccggtcaaggcgattagttcaggtaaaatcgtagaagctggttggtcaaactatggcggtgggaatcaaatcggtcttatcgagaatgatggggtgcatcgtcagtggtacatgcacctgagcaaatataatgtgaaagttggcgactatgtaaaggccgggcaaatcattgggtggagcggctctactggttattctacagctccgcatcttcatttccaacgcatggttaatagtttttcaaactctacagcccaagaccctatgccctttttgaagtctgcgggttacgggaaggccggtggaaccgttacccccacgccaaataccggctggaaaacgaataagtatggaacactgtataagtctgaatccgcctcgttcactcccaacacggatattatcacacgcaccactggaccattccgttccatgcctcagtcaggcgttttaaaggcgggtcagacaatccactacgacgaggttatgaagcaggacggacatgtctgggtcgggtacacgggtaactccgggcaacgtatctacttgccggtccgcacgtggaataaaagcaccaatacactgggcgtactttggggcacaattaagggagggggcggatcgctggttcctcgcgggtcaggagggggcgggagcggaaattctcttgctaaaaatgtattatccggtggaaaaggtaatgacaagttgtacggcagtgagggagcagacctgcttgatggcggagaagggaatgatcttctgaaaggtggatatggtaatgatatttatcgttatctttcaggatatggccatcatattattgacgatgaaggggggaaagacgataaactcagtttagctgatatagatttccgggacgttgcctttaagcgagaagggaatgacctcattatgtataaagctgaaggtaatgttctttctattggccacaaaaatggtattacatttaaaaactggtttgaaaaagagtcagatgatctctctaatcatcagatagagcagatttttgataaagacggcagggtaatcacaccagattctcttaaaaaagcatttgaatatcagcagagtaataacaaggtaagttatgtgtatggacatgatgcatcaacttatgggagccaggacaatcttaatccattaattaatgaaatcagcaaaatcatttcagccgcaggtaacttcgatgttaaggaggaaagatctgccgcttctttattgcagttgtccggtaatgccagtgatttttcatatggacggaactcaataactttgacagcatcagcacaccaccaccaccaccactga

SEQ ID NO: 88 - protein, 738 aa, synthetic construct
MSNDNEVPGSMVIVAQGPDDQYAYEVPPIDSAAVAGNMFGDLIQREIYLQKNIYYPVRSIFEQGTKEKKEINKKVSDQVDGLLKQITQGKREATRQERVDVMSAVLHKMESDLEGYKKTFTKGPFIDYEKQSSLSIYEAWVKIWEKNSWEERKKYPFQQLVRDELERAVAYYKQDSLSEAVKVLRQELNKQKALKEKEDLSQLERDYRTRKANLEMKVQSELDQAGSALPPLVSPTPEQWLERATRLVTQAIADKKQLQTTNNTLIKNSPTPLEKQKAIYNGELLVDEIASLQARLVKLNAETTRRRTEAERKAAEEQALQDAIKFTADFYKEVTEKFGARTSEMARQLAEGARGKNIRSSAEAIKSFEKHKDALNKKLSLKDRQAIAKAFDSLDKQMMAKSLEKFSKGFGVVGKAIDAASLYQEFKISTETGDWKPFFVKIETLAAGAAASWLVGIAFATATATPIGILGFALVMAVTGAMIDEDLLEKANNLVISIGGGGSLVPRGSGGGGSGNSLAKNVLSGGKGNDKLYGSEGADLLDGGEGNDLLKGGYGNDIYRYLSGYGHHIIDDEGGKDDKLSLADIDFRDVAFKREGNDLIMYKAEGNVLSIGHKNGITFKNWFEKESDDLSNHQIEQIFDKDGRVITPDSLKKAFEYQQSNNKVSYVYGHDASTYGSQDNLNPLINEISKIISAAGNFDVKEERSAASLLQLSGNASDFSYGRNSITLTASAHHHHHH

SEQ ID NO: 89 - DNA, 2217 bp, synthetic construct
  source 1..2217: mol_type = other DNA
atgtcaaatgataacgaagtcccaggtagtatggtgattgtagcccagggtccagacgaccagtatgcgtatgaggtaccgcctatcgactccgcggcggttgcaggcaatatgttcggcgatcttatccaacgcgagatctacctgcaaaagaatatctattatccggtacggtcgatctttgagcaagggactaaagagaagaaagaaattaacaagaaggtgtctgatcaggttgatggcctgctcaaacaaatcacgcaaggtaagcgcgaagcgactcggcaagaacgcgttgacgtaatgtctgcggttcttcataagatggaatcagacctcgaagggtataaaaagacattcacaaaaggcccatttattgactacgagaagcaatcatccttatcgatctacgaggcatgggtgaagatctgggagaaaaatagctgggaggaacgtaaaaaatatcctttccaacagctggtgcgtgacgaacttgagcgggccgtagcttattataaacaagactcgttgtcggaggctgtaaaggtgcttcgtcaggagttgaataaacagaaggcactcaaggagaaggaagaccttagtcagttggagcgtgattatcgtactcggaaagcaaatctcgaaatgaaagtgcagtccgaactcgaccaggctgggtccgcccttccgcctttggtttcaccaacaccagaacagtggcttgaacgggcaacacgtttagtaactcaagcgattgcggataagaagcagcttcagactacgaacaacactctgattaaaaattcgccgacaccacttgagaagcagaaggcgatttataatggggagttgcttgtggatgaaattgcaagtttacaagcacgccttgttaaactcaacgccgaaactacccggcggcgtacggaagcagagcgcaaggctgcggaagaacaggcacttcaggatgctatcaagtttacggcggacttctacaaggaggtcacagagaaattcggtgcgcgtacttcagagatggcccggcaattagcagagggggcacggggtaaaaacattcgttcgagtgcagaggccatcaaatctttcgaaaagcataaagacgcattgaataaaaagctaagcttaaaagaccggcaggctatcgcaaaggcatttgattcgttggacaaacaaatgatggcgaagagccttgaaaaattctcaaagggttttggcgttgtggggaaggctatcgacgcggcgagtctttaccaagagtttaaaatcagcaccgagactggtgactggaaaccattttttgttaagattgaaaccttagctgcgggcgccgcagcgtcatggctcgtcgggatcgcctttgccacggccacagccacgccaattggcattctcggttttgcactcgtgatggcagttaccggggctatgattgatgaggacctgctcgaaaaggcaaataatcttgtgatcagcattggagggggcggatcgctggttcctcgcgggtcaggagggggcgggagcggaaattctcttgctaaaaatgtattatccggtggaaaaggtaatgacaagttgtacggcagtgagggagcagacctgcttgatggcggagaagggaatgatcttctgaaaggtggatatggtaatgatatttatcgttatctttcaggatatggccatcatattattgacgatgaaggggggaaagacgataaactcagtttagctgatatagatttccgggacgttgcctttaagcgagagggtaacgacctcattatgtataaagctgaaggtaatgttctttctattggccacaaaaatggtattacatttaaaaactggtttgaaaaagagtcagatgatctctctaatcatcagatagagcagatttttgataaagacggcagggtaatcacaccagattctcttaaaaaagcatttgaatatcagcagagtaataacaaggtaagttatgtgtatggacatgatgcatcaacttatgggagccaggacaatcttaatccattaattaatgaaatcagcaaaatcatttcagccgcaggtaacttcgatgttaaggaggaaagatctgccgcttctttattgcagttgtccggtaatgccagtgatttttcatatggacggaactcaataactttgacagcatcagcacatcatcaccatcaccactga

SEQ ID NO: 90 - protein, 738 aa, synthetic construct; identical to SEQ ID NO: 88

SEQ ID NO: 91 - DNA, 2288 bp, synthetic construct
  source 1..2288: mol_type = other DNA
ctgacgctttttatcgcaactctctactgtttctccatacccgttttttaactttaagaaggagatataccatgtcaaatgataacgaagtcccaggtagtatggtgattgtagcccagggtccagacgaccagtatgcgtatgaggtaccgcctatcgactccgcggcggttgcaggcaatatgttcggcgatcttatccaacgcgagatctacctgcaaaagaatatctattatccggtacggtcgatctttgagcaagggactaaagagaagaaagaaattaacaagaaggtgtctgatcaggttgatggcctgctcaaacaaatcacgcaaggtaagcgcgaagcgactcggcaagaacgcgttgacgtaatgtctgcggttcttcataagatggaatcagacctcgaagggtataaaaagacattcacaaaaggcccatttattgactacgagaagcaatcatccttatcgatctacgaggcatgggtgaagatctgggagaaaaatagctgggaggaacgtaaaaaatatcctttccaacagctggtgcgtgacgaacttgagcgggccgtagcttattataaacaagactcgttgtcggaggctgtaaaggtgcttcgtcaggagttgaataaacagaaggcactcaaggagaaggaagaccttagtcagttggagcgtgattatcgtactcggaaagcaaatctcgaaatgaaagtgcagtccgaactcgaccaggctgggtccgcccttccgcctttggtttcaccaacaccagaacagtggcttgaacgggcaacacgtttagtaactcaagcgattgcggataagaagcagcttcagactacgaacaacactctgattaaaaattcgccgacaccacttgagaagcagaaggcgatttataatggggagttgcttgtggatgaaattgcaagtttacaagcacgccttgttaaactcaacgccgaaactacccggcggcgtacggaagcagagcgcaaggctgcggaagaacaggcacttcaggatgctatcaagtttacggcggacttctacaaggaggtcacagagaaattcggtgcgcgtacttcagagatggcccggcaattagcagagggggcacggggtaaaaacattcgttcgagtgcagaggccatcaaatctttcgaaaagcataaagacgcattgaataaaaagctaagcttaaaagaccggcaggctatcgcaaaggcatttgattcgttggacaaacaaatgatggcgaagagccttgaaaaattctcaaagggttttggcgttgtggggaaggctatcgacgcggcgagtctttaccaagagtttaaaatcagcaccgagactggtgactggaaaccattttttgttaagattgaaaccttagctgcgggcgccgcagcgtcatggctcgtcgggatcgcctttgccacggccacagccacgccaattggcattctcggttttgcactcgtgatggcagttaccggggctatgattgatgaggacctgctcgaaaaggcaaataatcttgtgatcagcattggagggggcggatcgctggttcctcgcgggtcaggagggggcgggagcggaaattctcttgctaaaaatgtattatccggtggaaaaggtaatgacaagttgtacggcagtgagggagcagacctgcttgatggcggagaagggaatgatcttctgaaaggtggatatggtaatgatatttatcgttatctttcaggatatggccatcatattattgacgatgaaggggggaaagacgataaactcagtttagctgatatagatttccgggacgttgcctttaagcgagagggtaacgacctcattatgtataaagctgaaggtaatgttctttctattggccacaaaaatggtattacatttaaaaactggtttgaaaaagagtcagatgatctctctaatcatcagatagagcagatttttgataaagacggcagggtaatcacaccagattctcttaaaaaagcatttgaatatcagcagagtaataacaaggtaagttatgtgtatggacatgatgcatcaacttatgggagccaggacaatcttaatccattaattaatgaaatcagcaaaatcatttcagccgcaggtaacttcgatgttaaggaggaaagatctgccgcttctttattgcagttgtccggtaatgccagtgatttttcatatggacggaactcaataactttgacagcatcagcacatcatcaccatcaccactga

SEQ ID NO: 92 - protein, 4 aa, synthetic construct
GGGG

SEQ ID NO: 93 - DNA, 12 bp, synthetic construct
  source 1..12: mol_type = other DNA
ggaggaggagga

SEQ ID NO: 94 - protein, 29 aa, synthetic construct
WRKRLRKKRLRKKRRLKKRRRKKQRRKRR

SEQ ID NO: 95 - DNA, 89 bp, synthetic construct
  source 1..89: mol_type = other DNA
tggcggaagcggctgcgaaagaagcggctgcgaaagaagcggcggctaaagaagcggcggcgaaagaagcagcggcgaaagcggcggcg

SEQ ID NO: 96 - protein, 5 aa, synthetic construct
GSGGS

SEQ ID NO: 97 - DNA, 15 bp, synthetic construct
  source 1..15: mol_type = other DNA
ggatctggtggaagt

SEQ ID NO: 98 - protein, 5 aa, synthetic construct
GSSGS

SEQ ID NO: 99 - DNA, 15 bp, synthetic construct
  source 1..15: mol_type = other DNA
ggcagctcgggatca

SEQ ID NO: 100 - protein, 22 aa, synthetic construct
LEGSGQGPGSGQGSGSPGSGQG

SEQ ID NO: 101 - DNA, 66 bp, synthetic construct
  source 1..66: mol_type = other DNA
ttggaaggatctggccaaggtcccggcagcgggcaagggtcgggaagcccagggtccggtcagggc

SEQ ID NO: 102 - protein, 9 aa, synthetic construct
GGSGGSGGS

SEQ ID NO: 103 - DNA, 27 bp, synthetic construct
  source 1..27: mol_type = other DNA
ggcggtagcggcggtagcggcggtagc

SEQ ID NO: 104 - protein, 36 aa, synthetic construct
GGSGGGSEGGGSEGGGSEGGGSEGGGSEGGGSGGGS

SEQ ID NO: 105 - DNA, 108 bp, synthetic construct; identical to SEQ ID NO: 79
  source 1..108: mol_type = other DNA

SEQ ID NO: 106 - protein, 36 aa, synthetic construct; identical to SEQ ID NO: 76

SEQ ID NO: 107 - DNA, 108 bp, synthetic construct; identical to SEQ ID NO: 77
  source 1..108: mol_type = other DNA

SEQ ID NO: 108 - protein, 64 aa, synthetic construct
VDNKFNKEMWAAWEEIRNLPNLNGWQMTAFIASLVDDPSQSANLLAEAKKLNDAQAPKTGGGSG

SEQ ID NO: 109 - DNA, 192 bp, synthetic construct
  source 1..192: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggt

SEQ ID NO: 110 - protein, 68 aa, synthetic construct
VDNKFNKEMWAAWEEIRNLPNLNGWQMTAFIASLVDDPSQSANLLAEAKKLNDAQAPKTGGGSGGGSG

SEQ ID NO: 111 - DNA, 204 bp, synthetic construct
  source 1..204: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggtggtggttctggt

SEQ ID NO: 112 - protein, 72 aa, synthetic construct
VDNKFNKEMWAAWEEIRNLPNLNGWQMTAFIASLVDDPSQSANLLAEAKKLNDAQAPKTGGGSGGGSGGGSG

SEQ ID NO: 113 - DNA, 216 bp, synthetic construct
  source 1..216: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggtggtggttctggtggtggttctggt

SEQ ID NO: 114 - protein, 96 aa, synthetic construct
VDNKFNKEMWAAWEEIRNLPNLNGWQMTAFIASLVDDPSQSANLLAEAKKLNDAQAPKTGGGSGGGSEGGGSEGGGSEGGGSEGGGSEGGGSGGGS

SEQ ID NO: 115 - DNA, 288 bp, synthetic construct
  source 1..288: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggcggcggttctgaaggtggcggctccgaaggcggcggcagcgagggcggtggtagcgaaggtggtggctccgagggtggcggttccggcggcggtagc

SEQ ID NO: 116 - protein, 15 aa, synthetic construct
GGGGSGGGGSGGGGS

SEQ ID NO: 117 - DNA, 45 bp, synthetic construct
  source 1..45: mol_type = other DNA
ggtggaggaggctctggtggaggcggtagcggaggcggagggtcg

SEQ ID NO: 118 - protein, 4 aa, synthetic construct
GGSG

SEQ ID NO: 119 - DNA, 12 bp, synthetic construct
  source 1..12: mol_type = other DNA
ggtggttctggt

SEQ ID NO: 120 - protein, 10 aa, synthetic construct
GENLYFQSGG

SEQ ID NO: 121 - DNA, 30 bp, synthetic construct; identical to SEQ ID NO: 39
  source 1..30: mol_type = other DNA

SEQ ID NO: 122 - protein, 6 aa, synthetic construct
GGSGGS

SEQ ID NO: 123 - DNA, 18 bp, synthetic construct
  source 1..18: mol_type = other DNA
ggaggttcaggaggcagc

SEQ ID NO: 124 - DNA, 2121 bp, synthetic construct
  source 1..2121: mol_type = other DNA
atgtgcggcgcatgcaaggaaaactactgtctgatgatcacattcgccatcttcctgagcctgatcatgctggtggaagtggccgctgccatcgccggatacgtgggaggcagcgggcctgtgcggcggtaccagggaggcccactgggagttagaggcggcatggaagtgcagctggtcgaatcgggaggcggcgtggtgagaccaggaggcagcctccggctgtcttgtgccgcgagtggtttcacctttgacgactacggcatgagctgggtgagacaggctcctggaaagggcctggaatgggtttccggcatcaactggaacggcggatcgactggctacgccgacagcgtgaaaggcagattcaccatcagcagagataacgccaagaacagcctgtacctgcagatgaactccctgagagccgaggacaccgccgtgtactactgcgccagaggcagaagcctgctgttcgactactggggacagggcacactggtgaccgtcagcagaggcggcggcggctctggcggcggcggaagcggaggcggcggatcctccgagctgacacaggaccccgctgttagcgtggctctgggccagaccgtgcggatcacctgtcagggcgacagcctccgctcctactacgccagctggtaccaacaaaaacctggccaagctcctgtgctggtcatctacggaaagaacaacagacctagtggcatccctgatagatttagcggcagcagcagcggcaacaccgcctcactgaccattacaggcgcccaggccgaggatgaggctgactattactgcaactccagggacagctctggaaatcatgtggtcttcggcggcggcacaaagctgaccgtgctgccttctggccaagccggtgccgccgcgtctgagagcctgttcgtgagcaaccacgcctacgaggccagcggcggccccgagcaggtgcagctggtgcagagcggcgctgaggtgaaaaagcccggctctagcgtgaaggtgtcctgcaaggccagcggctacaccttcacagattataatatgcactgggtcagacaggcccctgggcagggactggaatggatcggctatatctacccctacaacggcggaacaggctacaaccagaagttcaagagcaaggccaccatcacagccgacgagagcaccaacaccgcttatatggaactgtcttctctgcggagcgaagataccgccgtgtactactgtgccagaggccggcctgccatggactactggggccagggcaccctggtgacagtgtcctcaggcggcggcggtagcggcggcggcggcagcggcggcggtggttctgatatccagatgacccagagccccagcagcctgtccgccagtgtcggagatagggtgaccatcacctgcagagcctctgaaagcgttgacaattacggcatcagcttcatgaactggttccagcagaaacctggcaaagcccctaagcttcttatttacgccgcttccaaccagggctctggcgtgcccagccggttcagcggctcaggatctggaaccgactttacgctgacaatcagctctctgcagcctgatgacttcgccacatactactgccagcaaagcaaagaggtgccttggaccttcggccagggcaccaaggtggagattaagggcgggtccggagagcagaagctgatctctgaggaagacctgggaggccctctgggcatgacctctggaggcagcggccctaagaacaaccacaccgccagcatcctggaccggatgcaggccgactttaagtgctgcggcgccgctaattacaccgactgggagaaaatccccagcatgagcaagaatagagtgcccgatagctgttgcatcaacgtgacagtgggctgcggcatcaatttcaacgagaaggctatccacaaggagggctgcgtggaaaagatcggcggctggctgagaaagaacgtggtgctgagagacaagctcgtgaaatgcgagggcatcagcctgctggcccagaacacatcctggctgctgctgctgctgctcagcctgagtctgctgcaagccaccgatttcatgtctctgtaa

SEQ ID NO: 125 - protein, 10 aa, synthetic construct
MCGACKENYC

SEQ ID NO: 126 - DNA, 30 bp, synthetic construct
  source 1..30: mol_type = other DNA
atgtgcggcgcatgcaaggaaaactactgt

SEQ ID NO: 127 - protein, 21 aa, synthetic construct
LMITFAIFLSLIMLVEVAAAI

SEQ ID NO: 128 - DNA, 63 bp, synthetic construct
  source 1..63: mol_type = other DNA
ctgatgatcacattcgccatcttcctgagcctgatcatgctggtggaagtggccgctgccatc

SEQ ID NO: 129 - protein, 10 aa, synthetic construct
GGSGPVRRYQ

SEQ ID NO: 130 - DNA, 30 bp, synthetic construct
  source 1..30: mol_type = other DNA
ggaggcagcgggcctgtgcggcggtaccag

SEQ ID NO: 131 - protein, 9 aa, synthetic construct
GGPLGVRGG

SEQ ID NO: 132 - DNA, 27 bp, synthetic construct
  source 1..27: mol_type = other DNA
ggaggcccactgggagttagaggcggc

SEQ ID NO: 133 - protein, 118 aa, synthetic construct
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 134 - DNA, 354 bp, synthetic construct
  source 1..354: mol_type = other DNA
atggaagtgcagctggtcgaatcgggaggcggcgtggtgagaccaggaggcagcctccggctgtcttgtgccgcgagtggtttcacctttgacgactacggcatgagctgggtgagacaggctcctggaaagggcctggaatgggtttccggcatcaactggaacggcggatcgactggctacgccgacagcgtgaaaggcagattcaccatcagcagagataacgccaagaacagcctgtacctgcagatgaactccctgagagccgaggacaccgccgtgtactactgcgccagaggcagaagcctgctgttcgactactggggacagggcacactggtgaccgtcagcaga

SEQ ID NO: 135 - protein, 15 aa, synthetic construct
GGGGSGGGGSGGGGS

SEQ ID NO: 136 - DNA, 45 bp, synthetic construct
  source 1..45: mol_type = other DNA
ggcggcggcggctctggcggcggcggaagcggaggcggcggatcc

SEQ ID NO: 137 - protein, 107 aa, synthetic construct
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 138 - DNA, 321 bp, synthetic construct
  source 1..321: mol_type = other DNA
tccgagctgacacaggaccccgctgttagcgtggctctgggccagaccgtgcggatcacctgtcagggcgacagcctccgctcctactacgccagctggtaccaacaaaaacctggccaagctcctgtgctggtcatctacggaaagaacaacagacctagtggcatccctgatagatttagcggcagcagcagcggcaacaccgcctcactgaccattacaggcgcccaggccgaggatgaggctgactattactgcaactccagggacagctctggaaatcatgtggtcttcggcggcggcacaaagctgaccgtgctg

SEQ ID NO: 139 - protein, 20 aa, synthetic construct
PSGQAGAAASESLFVSNHAY

SEQ ID NO: 140 - DNA, 60 bp, synthetic construct
  source 1..60: mol_type = other DNA
ccttctggccaagccggtgccgccgcgtctgagagcctgttcgtgagcaaccacgcctac

SEQ ID NO: 141 - protein, 7 aa, synthetic construct
EASGGPE

SEQ ID NO: 142 - DNA, 21 bp, synthetic construct
  source 1..21: mol_type = other DNA
gaggccagcggcggccccgag

SEQ ID NO: 143 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 144 - DNA, 348 bp, synthetic construct
  source 1..348: mol_type = other DNA
caggtgcagctggtgcagagcggcgctgaggtgaaaaagcccggctctagcgtgaaggtgtcctgcaaggccagcggctacaccttcacagattataatatgcactgggtcagacaggcccctgggcagggactggaatggatcggctatatctacccctacaacggcggaacaggctacaaccagaagttcaagagcaaggccaccatcacagccgacgagagcaccaacaccgcttatatggaactgtcttctctgcggagcgaagataccgccgtgtactactgtgccagaggccggcctgccatggactactggggccagggcaccctggtgacagtgtcctca

SEQ ID NO: 145 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 146 - DNA, 333 bp, synthetic construct
  source 1..333: mol_type = other DNA
gatatccagatgacccagagccccagcagcctgtccgccagtgtcggagatagggtgaccatcacctgcagagcctctgaaagcgttgacaattacggcatcagcttcatgaactggttccagcagaaacctggcaaagcccctaagcttcttatttacgccgcttccaaccagggctctggcgtgcccagccggttcagcggctcaggatctggaaccgactttacgctgacaatcagctctctgcagcctgatgacttcgccacatactactgccagcaaagcaaagaggtgccttggaccttcggccagggcaccaaggtggagattaag

SEQ ID NO: 147 - protein, 10 aa, synthetic construct
EQKLISEEDL

SEQ ID NO: 148 - DNA, 30 bp, synthetic construct
  source 1..30: mol_type = other DNA
gagcagaagctgatctctgaggaagacctg

SEQ ID NO: 149 - protein, 8 aa, synthetic construct
GGPLGMTS

SEQ ID NO: 150 - DNA, 24 bp, synthetic construct
  source 1..24: mol_type = other DNA
ggaggccctctgggcatgacctct

SEQ ID NO: 151 - protein, 77 aa, synthetic construct
PKNNHTASILDRMQADFKCCGAANYTDWEKIPSMSKNRVPDSCCINVTVGCGINFNEKAIHKEGCVEKIGGWLRKNV

SEQ ID NO: 152 - DNA, 231 bp, synthetic construct
  source 1..231: mol_type = other DNA
cctaagaacaaccacaccgccagcatcctggaccggatgcaggccgactttaagtgctgcggcgccgctaattacaccgactgggagaaaatccccagcatgagcaagaatagagtgcccgatagctgttgcatcaacgtgacagtgggctgcggcatcaatttcaacgagaaggctatccacaaggagggctgcgtggaaaagatcggcggctggctgagaaagaacgtg

SEQ ID NO: 153 - protein, 40 aa, synthetic construct
VLRDKLVKCEGISLLAQNTSWLLLLLLSLSLLQATDFMSL

SEQ ID NO: 154 - DNA, 120 bp, synthetic construct
  source 1..120: mol_type = other DNA
gtgctgagagacaagctcgtgaaatgcgagggcatcagcctgctggcccagaacacatcctggctgctgctgctgctgctcagcctgagtctgctgcaagccaccgatttcatgtctctg

SEQ ID NO: 155 - protein, 701 aa, synthetic construct
MCGACKENYCLMITFAIFLSLIMLVEVAAAIAGYVGGSGPVRRYQGGPLGVRGGMEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGSGEQKLISEEDLGGPLGMTSGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 156 - DNA, 2103 bp, synthetic construct
  source 1..2103: mol_type = other DNA
atgtgcggcgcctgtaaagaaaactactgcctgatgatcacattcgcaatcttcctgtccctcatcatgctggtggaggtggccgccgccatcgctggctacgtgggcggcagcggccctgtgaggagataccagggcggtccactgggcgtgcggggcggcatggaagtgcagctggtggagagcggcggcggcgtggtgagacctggcgggtccctgcggctgagctgtgccgccagcggattcacattcgacgattacggcatgagctgggtcagacaggccccaggaaaaggcctggaatgggtgtctggcattaactggaacggcggcagcaccggctacgccgatagcgttaagggcagattcaccatcagcagagataacgccaagaactctctgtacctgcagatgaatagcctgcgggccgaagataccgccgtctactactgtgcccggggaagaagcttgctgtttgactactggggacagggcacactggtcaccgtgtctaggggcggcggcggcagcggaggaggcgggtcgggcggcggcggatccagcgagctgacacaagatcctgctgtgagcgtcgccctgggccagaccgtgcggatcacttgccagggagatagcctgaggtcctactacgccagttggtatcaacagaagcctggacaggcccctgtactggtgatctacggcaagaacaacagaccttctggcatccctgatagattcagcggctccagcagcggaaacaccgctagcctgacaatcaccggagctcaggctgaggacgaggccgactactactgcaacagccgggactcttcaggaaatcacgtggtgttcggcggcggcaccaagctgacagtgctgcctagcggccaggccggcgccgcggccagcgaaagcctgttcgtgtctaaccacgcctacgaggcttccggcggccccgagcaggtgcagctggtgcaaagcggcgccgaggtgaagaagcccggcagcagcgtgaaagtgtcctgcaaggcctccggctacacctttaccgactacaatatgcactgggttcggcaggcccctggccaaggtctcgagtggatcggctacatctatccttacaacggcggcaccggctacaaccagaagttcaaaagcaaggcaacaatcacagccgacgagagcactaacaccgcctacatggaactgagcagcctgagaagcgaggacaccgctgtttactactgcgccagaggcagaccagctatggactactggggccaaggaaccctggtgaccgtgtcatctgggggcggcggatctggcggcggcgggagcggcggcggaggaagtgatatccagatgacccagagccctagcagcctgtctgcctctgttggcgaccgggtgaccatcacctgcagagcttctgaatcagttgacaactacggcatcagcttcatgaactggttccagcagaaacctggcaaggcccccaagctgctgatctatgccgcttctaatcagggcagcggcgtgcctagcagatttagcggcagtggatcgggaaccgacttcaccctgaccatttcttcgcttcagcccgacgatttcgccacatactattgtcagcagagcaaggaggtgccatggacatttggccagggcactaaggtggaaatcaagggcggcagcggagagcagaagctgatctccgaggaagacctgggtggacccctgggcatgacctctggcggcagcggccctaagaacaaccacacagccagcatcctggacagaatgcaggccgacttcaagtgctgcggcgccgctaattacaccgactgggagaaaatcccctccatgagcaaaaaccgggtgcccgacagctgttgcatcaatgtgaccgtgggctgtggcattaacttcaacgagaaggctatccataaggagggctgcgtcgagaagatcggcggctggctgagaaagaacgtgctggtggtggccgccgccgccctgggtatcgccttcgtggaagtgctgggcatcgtgttcgcctgctgcctggtgaagtctatcagatctggctatgaggtgatg

SEQ ID NO: 157 - protein, 10 aa, synthetic construct
MCGACKENYC

SEQ ID NO: 158 - DNA, 30 bp, synthetic construct
  source 1..30: mol_type = other DNA
atgtgcggcgcctgtaaagaaaactactgc

SEQ ID NO: 159 - protein, 21 aa, synthetic construct
LMITFAIFLSLIMLVEVAAAI

SEQ ID NO: 160 - DNA, 63 bp, synthetic construct
  source 1..63: mol_type = other DNA
ctgatgatcacattcgcaatcttcctgtccctcatcatgctggtggaggtggccgccgccatc

SEQ ID NO: 161 - protein, 10 aa, synthetic construct
GGSGPVRRYQ

SEQ ID NO: 162 - DNA, 30 bp, synthetic construct
  source 1..30: mol_type = other DNA
ggcggcagcggccctgtgaggagataccag

SEQ ID NO: 163 - protein, 9 aa, synthetic construct
GGPLGVRGG

SEQ ID NO: 164 - DNA, 27 bp, synthetic construct
  source 1..27: mol_type = other DNA
ggcggtccactgggcgtgcggggcggc

SEQ ID NO: 165 - protein, 118 aa, synthetic construct; identical to SEQ ID NO: 133

SEQ ID NO: 166 - DNA, 354 bp, synthetic construct
  source 1..354: mol_type = other DNA
atggaagtgcagctggtggagagcggcggcggcgtggtgagacctggcgggtccctgcggctgagctgtgccgccagcggattcacattcgacgattacggcatgagctgggtcagacaggccccaggaaaaggcctggaatgggtgtctggcattaactggaacggcggcagcaccggctacgccgatagcgttaagggcagattcaccatcagcagagataacgccaagaactctctgtacctgcagatgaatagcctgcgggccgaagataccgccgtctactactgtgcccggggaagaagcttgctgtttgactactggggacagggcacactggtcaccgtgtctagg

SEQ ID NO: 167 - protein, 15 aa, synthetic construct
GGGGSGGGGSGGGGS

SEQ ID NO: 168 - DNA, 45 bp, synthetic construct
  source 1..45: mol_type = other DNA
ggcggcggcggcagcggaggaggcgggtcgggcggcggcggatcc

SEQ ID NO: 169 - protein, 107 aa, synthetic construct; identical to SEQ ID NO: 137

SEQ ID NO: 170 - DNA, 321 bp, synthetic construct
  source 1..321: mol_type = other DNA
agcgagctgacacaagatcctgctgtgagcgtcgccctgggccagaccgtgcggatcacttgccagggagatagcctgaggtcctactacgccagttggtatcaacagaagcctggacaggcccctgtactggtgatctacggcaagaacaacagaccttctggcatccctgatagattcagcggctccagcagcggaaacaccgctagcctgacaatcaccggagctcaggctgaggacgaggccgactactactgcaacagccgggactcttcaggaaatcacgtggtgttcggcggcggcaccaagctgacagtgctg

SEQ ID NO: 171 - protein, 20 aa, synthetic construct
PSGQAGAAASESLFVSNHAY

SEQ ID NO: 172 - DNA, 60 bp, synthetic construct
  source 1..60: mol_type = other DNA
cctagcggccaggccggcgccgcggccagcgaaagcctgttcgtgtctaaccacgcctac

SEQ ID NO: 173 - protein, 7 aa, synthetic construct
EASGGPE

SEQ ID NO: 174 - DNA, 21 bp, synthetic construct
  source 1..21: mol_type = other DNA
gaggcttccggcggccccgag

SEQ ID NO: 175 - protein, 116 aa, synthetic construct; identical to SEQ ID NO: 143

SEQ ID NO: 176 - DNA, 348 bp, synthetic construct
  source 1..348: mol_type = other DNA
caggtgcagctggtgcaaagcggcgccgaggtgaagaagcccggcagcagcgtgaaagtgtcctgcaaggcctccggctacacctttaccgactacaatatgcactgggttcggcaggcccctggccaaggtctcgagtggatcggctacatctatccttacaacggcggcaccggctacaaccagaagttcaaaagcaaggcaacaatcacagccgacgagagcactaacaccgcctacatggaactgagcagcctgagaagcgaggacaccgctgtttactactgcgccagaggcagaccagctatggactactggggccaaggaaccctggtgaccgtgtcatct

SEQ ID NO: 177 - protein, 111 aa, synthetic construct; identical to SEQ ID NO: 145

SEQ ID NO: 178 - DNA, 333 bp, synthetic construct
  source 1..333: mol_type = other DNA
gatatccagatgacccagagccctagcagcctgtctgcctctgttggcgaccgggtgaccatcacctgcagagcttctgaatcagttgacaactacggcatcagcttcatgaactggttccagcagaaacctggcaaggcccccaagctgctgatctatgccgcttctaatcagggcagcggcgtgcctagcagatttagcggcagtggatcgggaaccgacttcaccctgaccatttcttcgcttcagcccgacgatttcgccacatactattgtcagcagagcaaggaggtgccatggacatttggccagggcactaaggtggaaatcaag

SEQ ID NO: 179 - protein, 16 aa, synthetic construct
GGSGEQKLISEEDLGG

SEQ ID NO: 180 - DNA, 48 bp, synthetic construct
  source 1..48: mol_type = other DNA
ggcggcagcggagagcagaagctgatctccgaggaagacctgggtgga

SEQ ID NO: 181 - protein, 6 aa, synthetic construct
PLGMTS

SEQ ID NO: 182 - DNA, 18 bp, synthetic construct
  source 1..18: mol_type = other DNA
cccctgggcatgacctct

SEQ ID NO: 183 - protein, 77 aa, synthetic construct; identical to SEQ ID NO: 151

SEQ ID NO: 184 - DNA, 231 bp, synthetic construct
  source 1..231: mol_type = other DNA
cctaagaacaaccacacagccagcatcctggacagaatgcaggccgacttcaagtgctgcggcgccgctaattacaccgactgggagaaaatcccctccatgagcaaaaaccgggtgcccgacagctgttgcatcaatgtgaccgtgggctgtggcattaacttcaacgagaaggctatccataaggagggctgcgtcgagaagatcggcggctggctgagaaagaacgtg

SEQ ID NO: 185 - protein, 21 aa, synthetic construct
LVVAAAALGIAFVEVLGIVFA

SEQ ID NO: 186 - DNA, 63 bp, synthetic construct
  source 1..63: mol_type = other DNA
ctggtggtggccgccgccgccctgggtatcgccttcgtggaagtgctgggcatcgtgttcgcc

SEQ ID NO: 187 - protein, 14 aa, synthetic construct
CCLVKSIRSGYEVM

SEQ ID NO: 188 - protein, 693 aa, synthetic construct
MCGACKENYCLMITFAIFLSLIMLVEVAAAIAGYVGGSGPVRRYQGGPLGVRGGMEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGSGEQKLISEEDLGGPLGMTSGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKS

SEQ ID NO: 189 - DNA, 2079 bp, synthetic construct
  source 1..2079: mol_type = other DNA
atgtgcggcgcctgtaaagaaaactactgcctgatgatcacattcgcaatcttcctgtccctcatcatgctggtggaggtggccgccgccatcgctggctacgtgggcggcagcggccctgtgaggagataccagggcggtccactgggcgtgcggggcggcatggaagtgcagctggtggagagcggcggcggcgtggtgagacctggcgggtccctgcggctgagctgtgccgccagcggattcacattcgacgattacggcatgagctgggtcagacaggccccaggaaaaggcctggaatgggtgtctggcattaactggaacggcggcagcaccggctacgccgatagcgttaagggcagattcaccatcagcagagataacgccaagaactctctgtacctgcagatgaatagcctgcgggccgaagataccgccgtctactactgtgcccggggaagaagcttgctgtttgactactggggacagggcacactggtcaccgtgtctaggggcggcggcggcagcggaggaggcgggtcgggcggcggcggatccagcgagctgacacaagatcctgctgtgagcgtcgccctgggccagaccgtgcggatcacttgccagggagatagcctgaggtcctactacgccagttggtatcaacagaagcctggacaggcccctgtactggtgatctacggcaagaacaacagaccttctggcatccctgatagattcagcggctccagcagcggaaacaccgctagcctgacaatcaccggagctcaggctgaggacgaggccgactactactgcaacagccgggactcttcaggaaatcacgtggtgttcggcggcggcaccaagctgacagtgctgcctagcggccaggccggcgccgcggccagcgaaagcctgttcgtgtctaaccacgcctacgaggcttccggcggccccgagcaggtgcagctggtgcaaagcggcgccgaggtgaagaagcccggcagcagcgtgaaagtgtcctgcaaggcctccggctacacctttaccgactacaatatgcactgggttcggcaggcccctggccaaggtctcgagtggatcggctacatctatccttacaacggcggcaccggctacaaccagaagttcaaaagcaaggcaacaatcacagccgacgagagcactaacaccgcctacatggaactgagcagcctgagaagcgaggacaccgctgtttactactgcgccagaggcagaccagctatggactactggggccaaggaaccctggtgaccgtgtcatctgggggcggcggatctggcggcggcgggagcggcggcggaggaagtgatatccagatgacccagagccctagcagcctgtctgcctctgttggcgaccgggtgaccatcacctgcagagcttctgaatcagttgacaactacggcatcagcttcatgaactggttccagcagaaacctggcaaggcccccaagctgctgatctatgccgcttctaatcagggcagcggcgtgcctagcagatttagcggcagtggatcgggaaccgacttcaccctgaccatttcttcgcttcagcccgacgatttcgccacatactattgtcagcagagcaaggaggtgccatggacatttggccagggcactaaggtggaaatcaagggcggcagcggagagcagaagctgatctccgaggaagacctgggtggacccctgggcatgacctctggcggcagcggccctaagaacaaccacacagccagcatcctggacagaatgcaggccgacttcaagtgctgcggcgccgctaattacaccgactgggagaaaatcccctccatgagcaaaaaccgggtgcccgacagctgttgcatcaatgtgaccgtgggctgtggcattaacttcaacgagaaggctatccataaggagggctgcgtcgagaagatcggcggctggctgagaaagaacgtgctggtggtggccgccgccgccctgggtatcgccttcgtggaagtgctgggcatcgtgttcgcctgctgcctggtgaagtct

SEQ ID NO: 190 - protein, 10 aa, synthetic construct
MCGACKENYC

SEQ ID NO: 191 - DNA, 30 bp, synthetic construct; identical to SEQ ID NO: 158
  source 1..30: mol_type = other DNA

SEQ ID NO: 192 - protein, 21 aa, synthetic construct
LMITFAIFLSLIMLVEVAAAI

SEQ ID NO: 193 - DNA, 63 bp, synthetic construct; identical to SEQ ID NO: 160
  source 1..63: mol_type = other DNA

SEQ ID NO: 194 - protein, 10 aa, synthetic construct
GGSGPVRRYQ

SEQ ID NO: 195 - DNA, 28 bp, synthetic construct
  source 1..28: mol_type = other DNA
cggcagcggccctgtgaggagataccag

SEQ ID NO: 196 - protein, 9 aa, synthetic construct
GGPLGVRGG

SEQ ID NO: 197 - DNA, 27 bp, synthetic construct
  source 1..27: mol_type = other DNA
ggcggtccactgggcgtgcggggcggc

SEQ ID NO: 198 - protein, 118 aa, synthetic construct; identical to SEQ ID NO: 133

SEQ ID NO: 199 - DNA, 354 bp, synthetic construct; identical to SEQ ID NO: 166
  source 1..354: mol_type = other DNA

SEQ ID NO: 200 - protein, 15 aa, synthetic construct
GGGGSGGGGSGGGGS

SEQ ID NO: 201 - DNA, 45 bp, synthetic construct; identical to SEQ ID NO: 168
  source 1..45: mol_type = other DNA

SEQ ID NO: 202 - protein, 107 aa, synthetic construct; identical to SEQ ID NO: 137

SEQ ID NO: 203 - DNA, 321 bp, synthetic construct; identical to SEQ ID NO: 170
  source 1..321: mol_type = other DNA

SEQ ID NO: 204 - protein, 20 aa, synthetic construct
PSGQAGAAASESLFVSNHAY

SEQ ID NO: 205 - DNA, 60 bp, synthetic construct; identical to SEQ ID NO: 172
  source 1..60: mol_type = other DNA

SEQ ID NO: 206 - protein, 7 aa, synthetic construct
EASGGPE

SEQ ID NO: 207 - DNA, 21 bp, synthetic construct
  source 1..21: mol_type = other DNA
gaggcttccggcggccccgag

SEQ ID NO: 208 - protein, 116 aa, synthetic construct; identical to SEQ ID NO: 143

SEQ ID NO: 209 - DNA, 348 bp, synthetic construct; identical to SEQ ID NO: 176
  source 1..348: mol_type = other DNA

SEQ ID NO: 210 - protein, 111 aa, synthetic construct; identical to SEQ ID NO: 145

SEQ ID NO: 211 - DNA, 333 bp, synthetic construct; identical to SEQ ID NO: 178
  source 1..333: mol_type = other DNA

SEQ ID NO: 212 - protein, 947 aa, synthetic construct; identical to SEQ ID NO: 54

SEQ ID NO: 213 - DNA, 2761 bp, synthetic construct
  source 1..2761: mol_type = other DNA
atgagctgcagcgtggcgagagtagggctgggcctgctcctgctgctgatgggcgccggcctggccgtgcaaggatggttcctgctgcagcttcattggagactgggagagatggtgactagactgcctgacggccctgccggcagctgggagcagctgatccaggagagaaggagccacgaggtgaaccctgctgcaggtgggagcggccctgtgcggcggtaccaaatggaagtgcagctggtcgagtctgggggcggcgtggtccggccaggcggcagcctgcgtctgtcttgcgccgctagcggcttcactttcgatgactacggcatgagctgggtgcggcaggcccccggcaagggcctcgagtgggtttctggcatcaactggaacggcggctctaccggctacgccgacagcgtgaagggccgctttaccatctccagagataatgccaagaacagcctgtacctgcagatgaacagcctgcgggccgaggatacggccgtgtactattgtgcccggggcagaagcctcctcttcgactactggggccagggcaccctggttaccgtgagccggggcggcggcggttctggaggcggaggcagcggcggcgggggctcctctgagctgacccaggaccccgccgtgagcgtggccctgggccagacagtcagaattacatgccagggagatagcctgagatcctactatgccagctggtaccagcagaaacctggccaagctcctgtgctggtcatctacggcaaaaacaacagacctagtggaatccccgacagatttagcggatcctccagcggcaataccgcctccttgacaatcaccggcgctcaggccgaggatgaagccgactactactgcaacagcagagacagctctgggaaccacgtggtgttcggggggggaaccaagcttacagtgctgccttctgggcaggcgggagccgccgctagcgaatctctgtttgtgtctaatcacgcctacgaggccagcggcggccccgagcaggtgcagctggtgcagagtggggctgaagtgaagaaacctggcagctcggtgaaggtgagctgcaaggcctctggctacaccttcaccgactacaacatgcactgggtcagacaggcccctggccagggactggaatggatcggctacatctacccctacaacggcggcactggctataaccaaaaattcaagtctaaggcaaccatcacagccgacgagagcaccaacaccgcctatatggagctgtctagcctgaggagcgaggacaccgccgtgtactactgcgcccggggaagacctgccatggactactggggccaaggaacactggtgaccgtcagctctggaggcggcggatctggcggaggaggcagcggcggcggcggttcagacatccagatgacccagtcacctagctctctgagcgccagcgtgggcgaccgggtgaccatcacctgtagagccagcgagagcgtggacaactacggaatctcattcatgaactggttccagcagaagcccggaaaggccccgaagctgctgatctatgctgccagcaaccagggttcaggggtgcctagcagattttctgggagcggctctggcaccgactttaccctgacaatctctagcctgcagccagacgatttcgccacttactactgtcagcagagcaaggaagtgccatggaccttcggacaaggaacaaaggttgaaattaagggaggcagcggggaacaaaagctgatcagcgaagaggacctgagcggcggaagcggcagcctggagtgcaccaagctgtgtctgcctcagatcgagaatgtgaagggcacagaggattccggcggcagcggccaagtgcagctggtgcagagcggcgctgaggtgaagaagcccggcagcagcgtgaaggtgagctgtaaagccttcggcggaaccttcagctcctacgctatcagctgggtgagacaggcccctggccaggggctagaatggatgggcagaatcatccggttcctgggcatcgccaattacgcccagaagtttcagggccgggtgacgctgatcgccgataagagcaccaacaccgcctacatggaactgtcttccctgcgatcagaagacaccgccgtgtactactgcgccggcgaacctggaagagaagatcctgacgccgtggatatctggggacagggcacaatggtgacagtgtcgagcggcggaggcggctctggtggcggtggctctggcggcggcggctccgacattcagatgacacagtccccttcctctctgtccgcctccgtgggcgaccgggtgaccatcacctgcagagccagtcagggcatcagaagctggctggcttggtaccagcagaagcctgagaaggccaggaaaagcctgatctacgccgcgagcagcctgcagtctggggtgccctctagattcagcggttctggcagcggcacagatttcacactgacaataagttcgctgcagcctgaagacttcgccacctattactgccagcaatacaacagctaccctctgaccttcggcggcggaaccaaggttgagatcaagggcggcggcggtagcggcggcggcggctccggcggcggaggcagctccggcaccaccgtcctgctgcctctggtgatcttcttcggcctgtgcctgctgtcactgctgttcatcggactcatgtacagatacggcggaagcggcc

SEQ ID NO: 214 - protein, 7 aa, synthetic construct
MSCSVAR

SEQ ID NO: 215 - DNA, 21 bp, synthetic construct
  source 1..21: mol_type = other DNA
atgagctgcagcgtggcgaga

SEQ ID NO: 216 - protein, 21 aa, synthetic construct
VGLGLLLLLMGAGLAVQGWFL

SEQ ID NO: 217 - DNA, 72 bp, synthetic construct
  source 1..72: mol_type = other DNA
gtggcgagagtagggctgggcctgctcctgctgctgatgggcgccggcctggccgtgcaaggatggttcctg

SEQ ID NO: 218 - protein, 44 aa, synthetic construct
GSWEQLLQLHWRLGEMVTRLPDGPAGSWEQLIQERRSHEVNPAA

SEQ ID NO: 219 - DNA, 114 bp, synthetic construct
  source 1..114: mol_type = other DNA
ctgcagcttcattggagactgggagagatggtgactagactgcctgacggccctgccggcagctgggagcagctgatccaggagagaaggagccacgaggtgaaccctgctgca

SEQ ID NO: 220 - protein, 10 aa, synthetic construct
GGSGPVRRYQ

SEQ ID NO: 221 - DNA, 30 bp, synthetic construct
  source 1..30: mol_type = other DNA
ggtgggagcggccctgtgcggcggtaccaa

SEQ ID NO: 222 - protein, 118 aa, synthetic construct; identical to SEQ ID NO: 133

SEQ ID NO: 223 - DNA, 354 bp, synthetic construct
  source 1..354: mol_type = other DNA
atggaagtgcagctggtcgagtctgggggcggcgtggtccggccaggcggcagcctgcgtctgtcttgcgccgctagcggcttcactttcgatgactacggcatgagctgggtgcggcaggcccccggcaagggcctcgagtgggtttctggcatcaactggaacggcggctctaccggctacgccgacagcgtgaagggccgctttaccatctccagagataatgccaagaacagcctgtacctgcagatgaacagcctgcgggccgaggatacggccgtgtactattgtgcccggggcagaagcctcctcttcgactactggggccagggcaccctggttaccgtgagccgg

SEQ ID NO: 224 - protein, 15 aa, synthetic construct
GGGGSGGGGSGGGGS

SEQ ID NO: 225 - DNA, 45 bp, synthetic construct
  source 1..45: mol_type = other DNA
ggcggcggcggttctggaggcggaggcagcggcggcgggggctcc

SEQ ID NO: 226 - protein, 107 aa, synthetic construct; identical to SEQ ID NO: 137

SEQ ID NO: 227 - DNA, 321 bp, synthetic construct
  source 1..321: mol_type = other DNA
tctgagctgacccaggaccccgccgtgagcgtggccctgggccagacagtcagaattacatgccagggagatagcctgagatcctactatgccagctggtaccagcagaaacctggccaagctcctgtgctggtcatctacggcaaaaacaacagacctagtggaatccccgacagatttagcggatcctccagcggcaataccgcctccttgacaatcaccggcgctcaggccgaggatgaagccgactactactgcaacagcagagacagctctgggaaccacgtggtgttcggggggggaaccaagcttacagtgctg

SEQ ID NO: 228 - protein, 20 aa, synthetic construct
PSGQAGAAASESLFVSNHAY

SEQ ID NO: 229 - DNA, 60 bp, synthetic construct
  source 1..60: mol_type = other DNA
ccttctgggcaggcgggagccgccgctagcgaatctctgtttgtgtctaatcacgcctac

SEQ ID NO: 230 - protein, 7 aa, synthetic construct
EASGGPE

SEQ ID NO: 231 - DNA, 27 bp, synthetic construct
  source 1..27: mol_type = other DNA
gaggccagcggcggccccgagcaggtg

SEQ ID NO: 232 - protein, 116 aa, synthetic construct; identical to SEQ ID NO: 143

SEQ ID NO: 233 - DNA, 339 bp, synthetic construct
  source 1..339: mol_type = other DNA
cagctggtgcagagtggggctgaagtgaagaaacctggcagctcggtgaaggtgagctgcaaggcctctggctacaccttcaccgactacaacatgcactgggtcagacaggcccctggccagggactggaatggatcggctacatctacccctacaacggcggcactggctataaccaaaaattcaagtctaaggcaaccatcacagccgacgagagcaccaacaccgcctatatggagctgtctagcctgaggagcgaggacaccgccgtgtactactgcgcccggggaagacctgccatggactactggggccaaggaacactggtgaccgtcagc

SEQ ID NO: 234 - protein, 15 aa, synthetic construct
GGGGSGGGGSGGGGS

SEQ ID NO: 235 - DNA, 48 bp, synthetic construct
  source 1..48: mol_type = other DNA
tctggaggcggcggatctggcggaggaggcagcggcggcggcggttca

SEQ ID NO: 236 - protein, 111 aa, synthetic construct; identical to SEQ ID NO: 145

SEQ ID NO: 237 - DNA, 333 bp, synthetic construct
  source 1..333: mol_type = other DNA
gacatccagatgacccagtcacctagctctctgagcgccagcgtgggcgaccgggtgaccatcacctgtagagccagcgagagcgtggacaactacggaatctcattcatgaactggttccagcagaagcccggaaaggccccgaagctgctgatctatgctgccagcaaccagggttcaggggtgcctagcagattttctgggagcggctctggcaccgactttaccctgacaatctctagcctgcagccagacgatttcgccacttactactgtcagcagagcaaggaagtgccatggaccttcggacaaggaacaaaggttgaaattaag

SEQ ID NO: 238 - protein, 19 aa, synthetic construct
GGSGEQKLISEEDLSGGSG

SEQ ID NO: 239 - DNA, 42 bp, synthetic construct
  source 1..42: mol_type = other DNA
ggaggcagcggggaacaaaagctgatcagcgaagaggacctg

SEQ ID NO: 240 - protein, 21 aa, synthetic construct
SLECTKLCLPQIENVKGTEDS

SEQ ID NO: 241 - DNA, 60 bp, synthetic construct
  source 1..60: mol_type = other DNA
agcctggagtgcaccaagctgtgtctgcctcagatcgagaatgtgaagggcacagaggat

SEQ ID NO: 242 - protein, 121 aa, synthetic construct
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGREDPDAVDIWGQGTMVTVSS

SEQ ID NO: 243 - DNA, 363 bp, synthetic construct
  source 1..363: mol_type = other DNA
caagtgcagctggtgcagagcggcgctgaggtgaagaagcccggcagcagcgtgaaggtgagctgtaaagccttcggcggaaccttcagctcctacgctatcagctgggtgagacaggcccctggccaggggctagaatggatgggcagaatcatccggttcctgggcatcgccaattacgcccagaagtttcagggccgggtgacgctgatcgccgataagagcaccaacaccgcctacatggaactgtcttccctgcgatcagaagacaccgccgtgtactactgcgccggcgaacctggaagagaagatcctgacgccgtggatatctggggacagggcacaatggtgacagtgtcgagc

SEQ ID NO: 244 - protein, 15 aa, synthetic construct
GGGGSGGGGSGGGGS

SEQ ID NO: 245 - DNA, 45 bp, synthetic construct
  source 1..45: mol_type = other DNA
ggcggaggcggctctggtggcggtggctctggcggcggcggctcc

SEQ ID NO: 246 - protein, 107 aa, synthetic construct
DIQMTQSPSSLSASVGDRVTITCRASQGIRSWLAWYQQKPEKARKSLIYAASSLQSGVPSRFSGSGSGTDFTLTISSLQPEDFATYYCQQYNSYPLTFGGGTKVEIK

SEQ ID NO: 247 - DNA, 321 bp, synthetic construct
  source 1..321: mol_type = other DNA
gacattcagatgacacagtccccttcctctctgtccgcctccgtgggcgaccgggtgaccatcacctgcagagccagtcagggcatcagaagctggctggcttggtaccagcagaagcctgagaaggccaggaaaagcctgatctacgccgcgagcagcctgcagtctggggtgccctctagattcagcggttctggcagcggcacagatttcacactgacaataagttcgctgcagcctgaagacttcgccacctattactgccagcaatacaacagctaccctctgaccttcggcggcggaaccaaggttgagatcaag

SEQ ID NO: 248 - protein, 29 aa, synthetic construct
SGTTVLLPLVIFFGLCLLSLLFIGLMYRY

SEQ ID NO: 249 - DNA, 87 bp, synthetic construct
  source 1..87: mol_type = other DNA
tccggcaccaccgtcctgctgcctctggtgatcttcttcggcctgtgcctgctgtcactgctgttcatcggactcatgtacagatac

SEQ ID NO: 250 - protein, 27 aa, synthetic construct
KRGRKKLLYIFKQPFMRPVQTTQEEDG

SEQ ID NO: 251 - DNA, 81 bp, synthetic construct
  source 1..81: mol_type = other DNA
aaaagaggccgaaagaagctgctgtacatctttaaacagcccttcatgagacccgtgcaaacaacccaggaggaagatggc

SEQ ID NO: 252 - protein, 1097 aa, synthetic construct
MCGACKENYCLMITFAIFLSLIMLVEVAAAIAGYVGGSGPVRRYQIEPDGGSGMEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGSGEQKLISEEDLGGIEPDGGSGQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGREDPDAVDIWGQGTMVTVSSGGGGSGGGGSGGGGSDIQMTQSPSSLSASVGDRVTITCRASQGIRSWLAWYQQKPEKARKSLIYAASSLQSGVPSRFSGSGSGTDFTLTISSLQPEDFATYYCQQYNSYPLTFGGGTKVEIKAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 253 - DNA, 3291 bp, synthetic construct
  source 1..3291: mol_type = other DNA
atgtgcggcgcctgcaaggaaaactactgtctgatgatcactttcgccatctttctgtctctgatcatgctggtggaagtggccgctgccattgccggatacgtgggcggctctggccctgtgcgaagataccaaatcgagcctgacggcggcagcggcatggaagtgcagctggttgagtccggcggcggagtggtgcggcctggcggctctctgagactgtcttgcgctgccagcggtttcaccttcgacgattacggaatgagctgggtgcggcaggccccaggcaagggcctggagtgggtgtccggcatcaactggaacggcggaagcaccggctacgcagacagcgtgaagggcagattcaccatctccagagataacgccaagaattcactgtacctgcagatgaacagcctgagagctgaggacactgcagtttactactgcgccagaggccggagcctgctgttcgactactggggacagggaacactggtgaccgtgagcagaggcggcggcggctctggcggcggcggaagcggcggtggaggcagctccgagctgacccaggaccccgccgtgtccgtggccctgggccaaacagtgagaatcacctgtcagggcgactctctgcgctcttactacgccagctggtaccaacagaagcccggccaggcccctgtgctggtgatctacggcaagaacaacagaccttctggtatccccgacagatttagcggctcttctagcggcaacaccgcctccctgaccatcacaggcgcccaggccgaggacgaggctgactactattgcaatagtagagacagcagcggaaaccatgtggtgttcggcggtggcacaaagctgacagtgcttcccagcggacaggccggagccgccgcctctgaaagcctgttcgtgagtaatcacgcctacgaggcctccggaggccctgagcaggtgcagctggtgcagtccggcgccgaggtgaagaagccaggcagcagcgttaaggtgagttgtaaagcctccggatacaccttcaccgactacaacatgcactgggttagacaggcgcctggccagggcctggaatggatcggctacatctacccttacaacggcgggaccggctataatcagaagttcaagagcaaggccacgatcacagccgacgagagcaccaacacagcatacatggaactgagctccctgagatctgaggacaccgccgtgtactactgcgccaggggtcggccggccatggactattggggccagggcaccctggtcacagtgtctagcggcggcggcggctctggcggcggcggctcaggcggcggcggcagcgatattcagatgacacagagccctagcagcctgtctgcctctgttggagatcgggtgacaatcacatgtcgggcctccgagtctgtggacaactacggcatcagcttcatgaactggttccagcagaaaccaggcaaggcccctaagctgctgatctacgccgctagcaaccagggcagcggcgtgccttcccgcttcagcggcagcggcagcggaacagatttcaccctgaccatctcttctctccaacctgatgatttcgctacctactactgccagcagagcaaggaggtgccttggaccttcggacagggcaccaaagtggagatcaagggcggctctggggaacagaagctgatcagcgaggaagacctgggaggcatcgagcctgatggagggagcggacaagtgcagctggttcaaagcggcgctgaggtgaaaaaacctgggagcagcgtgaaggtgtcctgcaaggctttcgggggcaccttcagcagctacgcaatcagctgggtgcggcaggctcccggccagggcctggaatggatgggcagaatcatccggttcctcggaatcgccaactacgcccagaagttccagggtagagtgaccctgattgccgacaagagcacaaacaccgcttatatggaactgagctctctgcgcagcgaagataccgccgtgtactactgcgccggcgaacccggccgggaagatcctgatgccgtggatatctggggccagggcaccatggtgacagtgtcatccggtggcggcggaagcggcggcggcggtagtgggggaggcggcagcgacatccagatgacccagtcacctagctctttgtcggccagcgtcggagatagagtgaccattacctgtcgggcctctcagggaatcagaagctggctcgcctggtaccagcaaaagcctgagaaggccagaaaatccctaatctatgccgctagcagcctgcagtctggcgtgccctccagatttagcggaagcggaagcggcacagacttcacactgacaatcagctctctgcaacctgaggatttcgccacctactactgccaacagtacaacagctacccactgacatttggaggcggtacaaaggtggaaatcaaggccaagcccaccacaacccctgcccctagaccccccaccccagctcccaccatcgccagccagcctctgtccctgcggcctgaagcctgcagacccgctgccggcggcgccgtccacacccggggactggacttcgcccctagaaagatcgaggtgatgtacccccctccctacctggataatgagaagagcaatggcacaatcatccacgtgaaaggcaaacacctgtgcccttctcctctgtttcctggacctagcaagcccttctgggtcctggtcgtggtcggcggcgtgctggcttgctacagcctcctggtgaccgtggccttcatcattttttgggtgcgcagcaagagatccaggctgctgcactctgactacatgaacatgacccctagacggcctggtcctacaagaaagcactaccagccttacgcccctcccagagatttcgctgcctaccgtagccgggtgaagttcagcagatctgccgatgcccctgcttaccaacagggccagaaccagctgtacaacgagctgaacctgggccggcgggaagagtacgacgtgctggacaaaagaagaggcagagaccctgagatgggggggaagcctcggagaaagaacccccaggagggcctgtacaatgagctgcagaaggacaagatggccgaggcctattctgagatcggaatgaagggagaaagacggagaggcaaaggccacgacggcctgtaccagggtctgtcgaccgctaccaaggacacctacgacgccctgcacatgcaggctctgcctccaaga

SEQ ID NO: 254 - protein, 10 aa, synthetic construct
MCGACKENYC

SEQ ID NO: 255 - DNA, 30 bp, synthetic construct
  source 1..30: mol_type = other DNA
atgtgcggcgcctgcaaggaaaactactgt

SEQ ID NO: 256 - protein, 21 aa, synthetic construct
LMITFAIFLSLIMLVEVAAAI

SEQ ID NO: 257 - DNA, 57 bp, synthetic construct
  source 1..57: mol_type = other DNA
ctgatgatcactttcgccatctttctgtctctgatcatgctggtggaagtggccgct

SEQ ID NO: 258 - protein, 4 aa, synthetic construct
IEPD

SEQ ID NO: 259 - DNA, 12 bp, synthetic construct
  source 1..12: mol_type = other DNA
atcgagcctgac

SEQ ID NO: 260 - protein, 17 aa, synthetic construct
EEEEEVEADSEEEEEEE

SEQ ID NO: 261 - protein, 16 aa, synthetic construct
AQGVISADASNLDDFY

SEQ ID NO: 262 - protein, 11 aa, synthetic construct
LEADKGKLEYD

SEQ ID NO: 263 - protein, 4 aa, synthetic construct
GGSG

SEQ ID NO: 264 - DNA, 12 bp, synthetic construct
  source 1..12: mol_type = other DNA
ggcggcagcggc

SEQ ID NO: 265 - protein, 118 aa, synthetic construct; identical to SEQ ID NO: 133

SEQ ID NO: 266 - DNA, 354 bp, synthetic construct
  source 1..354: mol_type = other DNA
atggaagtgcagctggttgagtccggcggcggagtggtgcggcctggcggctctctgagactgtcttgcgctgccagcggtttcaccttcgacgattacggaatgagctgggtgcggcaggccccaggcaagggcctggagtgggtgtccggcatcaactggaacggcggaagcaccggctacgcagacagcgtgaagggcagattcaccatctccagagataacgccaagaattcactgtacctgcagatgaacagcctgagagctgaggacactgcagtttactactgcgccagaggccggagcctgctgttcgactactggggacagggaacactggtgaccgtgagcaga

SEQ ID NO: 267 - protein, 107 aa, synthetic construct; identical to SEQ ID NO: 137

SEQ ID NO: 268 - DNA, 321 bp, synthetic construct
  source 1..321: mol_type = other DNA
tccgagctgacccaggaccccgccgtgtccgtggccctgggccaaacagtgagaatcacctgtcagggcgactctctgcgctcttactacgccagctggtaccaacagaagcccggccaggcccctgtgctggtgatctacggcaagaacaacagaccttctggtatccccgacagatttagcggctcttctagcggcaacaccgcctccctgaccatcacaggcgcccaggccgaggacgaggctgactactattgcaatagtagagacagcagcggaaaccatgtggtgttcggcggtggcacaaagctgacagtgctt

SEQ ID NO: 269 - protein, 20 aa, synthetic construct
PSGQAGAAASESLFVSNHAY

SEQ ID NO: 270 - DNA, 60 bp, synthetic construct
  source 1..60: mol_type = other DNA
cccagcggacaggccggagccgccgcctctgaaagcctgttcgtgagtaatcacgcctac

SEQ ID NO: 271 - protein, 7 aa, synthetic construct
EASGGPE

SEQ ID NO: 272 - DNA, 21 bp, synthetic construct
  source 1..21: mol_type = other DNA
gaggcctccggaggccctgag

SEQ ID NO: 273 - protein, 116 aa, synthetic construct; identical to SEQ ID NO: 143

SEQ ID NO: 274 - DNA, 348 bp, synthetic construct
  source 1..348: mol_type = other DNA
caggtgcagctggtgcagtccggcgccgaggtgaagaagccaggcagcagcgttaaggtgagttgtaaagcctccggatacaccttcaccgactacaacatgcactgggttagacaggcgcctggccagggcctggaatggatcggctacatctacccttacaacggcgggaccggctataatcagaagttcaagagcaaggccacgatcacagccgacgagagcaccaacacagcatacatggaactgagctccctgagatctgaggacaccgccgtgtactactgcgccaggggtcggccggccatggactattggggccagggcaccctggtcacagtgtctagc

SEQ ID NO: 275 - protein, 111 aa, synthetic construct; identical to SEQ ID NO: 145

SEQ ID NO: 276 - DNA, 333 bp, synthetic construct
  source 1..333: mol_type = other DNA
gatattcagatgacacagagccctagcagcctgtctgcctctgttggagatcgggtgacaatcacatgtcgggcctccgagtctgtggacaactacggcatcagcttcatgaactggttccagcagaaaccaggcaaggcccctaagctgctgatctacgccgctagcaaccagggcagcggcgtgccttcccgcttcagcggcagcggcagcggaacagatttcaccctgaccatctcttctctccaacctgatgatttcgctacctactactgccagcagagcaaggaggtgccttggaccttcggacagggcaccaaagtggagatcaag

SEQ ID NO: 277 - protein, 16 aa, synthetic construct
GGSGEQKLISEEDLGG

SEQ ID NO: 278 - DNA, 48 bp, synthetic construct
  source 1..48: mol_type = other DNA
ggcggctctggggaacagaagctgatcagcgaggaagacctgggaggc

SEQ ID NO: 279 - protein, 4 aa, synthetic construct
IEPD

SEQ ID NO: 280 - DNA, 12 bp, synthetic construct
  source 1..12: mol_type = other DNA
atcgagcctgat

SEQ ID NO: 281 - protein, 121 aa, synthetic construct; identical to SEQ ID NO: 242

SEQ ID NO: 282 - DNA, 363 bp, synthetic construct
  source 1..363: mol_type = other DNA
caagtgcagctggttcaaagcggcgctgaggtgaaaaaacctgggagcagcgtgaaggtgtcctgcaaggctttcgggggcaccttcagcagctacgcaatcagctgggtgcggcaggctcccggccagggcctggaatggatgggcagaatcatccggttcctcggaatcgccaactacgcccagaagttccagggtagagtgaccctgattgccgacaagagcacaaacaccgcttatatggaactgagctctctgcgcagcgaagataccgccgtgtactactgcgccggcgaacccggccgggaagatcctgatgccgtggatatctggggccagggcaccatggtgacagtgtcatcc

SEQ ID NO: 283 - protein, 15 aa, synthetic construct
GGGGSGGGGSGGGGS

SEQ ID NO: 284 - DNA, 45 bp, synthetic construct
  source 1..45: mol_type = other DNA
ggtggcggcggaagcggcggcggcggtagtgggggaggcggcagc

SEQ ID NO: 285 - protein, 107 aa, synthetic construct; identical to SEQ ID NO: 246

SEQ ID NO: 286 - DNA, 321 bp, synthetic construct
  source 1..321: mol_type = other DNA
gacatccagatgacccagtcacctagctctttgtcggccagcgtcggagatagagtgaccattacctgtcgggcctctcagggaatcagaagctggctcgcctggtaccagcaaaagcctgagaaggccagaaaatccctaatctatgccgctagcagcctgcagtctggcgtgccctccagatttagcggaagcggaagcggcacagacttcacactgacaatcagctctctgcaacctgaggatttcgccacctactactgccaacagtacaacagctacccactgacatttggaggcggtacaaaggtggaaatcaag

SEQ ID NO: 287 - protein, 88 aa, synthetic construct
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 288 - DNA, 264 bp, synthetic construct
  source 1..264: mol_type = other DNA
gccaagcccaccacaacccctgcccctagaccccccaccccagctcccaccatcgccagccagcctctgtccctgcggcctgaagcctgcagacccgctgccggcggcgccgtccacacccggggactggacttcgcccctagaaagatcgaggtgatgtacccccctccctacctggataatgagaagagcaatggcacaatcatccacgtgaaaggcaaacacctgtgcccttctcctctgtttcctggacctagcaagccc

SEQ ID NO: 289 - protein, 27 aa, synthetic construct
FWVLVVVGGVLACYSLLVTVAFIIFWV

SEQ ID NO: 290 - DNA, 81 bp, synthetic construct
  source 1..81: mol_type = other DNA
ttctgggtcctggtcgtggtcggcggcgtgctggcttgctacagcctcctggtgaccgtggccttcatcattttttgggtg

SEQ ID NO: 291 - protein, 41 aa, synthetic construct
RSKRSRLLHSDYMNMTPRRPGPTRKHYQPYAPPRDFAAYRS

SEQ ID NO: 292 - DNA, 123 bp, synthetic construct
  source 1..123: mol_type = other DNA
cgcagcaagagatccaggctgctgcactctgactacatgaacatgacccctagacggcctggtcctacaagaaagcactaccagccttacgcccctcccagagatttcgctgcctaccgtagc

SEQ ID NO: 293 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 294 - DNA, 336 bp, synthetic construct
  source 1..336: mol_type = other DNA
cgggtgaagttcagcagatctgccgatgcccctgcttaccaacagggccagaaccagctgtacaacgagctgaacctgggccggcgggaagagtacgacgtgctggacaaaagaagaggcagagaccctgagatgggggggaagcctcggagaaagaacccccaggagggcctgtacaatgagctgcagaaggacaagatggccgaggcctattctgagatcggaatgaagggagaaagacggagaggcaaaggccacgacggcctgtaccagggtctgtcgaccgctaccaaggacacctacgacgccctgcacatgcaggctctgcctccaaga

SEQ ID NO: 295 - protein, 417 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVGGSGPVRRYQGGPLGMTSSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGMTSGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 296 - DNA, 1274 bp, synthetic construct
  source 1..1274: mol_type = other DNA
gatccgccgccaccatggccgtggagggcgggatgaagtgcgtgaaattcctgctgtacgtcctgctgctggccttttgcgcatgtgctgtgggactgatcgcagtgggagtcggagctcagctggtgctgagtcagactatcattcagggcgcaacccctgggtcactgctgccagtggtcatcattgccgtgggcgtcttcctgtttctggtggcattcgtcgggtgctgtggagcctgcaaggaaaactattgtctgatgatcaccttcgctatttttctgtctctgatcatgctggtggaggtcgccgctgcaattgccggctacgtgggaggcagtggaccagtccggagatatcaggggggacccctgggaatgacaagctcctctagtcggactccctctgacaagcctgtggcccacgtggtcgctaatcctcaggcagaaggccagctgcagtggctgaacaggcgagcaaatgcactgctggccaacggagtggaactgagagataatcagctggtggtcccatccgagggactgtacctgatctattctcaggtgctgttcaaaggacagggatgcccaagcacacacgtgctgctgacccatacaatctctagaattgctgtgagttaccagactaaggtcaacctgctgagcgccatcaaatccccctgtcagagggagacccctgaaggcgctgaggcaaagccatggtacgagcccatctatctgggcggggtgtttcagctggaaaaaggggaccgactgagcgcagagattaatcggcccgactacctggatttcgccgaatccggacaggtgtattttggcatcattgctctgggaggctcaggcgagcagaagctgatcagcgaggaagatctgggaggaccactgggaatgacctccggagggtctgggcctaagaacaatcacacagccagcattctggaccgcatgcaggcagatttcaaatgctgtggcgccgctaactacacagactgggaaaagatcccttcaatgagcaaaaaccgagtgccagattcctgctgtattaatgtgactgtcggatgcggcatcaactttaatgagaaggccattcataaagaaggctgcgtggagaagatcggaggctggctgaggaaaaatgtgctggtggtcgcagccgctgcactgggaatcgccttcgtggaagtcctgggcatcgtgttcgcttgctgtctggtcaaaagtattcgctcaggctatgaagtgatgtaatgtaca

SEQ ID NO: 297 - protein, 11 aa, synthetic construct
MAVEGGMKCVK

SEQ ID NO: 298 - protein, 21 aa, synthetic construct
FLLYVLLLAFCACAVGLIAVG

SEQ ID NO: 299 - protein, 19 aa, synthetic construct
VGAQLVLSQTIIQGATPGS

SEQ ID NO: 300 - protein, 21 aa, synthetic construct
LLPVVIIAVGVFLFLVAFVGC

SEQ ID NO: 301 - protein, 9 aa, synthetic construct
CGACKENYC

SEQ ID NO: 302 - protein, 21 aa, synthetic construct
LMITFAIFLSLIMLVEVAAAI

SEQ ID NO: 303 - protein, 10 aa, synthetic construct
GGSGPVRRYQ

SEQ ID NO: 304 - protein, 8 aa, synthetic construct
GGPLGMTS

SEQ ID NO: 305 - protein, 154 aa, synthetic construct
SSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIA

SEQ ID NO: 306 - protein, 14 aa, synthetic construct
GGSGEQKLISEEDL

SEQ ID NO: 307 - protein, 8 aa, synthetic construct
GGPLGMTS

SEQ ID NO: 308 - protein, 112 aa, synthetic construct
PKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 309 - protein, 21 aa, synthetic construct
LVVAAAALGIAFVEVLGIVFA

SEQ ID NO: 310 - protein, 12 aa, synthetic construct
LVKSIRSGYEVM

SEQ ID NO: 311 - protein, 409 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVGGSGPVRRYQGGPLGMTSSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGMTSGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKS

SEQ ID NO: 312 - protein, 6 aa, synthetic construct
HHHHHH

SEQ ID NO: 313 - protein, 106 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYV

SEQ ID NO: 314 - protein, 21 aa, synthetic construct
FLLYVLLLAFCACAVGLIAVG

SEQ ID NO: 315 - protein, 19 aa, synthetic construct
VGAQLVLSQTIIQGATPGS

SEQ ID NO: 316 - protein, 21 aa, synthetic construct
LLPVVIIAVGVFLFLVAFVGC

SEQ ID NO: 317 - protein, 9 aa, synthetic construct
CGACKENYC

SEQ ID NO: 318 - protein, 21 aa, synthetic construct
LMITFAIFLSLIMLVEVAAAI

SEQ ID NO: 319 - protein, 10 aa, synthetic construct
GGSGPVRRYQ

SEQ ID NO: 320 - protein, 8 aa, synthetic construct
GGPLGMTS

SEQ ID NO: 321 - protein, 154 aa, synthetic construct; identical to SEQ ID NO: 305

SEQ ID NO: 322 - protein, 14 aa, synthetic construct
GGSGEQKLISEEDL

SEQ ID NO: 323 - protein, 8 aa, synthetic construct
GGPLGMTS

SEQ ID NO: 324 - protein, 112 aa, synthetic construct; identical to SEQ ID NO: 308

SEQ ID NO: 325 - protein, 6 aa, synthetic construct
CCLVKS

SEQ ID NO: 326 - DNA, 915 bp, synthetic construct
  source 1..915: mol_type = other DNA
gatccgccgccaccatgagttccgaaaactgctttgtcgccgagaatagtagtctgcaccccgaatcaggacaggagaatgacgcaaccagcccccacttttcaacaaggcacgaagggagcttccaggtccctgtgctgtgcgcagtgatgaacgtggtctttatcactattctgatcattgctctgatcgcactgagtgtcggacagtacaatggcgggtcaggccccgtgcggagatatcagagctcctctcgcaccccttccgacaagccagtggcccatgtggtcgctaaccctcaggcagagggacagctgcagtggctgaacaggcgagccaatgctctgctggctaacggcgtggaactgcgagataatcagctggtggtccctagcgaggggctgtacctgatctattcccaggtcctgttcaaagggcagggatgcccatctacacacgtgctgctgacccatacaatctctagaattgccgtcagttaccagactaaggtgaacctgctgagcgccatcaagtcaccatgtcagagggagacacccgaaggagcagaggccaagccctggtacgagcctatctatctgggaggcgtgtttcagctggaaaaaggcgaccgactgtctgccgagattaatcggccagactacctggatttcgctgaaagtggccaggtgtattttgggatcattgcactgggaggcagcggagagcagaagctgatctctgaggaagatctgggagggagcggatgcccatcccctctgttcccaggacccagcaaacccttttgggtcctggtggtcgtgggaggcgtgctggcatgttatagcctgctggtcacagtggctttcattatcttttgggtccgctctaaacggtctcggctgctgtaatgtacaa

SEQ ID NO: 327 - protein, 297 aa, synthetic construct
MSSENCFVAENSSLHPESGQENDATSPHFSTRHEGSFQVPVLCAVMNVVFITILIIALIALSVGQYNGGSGPVRRYQSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGSGCPSPLFPGPSKPFWVLVVVGGVLACYSLLVTVAFIIFWVRSKRSRLL

SEQ ID NO: 328 - protein, 40 aa, synthetic construct
MSSENCFVAENSSLHPESGQENDATSPHFSTRHEGSFQVP

SEQ ID NO: 329 - protein, 21 aa, synthetic construct
VLCAVMNVVFITILIIALIAL

SEQ ID NO: 330 - protein, 16 aa, synthetic construct
SVGQYNGGSGPVRRYQ

SEQ ID NO: 331 - protein, 152 aa, synthetic construct
RTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 332 - protein, 4 aa, synthetic construct
GGSG

SEQ ID NO: 333 - protein, 10 aa, synthetic construct
EQKLISEEDL

SEQ ID NO: 334 - protein, 27 aa, synthetic construct
FWVLVVVGGVLACYSLLVTVAFIIFWV

SEQ ID NO: 335 - DNA, 972 bp, synthetic construct
  source 1..972: mol_type = other DNA
atgagttccgaaaactgctttgtcgccgagaatagtagtctgcaccccgaatcaggacaggagaatgacgcaaccagcccccacttttcaacaaggcacgaagggagcttccaggtccctgtgctgtgcgcagtgatgaacgtggtctttatcactattctgatcattgctctgatcgcactgagtgtcggacagtacaatggcgggtcaggccccgtgcggagatatcagggagggtctgggggaggaccactgggaatgacctccggagggtctgggagctcctctcgcaccccttccgacaagccagtggcccatgtggtcgctaaccctcaggcagagggacagctgcagtggctgaacaggcgagccaatgctctgctggctaacggcgtggaactgcgagataatcagctggtggtccctagcgaggggctgtacctgatctattcccaggtcctgttcaaagggcagggatgcccatctacacacgtgctgctgacccatacaatctctagaattgccgtcagttaccagactaaggtgaacctgctgagcgccatcaagtcaccatgtcagagggagacacccgaaggagcagaggccaagccctggtacgagcctatctatctgggaggcgtgtttcagctggaaaaaggcgaccgactgtctgccgagattaatcggccagactacctggatttcgctgaaagtggccaggtgtattttgggatcattgcactgggaggcagcggagagcagaagctgatctctgaggaagatctgggaggaccactgggaatgacctccggagggagcggatgcccatcccctctgttcccaggacccagcaaacccttttgggtcctggtggtcgtgggaggcgtgctggcatgttatagcctgctggtcacagtggctttcattatcttttgggtccgctctaaacggtctcggctgctgtaatgtaca

SEQ ID NO: 336 - protein, 321 aa, synthetic construct
MSSENCFVAENSSLHPESGQENDATSPHFSTRHEGSFQVPVLCAVMNVVFITILIIALIALSVGQYNGGSGPVRRYQGGSGGGPLGMTSGGSGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGMTSGGSGCPSPLFPGPSKPFWVLVVVGGVLACYSLLVTVAFIIFWVRSKRSRLL

SEQ ID NO: 337 - protein, 5 aa, synthetic construct
GYEVM

SEQ ID NO: 338 - protein, 40 aa, synthetic construct; identical to SEQ ID NO: 328

SEQ ID NO: 339 - protein, 21 aa, synthetic construct
VLCAVMNVVFITILIIALIAL

SEQ ID NO: 340 - protein, 16 aa, synthetic construct
SVGQYNGGSGPVRRYQ

SEQ ID NO: 341 - protein, 4 aa, synthetic construct
GGSG

SEQ ID NO: 342 - protein, 9 aa, synthetic construct
GGPLGMTSG

SEQ ID NO: 343 - protein, 6 aa, synthetic construct
GSGSSS

SEQ ID NO: 344 - protein, 152 aa, synthetic construct; identical to SEQ ID NO: 331

SEQ ID NO: 345 - protein, 10 aa, synthetic construct
EQKLISEEDL

SEQ ID NO: 346 - protein, 26 aa, synthetic construct
FWVLVVVGGVLACYSLLVTVAFIIFW

SEQ ID NO: 347 - DNA, 1251 bp, synthetic construct
  source 1..1251: mol_type = other DNA
ggatccgccgccaccatggccgtggagggcgggatgaagtgcgtgaaattcctgctgtacgtcctgctgctggccttttgcgcatgtgctgtgggactgatcgcagtgggagtcggagctcagctggtgctgtcccagactatcattcagggcgcaacccctgggtctctgctgccagtggtcatcattgccgtgggcgtcttcctgtttctggtggcattcgtcgggtgctgtggagcctgcaaggaaaactattgtctgatgatcaccttcgctatttttctgagcctgatcatgctggtggaggtcgccgctgcaattgcaggatacgtgggaggctccggaccagtccggagatatcaggggggacccctgggaatgacaagctcctctagtaggactcccagcgacaagcctgtggcccacgtggtcgctaatcctcaggcagaaggccagctgcagtggctgaacaggcgagcaaatgcactgctggccaacggagtggaactgcgagataatcagctggtggtcccatcagagggactgtacctgatctatagccaggtgctgttcaaaggacagggatgcccatccacacacgtgctgctgacccatacaatcagcagaattgccgtgagctaccagactaaggtcaacctgctgagcgccatcaagtcaccctgtcagagggagacccctgaaggcgctgaggcaaagccatggtacgagcccatctatctgggcggggtgtttcagctggaaaaaggggaccgactgagtgcagagattaatcggcccgactacctggatttcgccgaatcaggacaggtgtattttggcatcattgctctgggaggctctggcgagcagaagctgatcagtgaggaagatctgggaggaccactgggaatgacctccggagggtctgggcctaagaacaatcacacagcttctattctggaccgcatgcaggcagatttcaaatgctgtggcgccgctaactatacagactgggaaaagatcccttcaatgagcaaaaaccgagtgccagatagttgctgtattaatgtgactgtcggatgcggcatcaactttaatgagaaggccattcataaagaaggctgcgtggagaagatcggaggctggctgcggaaaaatgtgctggtggtcgcagccgctgcactgggaatcgccttcgtggaggtcctgggcatcgtgttcgcttgctgtctggtcaaatcttaatgtaca

SEQ ID NO: 348 - DNA, 1099 bp, synthetic construct
  source 1..1099: mol_type = other DNA
ggatccgccgccaccatggcagtggaaggcgggatgaagtgcgtcaaatgtggcgcctgcaaggagaactactgtctgatgatcaccttcgctatttttctgagcctgatcatgctggtggaagtcgccgctgcaattgcaggatacgtgggaggcagcggacccgtccggagatatcagggaggacctctgggcgtgcgaggagggagctcctctcgaacaccatccgacaagcctgtggctcacgtggtcgcaaatccacaggcagagggacagctgcagtggctgaacaggcgagccaatgctctgctggccaacggagtggaactgagggataatcagctggtggtccccagtgagggcctgtacctgatctattcacaggtgctgttcaaaggacagggctgtccttctactcacgtgctgctgacccatacaatctcaaggattgccgtgagctatcagactaaggtcaacctgctgtctgctatcaaaagtccttgccagcgagagaccccagaaggagcagaggccaagccatggtacgagcccatctatctgggaggcgtgtttcagctggaaaaaggagacagactgtctgcagagattaataggcctgactacctggatttcgccgaaagtggccaggtgtattttgggatcattgctctgggaggcagcggagagcagaagctgatctctgaggaagatctgggaggacctctgggggtgcgcggaggagggggaagcggaccaaagaacaatcacacagcttccattctggaccgaatgcaggcagatttcaaatgctgtggcgccgctaactacactgactgggaaaagatcccaagtatgtcaaaaaaccgggtgcccgatagctgctgtattaatgtgacagtcgggtgtggaatcaactttaatgagaaggccattcataaagaaggctgcgtggagaagatcggcgggtggctgcggaaaaatgtcctggtggtcgcagcagctgcactgggaatcgccttcgtggaagtcctggggatcgtgttcgcttgctgtctggtcaaaagcattagatccggctatgaagtgatgtaatgtacaa

SEQ ID NO: 349 - DNA, 2579 bp, synthetic construct
  source 1..2579: mol_type = other DNA
gatccgccgccaccatggccgtggagggcgggatgaagtgcgtcaaattcctgctgtacgtgctgctgctggccttttgcgcatgtgcagtcggactgatcgcagtcggagtgggagcccagctggtgctgtcccagactatcattcagggggcaacccctggatctctgctgccagtggtcatcattgctgtcggcgtgttcctgtttctggtcgctttcgtgggctgctgtggggcatgcaaggagaactactgtctgatgatcacattcgccatttttctgtccctgatcatgctggtcgaagtggccgctgcaattgctggctacgtcggaggctctggacctgtgcggagatatcaggggggaccactgggcatgacaagctcctctagtagaactcccagcgacaagcctgtggcccacgtggtcgctaatccacaggcagagggacagctgcagtggctgaacaggcgagctaatgcactgctggccaacggggtggagctgagggataatcagctggtggtcccttccgaaggactgtacctgatctattctcaggtcctgttcaaaggacagggctgcccaagcacccacgtgctgctgacccatacaatctcccgcattgccgtctcttaccagaccaaggtgaacctgctgagcgccatcaagtccccatgtcagcgggaaacacccgagggcgccgaagctaagccatggtacgagcccatctatctgggcggggtgtttcagctggagaaaggcgaccggctgagtgcagaaattaatagacctgactacctggatttcgccgagagcggacaggtgtattttggcatcattgctctgggaggctctggcgaacagaagctgatcagtgaggaagatctgggaggaccactgggaatgacatctggagggagtggaggagggagtggacaggtccagctggtggaatcaggaggaggactggtgcagcctggaggctctctgaagctgagttgcgccgcttcaggcttcgactttagtaggtactggatgtcatgggtgcgccaggcaccagggaaaggactggagtggatcggcgaaattaaccccacatcaagcactatcaatttcactcctagcctgaaggacaaagtgtttatttccagagataacgccaagaatacactgtacctgcagatgagcaaagtgaggtccgaggacactgctctgtactattgtgcacgaggcaactactatcggtacggggacgccatggattattggggccaggggacctccgtcacagtgtcaaagatcagcgggggaggagggagcggaggaggaggcagcggaggaggaggctctgggggaggcgggagtggaggcgggggcagctctgatatcgtcctgacacagtccccagcttctctggcagtgtctctgggccagcgcgcaactattagttgtcgagccagtaaatcagtgagcacctccggatacagctatctgcactggtatcagcagaagcccggccagccccctaaactgctgatctatctggcttctaacctggagagtggcgtgcctgcaagattctctggaagtggctcagggactgactttaccctgaatattcacccagtggaggaagaggatgcagccacctactattgccagcattcaagggagctgcccttcacctttggaagcggcacaaagctggaaatcaaaggcgggggaggctcaggaggcggcgggagcggcggcggaggcagcgccaagccaaccacaactccagctccacgaccaccaacacctgcaccaactatcgcttcacagcccctgagcctgagacctgaggcatgtaggccagctgcaggaggagcagtccatactcgaggactggacttcgcccctaggaagattgaagtgatgtaccctccaccctatctggataacgaaaaaagcaatggaaccatcattcacgtgaagggcaaacatctgtgcccctcacctctgttcccaggccccagcaagccattttgggtcctggtggtcgtgggaggcgtgctggcctgttacagcctgctggtcaccgtggctttcatcatcttctgggtgcggagcaaacggtcaagactgctgcacagcgactatatgaacatgaccccacgacggcctggaccaacacgaaagcattaccagccatatgcacctccacgagacttcgcagcttacaggtcccgcgtgaagttcagccgctccgcagatgcaccagcttaccagcagggacagaatcagctgtataacgagctgaatctggggagaagggaagagtatgacgtgctggataagcgacgaggacgggaccccgaaatgggaggaaagccacggagaaaaaaccctcaggaggggctttacaatgaactgcagaaggacaaaatggcagaggcctattccgaaatcggaatgaagggcgagaggcgccgagggaaaggacacgatggcctgtaccaggggctgtctactgccaccaaggacacctatgatgccctgcatatgcaggctctgccccctagataatgtaca

SEQ ID NO: 350 - DNA, 1254 bp, synthetic construct
  source 1..1254: mol_type = other DNA
gatccgccgccaccatggccgtggagggcgggatgaagtgcgtgaaattcctgctgtacgtcctgctgctggccttttgcgcatgtgctgtgggactgatcgcagtgggagtcggagctcagctggtgctgagccagactatcattcagggagcaacccctggcagcctgctgccagtggtcatcattgccgtgggcgtcttcctgtttctggtggcattcgtcggatgctgtggcgcctgcaaggaaaactattgtctgatgatcactttcgctatttttctgagcctgatcatgctggtggaggtcgccgctgcaattgcaggatacgtgggaggcagcggaccagtccggagatatcagggaggaccactgggcgtgcgaggagggagctcctctcgaaccccatccgacaagcctgtggcacacgtggtcgctaatcctcaggcagaagggcagctgcagtggctgaacaggcgcgcaaatgccctgctggccaacggagtggaactgagggataatcagctggtggtcccatccgagggcctgtacctgatctattctcaggtgctgttcaaagggcagggatgcccctctacacacgtgctgctgacccatacaatctctaggattgctgtgagttaccagacaaaggtcaacctgctgagcgccatcaagtccccctgtcagcgcgagactcctgaaggggctgaggcaaagccatggtacgagcccatctatctgggaggcgtgtttcagctggaaaaaggagacagactgtctgcagagattaataggcccgactacctggatttcgccgaaagtggccaggtgtattttgggatcattgctctggggggatcaggcgagcagaagctgatcagcgaggaagatctgggaggaccactgggggtgcgcggaggagggggaagtggacctaagaacaatcacaccgcttcaattctggaccgaatgcaggcagatttcaaatgctgtggcgccgctaactatacagactgggaaaagatccctagtatgtcaaaaaaccgggtgccagatagctgctgtattaatgtgaccgtcggctgcgggatcaactttaatgagaaggccattcataaagaaggctgcgtggagaagatcggcgggtggctgagaaaaaatgtcctggtggtcgcagcagctgcactgggaatcgccttcgtggaggtcctggggatcgtgttcgcttgctgtctggtcaaaagctaatgta

SEQ ID NO: 351 - DNA, 2586 bp, synthetic construct
  source 1..2586: mol_type = other DNA
ggatccgccgccaccatggccgtggagggcgggatgaagtgcgtcaaattcctgctgtacgtgctgctgctggccttttgcgcatgtgcagtcggactgatcgcagtcggagtgggagcacagctggtgctgagtcagactatcattcagggagcaacccctggcagcctgctgccagtggtcatcattgctgtcggcgtgttcctgtttctggtcgctttcgtggggtgctgtggagcatgcaaggagaactactgtctgatgatcactttcgccatttttctgtctctgatcatgctggtcgaagtggccgctgcaattgctggatacgtcggaggcagcggacctgtgcggagatatcagggaggaccactgggcgtccgcggagggagctcctctcgaaccccaagcgacaagcctgtggcacacgtggtcgctaatcctcaggcagagggacagctgcagtggctgaacaggcgagctaatgcactgctggccaacggagtggagctgagagataatcagctggtggtccctagcgaaggcctgtacctgatctattcccaggtcctgttcaaaggacagggatgcccatccacccacgtgctgctgacccatacaatcagcagaattgccgtctcctaccagacaaaggtgaacctgctgagcgccatcaagagtccatgtcagagggaaactcccgagggggccgaagctaagccatggtacgagcccatctatctgggaggcgtgtttcagctggagaaaggcgacaggctgagcgcagaaattaatcgccctgactacctggatttcgccgagtccggccaggtgtattttgggatcattgctctggggggcagcggcgaacagaagctgatctctgaggaagatctgggaggaccactgggggtgaggggaggagggggatcaggaggaggaagcggacaggtccagctggtggaaagcggaggaggactggtgcagccaggagggtccctgaagctgtcttgcgccgctagtggcttcgacttttctcgatactggatgagttgggtgaggcaggcccctggaaaaggcctggagtggatcggcgaaattaaccccacaagttcaactatcaatttcaccccttctctgaaggacaaagtgtttattagtcgcgataacgccaagaatactctgtacctgcagatgtcaaaagtgcggagcgaggacaccgctctgtactattgtgcacgagggaactactatcggtacggagacgccatggattattgggggcagggaacctctgtcacagtgtccaagatctctggcggaggaggatcaggaggaggaggcagcggcggaggagggtccggaggcgggggatctggcgggggaggcagctccgatatcgtcctgacacagagtcccgcttcactggcagtgtcactgggccagcgggcaaccattagctgtagagcctctaaaagcgtgagcaccagcggctacagctatctgcactggtatcagcagaagcctgggcagccccctaaactgctgatctatctggcttccaacctggagtctggcgtgcctgcacgcttctccggctctgggagtggaactgactttaccctgaatattcacccagtggaggaagaggatgcagccacttactattgccagcattcccgggagctgcccttcacctttggctctgggacaaagctggaaatcaaagggggaggcgggagtggcggcggcggatcaggcggaggaggcagcgccaagccaaccacaactccagctccacgaccaccaacacctgcaccaactatcgctagtcagcccctgtcactgagacctgaggcatgtaggccagctgcaggaggagcagtccatacccgaggactggacttcgcaccacgaaagattgaagtgatgtaccctccaccctatctggataacgaaaaaagcaatggcacaatcattcacgtgaaggggaaacatctgtgccccagtcctctgttcccaggcccctcaaagcccttttgggtcctggtggtcgtgggaggggtgctggcctgttacagcctgctggtcacagtggctttcatcatcttctgggtgcggagcaaacggtccagactgctgcactccgactatatgaacatgacaccacgacggcctggcccaactagaaagcattaccagccctatgcccctccaagggacttcgccgcttacaggtcccgcgtgaaattttcaagaagcgcagatgccccagcttaccagcagggacagaatcagctgtataacgagctgaatctgggcagaagggaagagtatgacgtgctggataagcgacgaggacgggaccccgaaatgggaggcaagccacggagaaaaaaccctcaggagggactttacaatgaactgcagaaggacaaaatggcagaggcctattctgaaatcggcatgaagggggagaggcgccgaggaaaaggccacgatgggctgtaccagggactgagcactgccaccaaggacacctatgatgccctgcatatgcaggctctgccccctcggtaatgtaca

SEQ ID NO: 352 - DNA, 1282 bp, synthetic construct
  source 1..1282: mol_type = other DNA
ggatccgccgccaccatggccgtggagggcgggatgaagtgcgtgaaattcctgctgtacgtcctgctgctggccttttgcgcatgtgctgtgggactgatcgcagtgggagtcggagctcagctggtgctgtctcagactatcattcagggggcaacccctggaagtctgctgccagtggtcatcattgccgtgggcgtcttcctgtttctggtggcattcgtcggatgctgtggcgcctgcaaggaaaactattgtctgatgatcactttcgctatttttctgtccctgatcatgctggtggaggtcgccgctgcaattgccggatacgtgggaggctctggcccagtccggagatatcagggaggaccactgggcgtgcgaggagggagctcctctcgaaccccaagtgacaagcctgtggcccacgtggtcgctaatcctcaggcagaagggcagctgcagtggctgaacaggcgcgcaaatgccctgctggccaacggagtggaactgagggataatcagctggtggtcccaagtgagggcctgtacctgatctattcacaggtgctgttcaaagggcagggatgccccagcacacacgtgctgctgacccatacaatctcaaggattgctgtgagctaccagacaaaggtcaacctgctgtctgccatcaaaagtccctgtcagcgcgagactcctgaaggggctgaggcaaagccatggtacgagcccatctatctgggaggcgtgtttcagctggaaaaaggagacagactgtcagcagagattaataggcccgactacctggatttcgccgaaagcggccaggtgtattttgggatcattgctctgggaggcagcggagagcagaagctgatctctgaggaagatctgggaggaccactgggggtgcgcggaggagggggaagcggacctaagaacaatcacaccgcttccattctggaccgaatgcaggcagatttcaaatgctgtggcgccgctaactacacagactgggaaaagatccctagtatgtcaaaaaaccgggtgccagattcctgctgtattaatgtgaccgtcggctgcgggatcaactttaatgagaaggccattcataaagaaggctgcgtggagaagatcggcgggtggctgcggaaaaatgtcctggtggtcgcagcagctgcactgggaatcgccttcgtggaagtcctggggatcgtgttcgcttgctgtctggtcaaaagcattagatccggctatgaagtgatgtaatgtacaa

SEQ ID NO: 353 - DNA, 1045 bp, synthetic construct
  source 1..1045: mol_type = other DNA
ggatccgccgccaccatggcagtggaaggcgggatgaagtgcgtcaaatgtggcgcctgcaaggagaactactgtctgatgatcaccttcgctatttttctgagcctgatcatgctggtggaagtcgccgctgcaattgcaggatacgtgggaggcagcggacccgtccggagatatcagagctcctctcgaacaccatccgacaagcctgtggctcacgtggtcgcaaatccacaggcagagggacagctgcagtggctgaacaggcgagccaatgctctgctggccaacggagtggaactgagggataatcagctggtggtccccagtgagggcctgtacctgatctattcacaggtgctgttcaaaggacagggctgtccttctactcacgtgctgctgacccatacaatctcaaggattgccgtgagctatcagactaaggtcaacctgctgtctgctatcaaaagtccttgccagcgagagaccccagaaggagcagaggccaagccatggtacgagcccatctatctgggaggcgtgtttcagctggaaaaaggagacagactgtctgcagagattaataggcctgactacctggatttcgccgaaagtggccaggtgtattttgggatcattgctctgggaggcagcggagagcagaagctgatctctgaggaagatctggggggaagcggaccaaagaacaatcacacagcttccattctggaccgaatgcaggcagatttcaaatgctgtggcgccgctaactacactgactgggaaaagatcccaagtatgtcaaaaaaccgggtgcccgatagctgctgtattaatgtgacagtcgggtgtggaatcaactttaatgagaaggccattcataaagaaggctgcgtggagaagatcggcgggtggctgcggaaaaatgtcctggtggtcgcagcagctgcactgggaatcgccttcgtggaagtcctggggatcgtgttcgcttgctgtctggtcaaaagcattagatccggctatgaagtgatgtaatgtacaa

SEQ ID NO: 354 - DNA, 2554 bp, synthetic construct
  source 1..2554: mol_type = other DNA
ggatccatgtcatccgagaactgctttgtcgccgaaaactcatcactgcaccccgaatcaggccaggaaaacgacgcaacctccccccacttctcaactagacacgagggcagcttccaggtgccagtgctgtgcgccgtgatgaacgtggtgtttatcaccattctgatcattgctctgatcgccctgagcgtggggcagtacaatggcgggtccggacccgtgcggagatatcagggaggctccggaggaggagaggaagaggaagaggtggaggccgactctgaagaggaagaggaagaggaatccgggggatctggcagctcctctagaaccccttccgataagccagtggctcatgtggtggccaaccctcaggctgagggacagctgcagtggctgaacaggagggctaatgctctgctggctaacggagtggagctgagggacaatcagctggtggtgccttctgaagggctgtacctgatctatagtcaggtgctgttcaaagggcagggatgcccatctacacacgtgctgctgacccatacaatcagtcgcattgccgtgagctaccagaccaaggtgaacctgctgagcgccatcaagagcccttgtcagcgggaaacaccagagggggccgaagctaagccctggtacgagcctatctatctgggcggggtgtttcagctggagaaaggcgatagactgagcgccgaaattaataggcccgactacctggatttcgctgagagcggccaggtgtattttgggatcattgccctggaacagaagctgatctccgaggaagacctgggaggctctggcgaagaggaagaggaggtggaggccgatagcgaggaagaagaggaagaggaaggaggcagcggaggaggatctggaggaggaagtggacaggtgcagctggtggagagcggcggaggactggtgcagccaggagggtcactgaagctgagctgcgccgcttccggcttcgactttagccgctactggatgtcctgggtgcggcaggctcctggcaaagggctggagtggatcggcgaaattaaccccactagttcaaccatcaatttcacaccttcactgaaggacaaagtgtttattagcagagataacgccaagaatacactgtacctgcagatgtctaaagtgaggagtgaggacactgccctgtactattgtgctcgcggaaattactatcggtacggcgacgccatggattattggggacagggcacaagcgtgactgtgagtaagatctcaggaggcggcggatctggaggaggaggcagtgggggaggagggtcaggaggaggaggaagcggcggaggaggcagctccgatatcgtgctgactcagagcccagcttccctggccgtgtccctgggacagcgcgccactatttcttgtcgggcttctaagagcgtgagcaccagcgggtactcctatctgcactggtaccagcagaagcctggacagccccctaaactgctgatctatctggcctcaaacctggagagcggcgtgcctgctagattctccgggtctggaagtggcaccgactttacactgaatattcacccagtggaggaagaggatgccgctacctactattgccagcatagtagggagctgcccttcacatttgggagcggaactaagctggagatcaagggaggaggagggtccggaggcgggggatctggcggcggaggcagtgccaagccaaccacaactccagctcctcgcccaccaactccagctccaaccatcgcttcccagccactgtctctgcggcccgaggcttgtaggcctgctgctgggggagctgtgcatacaagaggcctggacttcgccccaaggaagattgaagtgatgtaccctccaccctatctggataacgaaaaatccaatggcactatcattcacgtgaaggggaaacatctgtgccctagtccactgttccccggcccttcaaagcccttttgggtgctggtggtggtgggaggggtgctggcctgttactccctgctggtgaccgtggctttcatcatcttctgggtgcggagcaaacgcagtcggctgctgcactctgactatatgaacatgaccccaagacggcccggaccaacaaggaagcattaccagccatatgctcctccaagggacttcgctgcttacagaagcagggtgaaattttcacgcagcgccgatgctcctgcctaccagcagggacagaatcagctgtataacgagctgaatctgggcaggcgcgaagagtatgacgtgctggataagcggagaggacgggaccccgaaatgggaggcaagccaaggcgcaaaaacccccaggagggcctgtataacgaactgcagaaggacaaaatggctgaggcctatagcgaaatcggaatgaagggcgagcggagaaggggcaaagggcacgatgggctgtaccagggactgtccaccgccactaaagatacctatgatgctctgcacatgcaggctctgccaccccgataatgtacaa

SEQ ID NO: 355 - DNA, 988 bp, synthetic construct
  source 1..988: mol_type = other DNA
ggatccgccgccaccatgagttccgaaaactgctttgtcgccgagaatagtagtctgcaccccgaatcaggacaggagaatgacgcaaccagcccccacttttcaacaaggcacgaagggagcttccaggtccctgtgctgtgcgcagtgatgaacgtggtctttatcactattctgatcattgctctgatcgcactgagtgtcggacagtacaatggcgggtcaggccccgtgcggagatatcagggagggtctgggggaggaccactgggaatgacctccggagggtctgggagctcctctcgcaccccttccgacaagccagtggcccatgtggtcgctaaccctcaggcagagggacagctgcagtggctgaacaggcgagccaatgctctgctggctaacggcgtggaactgcgagataatcagctggtggtccctagcgaggggctgtacctgatctattcccaggtcctgttcaaagggcagggatgcccatctacacacgtgctgctgacccatacaatctctagaattgccgtcagttaccagactaaggtgaacctgctgagcgccatcaagtcaccatgtcagagggagacacccgaaggagcagaggccaagccctggtacgagcctatctatctgggaggcgtgtttcagctggaaaaaggcgaccgactgtctgccgagattaatcggccagactacctggatttcgctgaaagtggccaggtgtattttgggatcattgcactgggaggcagcggagagcagaagctgatctctgaggaagatctgggaggaccactgggaatgacctccggagggagcggatgcccatcccctctgttcccaggacccagcaaacccttttgggtcctggtggtcgtgggaggcgtgctggcatgttatagcctgctggtcacagtggctttcattatcttttgggtccgctctaaacggtctcggctgctgtaatgtacaa

SEQ ID NO: 356 - DNA, 916 bp, synthetic construct
  source 1..916: mol_type = other DNA
ggatccgccgccaccatgagttccgaaaactgctttgtcgccgagaatagtagtctgcaccccgaatcaggacaggagaatgacgcaaccagcccccacttttcaacaaggcacgaagggagcttccaggtccctgtgctgtgcgcagtgatgaacgtggtctttatcactattctgatcattgctctgatcgcactgagtgtcggacagtacaatggcgggtcaggccccgtgcggagatatcagagctcctctcgcaccccttccgacaagccagtggcccatgtggtcgctaaccctcaggcagagggacagctgcagtggctgaacaggcgagccaatgctctgctggctaacggcgtggaactgcgagataatcagctggtggtccctagcgaggggctgtacctgatctattcccaggtcctgttcaaagggcagggatgcccatctacacacgtgctgctgacccatacaatctctagaattgccgtcagttaccagactaaggtgaacctgctgagcgccatcaagtcaccatgtcagagggagacacccgaaggagcagaggccaagccctggtacgagcctatctatctgggaggcgtgtttcagctggaaaaaggcgaccgactgtctgccgagattaatcggccagactacctggatttcgctgaaagtggccaggtgtattttgggatcattgcactgggaggcagcggagagcagaagctgatctctgaggaagatctgggagggagcggatgcccatcccctctgttcccaggacccagcaaacccttttgggtcctggtggtcgtgggaggcgtgctggcatgttatagcctgctggtcacagtggctttcattatcttttgggtccgctctaaacggtctcggctgctgtaatgtacaa

SEQ ID NO: 357 - DNA, 2553 bp, synthetic construct
  source 1..2553: mol_type = other DNA
ggatccatgtcatccgagaactgctttgtcgccgaaaactcatcactgcaccccgaatcaggccaggaaaacgacgcaacctccccccacttctcaactagacacgagggcagcttccaggtgccagtgctgtgcgccgtgatgaacgtggtgtttatcaccattctgatcattgctctgatcgccctgagcgtggggcagtacaatggcgggtccggacccgtgcggagatatcagggaggctccggaggaggagaggaagaggaagaggtggaggccgactctgaagaggaagaggaagaggaatccgggggatctggcagctcctctagaaccccttccgataagccagtggctcatgtggtggccaaccctcaggctgagggacagctgcagtggctgaacaggagggctaatgctctgctggctaacggagtggagctgagggacaatcagctggtggtgccttctgaagggctgtacctgatctatagtcaggtgctgttcaaagggcagggatgcccatctacacacgtgctgctgacccatacaatcagtcgcattgccgtgagctaccagaccaaggtgaacctgctgagcgccatcaagagcccttgtcagcgggaaacaccagagggggccgaagctaagccctggtacgagcctatctatctgggcggggtgtttcagctggagaaaggcgatagactgagcgccgaaattaataggcccgactacctggatttcgctgagagcggccaggtgtattttgggatcattgccctggaacagaagctgatctccgaggaagacctgggaggctctggcgaagaggaagaggaggtggaggccgatagcgaggaagaagaggaagaggaaggaggcagcggaggaggatctggaggaggaagtggacaggtgcagctggtggagagcggcggaggactggtgcagccaggagggtcactgaagctgagctgcgccgcttccggcttcgactttagccgctactggatgtcctgggtgcggcaggctcctggcaaagggctggagtggatcggcgaaattaaccccactagttcaaccatcaatttcacaccttcactgaaggacaaagtgtttattagcagagataacgccaagaatacactgtacctgcagatgtctaaagtgaggagtgaggacactgccctgtactattgtgctcgcggaaattactatcggtacggcgacgccatggattattggggacagggcacaagcgtgactgtgagtaagatctcaggaggcggcggatctggaggaggaggcagtgggggaggagggtcaggaggaggaggaagcggcggaggaggcagctccgatatcgtgctgactcagagcccagcttccctggccgtgtccctgggacagcgcgccactatttcttgtcgggcttctaagagcgtgagcaccagcgggtactcctatctgcactggtaccagcagaagcctggacagccccctaaactgctgatctatctggcctcaaacctggagagcggcgtgcctgctagattctccgggtctggaagtggcaccgactttacactgaatattcacccagtggaggaagaggatgccgctacctactattgccagcatagtagggagctgcccttcacatttgggagcggaactaagctggagatcaagggaggaggagggtccggaggcgggggatctggcggcggaggcagtgccaagccaaccacaactccagctcctcgcccaccaactccagctccaaccatcgcttcccagccactgtctctgcggcccgaggcttgtaggcctgctgctgggggagctgtgcatacaagaggcctggacttcgccccaaggaagattgaagtgatgtaccctccaccctatctggataacgaaaaatccaatggcactatcattcacgtgaaggggaaacatctgtgccctagtccactgttccccggcccttcaaagcccttttgggtgctggtggtggtgggaggggtgctggcctgttactccctgctggtgaccgtggctttcatcatcttctgggtgcggagcaaacgcagtcggctgctgcactctgactatatgaacatgaccccaagacggcccggaccaacaaggaagcattaccagccatatgctcctccaagggacttcgctgcttacagaagcagggtgaaattttcacgcagcgccgatgctcctgcctaccagcagggacagaatcagctgtataacgagctgaatctgggcaggcgcgaagagtatgacgtgctggataagcggagaggacgggaccccgaaatgggaggcaagccaaggcgcaaaaacccccaggagggcctgtataacgaactgcagaaggacaaaatggctgaggcctatagcgaaatcggaatgaagggcgagcggagaaggggcaaagggcacgatgggctgtaccagggactgtccaccgccactaaagatacctatgatgctctgcacatgcaggctctgccaccccgataatgtaca

SEQ ID NO: 358 - protein, 857 aa, synthetic construct
MSSENCFVAENSSLHPESGQENDATSPHFSTRHEGSFQVPVLCAVMNVVFITILIIALIALSVGQYNGGSGPVRRYSRQGGSGGGEEEEEVEADSEEEEEEESGGSGSSSSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGEEEEEVEADSEEEEEEEGGSGGGSGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 359 - DNA, 1845 bp, synthetic construct
  source 1..1845: mol_type = other DNA
atgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagacagggcggctctggcggaggcgaagaggaagaagaggtggaagccgatagcgaggaagaagaagaagaggagagcggcggctccggcagcagcagctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggtagcggcgaggaggaagaagaagtggaagctgacagcgaagaagaggaagaggaggagggcgggagcggaggcggcagtggcctcgagcctaggggcggcagcggagttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataatgtaca

SEQ ID NO: 360 - DNA, 2385 bp, synthetic construct
  source 1..2385: mol_type = other DNA
atgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataatgtaca

SEQ ID NO: 361 - DNA, 2550 bp, synthetic construct
  source 1..2550: mol_type = other DNA
gccagtcctccgattgactgagtcgcccggatcccgccaccatgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagacagggcggctctggcggaggcatcgagcctgacagcggcggctccggctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggtagcggcatcgagcctgacggcgggagcggaggcggcagtggcctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataatgtacaagtaaagcggccggcc

SEQ ID NO: 362 - DNA, 2622 bp, synthetic construct
  source 1..2622: mol_type = other DNA
gccagtcctccgattgactgagtcgcccggatcccgccaccatgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagacagggcggctctggcggaggcgcccagggcgtgattagcgccagcgccagcaatctggacgacttttacagcggcggctccggctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggtagcggcgcccagggcgtgattagcgccagcgccagcaatctggacgacttttacggcgggagcggaggcggcagtggcctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataatgtacaagtaaagcggccggcc

SEQ ID NO: 363 - DNA, 2592 bp, synthetic construct
  source 1..2592: mol_type = other DNA
gccagtcctccgattgactgagtcgcccggatcccgccaccatgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagacagggcggctctggcggaggcctggaggccgacaagggcaagctggagtacgatagcggcggctccggctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggtagcggcctggaggccgacaagggcaagctggagtacgatggcgggagcggaggcggcagtggcctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataatgtacaagtaaagcggccggcc

SEQ ID NO: 364 - protein, 419 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVGGSGPVRRYQGGPLGVRGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGVRGGGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 365 - protein, 41 aa, synthetic construct
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
ADVGGTDYKDDDDKPGGRPTWPSSGGSGXXXXXXTASGAET

SEQ ID NO: 366 - protein, 954 aa, synthetic construct
MSCSVARVGLGLLLLLMGAGLAVQGWFLLQLHWRLGEMVTRLPDGPAGSWEQLIQERRSHEVNPAAGGSGPVRRYQMEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGSGEQKLISEEDLGGPLGMTSGGSGSLECTKLCLPQIENVKGTEDSGGSGQVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGREDPDAVDIWGQGTMVTVSSGGGGSGGGGSGGGGSDIQMTQSPSSLSASVGDRVTITCRASQGIRSWLAWYQQKPEKARKSLIYAASSLQSGVPSRFSGSGSGTDFTLTISSLQPEDFATYYCQQYNSYPLTFGGGTKVEIKGGGGSGGGGSGGGGSSGTTVLLPLVIFFGLCLLSLLFIGLMYRYGGSGKRGRKKLLYIFKQPFMRPVQTTQEEDG

SEQ ID NO: 367 - protein, 9 aa, synthetic construct
SGSGSGSGS

SEQ ID NO: 368 - protein, 358 aa, synthetic construct
MAVEGGMKCVKCGACKENYCLMITFAIFLSLIMLVEVAAAIAGYVGGSGPVRRYQGGPLGVRGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGVRGGGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 369 - protein, 820 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVGGSGPVRRYQGGPLGMTSSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGMTSGGSGGGSGPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 370 - protein, 297 aa, synthetic construct; identical to SEQ ID NO: 327

SEQ ID NO: 371 - protein, 411 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVGGSGPVRRYQGGPLGVRGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGVRGGGGSGPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKS

SEQ ID NO: 372 - protein, 822 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVGGSGPVRRYQGGPLGVRGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGEQKLISEEDLGGPLGVRGGGGSGGGSGPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 373 - protein, 824 aa, synthetic construct
MSSENCFVAENSSLHPESGQENDATSPHFSTRHEGSFQVPVLCAVMNVVFITILIIALIALSVGQYNGGSGPVRRYQGGSGGGEEEEEVEADSEEEEEEESGGSGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALEQKLISEEDLGGSGEEEEEVEADSEEEEEEEGGSGGGSGGGSGPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 374 - protein, 612 aa, synthetic construct
MSSENCFVAENSSLHPESGQENDATSPHFSTRHEGSFQVPVLCAVMNVVFITILIIALIALSVGQYNGGSGPVRRYSRQGGSGGGEEEEEVEADSEEEEEEESGGSGSSSSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGEEEEEVEADSEEEEEEEGGSGGGSGLEPRGGSGVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 375 - protein, 792 aa, synthetic construct
MSSENCFVAENSSLHPESGQENDATSPHFSTRHEGSFQVPVLCAVMNVVFITILIIALIALSVGQYNGGSGPVRRYSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 376 - protein, 852 aa, synthetic construct
MSSENCFVAENSSLHPESGQENDATSPHFSTRHEGSFQVPVLCAVMNVVFITILIIALIALSVGQYNGGSGPVRRYSRQGGSGGGAQGVISASASNLDDFYSGGSGSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGAQGVISASASNLDDFYGGSGGGSGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 377 - protein, 321 aa, synthetic construct; identical to SEQ ID NO: 336

SEQ ID NO: 378 - DNA, 42 bp, synthetic construct
  source 1..42: mol_type = other DNA
tgctgcctggtgaagtctatcagatctggctatgaggtgatg

SEQ ID NO: 379 - DNA, 18 bp, synthetic construct
  source 1..18: mol_type = other DNA
tgctgcctggtgaagtct

SEQ ID NO: 380 - protein, 16 aa, synthetic construct
GGSGEQKLISEEDLGG

SEQ ID NO: 381 - protein, 170 aa, synthetic construct
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 382 - DNA, 2637 bp, synthetic construct
  source 1..2637: mol_type = other DNA
gccagtcctccgattgactgagtcgcccggatcccgccaccatgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagacagggcggctctggcggaggcgaagaggaagaagaggtggaagccgatagcgaggaagaagaagaagaggagagcggcggctccggcagcagcagctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggtagcggcgaggaggaagaagaagtggaagctgacagcgaagaagaggaagaggaggagggcgggagcggaggcggcagtggcctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataatgtacaagtaaagcggccggcc

SEQ ID NO: 383 - protein, 828 aa, synthetic construct
MSSENCFVAENSSLHPESGQENDATSPHFSTRHEGSFQVPVLCAVMNVVFITILIIALIALSVGQYNGGSGPVRRYSRQGGSGGGIEPDSGGSGSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGIEPDGGSGGGSGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 384 - protein, 842 aa, synthetic construct
MSSENCFVAENSSLHPESGQENDATSPHFSTRHEGSFQVPVLCAVMNVVFITILIIALIALSVGQYNGGSGPVRRYSRQGGSGGGLEADKGKLEYDSGGSGSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGLEADKGKLEYDGGSGGGSGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 385 - protein, 5 aa, synthetic construct
DEVDG

SEQ ID NO: 386 - DNA, 2502 bp, synthetic construct
  source 1..2502: mol_type = other DNA
atgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagacagggcggctctggcggaggcgatgaagtggacggcagcggcggctccggcagcagcagctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggtagcggcgatgaagtggacggcggcgggagcggaggcggcagtggcctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataa

SEQ ID NO: 387 - protein, 833 aa, synthetic construct
MSSENCFVAENSSLHPESGQENDATSPHFSTRHEGSFQVPVLCAVMNVVFITILIIALIALSVGQYNGGSGPVRRYSRQGGSGGGDEVDGSGGSGSSSSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGDEVDGGGSGGGSGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 388 - protein, 11 aa, synthetic construct
EAALVDMVNDG

SEQ ID NO: 389 - DNA, 2529 bp, synthetic construct
  source 1..2529: mol_type = other DNA
atgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagaggcggctctggcggaggcgaggccgccctggtggatatggtgaacgacggcagcggcggctccggcggatctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggctctggcggaggcgaggccgccctggtggatatggtgaacgacggcagcggcggctccggcggactcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataa

SEQ ID NO: 390 - protein, 842 aa, synthetic construct
MSSENCFVAENSSLHPESGQENDATSPHFSTRHEGSFQVPVLCAVMNVVFITILIIALIALSVGQYNGGSGPVRRYSRGGSGGGEAALVDMVNDGSGGSGGSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGGGEAALVDMVNDGSGGSGGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 391 - protein, 10 aa, synthetic construct
EDYGRDSGPP

SEQ ID NO: 392 - DNA, 2535 bp, synthetic construct
  source 1..2535: mol_type = other DNA
atgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagaggcggctctggcggaggcgaggactacggcagagatagcggccctcccagcggcggctccggcagcagcagctctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggctctggcggaggcgaggactacggcagagatagcggccctcccagcggcggctccggcagcagcagcctcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataa

SEQ ID NO: 393 - protein, 844 aa, synthetic construct
MSSENCFVAENSSLHPESGQENDATSPHFSTRHEGSFQVPVLCAVMNVVFITILIIALIALSVGQYNGGSGPVRRYSRGGSGGGEDYGRDSGPPSGGSGSSSSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGGGEDYGRDSGPPSGGSGSSSLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 394 - protein, 13 aa, synthetic construct
TPSEPDSGQGPPQ

SEQ ID NO: 395 - DNA, 2541 bp, synthetic construct
  source 1..2541: mol_type = other DNA
atgagctctgagaactgcttcgtggccgagaacagcagcctccaccccgagagtggtcaggagaacgacgccaccagcccccacttcagcacaagacacgagggctcattccaggtgcctgtgctgtgcgccgtgatgaacgtggtgttcatcaccatcctgatcatcgccctgattgccctttctgtgggccaatacaatggcggctccggtcctgtgcgcagatactctagaggcggctctggcggaggcacccctagcgagcccggatctggcagaggccctccacagagcggcggctccggcggatctagagtgttcaccctcgaagatttcgtgggcgactggagacagaccgccggctacaacctggaccaggtgctggaacagggcggcgtgagctctctgtttcagaatctgggggttagcgtgacaccaatccagaggatcgtgctgtccggcgagaacgggctgaagatcgacatccacgtgatcattccatacgagggcctgtctggcgatcagatgggccagatcgagaagatcttcaaggtggtgtaccctgtagacgaccaccattttaaggtgatcctgcactacggcaccctggtcatcgacggcgtgacacccaacatgatcgactacttcggcagaccatatgagggtattgccgtgtttgatggaaagaagatcacagtcaccggcaccctgtggaacggcaacaagattatcgatgaacggctgatcaaccccgacggctctctgctgtttagagtgaccatcaatggcgtgacaggctggagactgtgcgagagaatcctggctgagcagaaactgatcagcgaggaagacctgctcgagggcggctctggcggaggcacccctagcgagcccggatctggcagaggccctccacagagcggcggctccggcggactcgagcctaggggcggcagcggagttaaccccgatattcagatgactcagactaccagctccctgagcgcatccctgggcgaccgagtgacaatcagttgccgagccagccaggacatcagcaagtatctgaactggtaccagcagaagcctgacggaaccgtcaaactgctgatctatcacacatcccgcctgcattctggcgtgccaagtcggttctctggaagtggctcagggaccgactactcactgacaatcagcaacctggagcaggaagatattgccacctatttctgccagcagggcaatactctgccttacacctttggcgggggaactaagctggagattaccggcggaggaggcagcggaggaggagggtccggaggcgggggatctgaggtgaaactgcaggaaagcggacctggcctggtcgctccaagccagtccctgtctgtgacttgtaccgtcagcggagtgtccctgcccgattacggcgtctcttggatcagacagccccctagaaagggcctggagtggctgggcgtgatctggggaagtgaaaccacatactataattccgccctgaaaagccggctgacaatcattaaggacaacagtaaatcacaggtgttcctgaagatgaattccctgcagacagacgatactgcaatctactattgcgccaaacactactattacggcgggagctatgctatggattactgggggcagggaacctccgtcacagtgtctagtgttaactcaggcggcggcggcagcggaggcggaggcagtgccaagcctacaaccacccccgcccctagacctcctacacctgcccctaccatcgctagccagccactctctctcagacctgaagcctgtagacccgccgctggcggcgccgtgcacaccagaggcctggacttcgcgcctcggaagatcgaggtgatgtaccccccgccttacctggacaacgagaagtctaatggaacaatcatccacgtgaagggaaaacacctgtgtccttctcctctgttccctggccccagcaagcctttctgggtcctggtggtggtcggcggcgtgctggcgtgctacagcctgctcgtgaccgtggccttcatcattttctgggtgcggagcaaacggagcagactgctgcatagcgattacatgaacatgaccccaagaagacctgggccaaccagaaagcactaccagccttacgccccccccagagatttcgccgcttatagaagccgggtgaagttctccagatccgccgacgcccctgcttaccaacagggccagaaccagctgtataacgagctgaatctgggcagaagagaggagtacgacgtgctggataagcggagaggaagagaccctgagatgggcggcaagccccggcggaagaaccctcaggagggcctgtataacgagctgcaaaaagataagatggccgaggcttatagcgagatcggaatgaagggcgagcggcggaggggcaaaggccacgacggcctgtaccagggactgagcacagccacaaaggacacctacgacgctctgcacatgcaggccctgcctcctagataa

SEQ ID NO: 396 - protein, 846 aa, synthetic construct
MSSENCFVAENSSLHPESGQENDATSPHFSTRHEGSFQVPVLCAVMNVVFITILIIALIALSVGQYNGGSGPVRRYSRGGSGGGTPSEPGSGRGPPQSGGSGGSRVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAEQKLISEEDLLEGGSGGGTPSEPGSGRGPPQSGGSGGLEPRGGSGVNPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVNSGGGGSGGGGSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0315618 A1
(43) Pub. Date: Oct. 15, 2026

(54) PLASMA MEMBRANE EMBEDDED SYSTEM FOR SPACIOTEMPORAL CONTROLLED PAYLOAD DELIVERY OF BIOLOGICS

(71) Applicant: VYCELLIX, INC., Tampa, FL (US)

(72) Inventors: Evren ALICI, Huddinge (SE); Alamdar HUSSAIN, Huddinge (SE); Dara MOHAMMAD, Stockholm (SE)

(73) Assignee: VYCELLIX, INC., Tampa, FL (US)

(21) Appl. No.: 19/485,198

(22) PCT Filed: May 17, 2024
(86) PCT No.: PCT/US2024/030075

§ 371 Date: Nov. 17, 2025

Related U.S. Application Data
(60) Provisional application No. 63/467,518, filed on May 18, 2023.

(51) Int. Cl. A61K 47/69; A61K 47/65; A61K 47/68; C07K 14/47; C07K 14/705; C07K 16/28

(52) U.S. Cl. CPC A61K 47/6901; A61K 47/65; A61K 47/6879; C07K 14/4702; C07K 14/7051; C07K 14/70589; C07K 14/70596; C07K 16/2803; C07K 16/289; C07K 2317/31

(57) ABSTRACT

Disclosed is a peptide delivery system for delivering a peptide payload to a target cell or target locus and methods of delivering a peptide payload to a target locus. The peptide delivery system may include a cell, extracellular vesicle, or lipid-containing particle into which an exogenous polypeptide has been anchored, the exogenous polypeptide having a first and a second transmembrane domain connected by a loop region, which loop region comprises a first cleavage site, a peptide payload, and a second cleavage site and optionally, a targeting moiety for targeting the peptide delivery system to a target cell or target locus.

### CROSS-REFERENCE TO RELATED APPLICATIONS

This application claims the benefit under 35 U.S.C. § 119(e) of U.S. Provisional Application No. 63/467,518 filed May 18, 2023, which application is expressly incorporated by reference herein in its entirety.

### INCORPORATION BY REFERENCE STATEMENT REGARDING SEQUENCE LISTING

The instant application contains a Sequence Listing which has been submitted electronically in XML file format and is hereby incorporated by reference in its entirety. Said XML copy, created on May 16, 2024, is named “P71267_SL.xml” and is 451,938 bytes in size.

### BACKGROUND

Living organisms are increasingly being treated with peptide-based therapeutics. These therapeutics comprise hormones, cytokines, chemokines, pro-drugs, and antibodies in various formats, such as chimeric antigen receptors (CARs), bi-specific killer cell engager (BIKE), tri-specific killer cell engager (TRIKE), etc. Any peptide or protein that serves a function within the body is a potential therapeutic. While the therapeutic potential of peptides and proteins is promising, several factors limit their bioavailability, and thus their function in vivo.

One limiting factor is the in vitro production, stability, and multiple dose-delivery of these biological products. These biologics are often synthesized in mammalian cells, bacteria, or, more recently, in unicellular eukaryotes like Pichia pastoris. The biologics are purified from the producer cells, formulated in a delivery system, and administered to the patients. Therapeutic levels often require continuous delivery and/or repeated peptide dosing.

Systemic delivery of the biologics further lowers the efficacy depending on the drug's pharmacokinetics and dynamics because only a small fraction of the administered peptide may reach the target sites. Most of the biologic may be degraded and metabolized before reaching the intended site of action.

A third limiting factor is the biotoxicity related to the mechanism of action (MOA) of these biologics.

There is a need in the art for improved protein and peptide delivery methods to increase safety and efficacy of biologics.

### SUMMARY OF THE INVENTION

Disclosed herein is a peptide delivery system for delivering a peptide payload to a target cell or target locus, the peptide delivery system comprising: a cell, extracellular vesicle, or lipid-containing particle into which an exogenous polypeptide has been anchored, which exogenous polypeptide comprises: a first and a second transmembrane domain connected by a loop region, which loop region comprises a first cleavage site, a peptide payload, and a second cleavage site; and optionally, a targeting moiety for targeting the peptide delivery system to the target cell or the target locus.

In such a peptide delivery system, the first and/or second cleavage sites may comprise proteolytic cleavage substrates, preferably sheddase cleavage sites, MMP cleavage sites, GrzB cleavage sites, and Casp3 cleavage sites.

Such a peptide delivery system may be configured to release the peptide payload upon cleavage by a protease, preferably aby a sheddase, which may be endogenous or exogenous (e.g., included as a component of the peptide delivery system).

Such a peptide delivery system is also contemplated, wherein the sheddase is present at the target locus and/or released by the target cell.

In embodiments of the above peptide delivery system, the sheddase may comprise a full-time sheddase selected from ADAM proteases (metalloproteases), BACE proteases, serine-protease Granzyme-B and Site-1 proteases. The ADAM proteases may include membrane-anchored type 1 proteases such as ADAM8, ADAM9, ADAM10, ADAM12, ADAM15, ADAM17, ADAM19, ADAMthr20, ADAM21, ADAM28, ADAM30, and ADAM33.

Also contemplated herein is such a peptide delivery system, wherein the sheddase is a part-time sheddase selected from Meprin B, MT-MMPs (Membrane Type Matrix Metalloproteases), Pro-protein convertases, transmembrane serine proteases, Matrix Metalloproteases, Legumain and Cathepsin S and L. The MT-MMPs may be membrane-anchored type 1 or GPI-anchored and may include MT1-MMP, MT2-MMP, MT3-MMP, MT4-MMP, MT5-MMP, and MT6-MMP. The Pro-protein convertases may be selected from PCSK1/3, KCSK2, Furin, PCSK4, PSCK5/6, PACE4, PCSK7, and PCSK9. The transmembrane serine proteases may be selected from membrane-anchored type II proteases including Matriptase, Matriptase-2, Matriptase-3, Polymerase-1, Corin, Hepsin, TMPRSS2, TMPRSS3, TMPRSS4, MSPL, Spinesin, Enteropeptidase, HAT, DESCL1, TMPRSS11A, HAT-like 4 and HAT-like 5. The Matrix Metalloproteases may be selected from soluble proteases including MMP1, MMP2, MMP3, MMP4, MMP5, MMP6, MMP7, MMP8, MMP9, MMP10, MMPP11, MMP12, MMPP13, MMPP14, MMPP15, MMP16, MMPP17, MMPP18, MMP19, MMP20, MMP21, MMP21, MMP 23A-B, MMP24, MMP25, MMP26, MMP27 and MMP28. In an embodiment the Matrix Metalloproteases are selected from MMP2, MMP9 and/or MMP25. In an embodiment the MMP2 may be selected from SEQ ID NOS: 2-6. In an embodiment the MMP9 may be selected from SEQ ID NOS: 7-8. In an embodiment the MMP25 may be selected from SEQ ID NOS: 9-17, 18 or 155, and 19-29.

Also disclosed herein is such a peptide delivery system, wherein the transmembrane domain comprises a transmembrane domain from one or more of the following proteins: CD63, CD9, CD81, CD28, CD4, CD8, CD34, CD69, CD19, CD20, Integrins, TNFSF14, TNR1A, Aquaporins, NOTCH, NgR1, NRG1, GPi anchor, EGFR, or Rhodopsin.

In certain embodiments, a peptide delivery system as above may further comprise an intracellular or extracellular domain selected from or derived from one or more of the following proteins: OX-40, 4-1BB/CD137, CD2, CD7, CD27, CD30, B7-1 (CD80), B7-2 (CD86), PDL-1, Programmed Death-I (PD-1), Inducible T cell Co-Stimulator (ICOS), lymphocyte Function-associated Antigen-I (LFA-1 (CDI la/CD 18), CD3 gamma, CD3 delta, CD3 epsilon, CD247, CD276 (B7-H3), LIGHT, (TNFSF14), NKG2C, lg alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), The costimulatory ligand (PD-L2, 4-1 BBL, OX40L, Inducible Co-Stimulatory Ligand (ICOS-L), Inter-Cellular Adhesion Molecule (ICAM, CD30L, CD70, CD83, HLA-G, MICA, Ml CB, HVEM, lymphotoxin b receptor, 3/TR6, ILT3, ILT4), activating NK cell receptors, BTLA, a Toll ligand receptor, ICAM-1, B7-H3, CDS, ICAM-1, GITR, BAFFR, LIGHT, HVEM (LIGHTR), KIRDS2, NKp80 (KLRFI), NKp44, NKp30, NKp46, CD19, CD8 alpha, CD8 beta, IL-2R beta, IL-2R gamma, IL-7R alpha, ITGA4, VLAl, CD49a, ITGA4, CD49D, ITGA6, VLA-6, CD49f, ITGAD, CDl Id, ITGAE, CD103, ITGAL, CDl la, LFA-1, IT GAM, CDl lb, ITGAX, CDl le, ITGBI, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD 160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

In embodiments such a peptide delivery system may comprise a targeting moiety and the targeting moiety may bind to BCMA, MUC16 (also known as CA125), EGFR, EGFRvIII, MUCI, Flt-3, WT-1, CD38, CD70, CD90, CD133, MHC-WTI, TSPANI0, MHC-PRAME, MHC-NY-ESOI, HER2 (ERBB2), CA-IX (Carbonic anhydrase IX), LIVI, ADAMI0, CHRNA2, LeY, NKG2D, CSI, CD44v6, CD24, LGR5, ALDH, ALDH1, CD54, Sca1, CD271, CD123, CD36, CD109, CD110, CD71-negative, CCA, ABCG2, Claudin-18.2 (Claudin-18A2, or Claudinl8 isoform 2), PSCA, DLL3 (Delta-like protein 3, Drosophila Delta homolog 3, Delta3), Mud 7 (Mucinl 7, Muc3, Muc3), FAP alpha (Fibroblast Activation Protein alpha), Ly6G6D (Lymphocyte antigen 6 complex locus protein G6d, c6orf23, G6D, MEGTI, NG25), PSMA, MSLN, or RNF43 (E3 ubiquitin-protein ligase RNF43, RING finger protein 43). BAFF, C242 antigen, disialoganglioside (GD2), 4-IBB, 5T4, CD22, CD221, CD23 (IgE receptor), CD28, CD30 (TNFRSF8), CD33, CD4, CD40, CD44v6, CD44 CD51, CD52, CD56, CD74, CEA, CNT0888, CTLA-4, DR5, EpCAM, FAP, fibronectin extradomain-B, folate receptor 1, GD3 ganglioside, glycoprotein 75, GPNMB, HER2/neu, HGF, human scatter factor receptor kinase, IGF-1 receptor, IGF-I, IgGI, LI-CAM, integrin α5β1, integrin αvβ, legumain, MORAb-009, MS4A1, MUC1, mucin CanAg, C-MET, CCR4, CD152, CD10, CD19, CD20, CD200, N-glycolylneuraminic acid, NPC-IC, PDGF-R a, PDL192, phosphatidylserine, tumor antigen CTAA16.88, VEGF-A, VEGFR-1, VEGFR2, vimentin, RANKL, RON, ROR1, SCH900105, SDC1, SLAMF7, TAG-72, tenascin C, TGF-β, TRAIL-R1, TRAIL-R2, folic acid receptor, transferrin receptors, or any combination thereof.

Also contemplated herein is a peptide delivery system as above, wherein the second transmembrane domain is connected to an intracellular region comprising an intracellular domain from one or more of 41BB, ICOS, and CD3ζ. In certain embodiments, phosphorylation by a kinase within the intracellular domain of 41BB, ICOS, or CD3ζ may activate GrzB and allow GrzB to be transported out of the cell and cleave the first and/or second cleavage site.

In certain embodiments, the peptide delivery system as above may comprise a third and fourth transmembrane domain connected by a second loop region, wherein the third transmembrane domain is connected to an intracellular region comprising an intracellular domain from 41BB, ICOS, or CD3z. In such embodiments phosphorylation by a kinases within the intracellular domain of 41BB, ICOS or CD3ζ may activate the cell to release endogenous GrzB allowing the GrzB to be transported out of the cell and cleave GrzB cleavage present in the first or second loop region thereby releasing the peptide payload.

In an embodiment the second transmembrane domain may be from a NOTCH protein having a cleavage site configured to release a functional domain to which the NOTCH protein transmembrane domain is connected in the intracellular milieu.

Such a peptide delivery system as above is contemplated, wherein the payload may comprise: an antibody, an fragment of an antibody, a VHH, a cytokine or chemokine consisting a fraction or derivative of a sulfated xylan, which is an antagonist of a ligand selected from the list comprising IL-la, IL-1B, IL-2, IL-3, IL-4, IL-5, IL-6, IL-7, IL-8, IL-9, IL-10, IL-11, IL-12, IL-3, IL-14, IL-15 or IL-17 family of cytokines including IL-25, interferon, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, PDGF, SCF, TGFa, TGFB, TNFa, TNFB, TPO, VEGF, GH, NGF, NT3, NT4, NT5, NT6, NT7, Oncostatin M (OSM), insulin, MCP-1, MCP-2, MCP-3, MCP-4, MCP-5, a member of the MIP-1 family including MIP-la, MIP-2, eotaxin (eotaxin-1, -2 or -3), PBP (platelet basic protein), SDF-1, PBSF, PF4, RANTES, elastase; an enzyme of the cathepsin family, a cell adhesion molecules such as PECAM-1, a soluble or cell- or virus-bound receptor, cytokine-induced neutrophil chemoattractant (KC), TNF-α and IFN-g), and other soluble mediators of inflammation, such as reactive oxygen species and nitric oxide, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, thrombin, urokinase, a vaccine or any combination thereof.

In embodiments, a peptide delivery system as above may further comprise a linker between the first transmembrane domain and the first cleavage site. The linker may be 2-128 amino acids in length or 4-20 amino acids in length. In certain embodiments the linker may be selected from the linkers set forth in Table 4.

In an embodiment, the peptide delivery system may be configured to be sorted specifically into extracellular vesicles.

Also contemplated herein is a peptide delivery system as above which is configured to release two or more peptide payloads intracellularly or extracellularly.

Also contemplated herein is such a peptide delivery system, which comprises two or more sheddase cleavage sites, and each cleavage site can be the same or different from any of the other cleavage sites.

Also contemplated herein is such a peptide delivery system, which comprises an intracellular domain and the intracellular domain comprises a stalk domain of CD45 phosphatase.

Also contemplated herein is a peptide delivery system as above which comprises a single domain antibody or single chain of an antibody which binds a self-associated antigen (SAA) to bring the SAA in proximity, e.g., in cis or in trans.

Also contemplated herein is such a peptide delivery system, which comprises a single domain antibody or single chain of an antibody which binds the self-associated antigen (SAA), and which further comprises a CD45 phosphatase-binding moiety or an NKG2A-binding moiety, which activates an inhibitory signal in the target cell.

In embodiments of the above peptide delivery system, the peptide payload may be any peptide or protein that serves a function within the human body, including hormones, cytokines, chemokines, pro-drugs, and antibodies in various formats, such as chimeric antigen receptors (CARs), bi-specific killer cell engager (BIKE), tri-specific killer cell engager (TRIKE), etc.

In embodiments, the peptide delivery system as above may be selected from: CD63-MMP2-a-CD16-a-CD33-Myc-MMP9-CD63-GPi (SEQ ID NO: 124), CD63-MMP2-a-CD16-a-CD33-Myc-MMP9-CytCD63-EV Motif (SEQ ID NO: 155), CD63-MMP2-a-CD16-a-CD33-Myc-MMP9-CytCD63 (SEQ ID NO: 36 or 188), TNFSF14-a-CD16-a-CD33-Myc-TNR1A-ADAM17-a-CD38-41BB (SEQ ID NO: 54 or 366 or 367), CD63-GrzB-a-CD16-a-CD33-Myc-GrzB-a-CD38-CD28-CD3z (SEQ ID NO: 71 or 252), CD63-MMP9-2L-TNFa-Myc-MMP9-CD63-Cyt (SEQ ID NO: 88 or 295), CD63-MMP9-2L-TNFa-Myc-MMP9-CD63-D-Cyt (Lacks EVs sorting motif) (SEQ ID NO: 103 or 311), CD63-DL1-MMP2-2L-TNFa-Myc-MMP2-CD63-Cyt (SEQ ID NO: 104 or 368), CD63-MMP9-2L-TNFa-Myc-MMP9-aCD19CAR (SEQ ID NO: 105 or 369), CD63-MMP2-2L-TNFa-Myc-MMP2-CD63-D-Cyt (SEQ ID NO: 106) or 370 or 371, CD63-MMP2-2L-TNFa-Myc-MMP2-aCD19CAR (SEQ ID NO: 107 or 372 o4 373), CD69cyt-TMII-TNFa-Myctag-CD28TM-cyt (SEQ ID NO: 119 or 370), CD69cyt-TMII-MMP9-TNFa-Myc-MMP9-CD28TM-cyt (SEQ ID NO: 120 or 377), CD69Cyt-TM-GrzB-TNFa-Myc-GrzB-a-CD19sc-Stalk-CD28-CD3z (SEQ ID NO: 107 or 372 or 373), CD69Cyt-TM-GrzB-nLuc-Myc-GrzB-CD28-CD3z (Delta sc-GAGE B) (SEQ ID NO: 108 or 374), CD69Cyt-TM-nLuc-Myc-a-CD19sc-Stalk-CD28-CD3z (Delta GrzB, GAGE A) (SEQ ID NO: 109 or 375), CD69Cyt-TM-GrzB (Original)-nLuc-Myc-GrzB(Original)-a-CD19sc-Stalk-CD28-CD3z (SEQ ID NO: 358 or 382), CD69Cyt-TM-GrzB(IEPD)-nLuc-Myc-GrzB(IEPD)-a-CD19sc-Stalk-CD28-CD3z (SEQ ID NO: 110 or 383), CD69Cyt-TM-GrzB(SASA)-nLuc-Myc-, GrzB(SASA)-a-CD19sc-Stalk-CD28-CD3z (SEQ ID NO: 111 or 376), or CD69Cyt-TM-GrzB(ADKG)-nLuc-Myc-GrzB(ADKG)-a-CD19sc-Stalk-CD28-CD3z (SEQ ID NO: 112 or 384).

In certain embodiments, the above peptide delivery system may comprises a cell into which the exogenous polypeptide has been anchored. In certain embodiments, the cell may be selected from a Hematopoietic Stem Cells (HSC), an induced Pluripotent Stem Cell (iPSC) and their derived cell products, an Adoptive T cell, a Dendritic cell (DC), Natural killer (NK) cells, or any therapeutic immune or non-immune cells.

Also contemplated herein is an adoptive cell or engineered immune cell comprising a peptide payload of such a peptide delivery system, wherein the immune cells are autologous or allogeneic.

In embodiments, the exogenous polypeptide may be introduced into the cell individually via a CRISPR system, or in the form of viral or nonviral vectors, or one or more mRNAs that encode the exogenous polypeptide.

In embodiments, the exogenous polypeptide of the above peptide delivery system may be delivered using CPPs, micelles, liposomes, nanoparticles, dendrimers, nanotubes, electroporation, viral transduction, nucleofection, transfection, cell fusion, or microinjection.

Also contemplated herein is a genetic construct encoding the exogenous polypeptide of the peptide delivery system.

Also contemplated herein is a method of treating a patient in need thereof comprising administering the peptide delivery system as above to the patient.

Also disclosed herein is a method of delivering a peptide to a target locus in a patient in need thereof, the method comprising: administering to the patient a peptide delivery system comprising a lipid-comprising vescicle (e.g., cell, extracellular vesicle, lipid nanoparticle) into which an exogenous polypeptide has been anchored, the exogenous polypeptide comprising: a peptide payload; a first and a second transmembrane region; the first transmembrane region comprising a transmembrane domain connected to at least one sheddase or other proteolytic cleavage site; the second transmembrane region comprising a transmembrane domain connected to at least one sheddase or other proteolytic cleavage site; and optionally, a targeting moiety for targeting the exogenous polypeptide to a target locus, wherein when the lipid-comprising vescicle arrives at the target locus, a sheddase and/or other protease cleaves the cleavage sites of the first and second transmembrane regions releasing the peptide payload.

Also contemplated herein is such a method, wherein the cleaveage sites are derived from proteolytic cleavage substrates naturally present in Glycosylphosphatidylinositol (GPi) anchored proteins, membrane proteins with a single, two, three, four, or more transmembrane domains, intracellular domains, or extracellular domains in the extracellular matrix or in neighboring cells.

Also contemplated herein is such a method, wherein the sheddases are selected from full- or part-time sheddases. In embodiments, the full-time sheddases may be selected from ADAM proteases, BACE proteases, serine-protease Granzyme-B, and Site-1 proteases. The ADAM proteases may be selected from ADAM8, ADAM9, ADAM10, ADAM12, ADAM15, ADAM17, ADAM19, ADAM20, ADAM21, ADAM28, ADAM30, and ADAM33. The part-time sheddases may be selected from: Meprin B, MT-MMPs (Membrane Type Matrix Metalloproteases), Pro-protein convertases, transmembrane serine proteases, Matrix Metalloproteases, Legumain and Cathepsin S and L, wherein the Meprin B is a membrane-anchored type 1 metalloprotease, and wherein the MT-MMPs are membrane-anchored type 1 or GPI-anchored, and include MT1-MMP, MT2-MMP, MT3-MMP, MT4-MMP, MT5-MMP, and MT6-MMP. The Pro-protein convertases may be selected from PCSK1/3, KCSK2, Furin, PCSK4, PSCK5/6, PACE4, PCSK7, and PCSK9. The transmembrane serine proteases may be selected from membrane-anchored type II proteases, including, for example, Matriptase, Matriptase-2, Matriptase-3, Polymerase-1, Corin, Hepsin, TMPRSS2, TMPRSS3, TMPRSS4, MSPL, Spinesin, Enteropeptidase, HAT, DESCL1, TMPRSS11A, HAT-like 4 and HAT-like 5. The Matrix Metalloproteases may be soluble proteases and may include one or more of MMP1, MMP2, MMP3, MMP4, MMP5, MMP6, MMP7, MMP8, MMP9, MMP10, MMP11, MMP12, MMP13, MMP14, MMP15, MMP16, MMP17, MMP18, MMP19, MMP20, MMP21, MMP21, MMP23A-B, MMP24, MMP25, MMP26, MMP27, and MMP28. In certain embodiments, the Matrix Metalloproteases may be selected from MMP2, MMP9, and/or MMP25. For example, the MMP2 may be selected from SEQ ID NOS: 2-6, the MMP9 may be selected from SEQ ID NOS: 7-8, and the MMP25 may be selected from SEQ ID NOS: 9-17, 18 or 155, 19-29.

Also contemplated herein is a method as above, wherein the transmembrane domain of the first and/or second transmembrane region comprises a transmembrane domain derived from one or more of CD63, CD9, CD81, CD28, CD4, CD8, CD34, CD69, CD19, CD20, Integrins, TNFSF14, TNR1A, Aquaporins, NOTCH, NgR1, NRG1, GPi anchor, EGFR, and Rhodopsin.

Also contemplated herein is a method as above, wherein cleavage at one or more of the cleavage sites occurs by endogenous sheddases.

Also contemplated herein is a method as above, wherein the peptide delivery system further comprises a sheddase, and cleavage occurs at one or more of the cleavage sites by the peptide delivery system sheddase.

Also contemplated herein is a method as above, wherein an intracellular or extracellular domain of the exogenous polypeptide is selected from an intracellular or extracellular domain from one or more of the following: OX-40, 4-1BB/CD137, CD2, CD7, CD27, CD30, B7-1 (CD80), B7-2 (CD86), PDL-1, Programmed Death-I (PD-1), Inducible T cell Co-Stimulator (ICOS), lymphocyte Function-associated Antigen-I (LFA-1 (CDI la/CD 18), CD3 gamma, CD3 delta, CD3 epsilon, CD247, CD276 (B7-H3), LIGHT, (TNFSF14), NKG2C, lg alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), The costimulatory ligand (PD-L2, 4-1 BBL, OX40L, Inducible Co-Stimulatory Ligand (ICOS-L), Inter-Cellular Adhesion Molecule (ICAM, CD30L, CD70, CD83, HLA-G, MICA, Ml CB, HVEM, lymphotoxin b receptor, 3/TR6, ILT3, ILT4), activating NK cell receptors, BTLA, a Toll ligand receptor, ICAM-1, B7-H3, CDS, ICAM-1, GITR, BAFFR, LIGHT, HVEM (LIGHTR), KIRDS2, NKp80 (KLRFI), NKp44, NKp30, NKp46, CD19, CD8 alpha, CD8 beta, IL-2R beta, IL-2R gamma, IL-7R alpha, ITGA4, VLAl, CD49a, ITGA4, CD49D, ITGA6, VLA-6, CD49f, ITGAD, CDl Id, ITGAE, CD103, ITGAL, CDl la, LFA-1, IT GAM, CDl lb, ITGAX, CDl le, ITGBI, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD 160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

Also contemplated herein is a method as above, wherein the targeting moiety binds BCMA, MUC16 (also known as CA125), EGFR, EGFRvIII, MUCI, Flt-3, WT-1, CD38, CD70, CD90, CD133, MHC-WTI, TSPANI0, MHC-PRAME, MHC-NY-ESOI, HER2 (ERBB2), CA-IX (Carbonic anhydrase IX), LIVI, ADAMI0, CHRNA2, LeY, NKG2D, CSI, CD44v6, CD24, LGR5, ALDH, ALDH1, CD54, Sca1, CD271, CD123, CD36, CD109, CD110, CD71-negative, CCA, ABCG2, Claudin-18.2 (Claudin-18A2, or Claudinl8 isoform 2), PSCA, DLL3 (Delta-like protein 3, Drosophila Delta homolog 3, Delta3), Mud 7 (Mucinl 7, Muc3, Muc3), FAP alpha (Fibroblast Activation Protein alpha), Ly6G6D (Lymphocyte antigen 6 complex locus protein G6d, c6orf23, G6D, MEGTI, NG25), PSMA, MSLN, or RNF43 (E3 ubiquitin-protein ligase RNF43, RING finger protein 43). BAFF, C242 antigen, disialoganglioside (GD2), 4-IBB, 5T4, CD22, CD221, CD23 (IgE receptor), CD28, CD30 (TNFRSF8), CD33, CD4, CD40, CD44v6, CD44 CD51, CD52, CD56, CD74, CEA, CNT0888, CTLA-4, DR5, EpCAM, FAP, fibronectin extradomain-B, folate receptor 1, GD3 ganglioside, glycoprotein 75, GPNMB, HER2/neu, HGF, human scatter factor receptor kinase, IGF-1 receptor, IGF-I, IgGI, LI-CAM, integrin α5β1, integrin αvβ, legumain, MORAb-009, MS4A1, MUCI, mucin CanAg, C-MET, CCR4, CD152, CD10, CD19, CD20, CD200, N-glycolylneuraminic acid, NPC-IC, PDGF-R a, PDL192, phosphatidylserine, tumor antigen CTAA16.88, VEGF-A, VEGFR-1, VEGFR2, vimentin, RANKL, RON, ROR1, SCH900105, SDC1, SLAMF7, TAG-72, tenascin C, TGF-β, TRAIL-R1, TRAIL-R2, folic acid receptor, transferrin receptors, and any combination thereof.

Also contemplated herein is a method as above, wherein the first and/or second transmembrane region comprises (i) an intracellular region comprising an intracellular region sequence from 41BB, ICOS, or CD3ζ, and (ii) a GrzB cleavage site; and wherein the action of kinases on the intracellular region sequence from 41BB, ICOS, or CD3ζ induces the release of the GrzB allowing it to be transported out of the cell and cleave GrzB cleavage sites, thereby releasing the peptide payload, the peptide payload optionally comprising a bispecific antibody.

Also contemplated herein is a method as above, wherein the second transmembrane region comprises a transmembrane domain from a NOTCH protein and a cleavage site inside the cell membrane and cleavage at the cleavage site inside the cell membrane releases a functional domain in the intracellular milieu.

Also contemplated herein is a method as above, wherein the peptide payload comprises: a cytokine or chemokine consisting a fraction or derivative of a sulfated xylan, which is an antagonist of a ligand selected from the list comprising IL-la, IL-1B, IL-2, IL-3, IL-4, IL-5, IL-6, IL-7, IL-8, IL-9, IL-10, IL-11, IL-12, IL-3, IL-14, IL-15 or IL-17 family of cytokines including IL-25, interferon, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, PDGF, SCF, TGFa, TGFB, TNFa, TNFB, TPO, VEGF, GH, NGF, NT3, NT4, NT5, NT6, NT7, Oncostatin M (OSM), insulin, MCP-1, MCP-2, MCP-3, MCP-4, MCP-5, a member of the MIP-1 family including MIP-la, MIP-2, eotaxin (eotaxin-1, -2 or -3), PBP (platelet basic protein), SDF-1, PBSF, PF4, RANTES, elastase; an enzyme of the cathepsin family, a cell adhesion molecules such as PECAM-1, a soluble or cell- or virus-bound receptor, cytokine-induced neutrophil chemoattractant (KC), TNF-α and IFN-g), and other soluble mediators of inflammation, such as reactive oxygen species and nitric oxide, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, thrombin, urokinase, a vaccine or any combination thereof.

Also contemplated herein is a method as above, wherein the exogenous polypeptide further comprises at least one linker, which at least one linker is optionally positioned between the cleavage site present in the first transmembrane region and a first binding moiety (e.g., anti-CD3 VHH domain), between a cleavage site present in the second transmembrane region and a second binding moiety (e.g., anti-TAA VHH domain), and/or between the first and second binding moieties.

Also contemplated herein is a method as above, wherein the linker is 2-128 amino acids in length, or preferably 4-20 amino acids in length.

Also contemplated herein is a method as above, wherein the linker is selected from Table 4.

Also contemplated herein is a method as above, wherein the exogenous polypeptide can also be configure for sorting specifically into extracellular vesicles (EVs).

Also contemplated herein is a method as above, wherein the peptide payload comprises two or more bispecific engagers, and the bispecific engagers are released intracellularly or extracellularly.

Also contemplated herein is a method as above, wherein the peptide delivery system comprises sheddase cleavage sites, and each cleavage site can be the same or different from any of the other cleavage sites.

Also contemplated herein is a method as above, wherein the peptide delivery system comprises and intracellular domain, and the intracellular domain comprises a stalk domain of CD45 phosphatase.

Also contemplated herein is a method as above, wherein the peptide delivery system comprises a single domain antibody or single chain of an antibody which binds a self-associated antigen (SAA) to bring the SAA in proximity, e.g., in cis or in trans.

Also contemplated herein is a method as above, which comprises use of a peptide delivery system comprising a single domain antibody or single chain of an antibody which binds the self-associated antigen (SAA), and which further comprises a CD45 phosphatase- or an NKG2A-binding moiety, which activates an inhibitory signal in the target cell.

Also contemplated herein is a method as above, wherein the peptide payload is any peptide that serves a function within the body is a potential therapeutic, including hormones, cytokines, chemokines, pro-drugs, and antibodies in various formats, such as chimeric antigen receptors (CARs), bi-specific killer cell engager (BIKE), tri-specific killer cell engager (TRIKE), etc.

Also contemplated herein is a method as above, wherein the peptide delivery system is selected from: CD63-MMP2-a-CD16-a-CD33-Myc-MMP9-CD63-GPi (SEQ ID NO: 124), CD63-MMP2-a-CD16-a-CD33-Myc-MMP9-CytCD63-EV Motif (SEQ ID NO: 155), CD63-MMP2-a-CD16-a-CD33-Myc-MMP9-CytCD63 (SEQ ID NO: 36 or 188), TNFSF14-a-CD16-a-CD33-Myc-TNR1A-ADAM17-a-CD38-41BB (SEQ ID NO: 54 or 366 or 367), CD63-GrzB-a-CD16-a-CD33-Myc-GrzB-a-CD38-CD28-CD3z (SEQ ID NO: 71 or 252), CD63-MMP9-2L-TNFa-Myc-MMP9-CD63-Cyt (SEQ ID NO: 88 or 295), CD63-MMP9-2L-TNFa-Myc-MMP9-CD63-D-Cyt (Lacks EVs sorting motif) (SEQ ID NO: 103 or 311), CD63-DL1-MMP2-2L-TNFa-Myc-MMP2-CD63-Cyt (SEQ ID NO: 104 or 368), CD63-MMP9-2L-TNFa-Myc-MMP9-aCD19CAR (SEQ ID NO: 105 or 369), CD63-MMP2-2L-TNFa-Myc-MMP2-CD63-D-Cyt (SEQ ID NO: 106 or 370 or 371), CD63-MMP2-2L-TNFa-Myc-MMP2-aCD19CAR (SEQ ID NO: 107 or 372 or 373), CD69cyt-TMII-TNFa-Myctag-CD28TM-cyt (SEQ ID NO: 119 or 370), CD69cyt-TMII-MMP9-TNFa-Myc-MMP9-CD28TM-cyt (SEQ ID NO: 120 or 377), CD69Cyt-TM-GrzB-TNFa-Myc-GrzB-a-CD19sc-Stalk-CD28-CD3z (SEQ ID NO: 107 or 372 or 373), CD69Cyt-TM-GrzB-nLuc-Myc-GrzB-CD28-CD3z (Delta sc-GAGE B) (SEQ ID NO: 108 or 374), CD69Cyt-TM-nLuc-Myc-a-CD19sc-Stalk-CD28-CD3z (Delta GrzB, GAGE A) (SEQ ID NO: 109 or 375), CD69Cyt-TM-GrzB, (Original)-nLuc-Myc-GrzB(Original)-a-CD19sc-Stalk-CD28-CD3z (SEQ ID NO: 358 or 382), CD69Cyt-TM-GrzB(IEPD)-nLuc-Myc-GrzB(IEPD)-a-CD19sc-Stalk-CD28-CD3z (SEQ ID NO: 110 or 383), CD69Cyt-TM-GrzB(SASA)-nLuc-Myc-, GrzB(SASA)-a-CD19sc-Stalk-CD28-CD3z (SEQ ID NO: 111 or 376), or CD69Cyt-TM-GrzB(ADKG)-nLuc-Myc-GrzB(ADKG)-a-CD19sc-Stalk-CD28-CD3z (SEQ ID NO: 112 or 384).

Also contemplated herein is a method as above, wherein the lipid-comprising vesicle comprises a cell, and the cell is selected from a Hematopoietic Stem Cells (HSC), induced Pluripotent Stem Cell (iPSC) and a derived cell product, an Adoptive T cell, a Dendritic cell (DC), a Natural killer (NK) cell, or any therapeutic immune cell or non-immune cell. In certain embodiments, the immune cell may be autologous or allogeneic.

Also contemplated herein is a method as above, wherein the peptide delivery system is introduced into a cell individually in the form of viral or nonviral vectors, mRNAs, peptides, proteins, antibodies, nanobodies, oligonucleotides, or extracellular vesicles (EVs).

Also contemplated herein is a method as above, wherein the peptide delivery system is delivered using CPPs, micelle, liposomes, nanoparticles, dendrimer, nanotube, electroporation, viral transduction, nucleofection, transfection, cell fusion or microinjection.

It is an object of the invention to deliver a peptide.

It is an object of the invention to target the payload to a specific cell type.

It is an object of the invention to deliver multiple peptides.

It is an object of the invention to introduce the peptide delivery system into any cell that can deliver at least one peptide to a target cell or target locus.

It is an object of the invention to create a single delivery system which can be incorporated into any cell.

### BRIEF DESCRIPTION OF THE DRAWINGS

FIG. 1 is a drawing of a generic embodiment of the invention comprising two transmembrane domains, i.e., a “dispanin”.

FIG. 2 is a drawing of an embodiment of a “dispanin” delivery system comprising, in order, a Type-II transmembrane domain, a first MMP cleavage sequence, a first linker (“Linker-a”), an anti-CD3 VHH domain, a second linker (“Linker-b”), an anti-TAA VHH domain, a third linker (“Linker-c”), a second MMP cleavage sequence, and a GPI anchor.

FIG. 3 shows the amino acid sequence and various domains/motifs of the embodiment shown in FIG. 2.

FIG. 4A is a drawing of a generic embodiment of the invention using a modified tetraspanin protein (CD63) linked to a specific engager (GAGE) (here, TNF-α) and comprising a particular motif, GYEVM, for the EVs specific delivery.

FIG. 4B shows the amino acid sequence and various domains/motifs of the embodiment shown in FIG. 4A.

FIG. 5A shows a generic embodiment of the invention using a modified tetraspanin protein (CD63) without an EV sorting motif.

FIG. 5B shows the amino acid sequence and various domains/motifs of the embodiment shown in FIG. 5A.

FIG. 6 shows an embodiment of the invention using, among other things, a modified transmembrane C-Type lectin protein (CD69).

FIG. 7 shows a generic embodiment of the invention using a modified tetraspanin comprising four different types of transmembrane domains (TMI-TMIV).

FIG. 8 shows a generic embodiment of the invention using a modified tetraspanin.

FIG. 9 is a drawing of a dispanin embodiment of the invention.

FIG. 10 is the amino acid sequence for a CD63-loop-EVs sorting embodiment and shows corresponding domains and motifs of the embodiment shown in FIG. 9.

FIG. 11 is a drawing of a dispanin embodiment of the invention.

FIG. 12 is the amino acid sequence for the embodiment shown in FIG. 11 and shows corresponding domains and motifs.

FIG. 13 is a drawing of a dispanin embodiment of the invention.

FIG. 14 is the amino acid sequence for the embodiment shown in FIG. 13 and shows corresponding domains and motifs.

FIG. 15 is a drawing of an dispanin embodiment of the invention.

FIG. 16 is the amino acid sequence for a CD63-Grn-B-a-CD16-L-a-CD33-Grn-B-CD38CAR embodiment and shows corresponding domains and motifs of the embodiment shown in FIG. 15.

FIG. 17A is a drawing of a dispanin embodiment of the invention encoded by CD69Cyt-TM-II-TNFa-Myc-CD28TM-CYT for delivery of TNFa.

FIG. 17B is the Amino Acid Sequence of CD69Cyt-TM-II-TNFa-Myc-CD28TM-CYT.

FIG. 18A is a drawing showing an embodiment of the present invention encoded by CD69cyt-TMII-9-TNFa-Myctag-9-CD28TM-cyt containing two MMP9 cleavage sites.

FIG. 18B is the amino acid sequence of CD69cyt-TMII-9-TNFa-Myctag-9-CD28TM-cyt.

FIG. 19 is a drawing of an embodiment of the invention with αTAA-αSAA as a payload that can be delivered upon cleavage.

FIG. 20 is a drawing showing the plasmid transmembrane domains, protease motifs and payloads of various embodiments disclosed in this application.

FIG. 21 is a drawing showing the experimental setup/model of an embodiment of the invention shown in this application.

FIGS. 22A, 22B, and 22C, show three options for experimental setups/models involving TNFa and measurement of NF-κB Luciferase activity.

FIG. 23A is a drawing showing the experimental setup/model of an embodiment disclosed in this application in which Granzyme B results in release of TNFa payload from the surface of an NK cell.

FIG. 23B is a drawing showing the experimental setup/model of an embodiment disclosed in this application in which Granzyme B results in release of TNFa payload from the surface of a K562 cell.

FIG. 24 is a graph showing that the TNFa payload is functional upon direct cell-to-cell contact with the HEK293-NF-κB-Luc reporter cells.

FIG. 25 is a graph showing the delivery of TNFa payload after cleavage by MMP9 Note: co-transfection of GAGE and MMP9. We show EVs depleted vs. not depleted to control for transfer of the MMPs through EVs.

FIG. 26 is a graph showing the delivery of TNFa payload mainly into EVs.

FIG. 27 is a graph showing the delivery of TNFa payload after cleavage by MMP2. In this experiment, supernatant from HEK293 cells expressing GAGE constructs was utilized.

FIG. 28 is a graph showing the delivery of TNFa payload mainly into EVs. Cleavage is again by MMP2. Note: EVs depleted vs. not depleted.

FIG. 29 is a graph showing the differential CD63 and CD69 transmembrane GAGE expression on HEK293 cells.

FIG. 30 is a graph showing the results of an in vitro responsive assay to TNFa payload delivery upon MMP9 cleavage and NF-κB activation.

FIG. 31 shows the percentage of K562 cells positive for surface TNFa expression 3 days after transduction.

FIG. 32 is a graph showing the delivery of TNFa payload from K562 cells upon cleavage by Granzyme B (GrzB) secreted by NK92 cells.

FIG. 33 shows the percentage of K562 cells positive for surface TNFa and aCD19/CAR expression 6 days after sorting the cells. (Surface expression of GAGE Nanoluciferase in K562 transduced cells (MOI 6)).

FIGS. 34A and 34B are graphs showing the release of NanoLuciferase (nLuc) payload from K562 cells upon cleavage by Granzyme B (GrzB) secreted by NK92 cells. E:T 3:1, 4 hr incubation.

FIG. 35 shows the percentage of KHYG-1 cells positive for surface NanoLuciferase (nLuc) expression 3 days after transduction.

FIG. 36 is a data graph showing the release of NanoLuciferase (nLuc) payload from KHYG-1 cells upon cleavage by Granzyme B (GrzB) after 1-3 hrs co-culturing with Nalm-6 cells. Different time point after normalization to MFI.

FIGS. 37A and 37B are graphs showing the percentage and MFI of KHYG-1 cells positive for surface NanoLuciferase 6 days after sorting the cells.

FIG. 38 is a graph showing the release of NanoLuciferase (nLuc) payload from KHYG-1 cells upon cleavage by Granzyme B (GrzB) after 4 and 6 hrs co-culturing with K562 cells.

FIGS. 39A and 39B are graphs showing the percentage and MFI of NK92 cells positive for surface NanoLuciferase 10 days after transduction of NK92 cells.

FIG. 40 is a graph showing the release of NanoLuciferase (nLuc) payload from NK92 cells upon cleavage by Granzyme B (GrzB) after 4 hrs co-culturing with Raji cells.

FIG. 41 is a graph showing the percentage of NK92 cells positive for surface NanoLuciferase 3 days after transduction of NK92 cells (unsorted cells) and sorted cells.

FIG. 42 is a graph showing the release of NanoLuciferase (nLuc) payload from NK92 cells upon cleavage by Caspase 3 after 6 and 24 hrs co-culturing with K562 cells.

### DETAILED DESCRIPTION OF THE INVENTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Applicant also contemplates ranges derived from data points and express ranges disclosed herein.

Each and every reference cited herein is incorporated by reference in its entirety.

Contemplated herein is a peptide or protein cargo delivery system having directed delivery and/or site-directed release and site-directed activation of the cargo. The cargo is targeted to its intended site and/or activated when it reaches the target. This approach enables increasing the local or target site dose while limiting the number of off-target side effects. The present peptide or protein delivery system may include certain safety and specificity mechanisms, so the biological is only released in high doses at the target site, but not in other tissues (e.g., tissue where antigens for a specific antibody-derived biological may also be expressed). In addition to making these peptide-based drugs safer, and more broadly applicable, our system could reduce treatment costs and be customized for more personalized or patient population-specific uses. Furthermore, delivery systems and methods as disclosed herein enable the equivalent of more frequent dosing without needing to administer the peptide repetitively. For example, delivery via cargo-carrying cells that store the peptides or proteins to be delivered may provide an in vivo reservoir of inactive peptides or proteins (e.g., prodrugs).

Mammalian cells have the hardware required to shed many cell membrane-bound proteins. Shedding is a controlled, irreversible posttranslational process performed by families of proteolytic enzymes, among them sheddases, which are further described below. Cell biology allows cells to control the expression levels and functions of the substrate proteins and their location on the cell surface.

Proteolytic cleavage substrates include Glycosylphosphatidylinositol (GPi) anchored proteins, membrane proteins with a single, two, three, four, or more transmembrane domains, intracellular domains, extracellular domains in the extracellular matrix or even in neighboring cells. Membrane proteins of the delivery systems contemplated herein having 2 transmembrane domains are referred to herein as “dispanins”. Membrane proteins of the delivery systems contemplated herein having 4 transmembrane domains are referred to herein as “tetraspanins.” Cleavage can target the juxta-membrane or distal regions of the membrane-bound proteins and can result in the release of protein domains inside and/or outside the cells (ectodomain and/or endodomain release).

We have designed a cell-based delivery system that allows the delivery and release of at least one peptide or protein payload directly at a desired site of action. The payloads are characteristically anchored to the cell through transmembrane domains (TMD). A TMD is a part of a protein molecule that spans across the cell membrane and typically consists of one or more alpha-helices or beta-sheets that cross the lipid bilayer of the membrane, anchoring the protein in place and allowing it to interact with molecules on both sides of the membrane. Many important proteins have transmembrane domains, including receptors, transporters, and enzymes. These domains are often involved in the transduction of signals across the membrane or in the transport of molecules into or out of the cell.

Delivery systems as contemplated herein consist of or comprise single or multi-pass transmembrane (TM) proteins embedded into the membrane of a vehicle (e.g., cell, extracellular vesicle, or other lipid-containing particle or composition). Additional motifs may be present that regulate the delivery/release of the payload in the required milieu. Thus, instead of direct systemic biologics injections, we aimed to transfuse engineered cells or EVs configured to deliver the cargo straight to the site where it is needed and release it at the right time.

The delivery systems contemplated herein are applicable to any cell type and can be used to deliver any peptide of interest. Suitable cell types include but are not restricted to Hematopoietic Stem Cells (HSCs), induced Pluripotent Stem Cells (iPSCs) and their derived cell products, Adoptive T cell, Dendritic cell (DC), Natural killer cells (NK), NKT cell, or any therapeutic immune or non-immune cells. Such cells may be utilized in autologous or allogenic therapy.

In one embodiment we have modified NK cells to produce and deliver a bi-specific engager (GAGE) in the tumor microenvironment (TME). The TME is composed of cellular and acellular partners, which can regulate various aspects of oncogenesis, including tumor initiation, invasion, and metastasis (The updated landscape of tumor microenvironment and drug repurposing. Ming-Zhu Jin & Wei-Lin Jin. Signal Transduction and Targeted Therapy volume 5, Article number: 166 (2020)). This delivery is further regulated and can be induced upon either NK cell activation (by activation-induced cleavage) or the tumor milieu (by cleavage upon encountering proteases enriched specifically in the TME. In addition, such an activation-stimulated release may lead to NK cell expansion and activation depending on the tumor antigen availability. In embodiments delivery systems as described herein include next-generation cancer immunotherapy systems with an augmented capacity to produce and release the payload under the desired conditions and at specific sites.

The sheddases which can be used for this payload delivery system are not particularly limited and further described below. For example, the sheddases may be selected from matrix metalloproteinases (MMPs) and Granzyme-B (GrzB), which allow the milieu-specific cargo release. Also, a model based on the ADAM family of MMPs, and the serine-protease Granzyme-B have been presented. In these models, additional functions like tumor targeting, expansion, and/or activation were modulated by adding extracellular and/or intracellular domains. These additional domains can include anything from extracellular cytokines, drugs or pro-drugs, chimeric antigen receptor (CAR)-like or dual antigen receptor (DAR)-like domains or intracellular modulators, inhibitors, signaling domain-containing proteins, etc.

Moreover, the system has been devised for passive release in the tumor milieu depending on the specific MMPs released by the cancer cells or active release induced by the activation of producer cells due to receptor engagement.

These systems can also be designed in such a way that they are sorted into specific subcellular compartments. We have designed a protein molecule that can be actively sorted into extracellular vesicles (EVs), such as exosomes. The cell releases these exosomes in huge quantities and can release the payload upon cleavage by proteases within the TME or other target sites. These EVs can also be harvested from in-vitro culture and given to patients as an off-the-shelf therapeutic.

In its simplest form, the present invention comprises a prodrug form of a peptide comprising: a first and a second transmembrane region; the first transmembrane region comprising at least one transmembrane unit connected to at least one cleavage site; the second transmembrane region consisting of one transmembrane unit connected to at least one cleavage site; optionally the delivery system may include a targeting moiety for targeting the prodrug to a target cell; wherein when the prodrug contacts the target cell, sheddases cleave the cleavage sites releasing the peptide.

We mainly used a set of TMDs in this multi-pass payload delivery system The N-terminus of the TMDs consist of a type II TMD harboring linker/payload that is connected to a type I TMD domain at the C-terminus. TMDs are preferably at least one or more of CD63, CD9, CD81, CD28, CD4, CD8, CD34, CD69, CD19, CD20, integrins, TNFSF14, TNR1A, aquaporins, NOTCH, NgR1, NRG1, GPi anchor, EGFR, Rhodopsin or any relevant TMDs that provide similar function to the system can be used for the expression of our invention.

Optionally, the second transmembrane region has an intracellular region comprising 41BB, ICOS, and CD3ζ and an MMP/GrzB that cleaves the MMP/GrzB cleavage sites on the exterior of the cell, wherein the action of phosphatases on 41BB, ICOS, and CD3ζ cleaves and activates the MMP/GrzB allowing it to be transported out of the cell and cleave the MMP/GrzB cleavage sites thereby releasing the bispecific antibody/payloads. Optionally, the first and second transmembrane regions are embedded with a NOTCH protein-dependent pulling system with a secretase cleavage site inside the cell membrane with multiple cargos/payloads connected to several cleavage sites on the exterior or interior of the cell. Optionally, the system can be embedded by recruiting the self-associated antigen via adding a single-chain (a-SAA).

The engineered system may be introduced into the cell individually in the form of viral or nonviral vectors, mRNAs, peptides, proteins, antibodies, nanobodies, oligonucleotides, or extracellular vesicles. (EVs), which can be delivered using CPPs, micelle, liposomes, nanoparticles, dendrimer, nanotube, electroporation, viral transduction, nucleofection, transfection, cell fusion or microinjection. Viral vectors may contain the gene/s encoding the GAGE and viral particles thus produced may carry the gene(s). When viruses infect the cells, gene/s get stably integrated and express the protein encoded. Non-viral vectors may be in the form plasmids carrying the GAGE gene/s. Cells are transfected and this results in the expression of the gene/s encoding the GAGE protein/s. Cells can be selected for stable expression. It could be envisioned that one of the gene delivery strategies that could be utilized is site-specific integration utilizing strategies such as CRISPR (e.g., Cas9, Cpf1, or other Cas effector protein complex) or transposon-transposase strategy. The CRISPR Cas system can be introduced through electroporation or via LNPs and can integrate the gene coding for the GAGE.

The present invention is a peptide delivery system having a site-directed release and activation of the peptide. Site-directed means that the peptide-producing cell or EV comprising the peptide delivery system is targeted to its intended site. The cell or EV is then activated to release the peptide at the target. This approach enables increasing the delivered dose while limiting the number of off-target side effects. The present peptide delivery system includes certain safety and specificity mechanisms so that the biological/payload is only released in high doses at the target site, but not in other tissues where antigens for the specific biological (e.g., antibody-derived biological) may also be expressed. In addition to making these peptide-based drugs safer, more applicable, and more specific, our system could reduce treatment costs. Furthermore, this invention enables the equivalent of more frequent dosing without needing to administer the peptide repetitively because delivery via cargo cells which will produce and store the peptides, provides an in vivo reservoir of inactive peptides.

Mammalian cells have the hardware required to shed many cells' membrane-bound proteins. Shedding is a controlled, irreversible posttranslational modification performed by families of proteolytic enzymes called proteases or sheddases. Cell biology allows cells to control the expression levels and functions of the substrate proteins and their location on the cell surface. Sheddases as a class are known and comprised of full-time sheddases and part-time sheddases. Lichtenthaler SF, Lemberg MK, Fluhrer R. Proteolytic ectodomain shedding of membrane proteins in mammals-hardware, concepts, and recent developments. EMBO J. 2018; 37(15): e99456. doi: 10.15252/embj.201899456.

Full-time sheddases include ADAM proteases, BACE proteases, serine-protease Granzyme-B and Site-1 proteases. ADAM proteases (metalloproteases) are membrane-anchored type 1 protease and include ADAM8, ADAM9, ADAM10, ADAM12, ADAM15, ADAM17, ADAM19, ADAM20, ADAM21, ADAM28, ADAM30, and ADAM33. The BACE (aspartyl) proteases are also membrane-anchored type1 proteases which include BACE1 and BACE2. The Site-1 (serine) proteases are membrane-anchored type 1 protease, including SKI-a or S1P.

Part-Time sheddases include Meprin B, MT-MMPs (Membrane Type Matrix Metalloproteases), Pro-protein convertases, Transmembrane serine proteases, Matrix Metalloproteases, Legumain and Cathepsin S and L. Meprin B is a membrane-anchored type 1 metalloprotease. MT-MMPs are membrane-anchored type 1 or GPI-anchored and include MT1-MMP, MT2-MMP, MT3-MMP, MT4-MMP, MT5-MMP, and MT6-MMP. The Pro-protein convertases are membrane-anchored type 1 or soluble proteases and include PCSK1/3, KCSK2, furin, PCSK4, PSCK5/6, PACE4, PCSK7, and PCSK9. Transmembrane serine proteases are membrane-anchored type II proteases and include Matriptase, Matriptase-2, Matriptase-3, Polymerase-1, Corin, Hepsin, TMPRSS2, TMPRSS3, TMPRSS4, MSPL, Spinesin, Enteropeptidase, HAT, DESCL1, TMPRSS11A, HAT-like 4 and HAT-like 5. The Matrix Metalloproteases are soluble proteases including MMP1, MMP2, MMP3, MMP4, MMP5, MMP6, MMP7, MMP8, MMP9, MMP10, MMP11, MMP12, MMP13, MMP14, MMP15, MMP16, MMP17, MMP18, MMP19, MMP20, MMP21, MMP21, MMP 23A-B, MMP24, MMP25, MMP26, MMP27 and MMP28.

For evaluation, the substrate targets with the highest cleavage efficiency for one MMP and lower than 4000 k (obs) for the other MMPs were considered “specific” substrate sequences for that particular MMP. Substrate hexapeptide sequences are sandwiched between constant regions from M13 phage gene III protein containing FLAG epitope:

(SEQ ID NO: 365)
ADVGGTDYKDDDDKPGGRPTWPSSGGSGXXXXXXTASGAET.

Tables 1-3 below show the selected MMP substrates and their binding affinity sequences specific for MMP2, MMP9, and MMP25.

TABLE 1
MMP2 Specific Target Substrates* k(obs) (M⁻¹sec⁻¹)

| SEQ | | | | | | | | | |
|---|---|---|---|---|---|---|---|---|---|
| ID | | | | | | | | | |
| NO: | Sequence | MMP2 | MMP9 | MMP14 | MMP15 | MMP16 | MMP24 | MMP17 | MMP25 |
| 2 | x-LYSTLR | 12792 | 1247 | 991 | 1812 | 1847 | 945 | 1108 | 3610 |
| 3 | PAS-VRN | 12792 | 2853 | 3 | 482 | 3 | 243 | 17 | 2674 |
| 4 | RPSP-FW | 12792 | 3850 | 2 | 6 | 8 | 4 | 6 | 4 |
| 5 | PAY-WRT | 12792 | 1593 | 32 | 1116 | 489 | 286 | 10 | 726 |
| 6 | NRPAS-W | 12792 | 3240 | 1113 | 5 | 2876 | 675 | 347 | 1394 |

TABLE 2
MMP 9 Specific Target Substrates* k(obs) (M⁻¹sec⁻¹)

| SEQ | | | | | | | | | |
|---|---|---|---|---|---|---|---|---|---|
| ID | | | | | | | | | |
| NO: | Sequence | MMP2 | MMP9 | MMP14 | MMP15 | MMP16 | MMP24 | MMP17 | MMP25 |
| 7 | VRA-VYS | 3097 | 12792 | 429 | 1 | 910 | 112 | 6 | 6 |
| 8 | YY-LRA | 876 | 12792 | 436 | 2434 | 417 | 1 | 1 | 13 |

TABLE 3
MMP 25 Specific Target Substrates* k(obs) (M⁻¹sec⁻¹)

| SEQ | | | | | | | | | |
|---|---|---|---|---|---|---|---|---|---|
| ID | | | | | | | | | |
| NO: | Sequence | MMP2 | MMP9 | MMP14 | MMP15 | MMP16 | MMP24 | MMP17 | MMP25 |
| 9 | PLK-ITR | 1 | 2800 | 1 | 192 | 23 | 1 | 1183 | 12792 |
| 10 | VSF-LQQ | 1392 | 5 | 43 | 3554 | 1004 | 1 | 2493 | 12792 |
| 11 | TVLNS-L | 3402 | 6 | 1229 | 1777 | 1552 | 2 | 2323 | 12792 |
| 12 | NF- | 1183 | 481 | 63 | 676 | 717 | 297 | 4 | 12792 |
| | LRKM | | | | | | | | |
| 13 | NLA- | 4 | 0 | 750 | 3738 | 2556 | 1330 | 850 | 12792 |
| | LKQ | | | | | | | | |
| 14 | LA- | 98 | 3 | 398 | 2933 | 2073 | 645 | 1070 | 12792 |
| | LKMG | | | | | | | | |
| 15 | QQMS- | 69 | 1 | 287 | 1923 | 1165 | 602 | 3087 | 12792 |
| | LR | | | | | | | | |
| 16 | M- | 4 | 6 | 1 | 1 | 2 | 5 | 2255 | 12792 |
| | LKLQP | | | | | | | | |
| 17 | SWS-LIL | 9 | 5 | 208 | 3437 | 2574 | 1606 | 2926 | 12792 |
| 18 | A-LQRLP | 99 | 2 | 1251 | 463 | 1563 | 421 | 1993 | 12792 |
| 19 | YYGE- | 1001 | 41 | 618 | 302 | 99 | 27 | 3537 | 12792 |
| | LL | | | | | | | | |
| 20 | LE- | 2 | 234 | 0 | 533 | 1 | 45 | 2165 | 12792 |
| | LRVN | | | | | | | | |
| 21 | SGVNS- | 733 | 0 | 2 | 115 | 901 | 396 | 1782 | 12792 |
| | LR | | | | | | | | |
| 22 | M- | 2 | 3 | 1 | 138 | 0 | 48 | 1882 | 12792 |
| | LLMKQ | | | | | | | | |
| 23 | VTVIA-L | 1698 | 960 | 8 | 1815 | 233 | 407 | 2774 | 12792 |
| 24 | ALD- | 3 | 1 | 0 | 691 | 1719 | 508 | 3973 | 12792 |
| | LKR | | | | | | | | |
| 25 | VGA- | 841 | 62 | 54 | 125 | 168 | 1 | 3265 | 12792 |
| | LNK | | | | | | | | |
| 26 | N- | 146 | 3 | 169 | 526 | 1532 | 1898 | 350 | 12792 |
| | LVRVR | | | | | | | | |
| 27 | TTSLR-L | 2 | 81 | 19 | 1407 | 571 | 158 | 1695 | 12792 |
| 28 | PSR-ILM | 1091 | 1491 | 3 | 348 | 0 | 3 | 618 | 12792 |
| 29 | PQA- | 1077 | 531 | 1 | 410 | 735 | 2 | 997 | 12792 |
| | VVK | | | | | | | | |

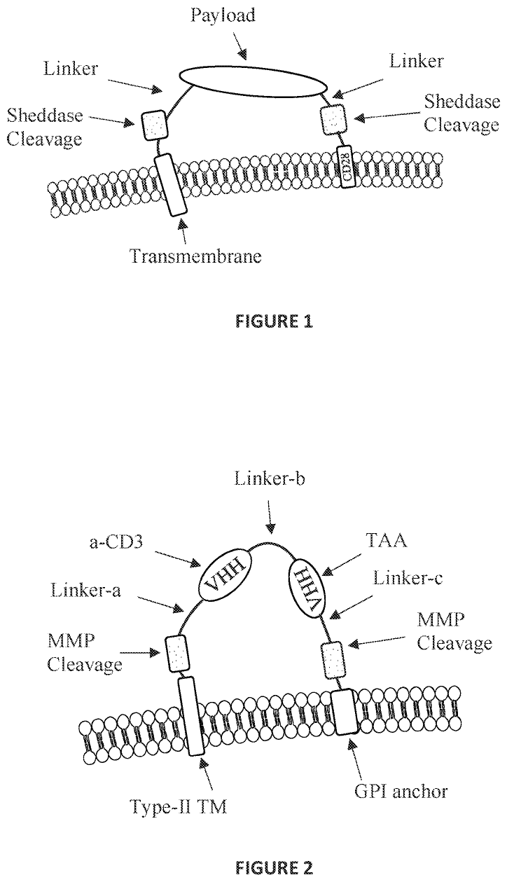

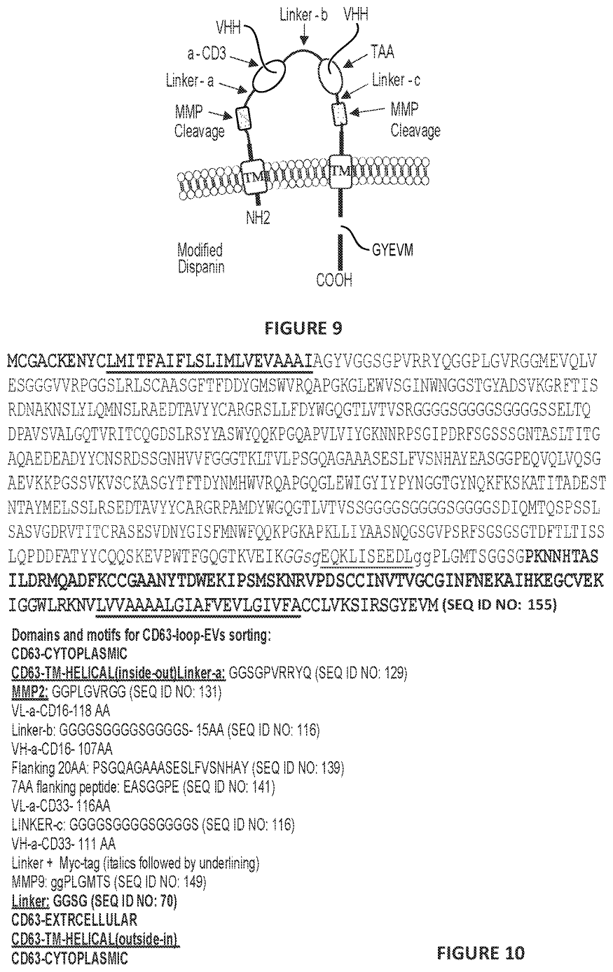

Proteolytic cleavage substrates include Glycosylphosphatidylinositol (GPi) anchored proteins, membrane proteins with a single, two, three, four, or more transmembrane domains, intracellular domains, extracellular domains in the extracellular matrix or even in neighboring cells. Cleavage can target the juxta-membrane or distal regions of the membrane-bound proteins and can result in the release of protein domains inside and outside the cells (ectodomain and/or endodomain release) (FIGS. 1-20).

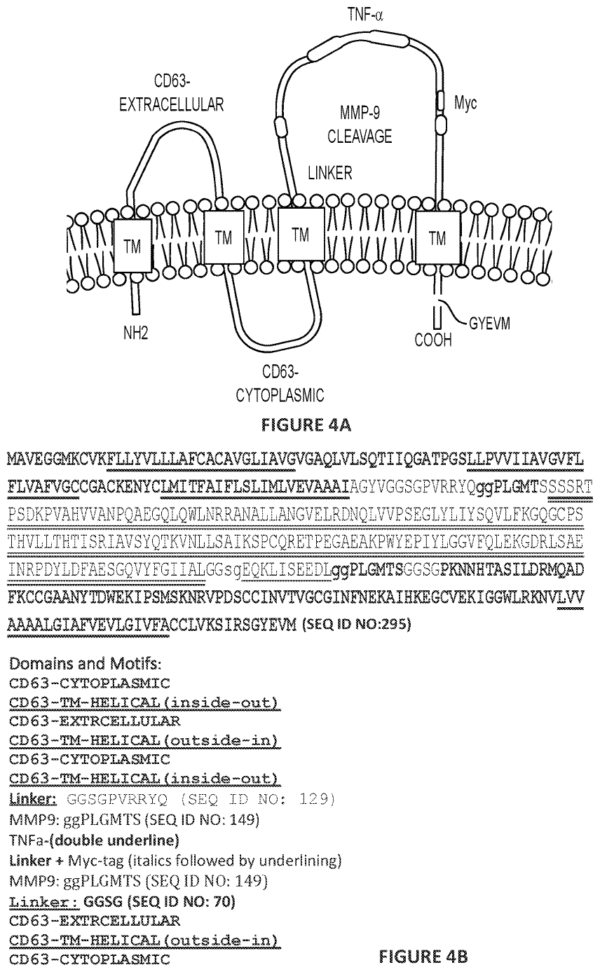

Membrane-bound proteins which cross the plasma membrane once are called “single-pass” molecules and consist of Type I, II, III, and IV members. Type I transmembrane proteins have an N-terminal signal peptide domain that targets them to the endoplasmic reticulum (ER) lumen during synthesis leading to expression in the extracellular space. A stop-transfer anchor sequence keeps Type I transmembrane proteins anchored to the lipid membrane. Although both Type II and III use a signal-anchor sequence, type II targets the ER lumen with its C-terminal domain, while N-terminal domains target type III to the ER lumen. Type IV can be either TV-A, with their N-terminal domains targeted to the cytosol, and IV-B, if their N-terminal domain is targeted to the lumen. The delivery system of the present invention can have one, two, three, four or more transmembrane proteins (see, e.g., FIGS. 4A, 4B, 5A, 5B, 6, 7, 8, 9, etc.).

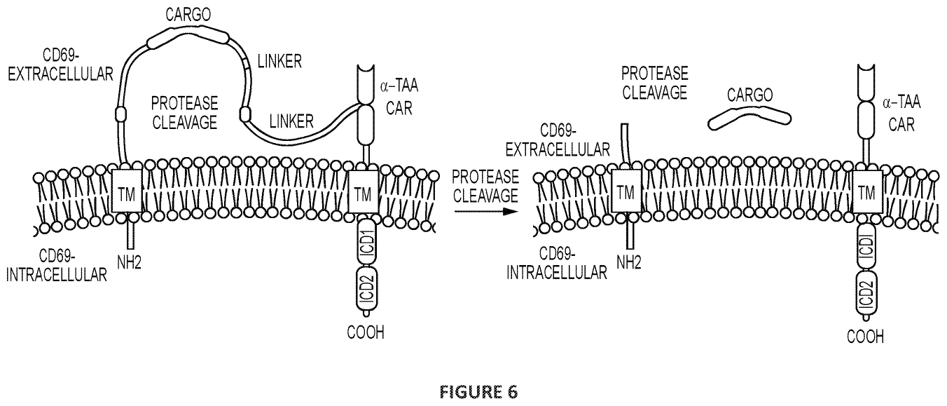

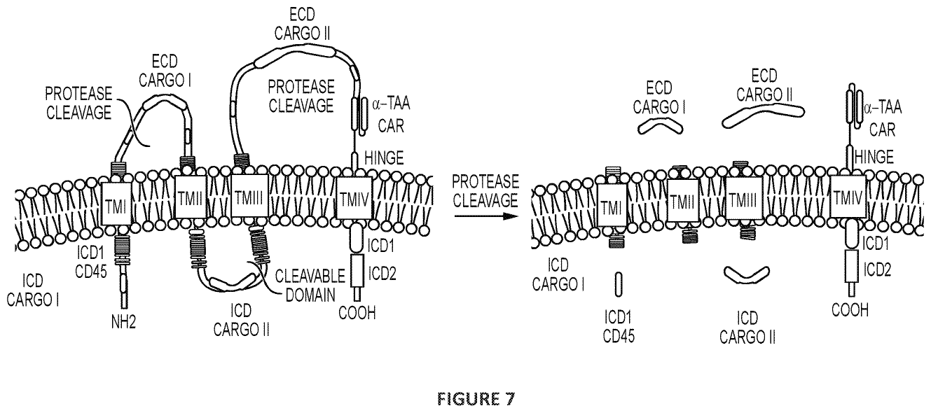

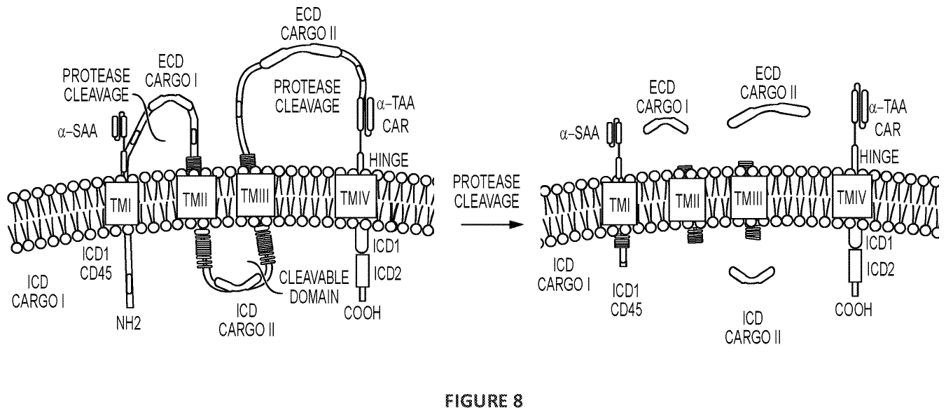

Optionally, the transmembrane regions following the first or second extra cellular loop (ECL) has an intracellular region comprising 41BB, ICOS, and CD3ζ, wherein the action of kinases on 41BB, ICOS, CD28 and CD3ζ activates the engineered cells (NK and T cells) to release their endogenous GrzB allowing it to cleave the GrzB cleavage sites thereby releasing the bispecific antibody/payloads (FIGS. 6, 7, 8, and 15). In NK cells, for example, the CD69 TM is embedded into the membrane with at least two protease cleavage site motifs that regulate the release of the payload in the required milieu. In this model, a single-chain CAR protein is also expressed at N-terminus for a specific delivery. Optionally, the first and second transmembrane regions are embedded with a NOTCH protein-dependent pulling system with a secretase cleavage site inside the cell membrane with multiple cargos/payloads connected to several cleavage sites on the exterior or interior of the cell (FIGS. 7 and 8). In this system, more than two protease cleavage site motifs are inserted with several potential cargo delivery/releases. This system also has the possibility of releasing intracellular payloads.

In addition to the model described in FIG. 7, optionally, the system can be embedded by recruiting the self-associated antigen via adding a single-chain (a-SAA) (FIG. 8). This system is a modification of FIG. 7 with an extra single-chain against the self-associated antigen (a-SAA) connected with the intracellular domain of CD45. “a-SAA” means a single chain antibody or antibody domain directed against or that binds to a self-associated antigen, preferably an scFv or camelid antibody (VHH domain or VHH domain antibody) or single domain antibody. Self-associated antigens include CD45, CD148, or CD43.

Linkers are peptide regions that separate functional units in a fusion protein. Chen X, Zaro J L, Shen WC. Fusion protein linkers: property, design, and functionality. Adv Drug Deliv Rev. 2013; 65(10):1357-1369. doi:10.1016/j.addr.2012.09.039 discloses various considerations for choosing linkers. One of the skill in the art will be able to follow the teachings of this reference and select linkers based on the payload. In certain cases, where polypeptides consisting of various functional domains are needed to be kept at a certain distance, non-cleavable rigid linkers are used. Human-muscle aldolase (HMA) is an example of a rigid linker used to keep the protein domains separated at a set distance. Similarly, flexible, non-cleavable linkers are frequently used in polypeptides harboring multiple functional domains with freedom of movement. These include polyglycine/serine linkers widely used in chimeric proteins. Linkers may be any size needed to perform the function but are preferably from 2 to 128 amino acids in length and most preferably 4-20 amino acids in length. Other contemplated linker lengths include 3-100 amino acids in length, 5-50 amino acids in length, 6-45 amino acids in length, 7-40 amino acids in length, 8-30 amino acids in length, 10-20 amino acids in length, and 5-15 amino acids in length. Table 4 below provides examples of linkers and certain constructs comprising linkers. Linkers as disclosed in iGEM (parts.igem.org/Protein_domains/Linker) are also contemplated.

TABLE 4
Linkers and Certain Constructs Comprising Linkers

| Name | Description | DNA Sequence | Length |
|---|---|---|---|
| BBa | PLrigid EAAAR EAAAR EAAAR | GAAGCTGCTGCAAGAGAAGCTGCAGCTAGGG | 60 |
| J176131 | EAAAR (SEQ ID NO: 30) | AGGCTGCAGCTAGGGAGGCTGCTGCAAGA | |
| | | (SEQ ID NO: 31) | |
| BBa | 2 aa GS linker GS | GGCAGC | 6 |
| J18920 | | | |
| BBa_ | 6 aa [GS]x linker GSGSGS (SEQ ID NO: | GGTAGCGGCAGCGGTAGC (SEQ ID NO: 35) | 18 |
| J18921 | 34) | | |
| BBa_ | 10 aa [GS]x linker GSGSGSGSGS (SEQ | GGTAGCGGCAGCGGTAGCGGTAGCGGCAGC | 30 |
| J18922 | ID NO: 36) | (SEQ ID NO: 37) | |
| BBa_ | 10 aa flexible protein domain | GGTGAAAATTTGTATTTTCAATCTGGTGGT | 30 |
| K105012 | linker GENLYFQSGG (SEQ ID NO: | SEQ ID NO: 39) | |
| | 38) | | |
| BBa_ | 8 aa protein domain linker SACYCELS | TCCGCTTGTTACTGTGAGCTTTCC (SEQ ID | 24 |
| K133132 | (SEQ ID NO: 40) | NO: 41) | |
| BBa_ | Flexible linker 2x (GGGS) GGSGGS | GGTGGTGGTTCTGGTGGTGGTTCT (SEQ ID | 24 |
| K1486003 | (SEQ ID NO: 42) | NO: 43) | |
| BBa_ | flexible linker 2x (GGGGS) | GGTGGTGGTGGTTCTGGTGGTGGTGGTTCT | 30 |
| K1486004 | GGGGSGGGGS (SEQ ID NO: 44) | (SEQ ID NO: 45) | |
| BBa_ | 13 amino acids linker [GGGS GGGGS | GGCGGTGGCTCTGGAGGTGGTGGGTCCGGCG | 39 |
| K1486037 | GGGS] (SEQ ID NO: 46) | GTGGCTCT (SEQ ID NO: 47) | |
| BBa_ | 10 aa linker GGGGSGGGGS (SEQ ID | GGTGGCGGTGGCTCTGGAGGTGGTGGGTCC | 30 |
| K1486053 | NO: 48) | (SEQ ID NO: 49) | |
| BBa_ | Split fluorophore linker; Freiburg | CGACCAGCCTGTAAGATTCCAAATGACCTGA | 51 |
| K157009 | standard RPACKIPNDLKQKVMNH | AGCAGAAAGTTATGAATCAC (SEQ ID NO: | |
| | (SEQ ID NO: 50) | 51) | |
| BBa_ | 15 aa flexible glycine-serine protein | GGTGGAGGAGGTTCTGGAGGCGGTGGAAGTG | 45 |
| K157013 | domain linker; Freiburg standard | GTGGCGGAGGTAGC (SEQ ID NO: 53) | |
| | RPACKIPNDLKQKVMNH (SEQ ID NO: 52) | | |
| BBa_ | protein linker, 9 aa SGSGSGSGS | TCTGGTTCTGGTTCTGGTTCTGGTTCT | 15 |
| K1680001 | (SEQ ID NO: 367) | (SEQ ID NO: 55) | |
| BBa_ | Protein linker, 12 aa SGSGSGSGSGSG | TCTGGTTCTGGTTCTGGTTCTGGTTCTGGTTCT | 24 |
| K1680002 | (SEQ ID NO: 56) | GGT | |
| | | (SEQ ID NO: 57) | |
| BBa_ | Protein linker, 15 aa | TCTGGTTCTGGTTCTGGTTCTGGTTCTGGTTCT | 33 |
| K1680003 | SGSGSGSGSGSGGSG (SEQ ID NO: 58) | GGTGGTTCTGGT | |
| | | (SEQ ID NO: 59) | |
| BBa_ | flexible linker with SV40 NLS | AGCCTCCTCCATACCAGCCTCTCCCCAAGAAG | 124 |
| K1777005 | SLLHTSLSPRRRERWAAAAATRASSI | AAGAGAAAGGTGGGCGGCGGCGGCAGCTACT | |
| | PASPQEEEKGGRRRQ (SEQ ID NO: 60) | AGAGCCTCCTCCATACCAGCCTCTCCCCAAGA | |
| | | AGAAGAGAAAGGTGGGCGGCGGCGGCAGC | |
| | | (SEQ ID NO: 61) | |
| BBa_ | flexible linker PPPYQPLGGGGS (SEQ | CCTCCTCCATACCAGCCTCTCGGCGGCGGCGG | 36 |
| K1777019 | ID NO: 62) | CAGC (SEQ ID NO: 63) | |
| BBa_ | Cleavable peptide linker for Bitis | GCCTTGAAAGGTGGGGCTTTAAAAGGGGGAG | 72 |
| K2355001 | arietans and Bitis gabonica venom | CCTTGAAGGGGGGTGCCTTGAAGGGTGGGGC | |
| | ALKGGALKGGALKGGALKGGALKG | ATTGAAAGGC (SEQ ID NO: 65) | |
| | (SEQ ID NO: 64) | | |
| BBa_ | Thioredoxin with polylinker | ATGAGCGATAAAATTATTCACCTGACTGACG | 384 |
| K2382004 | MSDKIIHLTDDSFDTDVLKADGAILV | ACAGTTTTGACACGGATGTACTCAAAGCGGA | |
| | DFWAEWCGPCKMIAPILDEIADEYQ | CGGGGCGATCCTCGTCGATTTCTGGGCAGAGT | |
| | GKLTVAKLNIDQNPGTAPKYGIRGIP | GGTGCGGTCCGTGCAAAATGATCGCCCCGATT | |
| | TLLLFKNGEVAATKVGALSKGQLKE | CTGGATGAAATCGCTGACGAATATCAGGGCA | |
| | FLDANLAGSGSGDDDDKGTRGSLEG | AACTGACCGTTGCAAAACTGAACATCGATCA | |
| | G (SEQ ID NO: 66) | AAACCCTGGCACTGCGCCGAAATATGGCATC | |
| | | CGTGGTATCCCGACTCTGCTGCTGTTCAAAAA | |
| | | CGGTGAAGTGGCGGCAACCAAAGTGGGTGCA | |
| | | CTGTCTAAAGGTCAGTTGAAAGAGTTCCTCGA | |
| | | CGCTAACCTGGCGGGTTCTGGTTCTGGTGATG | |
| | | ACGATGACAAGGGTACCCGGGGATCCCTCGA | |
| | | GGGTGGT (SEQ ID NO: 67) | |
| BBa_ | Glycine-Serine Linker GGSGGGSG SEQ | GGAGGCTCCGGCGGCGGATCCGGT (SEQ ID | 24 |
| K2429126 | ID NO: 68) | NO: 69) | |
| BBa_ | Short Linker GGSG (SEQ ID NO: 70) | GGTGGTTCTGGT (SEQ ID NO: 71) | 12 |
| K243004 | | | |
| BBa_ | Middle Linker GGSGGGSG (SEQ ID | GGTGGTTCTGGTGGTGGTTCTGGT (SEQ ID | 24 |
| K243005 | NO: 72) | NO: 73) | |
| BBa_ | Long Linker GGSGGGSGGGSG (SEQ | GGTGGTTCTGGTGGTGGTTCTGGTGGTGGTTC | 36 |
| K243006 | ID NO: 74) | TGGT (SEQ ID NO: 75) | |
| BBa_ | GSAT Linker | GGTGGTTCTGCCGGTGGCTCCGGTTCTGGCTC | 108 |
| K243029 | GGSAGGSGSGSSGGSSGASGTGTAG | CAGCGGTGGCAGCTCTGGTGCGTCCGGCACG | |
| | GTGSGSGTGSG (SEQ ID NO: 76) | GGTACTGCGGGTGGCACTGGCAGCGGTTCCG | |
| | | GTACTGGCTCTGGC (SEQ ID NO: 77) | |
| BBa_ | SEGGGSGGGSEGGGSEGGGSEGGGS | GGTGGTTCTGGCGGCGGTTCTGAAGGTGGCG | 108 |
| K243030 | EGGGSEGGGSGGGS (SEQ ID NO: 78) | GCTCCGAAGGCGGCGGCAGCGAGGGCGGTGG | |
| | | TAGCGAAGGTGGTGGCTCCGAGGGTGGCGGT | |
| | | TCCGGCGGCGGTAGC (SEQ ID NO: 79) | |
| BBa_ | the linker between GAL4 DNA binding | GCAGCTGCTGGTGGAAGTGGAGGTTCTGGCG | 36 |
| K2549045 | domain and VP64 transcription | GGAGC (SEQ ID NO: 81) | |
| | activator AAAGGSGGSGGS (SEQ ID NO: 80) | | |
| BBa_ | G4S linker GGGGS (SEQ ID NO: 82) | GGAGGAGGAGGTAGT (SEQ ID NO: 83) | 15 |
| K2549053 | | | |
| BBa_ | Coding sequence for trunctated | ATGACACACGAACATTCTGCTCAGTGGCTGA | 1458 |
| K2812004 | Lysostaphin fused to His-tagged HlyA | ATAATTATAAAAAAGGTTATGGTTACGGTCCC | |
| | MTHEHSAQWLNNYKKGYGYGPYPL | TACCCGCTGGGAATCAATGGAGGAATGCACT | |
| | GINGGMHYGVDFFMNIGTPVKAISS | ACGGTGTGGACTTCTTTATGAACATTGGCACA | |
| | GKIVEAGWSNYGGGNQIGLIENDGV | CCGGTCAAGGCGATTAGTTCAGGTAAAATCG | |
| | HRQWYMHLSKYNVKVGDYVKAGQ | TAGAAGCTGGTTGGTCAAACTATGGCGGTGG | |
| | IIGWSGSTGYSTAPHLHFQRMVNSFS | GAATCAAATCGGTCTTATCGAGAATGATGGG | |
| | NSTAQDPMPFLKSAGYGKAGGTVTP | GTGCATCGTCAGTGGTACATGCACCTGAGCA | |
| | TPNTGWKTNKYGTLYKSESASFTPN | AATATAATGTGAAAGTTGGCGACTATGTAAA | |
| | TDIITRTTGPFRSMPQSGVLKAGQTIH | GGCCGGGCAAATCATTGGGTGGAGCGGCTCT | |
| | YDEVMKQDGHVWVGYTGNSGQRIY | ACTGGTTATTCTACAGCTCCGCATCTTCATTTC | |
| | LPVRTWNKSTNTLGVLWGTIKGGG | CAACGCATGGTTAATAGTTTTTCAAACTCTAC | |
| | GSLVPRGSGGGGSGNSLAKNVLSGG | AGCCCAAGACCCTATGCCCTTTTTGAAGTCTG | |
| | KGNDKLYGSEGADLLDGGEGNDLL | CGGGTTACGGGAAGGCCGGTGGAACCGTTAC | |
| | KGGYGNDIYRYLSGYGHHIIDDEGG | CCCCACGCCAAATACCGGCTGGAAAACGAAT | |
| | KDDKLSLADIDFRDVAFKREGNDLI | AAGTATGGAACACTGTATAAGTCTGAATCCG | |
| | MYKAEGNVLSIGHKNGITFKNWFEK | CCTCGTTCACTCCCAACACGGATATTATCACA | |
| | ESDDLSNHQIEQIFDKDGRVITPDSLK | CGCACCACTGGACCATTCCGTTCCATGCCTCA | |
| | KAFEYQQSNNKVSYVYGHDASTYG | GTCAGGCGTTTTAAAGGCGGGTCAGACAATC | |
| | SQDNLNPLINEISKIISAAGNFDVKEE | CACTACGACGAGGTTATGAAGCAGGACGGAC | |
| | RSAASLLQLSGNASDFSYGRNSITLT | ATGTCTGGGTCGGGTACACGGGTAACTCCGG | |
| | ASAHHHHHH (SEQ ID NO: 84) | GCAACGTATCTACTTGCCGGTCCGCACGTGGA | |
| | | ATAAAAGCACCAATACACTGGGCGTACTTTG | |
| | | GGGCACAATTAAGGGAGGGGGCGGATCGCTG | |
| | | GTTCCTCGCGGGTCAGGAGGGGGCGGGAGCG | |
| | | GAAATTCTCTTGCTAAAAATGTATTATCCGGT | |
| | | GGAAAAGGTAATGACAAGTTGTACGGCAGTG | |
| | | AGGGAGCAGACCTGCTTGATGGCGGAGAAGG | |
| | | GAATGATCTTCTGAAAGGTGGATATGGTAAT | |
| | | GATATTTATCGTTATCTTTCAGGATATGGCCA | |
| | | TCATATTATTGACGATGAAGGGGGGAAAGAC | |
| | | GATAAACTCAGTTTAGCTGATATAGATTTCCG | |
| | | GGACGTTGCCTTTAAGCGAGAAGGGAATGAC | |
| | | CTCATTATGTATAAAGCTGAAGGTAATGTTCT | |
| | | TTCTATTGGCCACAAAAATGGTATTACATTTA | |
| | | AAAACTGGTTTGAAAAAGAGTCAGATGATCT | |
| | | CTCTAATCATCAGATAGAGCAGATTTTTGATA | |
| | | AAGACGGCAGGGTAATCACACCAGATTCTCT | |
| | | TAAAAAAGCATTTGAATATCAGCAGAGTAAT | |
| | | AACAAGGTAAGTTATGTGTATGGACATGATG | |
| | | CATCAACTTATGGGAGCCAGGACAATCTTAAT | |
| | | CCATTAATTAATGAAATCAGCAAAATCATTTC | |
| | | AGCCGCAGGTAACTTCGATGTTAAGGAGGAA | |
| | | AGATCTGCCGCTTCTTTATTGCAGTTGTCCGG | |
| | | TAATGCCAGTGATTTTTCATATGGACGGAACT | |
| | | CAATAACTTTGACAGCATCAGCACACCACCA | |
| | | CCACCACCACTGA (SEQ ID NO: 85) | |
| BBa_ | Coding sequence for truncated | TAATACGACTCACTATAGGGAAAGAGGAGAA | 1496 |
| K2812005 | Lysostaphin with HlyA and His6-tag | ATACTAGATGACACACGAACATTCTGCTCAGT | |
| | (SEQ ID NO: 312) regulated by T7- | GGCTGAATAATTATAAAAAAGGTTATGGTTA | |
| | promoter | CGGTCCCTACCCGCTGGGAATCAATGGAGGA | |
| | MTHEHSAQWLNNYKKGYGYGPYPL | ATGCACTACGGTGTGGACTTCTTTATGAACAT | |
| | GINGGMHYGVDFFMNIGTPVKAISS | TGGCACACCGGTCAAGGCGATTAGTTCAGGT | |
| | GKIVEAGWSNYGGGNQIGLIENDGV | AAAATCGTAGAAGCTGGTTGGTCAAACTATG | |
| | HRQWYMHLSKYNVKVGDYVKAGQ | GCGGTGGGAATCAAATCGGTCTTATCGAGAA | |
| | IIGWSGSTGYSTAPHLHFQRMVNSFS | TGATGGGGTGCATCGTCAGTGGTACATGCACC | |
| | NSTAQDPMPFLKSAGYGKAGGTVTP | TGAGCAAATATAATGTGAAAGTTGGCGACTA | |
| | TPNTGWKTNKYGTLYKSESASFTPN | TGTAAAGGCCGGGCAAATCATTGGGTGGAGC | |
| | TDIITRTTGPFRSMPQSGVLKAGQTIH | GGCTCTACTGGTTATTCTACAGCTCCGCATCT | |
| | YDEVMKQDGHVWVGYTGNSGQRIY | TCATTTCCAACGCATGGTTAATAGTTTTTCAA | |
| | LPVRTWNKSTNTLGVLWGTIKGGG | ACTCTACAGCCCAAGACCCTATGCCCTTTTTG | |
| | GSLVPRGSGGGGSGNSLAKNVLSGG | AAGTCTGCGGGTTACGGGAAGGCCGGTGGAA | |
| | KGNDKLYGSEGADLLDGGEGNDLL | CCGTTACCCCCACGCCAAATACCGGCTGGAA | |
| | KGGYGNDIYRYLSGYGHHIIDDEGG | AACGAATAAGTATGGAACACTGTATAAGTCT | |
| | KDDKLSLADIDFRDVAFKREGNDLI | GAATCCGCCTCGTTCACTCCCAACACGGATAT | |
| | MYKAEGNVLSIGHKNGITFKNWFEK | TATCACACGCACCACTGGACCATTCCGTTCCA | |
| | ESDDLSNHQIEQIFDKDGRVITPDSLK | TGCCTCAGTCAGGCGTTTTAAAGGCGGGTCAG | |
| | KAFEYQQSNNKVSYVYGHDASTYG | ACAATCCACTACGACGAGGTTATGAAGCAGG | |
| | SQDNLNPLINEISKIISAAGNFDVKEE | ACGGACATGTCTGGGTCGGGTACACGGGTAA | |
| | RSAASLLQLSGNASDFSYGRNSITLT | CTCCGGGCAACGTATCTACTTGCCGGTCCGCA | |
| | ASAHHHHHH (SEQ ID NO: 86) | CGTGGAATAAAAGCACCAATACACTGGGCGT | |
| | | ACTTTGGGGCACAATTAAGGGAGGGGGCGGA | |
| | | TCGCTGGTTCCTCGCGGGTCAGGAGGGGGCG | |
| | | GGAGCGGAAATTCTCTTGCTAAAAATGTATTA | |
| | | TCCGGTGGAAAAGGTAATGACAAGTTGTACG | |
| | | GCAGTGAGGGAGCAGACCTGCTTGATGGCGG | |
| | | AGAAGGGAATGATCTTCTGAAAGGTGGATAT | |
| | | GGTAATGATATTTATCGTTATCTTTCAGGATA | |
| | | TGGCCATCATATTATTGACGATGAAGGGGGG | |
| | | AAAGACGATAAACTCAGTTTAGCTGATATAG | |
| | | ATTTCCGGGACGTTGCCTTTAAGCGAGAAGG | |
| | | GAATGACCTCATTATGTATAAAGCTGAAGGT | |
| | | AATGTTCTTTCTATTGGCCACAAAAATGGTAT | |
| | | TACATTTAAAAACTGGTTTGAAAAAGAGTCA | |
| | | GATGATCTCTCTAATCATCAGATAGAGCAGAT | |
| | | TTTTGATAAAGACGGCAGGGTAATCACACCA | |
| | | GATTCTCTTAAAAAAGCATTTGAATATCAGCA | |
| | | GAGTAATAACAAGGTAAGTTATGTGTATGGA | |
| | | CATGATGCATCAACTTATGGGAGCCAGGACA | |
| | | ATCTTAATCCATTAATTAATGAAATCAGCAAA | |
| | | ATCATTTCAGCCGCAGGTAACTTCGATGTTAA | |
| | | GGAGGAAAGATCTGCCGCTTCTTTATTGCAGT | |
| | | TGTCCGGTAATGCCAGTGATTTTTCATATGGA | |
| | | CGGAACTCAATAACTTTGACAGCATCAGCAC | |
| | | ACCACCACCACCACCACTGA | |
| | | SEQ ID NO: 87 | |
| BBa_ | Coding sequence for Pyocin S5 with | ATGTCAAATGATAACGAAGTCCCAGGTAGTA | 2217 |
| K2812006 | HlyA and His6-tag (SEQ ID NO: 312) | TGGTGATTGTAGCCCAGGGTCCAGACGACCA | |
| | MSNDNEVPGSMVIVAQGPDDQYAY | GTATGCGTATGAGGTACCGCCTATCGACTCCG | |
| | EVPPIDSAAVAGNMFGDLIQREIYLQ | CGGCGGTTGCAGGCAATATGTTCGGCGATCTT | |
| | KNIYYPVRSIFEQGTKEKKEINKKVS | ATCCAACGCGAGATCTACCTGCAAAAGAATA | |
| | DQVDGLLKQITQGKREATRQERVDV | TCTATTATCCGGTACGGTCGATCTTTGAGCAA | |
| | MSAVLHKMESDLEGYKKTFTKGPFI | GGGACTAAAGAGAAGAAAGAAATTAACAAG | |
| | DYEKQSSLSIYEAWVKIWEKNSWEE | AAGGTGTCTGATCAGGTTGATGGCCTGCTCAA | |
| | RKKYPFQQLVRDELERAVAYYKQD | ACAAATCACGCAAGGTAAGCGCGAAGCGACT | |
| | SLSEAVKVLRQELNKQKALKEKEDL | CGGCAAGAACGCGTTGACGTAATGTCTGCGG | |
| | SQLERDYRTRKANLEMKVQSELDQ | TTCTTCATAAGATGGAATCAGACCTCGAAGG | |
| | AGSALPPLVSPTPEQWLERATRLVTQ | GTATAAAAAGACATTCACAAAAGGCCCATTT | |
| | AIADKKQLQTTNNTLIKNSPTPLEKQ | ATTGACTACGAGAAGCAATCATCCTTATCGAT | |
| | KAIYNGELLVDEIASLQARLVKLNAE | CTACGAGGCATGGGTGAAGATCTGGGAGAAA | |
| | LSLKDRQAIAKAFDSLDKQMMAKSL | AATAGCTGGGAGGAACGTAAAAAATATCCTT | |
| | ADFYKEVTEKFGARTSEMARQLAEG | TCCAACAGCTGGTGCGTGACGAACTTGAGCG | |
| | ARGKNIRSSAEAIKSFEKHKDALNKK | GGCCGTAGCTTATTATAAACAAGACTCGTTGT | |
| | TTRRRTEAERKAAEEQALQDAIKFT | CGGAGGCTGTAAAGGTGCTTCGTCAGGAGTT | |
| | EKFSKGFGVVGKAIDAASLYQEFKIS | GAATAAACAGAAGGCACTCAAGGAGAAGGA | |
| | TETGDWKPFFVKIETLAAGAAASWL | AGACCTTAGTCAGTTGGAGCGTGATTATCGTA | |
| | VGIAFATATATPIGILGFALVMAVTG | CTCGGAAAGCAAATCTCGAAATGAAAGTGCA | |
| | AMIDEDLLEKANNLVISIGGGGSLVP | GTCCGAACTCGACCAGGCTGGGTCCGCCCTTC | |
| | RGSGGGGSGNSLAKNVLSGGKGND | CGCCTTTGGTTTCACCAACACCAGAACAGTGG | |
| | KLYGSEGADLLDGGEGNDLLKGGY | CTTGAACGGGCAACACGTTTAGTAACTCAAG | |
| | GNDIYRYLSGYGHHIIDDEGGKDDK | CGATTGCGGATAAGAAGCAGCTTCAGACTAC | |
| | LSLADIDFRDVAFKREGNDLIMYKA | GAACAACACTCTGATTAAAAATTCGCCGACA | |
| | EGNVLSIGHKNGITFKNWFEKESDDL | CCACTTGAGAAGCAGAAGGCGATTTATAATG | |
| | SNHQIEQIFDKDGRVITPDSLKKAFE | GGGAGTTGCTTGTGGATGAAATTGCAAGTTTA | |
| | YQQSNNKVSYVYGHDASTYGSQDN | CTACCCGGCGGCGTACGGAAGCAGAGCGCAA | |
| | LNPLINEISKIISAAGNFDVKEERSAA | CAAGCACGCCTTGTTAAACTCAACGCCGAAA | |
| | SLLQLSGNASDFSYGRNSITLTASAH | GGCTGCGGAAGAACAGGCACTTCAGGATGCT | |
| | HHHHH (SEQ ID NO: 88) | ATCAAGTTTACGGCGGACTTCTACAAGGAGG | |
| | | TCACAGAGAAATTCGGTGCGCGTACTTCAGA | |
| | | GATGGCCCGGCAATTAGCAGAGGGGGCACGG | |
| | | GGTAAAAACATTCGTTCGAGTGCAGAGGCCA | |
| | | TCAAATCTTTCGAAAAGCATAAAGACGCATT | |
| | | GAATAAAAAGCTAAGCTTAAAAGACCGGCAG | |
| | | GCTATCGCAAAGGCATTTGATTCGTTGGACAA | |
| | | ACAAATGATGGCGAAGAGCCTTGAAAAATTC | |
| | | TCAAAGGGTTTTGGCGTTGTGGGGAAGGCTAT | |
| | | CGACGCGGCGAGTCTTTACCAAGAGTTTAAA | |
| | | ATCAGCACCGAGACTGGTGACTGGAAACCAT | |
| | | TTTTTGTTAAGATTGAAACCTTAGCTGCGGGC | |
| | | GCCGCAGCGTCATGGCTCGTCGGGATCGCCTT | |
| | | TGCCACGGCCACAGCCACGCCAATTGGCATTC | |
| | | TCGGTTTTGCACTCGTGATGGCAGTTACCGGG | |
| | | GCTATGATTGATGAGGACCTGCTCGAAAAGG | |
| | | CAAATAATCTTGTGATCAGCATTGGAGGGGG | |
| | | CGGATCGCTGGTTCCTCGCGGGTCAGGAGGG | |
| | | GGCGGGAGCGGAAATTCTCTTGCTAAAAATG | |
| | | TATTATCCGGTGGAAAAGGTAATGACAAGTT | |
| | | GTACGGCAGTGAGGGAGCAGACCTGCTTGAT | |
| | | GGCGGAGAAGGGAATGATCTTCTGAAAGGTG | |
| | | GATATGGTAATGATATTTATCGTTATCTTTCA | |
| | | GGATATGGCCATCATATTATTGACGATGAAG | |
| | | GGGGGAAAGACGATAAACTCAGTTTAGCTGA | |
| | | TATAGATTTCCGGGACGTTGCCTTTAAGCGAG | |
| | | AGGGTAACGACCTCATTATGTATAAAGCTGA | |
| | | AGGTAATGTTCTTTCTATTGGCCACAAAAATG | |
| | | GTATTACATTTAAAAACTGGTTTGAAAAAGA | |
| | | GTCAGATGATCTCTCTAATCATCAGATAGAGC | |
| | | AGATTTTTGATAAAGACGGCAGGGTAATCAC | |
| | | ACCAGATTCTCTTAAAAAAGCATTTGAATATC | |
| | | AGCAGAGTAATAACAAGGTAAGTTATGTGTA | |
| | | TGGACATGATGCATCAACTTATGGGAGCCAG | |
| | | GACAATCTTAATCCATTAATTAATGAAATCAG | |
| | | CAAAATCATTTCAGCCGCAGGTAACTTCGATG | |
| | | TTAAGGAGGAAAGATCTGCCGCTTCTTTATTG | |
| | | CAGTTGTCCGGTAATGCCAGTGATTTTTCATA | |
| | | TGGACGGAACTCAATAACTTTGACAGCATCA | |
| | | GCACATCATCACCATCACCACTGA (SEQ ID | |
| | | NO: 89) | |
| BBa_ | Coding sequence for Pyocin S5 with | CTGACGCTTTTTATCGCAACTCTCTACTGTTT | 2288 |
| K2812007 | HlyA and His6-tag (SEQ ID NO: 312) | CTCCATACCCGTTTTTTAACTTTAAGAAGGAG | |
| | regulated by pBAD-ara promoter | ATATACCATGTCAAATGATAACGAAGTCCCA | |
| | MSNDNEVPGSMVIVAQGPDDQYAY | GGTAGTATGGTGATTGTAGCCCAGGGTCCAG | |
| | EVPPIDSAAVAGNMFGDLIQREIYLQ | ACGACCAGTATGCGTATGAGGTACCGCCTATC | |
| | KNIYYPVRSIFEQGTKEKKEINKKVS | GACTCCGCGGCGGTTGCAGGCAATATGTTCG | |
| | DQVDGLLKQITQGKREATRQERVDV | GCGATCTTATCCAACGCGAGATCTACCTGCAA | |
| | MSAVLHKMESDLEGYKKTFTKGPFI | AAGAATATCTATTATCCGGTACGGTCGATCTT | |
| | DYEKQSSLSIYEAWVKIWEKNSWEE | TGAGCAAGGGACTAAAGAGAAGAAAGAAATT | |
| | RKKYPFQQLVRDELERAVAYYKQD | AACAAGAAGGTGTCTGATCAGGTTGATGGCC | |
| | SLSEAVKVLRQELNKQKALKEKEDL | TGCTCAAACAAATCACGCAAGGTAAGCGCGA | |
| | SQLERDYRTRKANLEMKVQSELDQ | AGCGACTCGGCAAGAACGCGTTGACGTAATG | |
| | AGSALPPLVSPTPEQWLERATRLVTQ | TCTGCGGTTCTTCATAAGATGGAATCAGACCT | |
| | AIADKKQLQTTNNTLIKNSPTPLEKQ | CGAAGGGTATAAAAAGACATTCACAAAAGGC | |
| | KAIYNGELLVDEIASLQARLVKLNAE | CCATTTATTGACTACGAGAAGCAATCATCCTT | |
| | ADFYKEVTEKFGARTSEMARQLAEG | ATCGATCTACGAGGCATGGGTGAAGATCTGG | |
| | LSLKDRQAIAKAFDSLDKQMMAKSL | GAGAAAAATAGCTGGGAGGAACGTAAAAAAT | |
| | TTRRRTEAERKAAEEQALQDAIKFT | ATCCTTTCCAACAGCTGGTGCGTGACGAACTT | |
| | ARGKNIRSSAEAIKSFEKHKDALNKK | GAGCGGGCCGTAGCTTATTATAAACAAGACT | |
| | EKFSKGFGVVGKAIDAASLYQEFKIS | CGTTGTCGGAGGCTGTAAAGGTGCTTCGTCAG | |
| | TETGDWKPFFVKIETLAAGAAASWL | GAGTTGAATAAACAGAAGGCACTCAAGGAGA | |
| | VGIAFATATATPIGILGFALVMAVTG | AGGAAGACCTTAGTCAGTTGGAGCGTGATTA | |
| | AMIDEDLLEKANNLVISIGGGGSLVP | TCGTACTCGGAAAGCAAATCTCGAAATGAAA | |
| | RGSGGGGSGNSLAKNVLSGGKGND | GTGCAGTCCGAACTCGACCAGGCTGGGTCCG | |
| | KLYGSEGADLLDGGEGNDLLKGGY | CCCTTCCGCCTTTGGTTTCACCAACACCAGAA | |
| | GNDIYRYLSGYGHHIIDDEGGKDDK | CAGTGGCTTGAACGGGCAACACGTTTAGTAA | |
| | LSLADIDFRDVAFKREGNDLIMYKA | CTCAAGCGATTGCGGATAAGAAGCAGCTTCA | |
| | EGNVLSIGHKNGITFKNWFEKESDDL | GACTACGAACAACACTCTGATTAAAAATTCG | |
| | SNHQIEQIFDKDGRVITPDSLKKAFE | AAGTTTACAAGCACGCCTTGTTAAACTCAACG | |
| | YQQSNNKVSYVYGHDASTYGSQDN | ATAATGGGGAGTTGCTTGTGGATGAAATTGC | |
| | LNPLINEISKIISAAGNFDVKEERSAA | CCGAAACTACCCGGCGGCGTACGGAAGCAGA | |
| | SLLQLSGNASDFSYGRNSITLTASAH | CCGACACCACTTGAGAAGCAGAAGGCGATTT | |
| | HHHHH | GCGCAAGGCTGCGGAAGAACAGGCACTTCAG | |
| | (SEQ ID NO: 90) | GATGCTATCAAGTTTACGGCGGACTTCTACAA | |
| | | GGAGGTCACAGAGAAATTCGGTGCGCGTACT | |
| | | TCAGAGATGGCCCGGCAATTAGCAGAGGGGG | |
| | | CACGGGGTAAAAACATTCGTTCGAGTGCAGA | |
| | | GGCCATCAAATCTTTCGAAAAGCATAAAGAC | |
| | | GCATTGAATAAAAAGCTAAGCTTAAAAGACC | |
| | | GGCAGGCTATCGCAAAGGCATTTGATTCGTTG | |
| | | GACAAACAAATGATGGCGAAGAGCCTTGAAA | |
| | | AATTCTCAAAGGGTTTTGGCGTTGTGGGGAAG | |
| | | GCTATCGACGCGGCGAGTCTTTACCAAGAGTT | |
| | | TAAAATCAGCACCGAGACTGGTGACTGGAAA | |
| | | CCATTTTTTGTTAAGATTGAAACCTTAGCTGC | |
| | | GGGCGCCGCAGCGTCATGGCTCGTCGGGATC | |
| | | GCCTTTGCCACGGCCACAGCCACGCCAATTGG | |
| | | CATTCTCGGTTTTGCACTCGTGATGGCAGTTA | |
| | | CCGGGGCTATGATTGATGAGGACCTGCTCGA | |
| | | AAAGGCAAATAATCTTGTGATCAGCATTGGA | |
| | | GGGGGCGGATCGCTGGTTCCTCGCGGGTCAG | |
| | | GAGGGGGCGGGAGCGGAAATTCTCTTGCTAA | |
| | | AAATGTATTATCCGGTGGAAAAGGTAATGAC | |
| | | AAGTTGTACGGCAGTGAGGGAGCAGACCTGC | |
| | | TTGATGGCGGAGAAGGGAATGATCTTCTGAA | |
| | | AGGTGGATATGGTAATGATATTTATCGTTATC | |
| | | TTTCAGGATATGGCCATCATATTATTGACGAT | |
| | | GAAGGGGGGAAAGACGATAAACTCAGTTTAG | |
| | | CTGATATAGATTTCCGGGACGTTGCCTTTAAG | |
| | | CGAGAGGGTAACGACCTCATTATGTATAAAG | |
| | | CTGAAGGTAATGTTCTTTCTATTGGCCACAAA | |
| | | AATGGTATTACATTTAAAAACTGGTTTGAAAA | |
| | | AGAGTCAGATGATCTCTCTAATCATCAGATAG | |
| | | AGCAGATTTTTGATAAAGACGGCAGGGTAAT | |
| | | CACACCAGATTCTCTTAAAAAAGCATTTGAAT | |
| | | ATCAGCAGAGTAATAACAAGGTAAGTTATGT | |
| | | GTATGGACATGATGCATCAACTTATGGGAGC | |
| | | CAGGACAATCTTAATCCATTAATTAATGAAAT | |
| | | CAGCAAAATCATTTCAGCCGCAGGTAACTTCG | |
| | | ATGTTAAGGAGGAAAGATCTGCCGCTTCTTTA | |
| | | TTGCAGTTGTCCGGTAATGCCAGTGATTTTTC | |
| | | ATATGGACGGAACTCAATAACTTTGACAGCA | |
| | | TCAGCACATCATCACCATCACCACTGA (SEQ | |
| | | ID NO: 91) | |
| BBa_ | Glycine linker GGGG (SEQ ID NO: 92) | GGAGGAGGAGGA (SEQ ID NO: 93) | 12 |
| K3120002 | | | |
| BBa | (HL5)2 peptide helical linker, | TGGCGGAAGCGGCTGCGAAAGAAGCGGCTGC | 89 |
| K3672000 | rigid; separate the domains of fusion | GAAAGAAGCGGCGGCTAAAGAAGCGGCGGC | |
| | proteins | GAAAGAAGCAGCGGCGAAAGCGGCGGCG | |
| | WRKRLRKKRLRKKRRLKKRRRKKQ | (SEQ ID NO: 95) | |
| | RRKRR (SEQ ID NO: 94) | | |
| BBa_ | Glycine-Serine linker GSGGS (SEQ ID | GGATCTGGTGGAAGT (SEQ ID NO: 97) | 15 |
| K3765005 | NO: 96) | | |
| BBa | Glycine-Serine linker GSSGS (SEQ ID | GGCAGCTCGGGATCA (SEQ ID NO: 99) | 15 |
| K3765006 | NO: 98) | | |
| BBa_ | 22 Amino Acid | TTGGAAGGATCTGGCCAAGGTCCCGGCAGCG | 66 |
| K3765009 | Linker LEGSGQGPGSGQGSGSPGSG | GGCAAGGGTCGGGAAGCCCAGGGTCCGGTCA | |
| | QG (SEQ ID NO: 100) | GGGC (SEQ ID NO: 101) | |
| BBa_ | (G2S)3 GGSGGSGGS (SEQ ID NO: | GGCGGTAGCGGCGGTAGCGGCGGTAGC (SEQ | 27 |
| K3790007 | 102) | ID NO: 103) | |
| BBa_ | SEG-Linker | GGTGGTTCTGGCGGCGGTTCTGAAGGTGGCG | 108 |
| K404300 | GGSGGGSEGGGSEGGGSEGGGSEGG | GCTCCGAAGGCGGCGGCAGCGAGGGCGGTGG | |
| | GSEGGGSGGGS (SEQ ID NO: 104) | TAGCGAAGGTGGTGGCTCCGAGGGTGGCGGT | |
| | | TCCGGCGGCGGTAGC (SEQ ID NO: 105) | |
| BBa_ | GSAT-Linker | GGTGGTTCTGCCGGTGGCTCCGGTTCTGGCTC | 108 |
| K404301 | GGSAGGSGSGSSGGSSGASGTGTAG | CAGCGGTGGCAGCTCTGGTGCGTCCGGCACG | |
| | GTGSGSGTGSG (SEQ ID NO: 106) | GGTACTGCGGGTGGCACTGGCAGCGGTTCCG | |
| | | GTACTGGCTCTGGC (SEQ ID NO: 107) | |
| BBa_ | Z-EGFR-1907 Short- | GTGGATAACAAATTTAACAAAGAAATGTGGG | 192 |
| K404303 | Linker VDNKFNKEMWAAWEEIRNLP | CGGCGTGGGAAGAAATTCGTAACCTGCCGAA | |
| | NLNGWQMTAFIASLVDDPSQSANLL | CCTGAACGGCTGGCAGATGACCGCGTTTATTG | |
| | AEAKKLNDAQAPKTGGGSG (SEQ ID | CGAGCCTGGTGGATGATCCGAGCCAGAGCGC | |
| | NO: 108) | GAACCTGCTGGCGGAAGCGAAAAAACTGAAC | |
| | | GATGCGCAGGCGCCGAAAACCGGCGGTGGTT | |
| | | CTGGT (SEQ ID NO: 109) | |
| BBa_ | Z-EGFR-1907 Middle- | GTGGATAACAAATTTAACAAAGAAATGTGGG | 204 |
| K404304 | Linker VDNKFNKEMWAAWEEIRNL | CGGCGTGGGAAGAAATTCGTAACCTGCCGAA | |
| | PNLNGWQMTAFIASLVDDPSQSANL | CCTGAACGGCTGGCAGATGACCGCGTTTATTG | |
| | LAEAKKLNDAQAPKTGGGSGGGSG | CGAGCCTGGTGGATGATCCGAGCCAGAGCGC | |
| | (SEQ ID NO: 110) | GAACCTGCTGGCGGAAGCGAAAAAACTGAAC | |
| | | GATGCGCAGGCGCCGAAAACCGGCGGTGGTT | |
| | | CTGGTGGTGGTTCTGGT (SEQ ID NO: 111) | |
| BBa__ | Z-EGFR-1907_Long- | GTGGATAACAAATTTAACAAAGAAATGTGGG | 216 |
| K404 | Linker VDNKFNKEMWAAWEEIRNLP | CGGCGTGGGAAGAAATTCGTAACCTGCCGAA | |
| 305 | NLNGWQMTAFIASL VDDPSQSANLL | CCTGAACGGCTGGCAGATGACCGCGTTTATTG | |
| | AEAKKLNDAQAPKTGGGSGGGSGG | CGAGCCTGGTGGATGATCCGAGCCAGAGCGC | |
| | GSG (SEQ ID NO: 112) | GAACCTGCTGGCGGAAGCGAAAAAACTGAAC | |
| | | GATGCGCAGGCGCCGAAAACCGGCGGTGGTT | |
| | | CTGGTGGTGGTTCTGGTGGTGGTTCTGGT | |
| | | (SEQ ID NO: 113) | |
| BBa | Z-EGFR-1907 SEG- | GTGGATAACAAATTTAACAAAGAAATGTGGG | 288 |
| K404 | Linker VDNKFNKEMWAAWEEIRNL | CGGCGTGGGAAGAAATTCGTAACCTGCCGAA | |
| 306 | PNLNGWQMTAFIASLVDDPSQSANL | CCTGAACGGCTGGCAGATGACCGCGTTTATTG | |
| | LAEAKKLNDAQAPKTGGGSGGGSE | CGAGCCTGGTGGATGATCCGAGCCAGAGCGC | |
| | GGGSEGGGSEGGGSEGGGSEGGGSG | GAACCTGCTGGCGGAAGCGAAAAAACTGAAC | |
| | GGS (SEQ ID NO: 114) | GATGCGCAGGCGCCGAAAACCGGCGGTGGTT | |
| | | CTGGCGGCGGTTCTGAAGGTGGCGGCTCCGA | |
| | | AGGCGGCGGCAGCGAGGGCGGTGGTAGCGAA | |
| | | GGTGGTGGCTCCGAGGGTGGCGGTTCCGGCG | |
| | | GCGGTAGC (SEQ ID NO: 115) | |
| BBa_ | (Gly4Ser)3 Flexible Peptide Linker | GGTGGAGGAGGCTCTGGTGGAGGCGGTAGCG | 45 |
| K416001 | GGGGSGGGGSGGGGS (SEQ ID NO: | GAGGCGGAGGGTCG (SEQ ID NO: 117) | |
| | 116) | | |
| BBa_ | Short Fusion Protein Linker: GGSG | GGTGGTTCTGGT (SEQ ID NO: 119) | 12 |
| K648005 | (SEQ ID NO: 118) with standard 25 | | |
| | prefix/suffix | | |
| BBa_ | Long 10AA Fusion Protein Linker with | GGTGAAAATTTGTATTTTCAATCTGGTGGT | 30 |
| K648006 | Standard 25 Prefix/Suffix | (SEQ ID NO: 121) | |
| | GENL YFQSGG (SEQ ID NO: 120) | | |
| BBa_ | Medium 6AA Fusion Protein Linker: | GGAGGTTCAGGAGGCAGC | 18 |
| K648007 | GGSGGS (SEQ ID NO: 122) with | (SEQ ID NO: 123) | |
| | Standard 25 Prefix/Suffix | | |

All the relevant sequences disclosed in the examples below are listed in Table 5.

TABLE 5
Exemplary Protein Sequences

| SEQ | | |
|---|---|---|
| ID | | |
| NO: | Name | Amino Acid Sequence |
| 1 | CD63-MMP2-a-CD16-a-CD33- | MCGACKENYCLMITFAIFLSLIMLVEVAAAIAGYVGGSGPVRRY |
| | Myc-MMP9-CD63-GPi | QGGPLGVRGGMEVQLVESGGGVVRPGGSLRLSCAASGFTFDDY |
| | | GMSWVRQAPGKGLEWVSGINWNGGSTGYADSVKGRFTISRDNA |
| | | KNSLYLQMNSLRAEDTAVYYCARGRSLLFDYWGQGTLVTVSRG |
| | | GGGSGGGGSGGGGSSELTQDPAVSVALGQTVRITCQGDSLRSYY |
| | | ASWYQQKPGQAPVLVIYGKNNRPSGIPDRFSGSSSGNTASLTITG |
| | | AQAEDEADYYCNSRDSSGNHVVFGGGTKLTVLPSGQAGAAASE |
| | | SLFVSNHAYEASGGPEQVQLVQSGAEVKKPGSSVKVSCKASGYT |
| | | FTDYNMHWVRQAPGQGLEWIGYIYPYNGGTGYNQKFKSKATIT |
| | | ADESTNTAYMELSSLRSEDTAVYYCARGRPAMDYWGQGTLVTV |
| | | SSGGGGSGGGGSGGGGSDIQMTQSPSSLSASVGDRVTITCRASES |
| | | VDNYGISFMNWFQQKPGKAPKLLIYAASNQGSGVPSRFSGSGSG |
| | | TDFTLTISSLQPDDFATYYCQQSKEVPWTFGQGTKVEIKGGsgEQK |
| | | LISEEDLggPLGMTSGGSGPKNNHTASILDRMQADFKCCGAANYT |
| | | DWEKIPSMSKNRVPDSCCINVTVGCGINFNEKAIHKEGCVEKIGG |
| | | WLRKNVVLRDKLVKCEGISLLAQNTSWLLLLLLSLSLLQATDFM |
| | | SL |
| 155 | CD63-MMP2-a-CD16-a-CD33- | MCGACKENYCLMITFAIFLSLIMLVEVAAAIAGYVGGSGPVRRY |
| | Myc-MMP9-CytCD63-EV Motif | QGGPLGVRGGMEVQLVESGGGVVRPGGSLRLSCAASGFTFDDY |
| | | GMSWVRQAPGKGLEWVSGINWNGGSTGYADSVKGRFTISRDNA |
| | | KNSLYLQMNSLRAEDTAVYYCARGRSLLFDYWGQGTLVTVSRG |
| | | GGGSGGGGSGGGGSSELTQDPAVSVALGQTVRITCQGDSLRSYY |
| | | ASWYQQKPGQAPVLVIYGKNNRPSGIPDRFSGSSSGNTASLTITG |
| | | AQAEDEADYYCNSRDSSGNHVVFGGGTKLTVLPSGQAGAAASE |
| | | SLFVSNHAYEASGGPEQVQLVQSGAEVKKPGSSVKVSCKASGYT |
| | | FTDYNMHWVRQAPGQGLEWIGYIYPYNGGTGYNQKFKSKATIT |
| | | ADESTNTAYMELSSLRSEDTAVYYCARGRPAMDYWGQGTLVTV |
| | | SSGGGGSGGGGSGGGGSDIQMTQSPSSLSASVGDRVTITCRASES |
| | | VDNYGISFMNWFQQKPGKAPKLLIYAASNQGSGVPSRFSGSGSG |
| | | TDFTLTISSLQPDDFATYYCQQSKEVPWTFGQGTKVEIKGGsgEQK |
| | | LISEEDLggPLGMTSGGSGPKNNHTASILDRMQADFKCCGAANYT |
| | | DWEKIPSMSKNRVPDSCCINVTVGCGINFNEKAIHKEGCVEKIGG |
| | | WLRKNVLVVAAAALGIAFVEVLGIVFACCLVKSIRSGYEVM |
| 188 | CD63-MMP2-a-CD16-a-CD33- | MCGACKENYCLMITFAIFLSLIMLVEVAAAIAGYVGGSGPVRRY |
| | Myc-MMP9-CytCD63 | QGGPLGVRGGMEVQLVESGGGVVRPGGSLRLSCAASGFTFDDY |
| | | GMSWVRQAPGKGLEWVSGINWNGGSTGYADSVKGRFTISRDNA |
| | | KNSLYLQMNSLRAEDTAVYYCARGRSLLFDYWGQGTLVTVSRG |
| | | GGGSGGGGSGGGGSSELTQDPAVSVALGQTVRITCQGDSLRSYY |
| | | ASWYQQKPGQAPVLVIYGKNNRPSGIPDRFSGSSSGNTASLTITG |
| | | AQAEDEADYYCNSRDSSGNHVVFGGGTKLTVLPSGQAGAAASE |
| | | SLFVSNHAYEASGGPEQVQLVQSGAEVKKPGSSVKVSCKASGYT |
| | | FTDYNMHWVRQAPGQGLEWIGYIYPYNGGTGYNQKFKSKATIT |
| | | ADESTNTAYMELSSLRSEDTAVYYCARGRPAMDYWGQGTLVTV |
| | | SSGGGGGGGGSGGGGSDIQMTQSPSSLSASVGDRVTITCRASES |
| | | VDNYGISFMNWFQQKPGKAPKLLIYAASNQGSGVPSRFSGSGSG |
| | | TDFTLTISSLQPDDFATYYCQQSKEVPWTFGQGTKVEIKGGsgEQK |
| | | LISEEDLggPLGMTSGGSGPKNNHTASILDRMQADFKCCGAANYT |
| | | DWEKIPSMSKNRVPDSCCINVTVGCGINFNEKAIHKEGCVEKIGG |
| | | WLRKNVLVVAAAALGIAFVEVLGIVFACCLVKS |
| 54 | TNFSF14-a-CD16-a-CD33- | MSCSVARVGLGLLLLLMGAGLAVQGWELLQLHWRLGEMVTRL |
| | Myc-TNR1A-ADAM17-a- | PDGPAGSWEQLIQERRSHEVNPAAGGSGPVRRYQMEVQLVESGG |
| | CD38-41BB | GVVRPGGSLRLSCAASGFTFDDYGMSWVRQAPGKGLEWVSGIN |
| | | WNGGSTGYADSVKGRFTISRDNAKNSLYLQMNSLRAEDTAVYY |
| | | CARGRSLLFDYWGQGTLVTVSRGGGGSGGGGSGGGGSSELTQD |
| | | PAVSVALGQTVRITCQGDSLRSYYASWYQQKPGQAPVLVIYGKN |
| | | NRPSGIPDRFSGSSSGNTASLTITGAQAEDEADYYCNSRDSSGNH |
| | | VVFGGGTKLTVLPSGQAGAAASESLFVSNHAYEASGGPEQVQLV |
| | | QSGAEVKKPGSSVKVSCKASGYTFTDYNMHWVRQAPGQGLEWI |
| | | GYIYPYNGGTGYNQKFKSKATITADESTNTAYMELSSLRSEDTAV |
| | | YYCARGRPAMDYWGQGTLVTVSSGGGGGGGGSGGGGSDIQM |
| | | TQSPSSLSASVGDRVTITCRASESVDNYGISFMNWFQQKPGKAPK |
| | | LLIYAASNQGSGVPSRFSGSGSGTDFTLTISSLQPDDFATYYCQQS |
| | | KEVPWTFGQGTKVEIKGGsgEQKLISEEDLSGGSGSLECTKLCLPQ |
| | | IENVKGTEDSGGSGQVQLVQSGAEVKKPGSSVKVSCKAFGGTFS |
| | | SYAISWVRQAPGQGLEWMGRIIRFLGIANYAQKFQGRVTLIADKS |
| | | TNTAYMELSSLRSEDTAVYYCAGEPGREDPDAVDIWGQGTMVT |
| | | VSSGGGGSGGGGSGGGGSDIQMTQSPSSLSASVGDRVTITCRASQ |
| | | GIRSWLAWYQQKPEKARKSLIYAASSLQSGVPSRFSGSGSGTDFT |
| | | LTISSLQPEDFATYYCQQYNSYPLTFGGGTKVEIKGGGGSGGGGS |
| | | GGGGSSGTTVLLPLVIFFGLCLLSLLFIGLMYRYGGSGKRGRKKL |
| | | LYIFKQPFMRPVQTTQEEDG |
| 252 | CD63-GrzB-a-CD16-a-CD33- | MCGACKENYCLMITFAIFLSLIMLVEVAAAIAGYVGGSGPVRRY |
| | Myc-GrzB-a-CD38-CD28-CD3z | QIEPDggsgMEVQLVESGGGVVRPGGSLRLSCAASGFTFDDYGMS |
| | | WVRQAPGKGLEWVSGINWNGGSTGYADSVKGRFTISRDNAKNS |
| | | LYLQMNSLRAEDTAVYYCARGRSLLFDYWGQGTLVTVSRGGGG |
| | | SGGGGSGGGGSSELTQDPAVSVALGQTVRITCQGDSLRSYYASW |
| | | YQQKPGQAPVLVIYGKNNRPSGIPDRFSGSSSGNTASLTITGAQAE |
| | | DEADYYCNSRDSSGNHVVFGGGTKLTVLPSGQAGAAASESLFVS |
| | | NHAYEASGGPEQVQLVQSGAEVKKPGSSVKVSCKASGYTFTDY |
| | | NMHWVRQAPGQGLEWIGYIYPYNGGTGYNQKFKSKATITADES |
| | | TNTAYMELSSLRSEDTAVYYCARGRPAMDYWGQGTLVTVSSGG |
| | | GGSGGGGSGGGGSDIQMTQSPSSLSASVGDRVTITCRASESVDNY |
| | | GISFMNWFQQKPGKAPKLLIYAASNQGSGVPSRFSGSGSGTDFTL |
| | | TISSLQPDDFATYYCQQSKEVPWTFGQGTKVEIKGGsgEQKLISEE |
| | | DLggIEPDGGSGQVQLVQSGAEVKKPGSSVKVSCKAFGGTFSSYA |
| | | ISWVRQAPGQGLEWMGRIIRFLGIANYAQKFQGRVTLIADKSTNT |
| | | AYMELSSLRSEDTAVYYCAGEPGREDPDAVDIWGQGTMVTVSS |
| | | GGGGSGGGGSGGGGSDIQMTQSPSSLSASVGDRVTITCRASQGIR |
| | | SWLAWYQQKPEKARKSLIYAASSLQSGVPSRFSGSGSGTDFTLTI |
| | | SSLQPEDFATYYCQQYNSYPLTFGGGTKVEIKAKPTTTPAPRPPTP |
| | | APTIASQPLSLRPEACRPAAGGAVHTRGLDFAPRKIEVMYPPPYL |
| | | DNEKSNGTIIHVKGKHLCPSPLFPGPSKPFWVLVVVGGVLACYSL |
| | | LVTVAFIIFWVRSKRSRLLHSDYMNMTPRRPGPTRKHYQPYAPPR |
| | | DFAAYRSRVKFSRSADAPAYQQGQNQLYNELNLGRREEYDVLD |
| | | KRRGRDPEMGGKPRRKNPQEGLYNELQKDKMAEAYSEIGMKGE |
| | | RRRGKGHDGLYQGLSTATKDTYDALHMQALPPR |
| 295 | CD63-MMP9-2L-TNFa-Myc- | MAVEGGMKCVKFLLYVLLLAFCACAVGLIAVGVGAQLVLSQTII |
| | MMP9-CD63-Cyt | QGATPGSLLPVVIIAVGVFLFLVAFVGCCGACKENYCLMITFAIFL |
| | | SLIMLVEVAAAIAGYVGGSGPVRRYQggPLGMTSSSSRTPSDKPV |
| | | AHVVANPQAEGQLQWLNRRANALLANGVELRDNQLVVPSEGLY |
| | | LIYSQVLFKGQGCPSTHVLLTHTISRIAVSYQTKVNLLSAIKSPCQ |
| | | RETPEGAEAKPWYEPIYLGGVFQLEKGDRLSAEINRPDYLDFAES |
| | | GQVYFGIIALGGsgEQKLISEEDLggPLGMTSGGSGPKNNHTASILD |
| | | RMQADFKCCGAANYTDWEKIPSMSKNRVPDSCCINVTVGCGINF |
| | | NEKAIHKEGCVEKIGGWLRKNVLVVAAAALGIAFVEVLGIVFAC |
| | | CLVKSIRSGYEVM |
| 311 | CD63-MMP9-2L-TNFa-Myc- | MAVEGGMKCVKFLLYVLLLAFCACAVGLIAVGVGAQLVLSQTII |
| | MMP9-CD63-D-Cyt (Lacks EVs | QGATPGSLLPVVIIAVGVFLFLVAFVGCCGACKENYCLMITFAIFL |
| | sorting motif) | SLIMLVEVAAAIAGYVGGSGPVRRYQggPLGMTSSSSRTPSDKPV |
| | | AHVVANPQAEGQLQWLNRRANALLANGVELRDNQLVVPSEGLY |
| | | LIYSQVLFKGQGCPSTHVLLTHTISRIAVSYQTKVNLLSAIKSPCQ |
| | | RETPEGAEAKPWYEPIYLGGVFQLEKGDRLSAEINRPDYLDFAES |
| | | GQVYFGIIALGGsgEQKLISEEDLggPLGMTSGGSGPKNNHTASILD |
| | | RMQADFKCCGAANYTDWEKIPSMSKNRVPDSCCINVTVGCGINF |
| | | NEKAIHKEGCVEKIGGWLRKNVLVVAAAALGIAFVEVLGIVFAC |
| | | CLVKS |
| 368 | CD63-DL 1-MMP2-2L-TNFa- | MAVEGGMKCVKCGACKENYCLMITFAIFLSLIMLVEVAAAIAGY |
| | Myc-MMP2-CD63-Cyt | VGGSGPVRRYQGGPLGVRGGSSSRTPSDKPVAHVVANPQAEGQL |
| | | QWLNRRANALLANGVELRDNQLVVPSEGLYLIYSQVLFKGQGCP |
| | | STHVLLTHTISRIAVSYQTKVNLLSAIKSPCQRETPEGAEAKPWYE |
| | | PIYLGGVFQLEKGDRLSAEINRPDYLDFAESGQVYFGIIALGGsgE |
| | | QKLISEEDLGGPLGVRGGGGSGPKNNHTASILDRMQADFKCCGA |
| | | ANYTDWEKIPSMSKNRVPDSCCINVTVGCGINFNEKAIHKEGCVE |
| | | KIGGWLRKNVLVVAAAALGIAFVEVLGIVFACCLVKSIRSGYEV |
| | | M |
| 369 | CD63-MMP9-2L-TNFa-Myc- | MAVEGGMKCVKFLLYVLLLAFCACAVGLIAVGVGAQLVLSQTII |
| | MMP9-aCD19CAR | QGATPGSLLPVVIIAVGVFLFLVAFVGCCGACKENYCLMITFAIFL |
| | | SLIMLVEVAAAIAGYVGGSGPVRRYQggPLGMTSSSSRTPSDKPV |
| | | AHVVANPQAEGQLQWLNRRANALLANGVELRDNQLVVPSEGLY |
| | | LIYSQVLFKGQGCPSTHVLLTHTISRIAVSYQTKVNLLSAIKSPCQ |
| | | RETPEGAEAKPWYEPIYLGGVFQLEKGDRLSAEINRPDYLDFAES |
| | | GQVYFGIIALGGsgEQKLISEEDLggPLGMTSGGSGGGSGPDIQMT |
| | | QTTSSLSASLGDRVTISCRASQDISKYLNWYQQKPDGTVKLLIYH |
| | | TSRLHSGVPSRFSGSGSGTDYSLTISNLEQEDIATYFCQQGNTLPY |
| | | TFGGGTKLEITGGGGSGGGGSGGGGSEVKLQESGPGLVAPSQSLS |
| | | VTCTVSGVSLPDYGVSWIRQPPRKGLEWLGVIWGSETTYYNSAL |
| | | KSRLTIIKDNSKSQVFLKMNSLQTDDTAIYYCAKHYYYGGSYAM |
| | | DYWGQGTSVTVSSAKPTTTPAPRPPTPAPTIASQPLSLRPEACRPA |
| | | AGGAVHTRGLDFAPRKIEVMYPPPYLDNEKSNGTIIHVKGKHLCP |
| | | SPLFPGPSKPFWVLVVVGGVLACYSLLVTVAFIIFWVRSKRSRLL |
| | | HSDYMNMTPRRPGPTRKHYQPYAPPRDFAAYRSRVKFSRSADAP |
| | | AYQQGQNQLYNELNLGRREEYDVLDKRRGRDPEMGGKPRRKNP |
| | | QEGLYNELQKDKMAEAYSEIGMKGERRRGKGHDGLYQGLSTAT |
| | | KDTYDALHMQALPPR |
| 371 | CD63-MMP2-2L-TNFa-Myc- | MAVEGGMKCVKFLLYVLLLAFCACAVGLIAVGVGAQLVLSQTII |
| | MMP2-CD63-D-Cyt | QGATPGSLLPVVIIAVGVFLFLVAFVGCCGACKENYCLMITFAIFL |
| | | SLIMLVEVAAAIAGYVGGSGPVRRYQGGPLGVRGGSSSRTPSDKP |
| | | VAHVVANPQAEGQLQWLNRRANALLANGVELRDNQLVVPSEGL |
| | | YLIYSQVLFKGQGCPSTHVLLTHTISRIAVSYQTKVNLLSAIKSPC |
| | | QRETPEGAEAKPWYEPIYLGGVFQLEKGDRLSAEINRPDYLDFAE |
| | | SGQVYFGIIALGGsgEQKLISEEDLGGPLGVRGGGGSGPKNNHTAS |
| | | ILDRMQADFKCCGAANYTDWEKIPSMSKNRVPDSCCINVTVGCG |
| | | INFNEKAIHKEGCVEKIGGWLRKNVLVVAAAALGIAFVEVLGIVF |
| | | ACCLVKS |
| 372 | CD63-MMP2-2L-TNFa-Myc- | MAVEGGMKCVKFLLYVLLLAFCACAVGLIAVGVGAQLVLSQTII |
| | MMP2-aCD19CAR | QGATPGSLLPVVIIAVGVFLFLVAFVGCCGACKENYCLMITFAIFL |
| | | SLIMLVEVAAAIAGYVGGSGPVRRYQGGPLGVRGGSSSRTPSDKP |
| | | VAHVVANPQAEGQLQWLNRRANALLANGVELRDNQLVVPSEGL |
| | | YLIYSQVLFKGQGCPSTHVLLTHTISRIAVSYQTKVNLLSAIKSPC |
| | | QRETPEGAEAKPWYEPIYLGGVFQLEKGDRLSAEINRPDYLDFAE |
| | | SGQVYFGIIALGGsgEQKLISEEDLGGPLGVRGGGGSGGGSGPDIQ |
| | | MTQTTSSLSASLGDRVTISCRASQDISKYLNWYQQKPDGTVKLLI |
| | | YHTSRLHSGVPSRFSGSGSGTDYSLTISNLEQEDIATYFCQQGNTL |
| | | PYTFGGGTKLEITGGGGSGGGGSGGGGSEVKLQESGPGLVAPSQS |
| | | LSVTCTVSGVSLPDYGVSWIRQPPRKGLEWLGVIWGSETTYYNS |
| | | ALKSRLTIIKDNSKSQVFLKMNSLQTDDTAIYYCAKHYYYGGSY |
| | | AMDYWGQGTSVTVSSAKPTTTPAPRPPTPAPTIASQPLSLRPEAC |
| | | RPAAGGAVHTRGLDFAPRKIEVMYPPPYLDNEKSNGTIIHVKGKH |
| | | LCPSPLFPGPSKPFWVLVVVGGVLACYSLLVTVAFIIFWVRSKRSR |
| | | LLHSDYMNMTPRRPGPTRKHYQPYAPPRDFAAYRSRVKFSRSAD |
| | | APAYQQGQNQLYNELNLGRREEYDVLDKRRGRDPEMGGKPRRK |
| | | NPQEGLYNELQKDKMAEAYSEIGMKGERRRGKGHDGLYQGLST |
| | | ATKDTYDALHMQALPPR |
| 370 | CD69cyt-TMII-TNFa-Myctag- | MSSENCFVAENSSLHPESGQENDATSPHFSTRHEGSFQVPVLCAV |
| | CD28TM-cyt | MNVVFITILIIALIALSVGQYNGGSGPVRRYQSSSRTPSDKPVAHV |
| | | VANPQAEGQLQWLNRRANALLANGVELRDNQLVVPSEGLYLIY |
| | | SQVLFKGQGCPSTHVLLTHTISRIAVSYQTKVNLLSAIKSPCQRET |
| | | PEGAEAKPWYEPIYLGGVFQLEKGDRLSAEINRPDYLDFAESGQV |
| | | YFGIIALGGSGEQKLISEEDLGGSGCPSPLFPGPSKPFWVLVVVGG |
| | | VLACYSLLVTVAFIIFWVRSKRSRLL |
| 377 | CD69cyt-TMII-MMP9-TNFa- | MSSENCFVAENSSLHPESGQENDATSPHFSTRHEGSFQVPVLCAV |
| | Myc-MMP9-CD28TM-cyt | MNVVFITILIIALIALSVGQYNGGSGPVRRYQGGSGGGPLGMTSG |
| | | GSGSSSRTPSDKPVAHVVANPQAEGQLQWLNRRANALLANGVE |
| | | LRDNQLVVPSEGLYLIYSQVLFKGQGCPSTHVLLTHTISRIAVSYQ |
| | | TKVNLLSAIKSPCQRETPEGAEAKPWYEPIYLGGVFQLEKGDRLS |
| | | AEINRPDYLDFAESGQVYFGIIALGGSGEQKLISEEDLGGPLGMTS |
| | | GGSGCPSPLFPGPSKPFWVLVVVGGVLACYSLLVTVAFIIFWVRS |
| | | KRSRLL |
| 373 | CD69Cyt-TM-GrzB-TNFa- | MSSENCFVAENSSLHPESGQENDATSPHFSTRHEGSFQVPVLCAV |
| | Myc-GrzB-a-CD19sc-Stalk- | MNVVFITILIIALIALSVGQYNGGSGPVRRYQGGSGGGEEEEEVEA |
| | CD28-CD3z | DSEEEEEEESGGSGSSSRTPSDKPVAHVVANPQAEGQLQWLNRR |
| | | ANALLANGVELRDNQLVVPSEGLYLIYSQVLFKGQGCPSTHVLL |
| | | THTISRIAVSYQTKVNLLSAIKSPCQRETPEGAEAKPWYEPIYLGG |
| | | VFQLEKGDRLSAEINRPDYLDFAESGQVYFGIIALEQKLISEEDLG |
| | | GSGEEEEEVEADSEEEEEEEGGSGGGSGGGSGPDIQMTQTTSSLS |
| | | ASLGDRVTISCRASQDISKYLNWYQQKPDGTVKLLIYHTSRLHSG |
| | | VPSRFSGSGSGTDYSLTISNLEQEDIATYFCQQGNTLPYTFGGGTK |
| | | LEITGGGGSGGGGSGGGGSEVKLQESGPGLVAPSQSLSVTCTVSG |
| | | VSLPDYGVSWIRQPPRKGLEWLGVIWGSETTYYNSALKSRLTIIK |
| | | DNSKSQVFLKMNSLQTDDTAIYYCAKHYYYGGSYAMDYWGQG |
| | | TSVTVSSGGGGSGGGGSAKPTTTPAPRPPTPAPTIASQPLSLRPEA |
| | | CRPAAGGAVHTRGLDFAPRKIEVMYPPPYLDNEKSNGTIIHVKGK |
| | | HLCPSPLFPGPSKPFWVLVVVGGVLACYSLLVTVAFIIFWVRSKR |
| | | SRLLHSDYMNMTPRRPGPTRKHYQPYAPPRDFAAYRSRVKFSRS |
| | | ADAPAYQQGQNQLYNELNLGRREEYDVLDKRRGRDPEMGGKPR |
| | | RKNPQEGLYNELQKDKMAEAYSEIGMKGERRRGKGHDGLYQGL |
| | | STATKDTYDALHMQALPPR |
| 374 | CD69Cyt-TM-GrzB-nLuc-Myc- | MSSENCFVAENSSLHPESGQENDATSPHFSTRHEGSFQVPVLCAV |
| | GrzB-CD28-CD3z (Delta SC- | MNVVFITILIIALIALSVGQYNGGSGPVRRYSRQGGSGGGEEEEEV |
| | GAGE B) | EADSEEEEEEESGGSGSSSSRVFTLEDFVGDWRQTAGYNLDQVLE |
| | | QGGVSSLFQNLGVSVTPIQRIVLSGENGLKIDIHVIIPYEGLSGDQM |
| | | GQIEKIFKVVYPVDDHHFKVILHYGTLVIDGVTPNMIDYFGRPYE |
| | | GIAVFDGKKITVTGTLWNGNKIIDERLINPDGSLLFRVTINGVTGW |
| | | RLCERILAEQKLISEEDLLEGGSGEEEEEVEADSEEEEEEEGGSGG |
| | | GSGLEPRGGSGVNSGGGGSGGGGSAKPTTTPAPRPPTPAPTIASQP |
| | | LSLRPEACRPAAGGAVHTRGLDFAPRKIEVMYPPPYLDNEKSNGT |
| | | IIHVKGKHLCPSPLFPGPSKPFWVLVVVGGVLACYSLLVTVAFIIF |
| | | WVRSKRSRLLHSDYMNMTPRRPGPTRKHYQPYAPPRDFAAYRS |
| | | RVKFSRSADAPAYQQGQNQLYNELNLGRREEYDVLDKRRGRDP |
| | | EMGGKPRRKNPQEGLYNELQKDKMAEAYSEIGMKGERRRGKGH |
| | | DGLYQGLSTATKDTYDALHMQALPPR |
| 375 | CD69Cyt-TM-nLuc-Myc-a- | MSSENCFVAENSSLHPESGQENDATSPHFSTRHEGSFQVPVLCAV |
| | CD19sc-Stalk-CD28-CD3z | MNVVFITILIIALIALSVGQYNGGSGPVRRYSRVFTLEDFVGDWRQ |
| | (Delta GrzB, GAGE A) | TAGYNLDQVLEQGGVSSLFQNLGVSVTPIQRIVLSGENGLKIDIHV |
| | | IIPYEGLSGDQMGQIEKIFKVVYPVDDHHFKVILHYGTLVIDGVTP |
| | | NMIDYFGRPYEGIAVFDGKKITVTGTLWNGNKIIDERLINPDGSLL |
| | | FRVTINGVTGWRLCERILAEQKLISEEDLLEPRGGSGVNPDIQMTQ |
| | | TTSSLSASLGDRVTISCRASQDISKYLNWYQQKPDGTVKLLIYHTS |
| | | RLHSGVPSRFSGSGSGTDYSLTISNLEQEDIATYFCQQGNTLPYTF |
| | | GGGTKLEITGGGGSGGGGSGGGGSEVKLQESGPGLVAPSQSLSVT |
| | | CTVSGVSLPDYGVSWIRQPPRKGLEWLGVIWGSETTYYNSALKS |
| | | RLTIIKDNSKSQVFLKMNSLQTDDTAIYYCAKHYYYGGSYAMDY |
| | | WGQGTSVTVSSVNSGGGGSGGGGSAKPTTTPAPRPPTPAPTIASQ |
| | | PLSLRPEACRPAAGGAVHTRGLDFAPRKIEVMYPPPYLDNEKSNG |
| | | TIIHVKGKHLCPSPLFPGPSKPFWVLVVVGGVLACYSLLVTVAFII |
| | | FWVRSKRSRLLHSDYMNMTPRRPGPTRKHYQPYAPPRDFAAYRS |
| | | RVKFSRSADAPAYQQGQNQLYNELNLGRREEYDVLDKRRGRDP |
| | | EMGGKPRRKNPQEGLYNELQKDKMAEAYSEIGMKGERRRGKGH |
| | | DGLYQGLSTATKDTYDALHMQALPPR |
| 358 | CD69Cyt-TM-GrzB (Original)- | MSSENCFVAENSSLHPESGQENDATSPHFSTRHEGSFQVPV |
| | nLuc-Myc-GrzB(Original)-a- | LCAVMNVVFITILIIALIALSVGQYNGGSGPVRRYSRQGGSG |
| | CD19sc-Stalk-CD28-CD3z | GGEEEEEVEADSEEEEEEESGGSGSSSSRVFTLEDFVGDWR |
| | | QTAGYNLDQVLEQGGVSSLFQNLGVSVTPIQRIVLSGENGL |
| | | KIDIHVIIPYEGLSGDQMGQIEKIFKVVYPVDDHHFKVILHY |
| | | GTLVIDGVTPNMIDYFGRPYEGIAVFDGKKITVTGTLWNGN |
| | | KIIDERLINPDGSLLFRVTINGVTGWRLCERILAEQKLISEED |
| | | LLEGGSGEEEEEVEADSEEEEEEEGGSGGGSGLEPRGGSGV |
| | | NPDIQMTQTTSSLSASLGDRVTISCRASQDISKYLNWYQQK |
| | | PDGTVKLLIYHTSRLHSGVPSRFSGSGSGTDYSLTISNLEQE |
| | | DIATYFCQQGNTLPYTFGGGTKLEITGGGGSGGGGSGGGG |
| | | SEVKLQESGPGLVAPSQSLSVTCTVSGVSLPDYGVSWIRQP |
| | | PRKGLEWLGVIWGSETTYYNSALKSRLTIIKDNSKSQVFLK |
| | | MNSLQTDDTAIYYCAKHYYYGGSYAMDYWGQGTSVTVS |
| | | SVNSGGGGSGGGGSAKPTTTPAPRPPTPAPTIASQPLSLRPE |
| | | ACRPAAGGAVHTRGLDFAPRKIEVMYPPPYLDNEKSNGTII |
| | | HVKGKHLCPSPLFPGPSKPFWVLVVVGGVLACYSLLVTVA |
| | | FIIFWVRSKRSRLLHSDYMNMTPRRPGPTRKHYQPYAPPRD |
| | | FAAYRSRVKFSRSADAPAYQQGQNQLYNELNLGRREEYD |
| | | VLDKRRGRDPEMGGKPRRKNPQEGLYNELQKDKMAEAYS |
| | | EIGMKGERRRGKGHDGLYQGLSTATKDTYDALHMQALPP |
| | | R |
| 383 | CD69Cyt-TM-GrzB(IEPD)- | MSSENCFVAENSSLHPESGQENDATSPHFSTRHEGSFQVPV |
| | nLuc-Myc-GrzB(IEPD)-a- | LCAVMNVVFITILIIALIALSVGQYNGGSGPVRRYSRQGGSG |
| | CD19sc-Stalk-CD28-CD3z | GGIEPDSGGSGSRVFTLEDFVGDWRQTAGYNLDQVLEQGG |
| | | VSSLFQNLGVSVTPIQRIVLSGENGLKIDIHVIIPYEGLSGDQ |
| | | MGQIEKIFKVVYPVDDHHFKVILHYGTLVIDGVTPNMIDYF |
| | | GRPYEGIAVFDGKKITVTGTLWNGNKIIDERLINPDGSLLFR |
| | | VTINGVTGWRLCERILAEQKLISEEDLLEGGSGIEPDGGSGG |
| | | GSGLEPRGGSGVNPDIQMTQTTSSLSASLGDRVTISCRASQ |
| | | DISKYLNWYQQKPDGTVKLLIYHTSRLHSGVPSRFSGSGSG |
| | | TDYSLTISNLEQEDIATYFCQQGNTLPYTFGGGTKLEITGGG |
| | | GSGGGGSGGGGSEVKLQESGPGLVAPSQSLSVTCTVSGVS |
| | | LPDYGVSWIRQPPRKGLEWLGVIWGSETTYYNSALKSRLTI |
| | | IKDNSKSQVFLKMNSLQTDDTAIYYCAKHYYYGGSYAMD |
| | | YWGQGTSVTVSSVNSGGGGSGGGGSAKPTTTPAPRPPTPA |
| | | PTIASQPLSLRPEACRPAAGGAVHTRGLDFAPRKIEVMYPP |
| | | PYLDNEKSNGTIIHVKGKHLCPSPLFPGPSKPFWVLVVVGG |
| | | VLACYSLLVTVAFIIFWVRSKRSRLLHSDYMNMTPRRPGPT |
| | | RKHYQPYAPPRDFAAYRSRVKFSRSADAPAYQQGQNQLY |
| | | NELNLGRREEYDVLDKRRGRDPEMGGKPRRKNPQEGLYN |
| | | ELQKDKMAEAYSEIGMKGERRRGKGHDGLYQGLSTATKD |
| | | TYDALHMQALPPR |
| 376 | CD69Cyt-TM-GrzB(SASA)- | MSSENCFVAENSSLHPESGQENDATSPHFSTRHEGSFQVPVLCAV |
| | nLuc-Myc-GrzB(SASA)-a- | MNVVFITILIIALIALSVGQYNGGSGPVRRYSRQGGSGGGAQGVIS |
| | CD19sc-Stalk-CD28-CD3z | ASASNLDDFYSGGSGSRVFTLEDFVGDWRQTAGYNLDQVLEQG |
| | | GVSSLFQNLGVSVTPIQRIVLSGENGLKIDIHVIIPYEGLSGDQMGQ |
| | | IEKIFKVVYPVDDHHFKVILHYGTLVIDGVTPNMIDYFGRPYEGIA |
| | | VFDGKKITVTGTLWNGNKIIDERLINPDGSLLFRVTINGVTGWRL |
| | | CERILAEQKLISEEDLLEGGSGAQGVISASASNLDDFYGGSGGGSG |
| | | LEPRGGSGVNPDIQMTQTTSSLSASLGDRVTISCRASQDISKYLN |
| | | WYQQKPDGTVKLLIYHTSRLHSGVPSRFSGSGSGTDYSLTISNLE |
| | | QEDIATYFCQQGNTLPYTFGGGTKLEITGGGGSGGGGSGGGGSE |
| | | VKLQESGPGLVAPSQSLSVTCTVSGVSLPDYGVSWIRQPPRKGLE |
| | | WLGVIWGSETTYYNSALKSRLTIIKDNSKSQVFLKMNSLQTDDTA |
| | | IYYCAKHYYYGGSYAMDYWGQGTSVTVSSVNSGGGGSGGGGS |
| | | AKPTTTPAPRPPTPAPTIASQPLSLRPEACRPAAGGAVHTRGLDFA |
| | | PRKIEVMYPPPYLDNEKSNGTIIHVKGKHLCPSPLFPGPSKPFWVL |
| | | VVVGGVLACYSLLVTVAFIIFWVRSKRSRLLHSDYMNMTPRRPG |
| | | PTRKHYQPYAPPRDFAAYRSRVKFSRSADAPAYQQGQNQLYNEL |
| | | NLGRREEYDVLDKRRGRDPEMGGKPRRKNPQEGLYNELQKDKM |
| | | AEAYSEIGMKGERRRGKGHDGLYQGLSTATKDTYDALHMQALP |
| | | PR |
| 384 | CD69Cyt-TM-GrzB(ADKG)- | MSSENCFVAENSSLHPESGQENDATSPHFSTRHEGSFQVPVLCAV |
| | nLuc-Myc-GrzB(ADKG)-a- | MNVVFITILIIALIALSVGQYNGGSGPVRRYSRQGGSGGGLEADK |
| | CD19sc-Stalk-CD28-CD3z | GKLEYDSGGSGSRVFTLEDFVGDWRQTAGYNLDQVLEQGGVSS |
| | | LFQNLGVSVTPIQRIVLSGENGLKIDIHVIIPYEGLSGDQMGQIEKIF |
| | | KVVYPVDDHHFKVILHYGTLVIDGVTPNMIDYFGRPYEGIAVFDG |
| | | KKITVTGTLWNGNKIIDERLINPDGSLLFRVTINGVTGWRLCERIL |
| | | AEQKLISEEDLLEGGSGLEADKGKLEYDGGSGGGSGLEPRGGSG |
| | | VNPDIQMTQTTSSLSASLGDRVTISCRASQDISKYLNWYQQKPDG |
| | | TVKLLIYHTSRLHSGVPSRFSGSGSGTDYSLTISNLEQEDIATYFCQ |
| | | QGNTLPYTFGGGTKLEITGGGGSGGGGSGGGGSEVKLQESGPGL |
| | | VAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYYY |
| | | GGSYAMDYWGQGTSVTVSSVNSGGGGSGGGGSAKPTTTPAPRP |
| | | PTPAPTIASQPLSLRPEACRPAAGGAVHTRGLDFAPRKIEVMYPPP |
| | | YLDNEKSNGTIIHVKGKHLCPSPLFPGPSKPFWVLVVVGGVLACY |
| | | SLLVTVAFIIFWVRSKRSRLLHSDYMNMTPRRPGPTRKHYQPYAP |
| | | PRDFAAYRSRVKFSRSADAPAYQQGQNQLYNELNLGRREEYDVL |
| | | DKRRGRDPEMGGKPRRKNPQEGLYNELQKDKMAEAYSEIGMKG |
| | | ERRRGKGHDGLYQGLSTATKDTYDALHMQALPPR |
| 125 | CD63-CYTOPLASMIC | MCGACKENYC |
| 297 | CD63-CYTOPLASMIC | MAVEGGMKCVK |
| 127 | CD63-TM-HELICAL(inside- | LMITFAIFLSLIMLVEVAAAI |
| | out) 2 | |
| 298 | CD63-TM-HELICAL(inside- | FLLYVLLLAFCACAVGLIAVG |
| | out) 1 | |
| 129 | Linker-a | GGSGPVRRYQ |
| 131 | MMP2 | GGPLGVRGG |
| 133 | VL-a-CD16-118AA | MEVQLVESGGGVVRPGGSLRLSCAASGFTFDDYGMSWVRQAPG |
| | | KGLEWVSGINWNGGSTGYADSVKGRFTISRDNAKNSLYLQMNS |
| | | LRAEDTAVYYCARGRSLLFDYWGQGTLVTVSR |
| 135 | Linker-b and Linker-c | GGGGSGGGGSGGGGS |
| 303 | Linker | GGSGPVRRYQ |
| 332 | Linker | GGSG |
| 343 | Linker | GSGSSS |
| 137 | VH-a-CD16-107AA | SELTQDPAVSVALGQTVRITCQGDSLRSYYASWYQQKPGQAPVL |
| | | VIYGKNNRPSGIPDRFSGSSSGNTASLTITGAQAEDEADYYCNSRD |
| | | SSGNHVVFGGGTKLTVL |
| 139 | Flanking 20AA | PSGQAGAAASESLFVSNHAY |
| 141 | 7AA flanking peptide | EASGGPE |
| 143 | VL-a-CD33-116AA | QVQLVQSGAEVKKPGSSVKVSCKASGYTFTDYNMHWVRQAPG |
| | | QGLEWIGYIYPYNGGTGYNQKFKSKATITADESTNTAYMELSSLR |
| | | SEDTAVYYCARGRPAMDYWGQGTLVTVSS |
| 145 | VH-a-CD33 | DIQMTQSPSSLSASVGDRVTITCRASESVDNYGISFMNWFQQKPG |
| | | KAPKLLIYAASNQGSGVPSRFSGSGSGTDFTLTISSLQPDDFATYY |
| | | CQQSKEVPWTFGQGTKVEIK |
| 147 | Myc-tag | EQKLISEEDL |
| 149 | MMP9 | ggPLGMTS |
| 151 | CD63-EXTRACELLULAR | PKNNHTASILDRMQADFKCCGAANYTDWEKIPSMSKNRVPDSCC |
| | lacks EVs sorting motif | INVTVGCGINFNEKAIHKEGCVEKIGGWLRKNV |
| 308 | CD63-EXTRACELLULAR with | PKNNHTASILDRMQADFKCCGAANYTDWEKIPSMSKNRVPDSCC |
| | EVs sorting motif | INVTVGCGINFNEKAIHKEGCVEKIGGWLRKNVLVVAAAALGIAF |
| | | VEVLGIVFACCLVKSIRSGYEVM |
| 315 | CD63-EXTRACELLULAR 1 | VGAQLVLSQTIIQGATPGS |
| 153 | GPi-anchor | VLRDKLVKCEGISLLAQNTSWLLLLLLSLSLLQATDFMSL |
| 185 | CD63-TM-HELICAL(outside- | LVVAAAALGIAFVEVLGIVFA |
| | in) 2 | |
| 300 | CD63-TM-HELICAL(outside- | LLPVVIIAVGVFLFLVAFVGC |
| | in) 1 | |
| 187 | CD63-CYTOPLASMIC-with | CCLVKSIRSGYEVM |
| | EV Motif | |
| 325 | CD63-CYTOPLASMIC | CCLVKS |
| 254 | CD63-CYTOPLASMIC | MCGACKENYC |
| 214 | TNFSF14 cytoplasmic | MSCSVAR |
| 216 | TNFSF14 transmembrane | VGLGLLLLLMGAGLAVQGWFL |
| | (inside-out) | |
| 218 | TNFSF14 extracellular with | GSWEQLLQLHWRLGEMVTRLPDGPAGSWEQLIQERRSHEVNPA |
| | ADAM17 cleavage seq: | A |
| 240 | TNR1A-Extracellular with | SLECTKLCLPQIENVKGTEDS |
| | ADAM17 cleavage seq | |
| 242 | Vh-a-CD38 | QVQLVQSGAEVKKPGSSVKVSCKAFGGTFSSYAISWVRQAPGQG |
| | | LEWMGRIIRFLGIANYAQKFQGRVTLIADKSTNTAYMELSSLRSE |
| | | DTAVYYCAGEPGREDPDAVDIWGQGTMVTVSS |
| 246 | V1-a-CD38 | DIQMTQSPSSLSASVGDRVTITCRASQGIRSWLAWYQQKPEKARK |
| | | SLIYAASSLQSGVPSRFSGSGSGTDFTLTISSLQPEDFATYYCQQY |
| | | NSYPLTFGGGTKVEIK |
| 248 | TNR1A-Transmembrane | SGTTVLLPLVIFFGLCLLSLLFIGLMYRY |
| 250 | 41BB-intracellular | KRGRKKLLYIFKQPFMRPVQTTQEEDG |
| 287 | Hinge stalk region | AKPTTTPAPRPPTPAPTIASQPLSLRPEACRPAAGGAVHTRGLDFA |
| | | PRKIEVMYPPPYLDNEKSNGTIIHVKGKHLCPSPLFPGPSKP |
| 289 | CD28 transmembrane | FWVLVVVGGVLACYSLLVTVAFIIFWV |
| 291 | CD28 intracellular | RSKRSRLLHSDYMNMTPRRPGPTRKHYQPYAPPRDFAAYRS |
| 293 | CD3z intracellular | RVKFSRSADAPAYQQGQNQLYNELNLGRREEYDVLDKRRGRDP |
| | | EMGGKPRRKNPQEGLYNELQKDKMAEAYSEIGMKGERRRGKGH |
| | | DGLYQGLSTATKDTYDALHMQALPPR |
| 321 | TNFa | SSSRTPSDKPVAHVVANPQAEGQLQWLNRRANALLANGVELRD |
| | | NQLVVPSEGLYLIYSQVLFKGQGCPSTHVLLTHTISRIAVSYQTKV |
| | | NLLSAIKSPCQRETPEGAEAKPWYEPIYLGGVFQLEKGDRLSAEIN |
| | | RPDYLDFAESGQVYFGIIA |
| 328 | CD69-CYTOPLASMIC | MSSENCFVAENSSLHPESGQENDATSPHFSTRHEGSFQVP |
| 329 | CD69-TM-type-II(inside-out) | VLCAVMNVVFITILIIALIAL |
| 330 | CD69-Extracellular | SVGQYNGGSGPVRRYQ |
| 258 | Granzyme B cleavage site | IEPD |
| 260 | Granzyme B cleavage site | EEEEEVEADSEEEEEEE |
| 261 | Granzyme B cleavage site | AQGVISADASNLDDFY |
| 262 | Granzyme B cleavage site | LEADKGKLEYD |
| 381 | Nano-Luciferase | VFTLEDFVGDWRQTAGYNLDQVLEQGGVSSLFQNLGVSVTPIQR |
| | | IVLSGENGLKIDIHVIIPYEGLSGDQMGQIEKIFKVVYPVDDHHFK |
| | | VILHYGTLVIDGVTPNMIDYFGRPYEGIAVFDGKKITVTGTLWNG |
| | | NKIIDERLINPDGSLLFRVTINGVTGWRLCERILA |

In the context of this invention, a payload is any amino acid sequence intended for delivery inside a living organism. Suitable payloads include frill-length proteins and peptides, enzymes, cytokines, chemokines, signaling proteins, and many more proteins with various functions. In the present invention, the payload is often a therapeutic antibody, bi-specific engagers, and cytokines. The payload is preferably less than 2000 amino acids in size.

The payload delivery system of the present invention is intended to be part of cell therapy. The payload delivery system can be delivered to cells via_electroporation, extracellular vesicles, nanoparticles, and transient or stable genetic modification. The protein sequences disclosed here can be extracellular and/or intracellular. The location on the cell membrane is determined by the type of transmembrane system used.

In some embodiments, the sheddase is an actual part of the peptide delivery system. The sheddase is released when the cell is under the intended condition for the release of the sheddase, thereby allowing the sheddase to be delivered to treat conditions that are not associated with sheddases. An inactivated or split sheddase can be expressed on the surface of the cell which can be activated under the right conditions where a trigger can result in the activation and/or reorganization and assembley of the shedasse into activated one. Moreover, sheddase can also be expressed through an artificial promoter-based system which is activated under the right conditions including the receptor ligation and activation or cleavage of a membrane bound transcription factor following the receptor ligation. Certain sheddases maybe released from the target cells where due to extrinsic or intrinsic pathways lead to the apoptosis including Caspase 3. Caspase-3 is a key protein involved in apoptosis, or programmed cell death, in mammalian cells. It belongs to a family of cysteine proteases known as caspases, which play essential roles in initiating and executing apoptosis. Caspase-3 is considered an effector caspase because it acts downstream in the apoptotic signaling pathways and is responsible for executing the final stages of cell death. Once the Capsase-3 is released from the dying cells, it is released in the TME and releases the peptide.

Several sheddases have been found to be differentially expressed in the tumor microenvironment (TME) compared to normal tissue. For example, MMPs, GrzB and ADAMs, ADAMTSs are often overexpressed in many different types of cancer, including breast, lung, and pancreatic cancer. The differential expression of sheddases in the TME can have important implications for tumor progression and metastasis. For example, increased expression of MMP9, MMP2, ADAM10 and ADAM17 in the TME has been associated with increased release of pro-tumorigenic growth factors and cytokines, such as epidermal growth factor (EGF) and tumor necrosis factor alpha (TNF-α). This can lead to increased tumor cell proliferation, survival, and invasion. Overall, the differential expression of sheddases in the TME compared to normal tissue highlights the potential for targeting these enzymes as a therapeutic strategy for cancer treatment. Therefore, the differential expression of the sheddases can be exploited for the site directed specific delivery of the payloads.

We have designed a cell-based peptide delivery system that allows the delivery and release of the payload/cargo directly at the desired site of action. The system consists of single or multi pass transmembrane (TM), or C-type lectin proteins embedded into the membrane of the vehicle cell with additional motifs that regulate the delivery/release of the payload in the required milieu. Thus, instead of direct systemic biologics injections, we aimed to transfuse engineered cells or EVs derived thereof that could deliver the payload to the site where it is needed and release it at the right time.

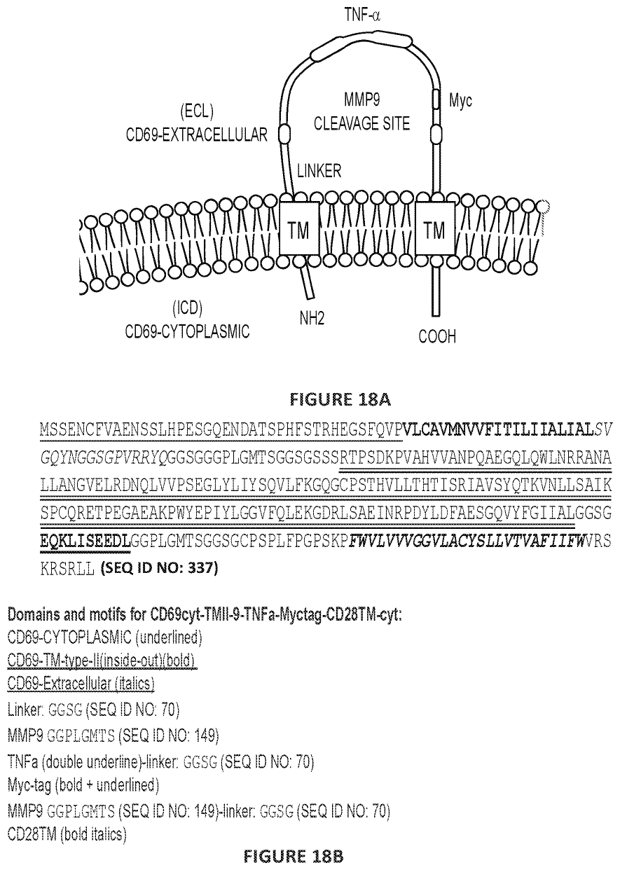

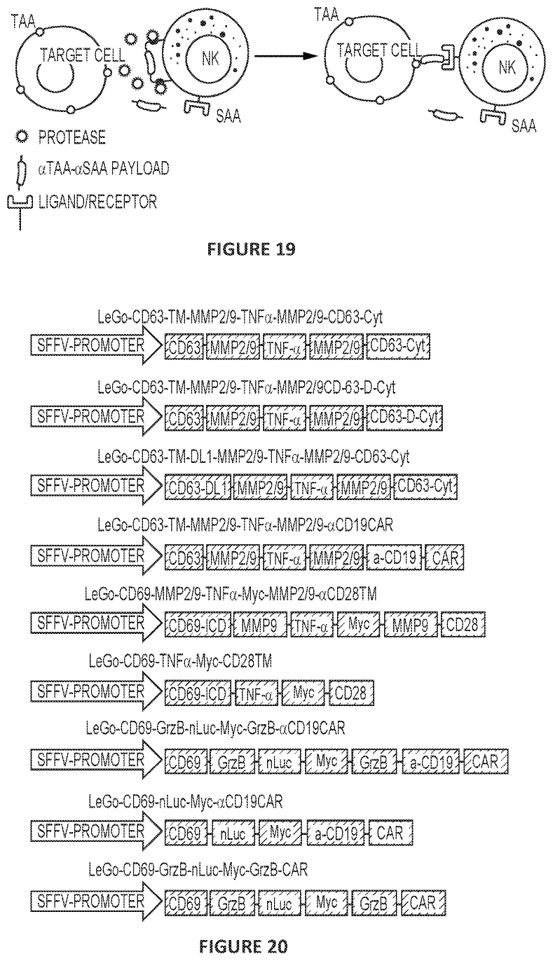

While the delivery system is independent of cell type, as an example, we have modified NK cells to produce and deliver a bi-specific engager in the tumor environment. This delivery is further regulated and can be induced upon either NK cell activation (activation-induced cleavage) or the tumor milieu (by cleavage upon encountering proteases enriched specifically in the TME) (FIG. 19). NK cell activation can be due to the interaction of its germline-encoded activation receptors or specifically engineered receptors, such as chimeric antigen receptor/s against tumor antigen/s. NK cell activation-induced cleavage can be achieved either through Granzyme-B, released following degranulation, or the physical pull, as in notch activation. In addition, such an activation-stimulated release will lead to NK cell expansion and activation depending on the tumor antigen availability. This is a next-generation cancer immunotherapy system with an augmented capacity to produce and release the payload under the desired conditions and at specific sites.

Although many sheddases can be used for this payload delivery system, the current work presents general examples of matrix metalloproteinases (MMPs), which may allow the milieu-specific cargo release. Also, a model based on the ADAM family of MMPs, and the serine-protease Granzyme-B has been presented. In these models, supplementary functions like tumor targeting, expansion, and/or activation were modulated by adding extracellular and/or intracellular domains. These additional domains can include anything from extracellular cytokines, drugs or pro-drugs, chimeric antigen receptor (CAR)-like or dual antigen receptor (DAR)-like domains or intracellular modulators, inhibitors, signaling domain-containing proteins, transcription factors, transcriptional activators or inhibitors, DNA modifying enzymes such as HDAC, proteins containing localization and dimerization domains, proteins containing destruction and dimerization domains, proteins containing hypoxia-responsive domains, proteins that activate immune cells, proteins that increase persistence and proliferation of immune cells, proteins that induce migration of immune cells, split receptors such as split CARs, etc.

Moreover, the system has been devised for passive release in the tumor milieu depending on the specific MMPs released by the cancer cells or active release induced by the activation of producer cells due to receptor engagement.

The membrane embedded systems can also be designed in such a way that they are sorted into specific subcellular compartments. In some embodiments, we engineered the payload that will be sorted into extracellular vesicles (EVs), such as exosomes. The cell releases these exosomes in huge quantities and can release the payload upon cleavage by proteases within the TME or other target sites (FIGS. 4A-B, 5A-B, 9, 10, and 24-28). In theory, these EVs can also be harvested from in-vitro culture and given to patients as an off-the-shelf therapeutic.

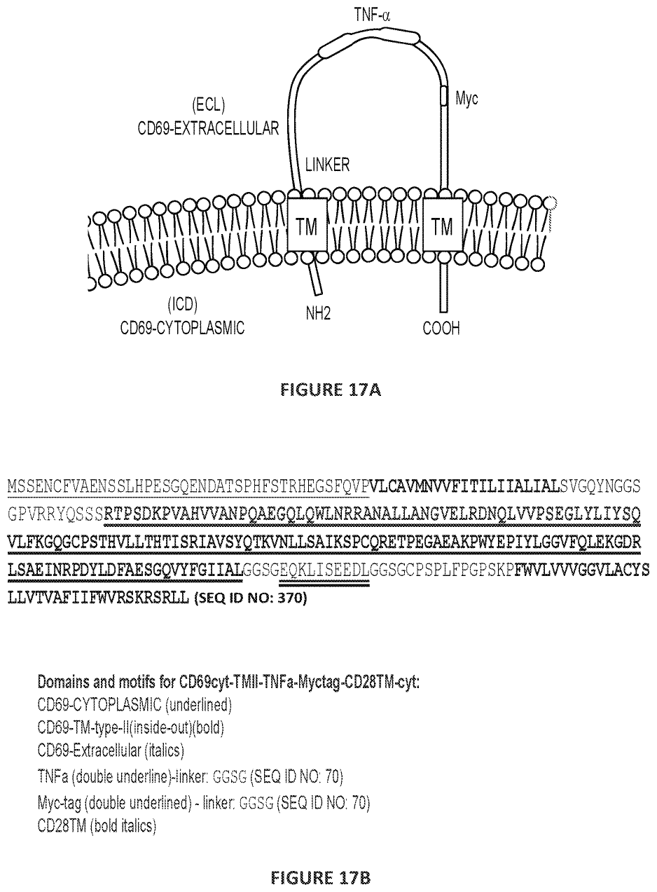

In some embodiments, in NK or T cells, the CD69 TM is embedded into the membrane with at least two protease cleavage site motifs that regulate the release of the payload in the required milieu (FIGS. 17A-B and 18A-B). In addition to the model described in FIGS. 17A-B and 18A-B, optionally, the system can be equipped with a single-chain CAR protein at N-terminus for a specific delivery (FIG. 6).

In some embodiment, a modified tetraspanin can be used, where more than two protease cleavage site motifs are inserted with several potential cargo delivery/releases. This system also has the possibility of releasing intracellular payloads (FIG. 7).

In some embodiment, further modification of tetraspanin can be used with an extra single-chain against the self-associated antigen (a-SAA) connected with the intracellular domain of CD45 (FIG. 8).

In some embodiments, the intracellular or extracellular domain comprises of OX-40, 4-1BB/CD137, CD2, CD7, CD27, CD30, B7-1 (CD80), B7-2 (CD86),PDL-1, programmed death-I (PD-1), inducible T cell co-stimulator (ICOS), lymphocyte function-associated antigen-I (LFA-1 (CDI la/CD 18), CD3 gamma, CD3 delta, CD3 epsilon, CD247, CD276 (B7-H3), LIGHT, (TNFSF14), NKG2C, lg alpha (CD79a), DAP-10, Fe gammareceptor, MHC class I molecule, TNF receptor proteins, an Immunoglobulin protein, cytokine receptor, integrins, Signaling Lymphocytic Activation Molecules (SLAM proteins), The costimulatory ligand (PD-L2, 4-1 BBL, OX40L, inducible costimulatory ligand (ICOS-L), intercellular adhesion molecule (ICAM, CD30L, CD70, CD83, HLA-G, MICA, Ml CB, HVEM, lymphotoxin b receptor, 3/TR6, ILT3, ILT4), activating NK cell receptors, BTLA, a Toll ligand receptor, ICAM-1, B7-H3, CDS, ICAM-1, GITR, BAFFR, LIGHT, HVEM (LIGHTR), KIRDS2, NKp80 (KLRFI), NKp44, NKp30, NKp46, CD19, CD8alpha, CD8beta, IL-2R beta, IL-2R gamma, IL-7R alpha, ITGA4, VLAl, CD49a, ITGA4, CD49D, ITGA6, VLA-6, CD49f, ITGAD, CDl Id, ITGAE, CD103, ITGAL, CDl la, LFA-1, IT GAM, CDl lb, ITGAX, CDl le, ITGB1, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD 160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

In some embodiments, the antigen binding domain specifically binds BCMA, MUC16 (also known as CA125), EGFR, EGFRvIII, MUCI, Flt-3, WT-1, CD38, CD70, CD90, CD133, MHC-WTI, TSPANI0, MHC-PRAME, MHC-NY-ESOI, HER2 (ERBB2), CA-IX (Carbonic anhydrase IX), LIVI, ADAMI0, CHRNA2, LeY, NKG2D, CSI, CD44v6, CD24, LGR5, ALDH, ALDH1, CD54, Sca1, CD271, CD123, CD36, CD109, CD110, CD71-negative, CCA, ABCG2, Claudin-18.2 (Claudin-18A2, or Claudinl8 isoform 2), PSCA, DLL3 (Delta-like protein 3, Drosophila Delta homolog 3, Delta3), Mud 7 (Mucinl 7, Muc3, Muc3), FAP alpha (Fibroblast Activation Protein alpha), Ly6G6D (Lymphocyte antigen 6 complex locus protein G6d, c6orf23, G6D, MEGTI, NG25), PSMA, MSLN, or RNF43 (E3 ubiquitin-protein ligase RNF43, RING finger protein 43). BAFF, C242 antigen, disialoganglioside (GD2), 4-IBB, 5T4, CD22, CD221, CD23 (IgE receptor), CD28, CD30 (TNFRSF8), CD33, CD4, CD40, CD44v6, CD44 CD51, CD52, CD56, CD74, CEA, CNT0888, CTLA-4, DR5, EpCAM, FAP, fibronectin extradomain-B, folate receptor 1, GD3 ganglioside, glycoprotein 75, GPNMB, HER2/neu, HGF, human scatter factor receptor kinase, IGF-1 receptor, IGF-I, IgGI, LI-CAM, integrin α5β1, integrin αvβ, legumain, MORAb-009, MS4A1, MUC1, mucin CanAg, C-MET, CCR4, CD152, CD10, CD19, CD20, CD200, N-glycolylneuraminic acid, NPC-IC, PDGF-R a, PDL192, phosphatidylserine, tumor antigen CTAA16.88, VEGF-A, VEGFR-1, VEGFR2, vimentin, RANKL, RON, ROR1, SCH900105, SDC1, SLAMF7, TAG-72, tenascin C, TGF-β, TRAIL-R1, TRAIL-R2, folic acid receptor, transferrin receptors, and any combination thereof. These antigen binding domains may be used in CARs and DARs for targeting the payload. CARs and/or antibodies that target the antigens are disclosed, for example, in the following: BCMA-WO201616630, WO2020150339, WO2019196713, WO2016014565, WO2017025038; MUC16: U.S. Pat. No. 9,169,328, WO2016149368, WO2020023888; EGFRvIII: WO2017125830, WO2016016341; Flt3: WO2018222935, WO2020010284, WO2017173410; CD20: WO2018145649, WO2020010235, WO2020123691; CD38: WO2017025323; CD70: WO2019152742, WO2018152181; CD33: WO2016014576; CD133: WO2018072025; CSI: WO2019030240; RORI: WO2016115559; CD19: WO2002077029, USI 1,077,144; Claudin: WO2018006882, WO2021008463; DLL3: WO2020180591; WTI: US20160152725A1, U.S. Pat. No. 7,622,119B2; CD23: U.S. Pat. No. 6,011,138A, CN1568198A; CD30: U.S. Ser. No. 10/815,301B2, U.S. Ser. No. 10/808,035B2; PRAME: US20180148503A, WO2020186204A1; LIVI: US20200231699A1; NKG2D: WO2021 1 79353A1, US20210269501A; FAP Alpha: US20200246383A1, US20210115102A1; PSMA: US20210277141A, WO2020108646A; MSLN: CN109680002A, CN109628492A.

In some embodiments, the sheddase cleavage site comprises one or more of the multiple protease cleavage sites and is recognized by one or more of thrombin, trypsin, plasmin, prostate-specific antigen (PSA), urokinase plasminogen activator (uPA), urokinase plasminogen activator receptor (uPAR), matrix metalloproteinase (MMP), matriptase (MT-SPl), legumain, a disintegrin and metalloproteinase (ADAM), and transmembrane Serine Protease (TMPRSS).

In some embodiments, the sheddase that recognizes the protease cleavage site or that recognizes one or more of the multiple protease cleavage sites, is one or more of thrombin, trypsin, plasmin, prostate-specific antigen (PSA), urokinase plasminogen activator (uP A), urokinase plasminogen activator receptor (uP AR), matrix metalloproteinase (MMP), matriptase (MT-SPl), legumain, a disintegrin and metalloproteinase (ADAM), transmembrane Serine Protease (TMPRSS), Granzyme B, activated protein C, Caspase, Cathepsin, Chymase, Elastase, Guanidinobenzoatase, HtrAl, Human Neutrophil Elastase, Lactoferrin, Marapsin, NS3/4A, PACE4, tissue plasminogen activator (tPA), DESCl, DPP-4, Hepsin, Matriptase-2, secretase, kallikrein-related peptidase (KLK), and tryptase, or a serine protease, a cysteine-type lysosomal protease, a metalloproteinase, a coagulation factor protease, or an aspartyl-type lysosomal protease.

In some embodiments, the payload comprise a cytokine or chemokine consisting a fraction or derivative of a sulfated xylan, which is an antagonist of a ligand selected from the list comprising IL-la, IL-1B, IL-2, IL-3, IL-4, IL-5, IL-6, IL-7, IL-8, IL-9, IL-10, IL-11, IL-12, IL-3, IL-14, IL-15 or IL-17 family of cytokines including IL-25, interferon, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, PDGF, SCF, TGFa, TGFB, TNFa, TNFB, TPO, VEGF, GH, NGF, NT3, NT4, NT5, NT6, NT7, oncostatin M (OSM), insulin, MCP-1, MCP-2, MCP-3, MCP-4, MCP-5, a member of the MIP-1 family including MIP-la, MIP-2, eotaxin (eotaxin-1, -2 or -3), PBP (platelet basic protein), SDF-1, PBSF, PF4, RANTES, elastase; an enzyme of the cathepsin family, a cell adhesion molecules such as PECAM-1, a soluble or cell- or virus-bound receptor, cytokine-induced neutrophil chemoattractant (KC), TNF-a and IFN-g), and other soluble mediators of inflammation, such as reactive oxygen species and nitric oxide, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, or any combination thereof.

In some embodiments the payload may comprise thrombin. In other embodiments the payload may be a vaccine. In yet another embodiment the payload can be Urokinase delivery. The present constructs may enable delivery of payloads through the blood brain barrier.

As used herein “a-TAA” means a single chain antibody or antibody domain directed against or that binds to a tumor-associated antigen, preferably an scFv or camelid antibody (VHH domain antibody or VHH domain) or single domain antibody or antibody binding domain. Exemplary tumor-associated antigens bound by the starting polypeptides used in the invention include for example, pan B antigens (e.g. CD20 found on the surface of both malignant and non-malignant B cells such as those in non-Hodgkin's lymphoma) and pan T cell antigens (e.g. CD2, CD3, CD5, CD6, CD7). Other exemplary tumor associated antigens comprise but are not limited to MAGE-1, MAGE-3, MUC-1, HPV 16, HPV E6 & E7, TAG-72, CEA, α-Lewisy, L6-Antigen, CD19, CD22, CD25, CD30, CD33, CD37, CD44, CD52, CD56, mesothelin, PSMA, HLA-DR, EGF Receptor, VEGF Receptor, and HER2 Receptor. Carcinoembryonic antigen (CEA), and a-fetoprotein (AFP) are two examples of such tumor associated antigens. Other targets include the MICA/B ligands of NKG2D. These molecules are expressed on many types of tumors, but not normally on healthy cells. Additional specific examples of tumor associated antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary tumor associated antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell tumor associated antigens described in U.S. Pat. No. 4,960,716.

Preferably, such a tumor-associated antigen is located on or in the cell membrane of a tumor cell. Examples of tumor-associated antigens are described, e.g., in DeVita et al. (Eds., “Biological Therapy of Cancer”, 2. Edition, Chapter 3: Biology of Tumor Antigens, Lippincott Company, ISBN 0-397-51416-6 (1995)). Not-limiting examples of such tumor antigens include CD19, CD20, CD30, CD33, CD38, CD133, BCMA, TEM8, EpCAM, ROR1, Folate Receptor, CD70, CEA, BAGE, CA-125, CDK-1, MART-1, MUC-1, MUM-1, PSA, PSMA, HER-2, IL13Ralpha, IL13Ralpha2, AIM-2, AIM-3, BRAP, RTN4, GLEA2, TNKS2, KIAA0376, RBPSUH, NKTR, EGFRvIII, LICAM, Livin, Livinβ, Nestin, OLIG2, ART1, ART4, Gli1, Cav-1, CD74, E-Cadherin, GAGE-1, Ganglioside/GD2, PROX1, PSCA, βhCG, WT1, mesothelin, melan-A, SSX-2, PLK1, VEGF-A, VEGFR2, and Tie-2.

### Spaciotemporal Regulation and Delivery

It is envisioned to utilize embodiments of the invention for targeted payload release in the tumor microenvironment or tissue-specific environment, and this can enable optimal time-point and anatomical location for release and activation of cargos such as release and unmasking of pro-inflammatory cytokines (e.g., TNF, IL2, IL15, IL18) in the tumor microenvironment, or conditional release of certain growth factors in non-malignant disorders (e.g., GMCSF in bone marrow transplantation, IL2 in certain immune deficiencies, Insulin in diabetes). Furthermore, temporal and conditional cargo release according to the invention is envisioned to limit the currently observed systemic off-target effects and/or limit continuous exposure and response. Conditional induction of responses is beneficial in numerous diseases where a clear circadian-like rhythm in symptoms formation, such as inflammatory bowel disease and Diabetes Mellitus.

It is also of great importance to be able to utilize bispecific engagers and other proteins with short half-lives and/or with high risk of off-target activity in other tissues in the right micro-environment at the right time. The embodiments of the invention are applicable in such conditions where spatio-temporal release is critical. One such example is the generation of a dispanin coding for a bispecific CD38-CD3 engager in the ectodomain-loop, which is cleavable only in the bone marrow micro-environment together with an anti-BCMA CAR and thus decrease off-target activity of the engager that is released as well as the CAR. In this model system, a bispecific CD38-CD3 engager with a very short half-life beneficially renders the systemic distribution and impact more unlikely. Furthermore, for embodiments of the invention, due to continuous protein turnover and release by the ferrying cells, one-time administration of the cells is estimated to be more potent and in some cases equivalent in efficacy to multiple-administration dosing regimes of other delivery systems while at the same time posing limited side effects.

### Example 1: General Structure

In one embodiment the present invention comprises a delivery system for a protein payload in which sheddases present in the body release the payload. Referring to FIGS. 1-2, the payload delivery system for delivery of a single payload comprises a first transmembrane anchor, a cytoplasmic region, a first site cleavable by a sheddase, a first linker, a payload sequence, a second linker, a second site cleavable by a sheddase, and optionally a single-chain specific CAR.

The transmembrane anchor can be selected from CD63, CD9, CD81, CD28, CD4, CD8, CD34, CD69, CD19, CD20, integrins, TNFSF14, TNR1A, aquaporins, NOTCH, NgR1, NRG1, GPi anchor, EGFR, Rhodopsin or any relevant TMDs that provide similar function such as membrane anchoring, and signal transduction modulation can be used for the expression of our invention.

The cytoplasmic domain can be selected from CD63, CD69, CD9, CD28, CD81, CD34, CD3, CD4, CD8 or any relevant cytoplasmic domain that provides similar function (e.g., with regard to anchoring, mobility within the plasma membrane, and/or the ability to sort to EVs, as well as signal transduction) to the system can be used. In some embodiments, the intracellular or extracellular domain comprises of OX-40, 4-1BB/CD137, CD2, CD7, CD27, CD30, B7-1 (CD80), B7-2 (CD86),PDL-1, programmed death-I (PD-1), inducible T cell co-stimulator (ICOS), lymphocyte function-associated antigen-I (LFA-1 (CDI la/CD 18), CD3 gamma, CD3 delta, CD3 epsilon, CD247, CD276 (B7-H3), LIGHT, (TNFSF14), NKG2C, lg alpha (CD79a), DAP-10, Fe gamma receptor, MHC class I molecule, TNF receptor proteins, an Immunoglobulin protein, cytokine receptor, integrins, Signaling Lymphocytic Activation Molecules (SLAM proteins), The costimulatory ligand (PD-L2, 4-1 BBL, OX40L, inducible costimulatory ligand (ICOS-L), intercellular adhesion molecule (ICAM, CD30L, CD70, CD83, HLA-G, MICA, Ml CB, HVEM, lymphotoxin b receptor, 3/TR6, ILT3, ILT4), activating NK cell receptors, BTLA, a Toll ligand receptor, ICAM-1, B7-H3, CDS, ICAM-1, GITR, BAFFR, LIGHT, HVEM (LIGHTR), KIRDS2, NKp80 (KLRFI), NKp44, NKp30, NKp46, CD19, CD8alpha, CD8beta, IL-2R beta, IL-2R gamma, IL-7R alpha, ITGA4, VLAl, CD49a, ITGA4, CD49D, ITGA6, VLA-6, CD49f, ITGAD, CDl Id, ITGAE, CD103, ITGAL, CDl la, LFA-1, IT GAM, CDl lb, ITGAX, CDl le, ITGBI, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD 160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

Linkers can be selected from any suitable linker with linkers as disclosed herein, e.g., the linkers set forth in Table 4, being preferred.

The sheddases and their cleavable sites may be selected from part or full-time sheddases. The full-time sheddases may be selected include ADAM proteases, BACE proteases, serine-protease Granzyme-B and Site-1 proteases. Adam proteases (metalloproteases) are membrane-anchored type 1 protease and include ADAM8, ADAM9, ADAM10, ADAM12, ADAM15, ADAM17, ADAM19, ADAM20, ADAM21, ADAM28, ADAM30, and ADAM33. The BACE (aspartyl) proteases are also membrane-anchored type1 proteases which include BACE1 and BACE2. The Site-1 (serine) proteases are membrane-anchored type 1 protease, including SKI-a or SIP.

Part-Time sheddases and their cleavable sites include Meprin B, MT-MMPs (membrane type matrix metalloproteases), Pro-protein convertases, Transmembrane serine proteases, Matrix metalloproteases, Legumain and Cathepsin S and L. Meprin B is a membrane-anchored type 1 metalloprotease. MT-MMPs are membrane-anchored type 1 or GPI-anchored and include MT1-MMP, MT2-MMP, MT3-MMP, MT4-MMP, MT5-MMP, and MT6-MMP. The Pro-protein convertases are membrane-anchored type 1 or soluble proteases and include PCSK1/3, KCSK2, furin, PCSK4, PSCK5/6, PACE4, PCSK7, and PCSK9. Transmembrane serine proteases are membrane-anchored type II proteases and include matriptase, Matriptase-2, matriptase-3, polymerase-1, Corin, Hepsin, TMPRSS2, TMPRSS3, TMPRSS4, MSPL, Spinesin, Enteropeptidase, HAT, DESCL1, TMPRSS11A, HAT-like 4 and HAT-like 5. The Matrix Metalloproteases are soluble proteases including MMP1, MMP2, MMP3, MMP4, MMP5, MMP6, MMP7, MMP8, MMP9, MMP10, MMP11, MMP12, MMP13, MMP14, MMP15, MMP16, MMP17, MMP18, MMP19, MMP20, MMP21, MMP21, MMP 23A-B, MMP24, MMP25, MMP26, MMP27 and MMP28.

In some embodiments, the payload comprise a cytokine or chemokine consisting a fraction or derivative of a sulfated xylan, which is an antagonist of a ligand selected from the list comprising IL-1a, IL-1B, IL-2, IL-3, IL-4, IL-5, IL-6, IL-7, IL-8, IL-9, IL-10, IL-11, IL-12, IL-3, IL-14, IL-15 or IL-17 family of cytokines including IL-25, interferon, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, PDGF, SCF, TGFa, TGFB, TNFa, TNFB, TPO, VEGF, GH, NGF, NT3, NT4, NT5, NT6, NT7, oncostatin M (OSM), insulin, MCP-1, MCP-2, MCP-3, MCP-4, MCP-5, a member of the MIP-1 family including MIP-la, MIP-2, eotaxin (eotaxin-1, -2 or -3), PBP (platelet basic protein), SDF-1, PBSF, PF4, RANTES, elastase; an enzyme of the cathepsin family, a cell adhesion molecules such as PECAM-1, a soluble or cell- or virus-bound receptor, cytokine-induced neutrophil chemoattractant (KC), TNF-a and IFN-g), and other soluble mediators of inflammation, such as reactive oxygen species and nitric oxide, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, or any combination thereof.

In other embodiments, further modifications can be applied to the system by adding more cleavable sheddases and more payloads on the exterior or interior of the cell. Wherein cleavage by the sheddase releases a peptide or protein. The peptide or protein may be active when cleaved or may still require additional activation via mechanisms present in the cell. The activation mechanisms can be naturally present or genetically engineered. FIGS. 4A-B and 5A-B show an embodiment of the present invention comprising 4 transmembrane domains. To generate an elaborate delivery system that can harbor different payloads in the intra- and extra-cellular environment needs to cross the plasma membrane multiple times. In this example we used tetraspanin TMDs linked through payloads spanning the sheddases sites to compose the delivery system. A first protease site cleavable MMP 9, a TNF-α payload and a second protease site cleavable by MMP9. FIGS. 4A-B and 5A-B differ by the presence of the EV sorting sequence.

### Example 2: Delivery of a BIKE

In one embodiment the delivery system of the present invention is configured to deliver a BIKE. Referring to FIG. 2 an embodiment of the invention comprises a BIKE flanked by protease cleavage sequences embedded in the extracellular loop between a Type-II transmembrane protein and a GPI anchor. This particular embodiment is encoded by the amino acid sequence set forth as SEQ ID NO: 123 or SEQ ID NO: 1 below. This construct consists of an N-terminal intracellular domain, followed by a Type-II transmembrane domain, cleavage sequence, and payload followed by another cleavage sequence. The C-terminal of this protein is bound to the plasma membrane through the GPi anchor. Sheddases result in cleavage, thus releasing the payload in the extracellular milieu.

(SEQ ID NO: 1)
MCGACKENYCLMITFAIFLSLIMLVEVAAAIAGYVGGSGPVRRYQGGPLG
VRGGMEVQLVESGGGVVRPGGSLRLSCAASGFTFDDYGMSWVRQAPGKGL
EWVSGINWNGGSTGYADSVKGRFTISRDNAKNSLYLQMNSLRAEDTAVYY
CARGRSLLFDYWGQGTLVTVSRGGGGSGGGGSGGGGSSELTQDPAVSVAL
GQTVRITCQGDSLRSYYASWYQQKPGQAPVLVIYGKNNRPSGIPDRFSGS
SSGNTASLTITGAQAEDEADYYCNSRDSSGNHVVFGGGTKLTVLPSGQAG
AAASESLFVSNHAYEASGGPEQVQLVQSGAEVKKPGSSVKVSCKASGYTF
TDYNMHWVRQAPGQGLEWIGYIYPYNGGTGYNQKFKSKATITADESTNTA
YMELSSLRSEDTAVYYCARGRPAMDYWGQGTLVTVSSGGGGSGGGGSGGG
GSDIQMTQSPSSLSASVGDRVTITCRASESVDNYGISFMNWFQQKPGKAP
KLLIYAASNQGSGVPSRFSGSGSGTDFTLTISSLQPDDFATYYCQQSKEV
PWTFGQGTKVEIKGGSGEQKLISEEDLGGPLGMTSGGSGPKNNHTASILD
RMQADFKCCGAANYTDWEKIPSMSKNRVPDSCCINVTVGCGINFNEKAIH
KEGCVEKIGGWLRKNVVLRDKLVKCEGISLLAQNTSWLLLLLLSLSLLQA
TDFMSL

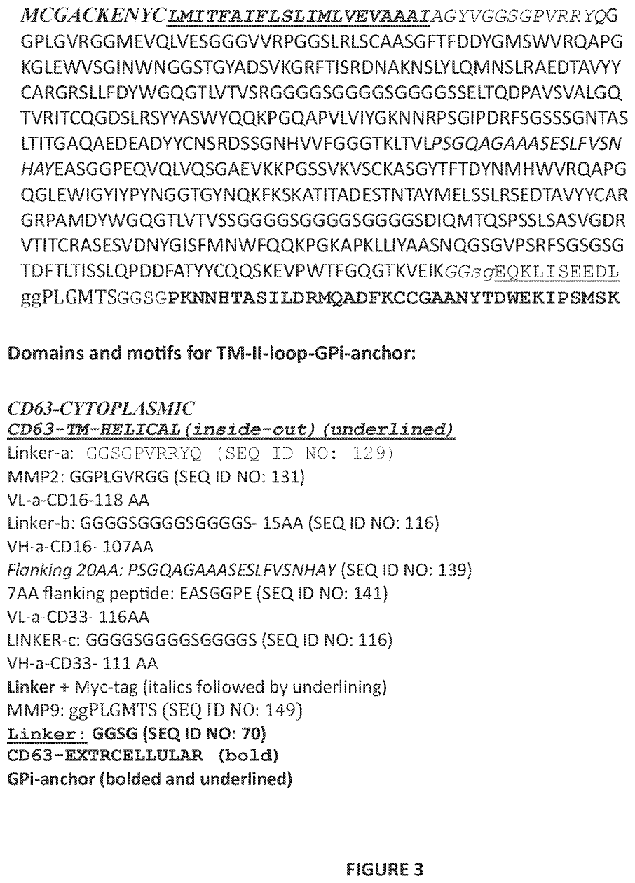

This sequence is also set out in FIG. 3, with a legend showing the domains and motifs.

The corresponding cDNA is set forth below:

(SEQ ID NO: 124)
ATGTGCGGCGCATGCAAGGAAAACTACTGTCTGATGATCACATTCGCCATC
TTCCTGAGCCTGATCATGCTGGTGGAAGTGGCCGCTGCCATCGCCGGATAC
GTGGGAGGCAGCGGGCCTGTGCGGCGGTACCAGGGAGGCCCACTGGGAGTT
AGAGGCGGCATGGAAGTGCAGCTGGTCGAATCGGGAGGCGGCGTGGTGAGA
CCAGGAGGCAGCCTCCGGCTGTCTTGTGCCGCGAGTGGTTTCACCTTTGAC
GACTACGGCATGAGCTGGGTGAGACAGGCTCCTGGAAAGGGCCTGGAATGG
GTTTCCGGCATCAACTGGAACGGCGGATCGACTGGCTACGCCGACAGCGTG
AAAGGCAGATTCACCATCAGCAGAGATAACGCCAAGAACAGCCTGTACCTG
CAGATGAACTCCCTGAGAGCCGAGGACACCGCCGTGTACTACTGCGCCAGA
GGCAGAAGCCTGCTGTTCGACTACTGGGGACAGGGCACACTGGTGACCGTC
AGCAGAGGCGGCGGCGGCTCTGGCGGCGGCGGAAGCGGAGGCGGCGGATCC
TCCGAGCTGACACAGGACCCCGCTGTTAGCGTGGCTCTGGGCCAGACCGTG
CGGATCACCTGTCAGGGCGACAGCCTCCGCTCCTACTACGCCAGCTGGTAC
CAACAAAAACCTGGCCAAGCTCCTGTGCTGGTCATCTACGGAAAGAACAAC
AGACCTAGTGGCATCCCTGATAGATTTAGCGGCAGCAGCAGCGGCAACACC
GCCTCACTGACCATTACAGGCGCCCAGGCCGAGGATGAGGCTGACTATTAC
TGCAACTCCAGGGACAGCTCTGGAAATCATGTGGTCTTCGGCGGCGGCACA
AAGCTGACCGTGCTGCCTTCTGGCCAAGCCGGTGCCGCCGCGTCTGAGAGC
CTGTTCGTGAGCAACCACGCCTACGAGGCCAGCGGCGGCCCCGAGCAGGTG
CAGCTGGTGCAGAGCGGCGCTGAGGTGAAAAAGCCCGGCTCTAGCGTGAAG
GTGTCCTGCAAGGCCAGCGGCTACACCTTCACAGATTATAATATGCACTGG
GTCAGACAGGCCCCTGGGCAGGGACTGGAATGGATCGGCTATATCTACCCC
TACAACGGCGGAACAGGCTACAACCAGAAGTTCAAGAGCAAGGCCACCATC
ACAGCCGACGAGAGCACCAACACCGCTTATATGGAACTGTCTTCTCTGCGG
AGCGAAGATACCGCCGTGTACTACTGTGCCAGAGGCCGGCCTGCCATGGAC
TACTGGGGCCAGGGCACCCTGGTGACAGTGTCCTCAGGCGGCGGCGGTAGC
GGCGGCGGCGGCAGCGGCGGCGGTGGTTCTGATATCCAGATGACCCAGAGC
CCCAGCAGCCTGTCCGCCAGTGTCGGAGATAGGGTGACCATCACCTGCAGA
GCCTCTGAAAGCGTTGACAATTACGGCATCAGCTTCATGAACTGGTTCCAG
CAGAAACCTGGCAAAGCCCCTAAGCTTCTTATTTACGCCGCTTCCAACCAG
GGCTCTGGCGTGCCCAGCCGGTTCAGCGGCTCAGGATCTGGAACCGACTTT
ACGCTGACAATCAGCTCTCTGCAGCCTGATGACTTCGCCACATACTACTGC
CAGCAAAGCAAAGAGGTGCCTTGGACCTTCGGCCAGGGCACCAAGGTGGAG
ATTAAGGGCGGGTCCGGAGAGCAGAAGCTGATCTCTGAGGAAGACCTGGGA
GGCCCTCTGGGCATGACCTCTGGAGGCAGCGGCCCTAAGAACAACCACACC
GCCAGCATCCTGGACCGGATGCAGGCCGACTTTAAGTGCTGCGGCGCCGCT
AATTACACCGACTGGGAGAAAATCCCCAGCATGAGCAAGAATAGAGTGCCC
GATAGCTGTTGCATCAACGTGACAGTGGGCTGCGGCATCAATTTCAACGAG
AAGGCTATCCACAAGGAGGGCTGCGTGGAAAAGATCGGCGGCTGGCTGAGA
AAGAACGTGGTGCTGAGAGACAAGCTCGTGAAATGCGAGGGCATCAGCCTG
CTGGCCCAGAACACATCCTGGCTGCTGCTGCTGCTGCTCAGCCTGAGTCTG
CTGCAAGCCACCGATTTCATGTCTCTGTAA

As shown in FIG. 3, SEQ ID NO: 123 or 1 is divided into functional motifs as follows:

CD63-CYTOPLASMIC
(SEQ ID NO: 125)
MCGACKENYC,
which is encoded by DNA
(SEQ ID NO: 126)
ATGTGCGGCGCATGCAAGGAAAACTACTGT
CD63-TM-HELICAL(inside-out)
(SEQ ID NO: 127)
LMITFAIFLSLIMLVEVAAAI,
which is encoded by DNA
(SEQ ID NO: 128)
CTGATGATCACATTCGCCATCTTCCTGAGCCTGATCATGCTGGTG
GAAGTGGCCGCTGCCATC.
Linker-a:
(SEQ ID NO: 129)
GGSGPVRRYQ,
which is encoded by DNA
(SEQ ID NO: 130)
GGAGGCAGCGGGCCTGTGCGGCGGTACCAG
MMP2:
(SEQ ID NO: 131)
GGPLGVRGG,
which is encoded by DNA
(SEQ ID NO: 132)
GGAGGCCCACTGGGAGTTAGAGGCGGC
VL-a-CD16-118 AA
(SEQ ID NO 133)
MEVQLVESGGGVVRPGGSLRLSCAASGFTFDDYGMSWVRQAPGKG
LEWVSGINWNGGSTGYADSVKGRFTISRDNAKNSLYLQMNSLRAE
DTAVYYCARGRSLLFDYWGQGTLVTVSR
which is encoded by DNA
(SEQ ID NO: 134)
ATGGAAGTGCAGCTGGTCGAATCGGGAGGCGGCGTGGTGAGACCA
GGAGGCAGCCTCCGGCTGTCTTGTGCCGCGAGTGGTTTCACCTTT
GACGACTACGGCATGAGCTGGGTGAGACAGGCTCCTGGAAAGGGC
CTGGAATGGGTTTCCGGCATCAACTGGAACGGCGGATCGACTGGC
TACGCCGACAGCGTGAAAGGCAGATTCACCATCAGCAGAGATAAC
GCCAAGAACAGCCTGTACCTGCAGATGAACTCCCTGAGAGCCGAG
GACACCGCCGTGTACTACTGCGCCAGAGGCAGAAGCCTGCTGTTC
GACTACTGGGGACAGGGCACACTGGTGACCGTCAGCAGA
Linker-b and Linker-c:
(SEQ ID NO: 135)
GGGGSGGGGSGGGGS 15 AA,
which is encoded by DNA
(SEQ ID NO: 136)
GGCGGCGGCGGCTCTGGCGGCGGCGGAAGCGGAGGCGGCGGATCC
VH-a-CD16-107 AA
(SEQ ID NO: 137)
SELTQDPAVSVALGQTVRITCQGDSLRSYYASWYQQKPGQAPVLV
IYGKNNRPSGIPDRFSGSSSGNTASLTITGAQAEDEADYYCNSRD
SSGNHVVFGGGTKLTVL,
which is encoded by DNA
(SEQ ID NO: 138)
TCCGAGCTGACACAGGACCCCGCTGTTAGCGTGGCTCTGGGCCAG
ACCGTGCGGATCACCTGTCAGGGCGACAGCCTCCGCTCCTACTAC
GCCAGCTGGTACCAACAAAAACCTGGCCAAGCTCCTGTGCTGGTC
ATCTACGGAAAGAACAACAGACCTAGTGGCATCCCTGATAGATTT
AGCGGCAGCAGCAGCGGCAACACCGCCTCACTGACCATTACAGGC
GCCCAGGCCGAGGATGAGGCTGACTATTACTGCAACTCCAGGGAC
AGCTCTGGAAATCATGTGGTCTTCGGCGGCGGCACAAAGCTGACC
GTGCTG
Flanking 20 AA:
(SEQ ID NO: 139)
PSGQAGAAASESLFVSNHAY,
which is encoded by DNA
(SEQ ID NO: 140)
CCTTCTGGCCAAGCCGGTGCCGCCGCGTCTGAGAGCCTGTTCGTG
AGCAACCACGCCTAC
7 AA flanking peptide:
(SEQ ID NO: 141)
EASGGPE,
which is encoded by DNA
(SEQ ID NO: 142)
GAGGCCAGCGGCGGCCCCGAG
VL-a-CD33-116 AA
(SEQ ID NO: 143)
QVQLVQSGAEVKKPGSSVKVSCKASGYTFTDYNMHWVRQAPGQGL
EWIGYIYPYNGGTGYNQKFKSKATITADESTNTAYMELSSLRSED
TAVYYCARGRPAMDYWGQGTLVTVSS
which is encoded by DNA
(SEQ ID NO: 144)
CAGGTGCAGCTGGTGCAGAGCGGCGCTGAGGTGAAAAAGCCCGGC
TCTAGCGTGAAGGTGTCCTGCAAGGCCAGCGGCTACACCTTCACA
GATTATAATATGCACTGGGTCAGACAGGCCCCTGGGCAGGGACTG
GAATGGATCGGCTATATCTACCCCTACAACGGCGGAACAGGCTAC
AACCAGAAGTTCAAGAGCAAGGCCACCATCACAGCCGACGAGAGC
ACCAACACCGCTTATATGGAACTGTCTTCTCTGCGGAGCGAAGAT
ACCGCCGTGTACTACTGTGCCAGAGGCCGGCCTGCCATGGACTAC
TGGGGCCAGGGCACCCTGGTGACAGTGTCCTCA
VH-a-CD33
(SEQ ID NO: 145)
DIQMTQSPSSLSASVGDRVTITCRASESVDNYGISFMNWFQQKPG
KAPKLLIYAASNQGSGVPSRFSGSGSGTDFTLTISSLQPDDFATY
YCQQSKEVPWTFGQGTKVEIK,
which is encoded by DNA
(SEQ ID NO: 146)
GATATCCAGATGACCCAGAGCCCCAGCAGCCTGTCCGCCAGTGTC
GGAGATAGGGTGACCATCACCTGCAGAGCCTCTGAAAGCGTTGAC
AATTACGGCATCAGCTTCATGAACTGGTTCCAGCAGAAACCTGGC
AAAGCCCCTAAGCTTCTTATTTACGCCGCTTCCAACCAGGGCTCT
GGCGTGCCCAGCCGGTTCAGCGGCTCAGGATCTGGAACCGACTTT
ACGCTGACAATCAGCTCTCTGCAGCCTGATGACTTCGCCACATAC
TACTGCCAGCAAAGCAAAGAGGTGCCTTGGACCTTCGGCCAGGGC
ACCAAGGTGGAGATTAAG
Myc-tag
(SEQ ID NO: 147)
EQKLISEEDL,
which is encoded by DNA
(SEQ ID NO: 148)
GAGCAGAAGCTGATCTCTGAGGAAGACCTG
MMP9:
(SEQ ID NO: 149)
GGPLGMTS,
which is encoded by DNA
(SEQ ID NO: 150)
GGAGGCCCTCTGGGCATGACCTCT
CD63-EXTRACELLULAR
(SEQ ID NO: 151)
PKNNHTASILDRMQADFKCCGAANYTDWEKIPSMSKNRVPDSCCI
NVTVGCGINFNEKAIHKEGCVEKIGGWLRKNV,
which is encoded by DNA
(SEQ ID NO: 152)
CCTAAGAACAACCACACCGCCAGCATCCTGGACCGGATGCAGGCC
GACTTTAAGTGCTGCGGCGCCGCTAATTACACCGACTGGGAGAAA
ATCCCCAGCATGAGCAAGAATAGAGTGCCCGATAGCTGTTGCATC
AACGTGACAGTGGGCTGCGGCATCAATTTCAACGAGAAGGCTATC
CACAAGGAGGGCTGCGTGGAAAAGATCGGCGGCTGGCTGAGAAAG
AACGTG
GPi-anchor:
(SEQ ID NO: 153)
VLRDKLVKCEGISLLAQNTSWLLLLLLSLSLLQATDFMSL,
which is encoded by DNA
(SEQ ID NO: 154)
GTGCTGAGAGACAAGCTCGTGAAATGCGAGGGCATCAGCCTGCTG
GCCCAGAACACATCCTGGCTGCTGCTGCTGCTGCTCAGCCTGAGT
CTGCTGCAAGCCACCGATTTCATGTCTCTG

### Example 3: Payload Delivered into Extracellular Vesicles

Referring to FIG. 4A-B, this embodiment is a payload with MMP cleavable sequences embedded in the extracellular loop between the third and fourth transmembrane domain of tetraspanin. This particular molecule will get sorted into extracellular vesicles due to the presence of the GYEVM motif (SEQ ID NO: 337).

The amino acid sequence in SEQ ID NO 155 below encodes the construct.

(SEQ ID NO: 155)
MCGACKENYCLMITFAIFLSLIMLVEVAAAIAGYVGGSGPVRRYQGGPLGV
RGGMEVQLVESGGGVVRPGGSLRLSCAASGFTFDDYGMSWVRQAPGKGLEW
VSGINWNGGSTGYADSVKGRFTISRDNAKNSLYLQMNSLRAEDTAVYYCAR
GRSLLFDYWGQGTLVTVSRGGGGSGGGGSGGGGSSELTQDPAVSVALGQTV
RITCQGDSLRSYYASWYQQKPGQAPVLVIYGKNNRPSGIPDRFSGSSSGNT
ASLTITGAQAEDEADYYCNSRDSSGNHVVFGGGTKLTVLPSGQAGAAASES
LFVSNHAYEASGGPEQVQLVQSGAEVKKPGSSVKVSCKASGYTFTDYNMHW
VRQAPGQGLEWIGYIYPYNGGTGYNQKFKSKATITADESTNTAYMELSSLR
SEDTAVYYCARGRPAMDYWGQGTLVTVSSGGGGSGGGGSGGGGSDIQMTQS
PSSLSASVGDRVTITCRASESVDNYGISFMNWFQQKPGKAPKLLIYAASNQ
GSGVPSRFSGSGSGTDFTLTISSLQPDDFATYYCQQSKEVPWTFGQGTKVE
IKGGSGEQKLISEEDLGGPLGMTSGGSGPKNNHTASILDRMQADFKCCGAA
NYTDWEKIPSMSKNRVPDSCCINVTVGCGINFNEKAIHKEGCVEKIGGWLR
KNVLVVAAAALGIAFVEVLGIVFACCLVKSIRSGYEVM

The corresponding cDNA is set forth below:

(SEQ ID NO: 156)
ATGTGCGGCGCCTGTAAAGAAAACTACTGCCTGATGATCACATTCGCAATC
TTCCTGTCCCTCATCATGCTGGTGGAGGTGGCCGCCGCCATCGCTGGCTAC
GTGGGCGGCAGCGGCCCTGTGAGGAGATACCAGGGCGGTCCACTGGGCGTG
CGGGGCGGCATGGAAGTGCAGCTGGTGGAGAGCGGCGGCGGCGTGGTGAGA
CCTGGCGGGTCCCTGCGGCTGAGCTGTGCCGCCAGCGGATTCACATTCGAC
GATTACGGCATGAGCTGGGTCAGACAGGCCCCAGGAAAAGGCCTGGAATGG
GTGTCTGGCATTAACTGGAACGGCGGCAGCACCGGCTACGCCGATAGCGTT
AAGGGCAGATTCACCATCAGCAGAGATAACGCCAAGAACTCTCTGTACCTG
CAGATGAATAGCCTGCGGGCCGAAGATACCGCCGTCTACTACTGTGCCCGG
GGAAGAAGCTTGCTGTTTGACTACTGGGGACAGGGCACACTGGTCACCGTG
TCTAGGGGCGGCGGCGGCAGCGGAGGAGGCGGGTCGGGCGGCGGCGGATCC
AGCGAGCTGACACAAGATCCTGCTGTGAGCGTCGCCCTGGGCCAGACCGTG
CGGATCACTTGCCAGGGAGATAGCCTGAGGTCCTACTACGCCAGTTGGTAT
CAACAGAAGCCTGGACAGGCCCCTGTACTGGTGATCTACGGCAAGAACAAC
AGACCTTCTGGCATCCCTGATAGATTCAGCGGCTCCAGCAGCGGAAACACC
GCTAGCCTGACAATCACCGGAGCTCAGGCTGAGGACGAGGCCGACTACTAC
TGCAACAGCCGGGACTCTTCAGGAAATCACGTGGTGTTCGGCGGCGGCACC
AAGCTGACAGTGCTGCCTAGCGGCCAGGCCGGCGCCGCGGCCAGCGAAAGC
CTGTTCGTGTCTAACCACGCCTACGAGGCTTCCGGCGGCCCCGAGCAGGTG
CAGCTGGTGCAAAGCGGCGCCGAGGTGAAGAAGCCCGGCAGCAGCGTGAAA
GTGTCCTGCAAGGCCTCCGGCTACACCTTTACCGACTACAATATGCACTGG
GTTCGGCAGGCCCCTGGCCAAGGTCTCGAGTGGATCGGCTACATCTATCCT
TACAACGGCGGCACCGGCTACAACCAGAAGTTCAAAAGCAAGGCAACAATC
ACAGCCGACGAGAGCACTAACACCGCCTACATGGAACTGAGCAGCCTGAGA
AGCGAGGACACCGCTGTTTACTACTGCGCCAGAGGCAGACCAGCTATGGAC
TACTGGGGCCAAGGAACCCTGGTGACCGTGTCATCTGGGGGCGGCGGATCT
GGCGGCGGCGGGAGCGGCGGCGGAGGAAGTGATATCCAGATGACCCAGAGC
CCTAGCAGCCTGTCTGCCTCTGTTGGCGACCGGGTGACCATCACCTGCAGA
GCTTCTGAATCAGTTGACAACTACGGCATCAGCTTCATGAACTGGTTCCAG
CAGAAACCTGGCAAGGCCCCCAAGCTGCTGATCTATGCCGCTTCTAATCAG
GGCAGCGGCGTGCCTAGCAGATTTAGCGGCAGTGGATCGGGAACCGACTTC
ACCCTGACCATTTCTTCGCTTCAGCCCGACGATTTCGCCACATACTATTGT
CAGCAGAGCAAGGAGGTGCCATGGACATTTGGCCAGGGCACTAAGGTGGAA
ATCAAGGGCGGCAGCGGAGAGCAGAAGCTGATCTCCGAGGAAGACCTGGGT
GGACCCCTGGGCATGACCTCTGGCGGCAGCGGCCCTAAGAACAACCACACA
GCCAGCATCCTGGACAGAATGCAGGCCGACTTCAAGTGCTGCGGCGCCGCT
AATTACACCGACTGGGAGAAAATCCCCTCCATGAGCAAAAACCGGGTGCCC
GACAGCTGTTGCATCAATGTGACCGTGGGCTGTGGCATTAACTTCAACGAG
AAGGCTATCCATAAGGAGGGCTGCGTCGAGAAGATCGGCGGCTGGCTGAGA
AAGAACGTGCTGGTGGTGGCCGCCGCCGCCCTGGGTATCGCCTTCGTGGAA
GTGCTGGGCATCGTGTTCGCCTGCTGCCTGGTGAAGTCTATCAGATCTGGC
TATGAGGTGATG

SEQ ID NO 155 is divided into functional motifs as follows:

CD63-CYTOPLASMIC:
(SEQ ID NO: 157)
MCGACKENYC,
which is encoded by DNA
(SEQ ID NO: 158)
ATGTGCGGCGCCTGTAAAGAAAACTACTGC
CD63-TM-HELICAL(inside-out)
(SEQ ID NO: 159)
LMITFAIFLSLIMLVEVAAAI,
which is encoded by DNA
(SEQ ID NO: 160
CTGATGATCACATTCGCAATCTTCCTGTCCCTCATCATGCTGGTG
GAGGTGGCCGCCGCCATC
Linker-a:
(SEQ ID NO: 161)
GGSGPVRRYQ,
which is encoded by DNA
(SEQ ID NO: 162)
GGCGGCAGCGGCCCTGTGAGGAGATACCAG
MMP2:
(SEQ ID NO: 163)
GGPLGVRGG,
which is encoded by DNA
(SEQ ID NO: 164)
GGCGGTCCACTGGGCGTGCGGGGCGGC
VL-a-CD16-118 AA
(SEQ ID NO: 165)
MEVQLVESGGGVVRPGGSLRLSCAASGFTFDDYGMSWVRQAPGKG
LEWVSGINWNGGSTGYADSVKGRFTISRDNAKNSLYLQMNSLRAE
DTAVYYCARGRSLLFDYWGQGTLVTVSR
which is encoded by DNA
(SEQ ID NO: 166)
ATGGAAGTGCAGCTGGTGGAGAGCGGCGGCGGCGTGGTGAGACCT
GGCGGGTCCCTGCGGCTGAGCTGTGCCGCCAGCGGATTCACATTC
GACGATTACGGCATGAGCTGGGTCAGACAGGCCCCAGGAAAAGGC
CTGGAATGGGTGTCTGGCATTAACTGGAACGGCGGCAGCACCGGC
TACGCCGATAGCGTTAAGGGCAGATTCACCATCAGCAGAGATAAC
GCCAAGAACTCTCTGTACCTGCAGATGAATAGCCTGCGGGCCGAA
GATACCGCCGTCTACTACTGTGCCCGGGGAAGAAGCTTGCTGTTT
GACTACTGGGGACAGGGCACACTGGTCACCGTGTCTAGG
Linker-b and Linker-c:
(SEQ ID NO: 167)
GGGGSGGGGSGGGGS 15 AA,
which is encoded by DNA
(SEQ ID NO: 168)
GGCGGCGGCGGCAGCGGAGGAGGCGGGTCGGGCGGCGGCGGATCC
VH-a-CD16-107 AA
(SEQ ID NO: 169)
SELTQDPAVSVALGQTVRITCQGDSLRSYYASWYQQKPGQAPVLV
IYGKNNRPSGIPDRFSGSSSGNTASLTITGAQAEDEADYYCNSRD
SSGNHVVFGGGTKLTVL
which is encoded by DNA
(SEQ ID NO: 170)
AGCGAGCTGACACAAGATCCTGCTGTGAGCGTCGCCCTGGGCCAG
ACCGTGCGGATCACTTGCCAGGGAGATAGCCTGAGGTCCTACTAC
GCCAGTTGGTATCAACAGAAGCCTGGACAGGCCCCTGTACTGGTG
ATCTACGGCAAGAACAACAGACCTTCTGGCATCCCTGATAGATTC
AGCGGCTCCAGCAGCGGAAACACCGCTAGCCTGACAATCACCGGA
GCTCAGGCTGAGGACGAGGCCGACTACTACTGCAACAGCCGGGAC
TCTTCAGGAAATCACGTGGTGTTCGGCGGCGGCACCAAGCTGACA
GTGCTG
Flanking 20 AA:
(SEQ ID NO: 171)
PSGQAGAAASESLFVSNHAY,
which is encoded by DNA
(SEQ ID NO: 172)
CCTAGCGGCCAGGCCGGCGCCGCGGCCAGCGAAAGCCTGTTCGTG
TCTAACCACGCCTAC
7 AA flanking peptide:
(SEQ ID NO: 173)
EASGGPE,
which is encoded by DNA
(SEQ ID NO: 174)
GAGGCTTCCGGCGGCCCCGAG
VL-a-CD33-116 AA
(SEQ ID NO: 175)
QVQLVQSGAEVKKPGSSVKVSCKASGYTFTDYNMHWVRQAPGQGL
EWIGYIYPYNGGTGYNQKFKSKATITADESTNTAYMELSSLRSED
TAVYYCARGRPAMDYWGQGTLVTVSS
which is encoded by DNA
(SEQ ID NO: 176)
CAGGTGCAGCTGGTGCAAAGCGGCGCCGAGGTGAAGAAGCCCGGC
AGCAGCGTGAAAGTGTCCTGCAAGGCCTCCGGCTACACCTTTACC
GACTACAATATGCACTGGGTTCGGCAGGCCCCTGGCCAAGGTCTC
GAGTGGATCGGCTACATCTATCCTTACAACGGCGGCACCGGCTAC
AACCAGAAGTTCAAAAGCAAGGCAACAATCACAGCCGACGAGAGC
ACTAACACCGCCTACATGGAACTGAGCAGCCTGAGAAGCGAGGAC
ACCGCTGTTTACTACTGCGCCAGAGGCAGACCAGCTATGGACTAC
TGGGGCCAAGGAACCCTGGTGACCGTGTCATCT
VH-a-CD33
(SEQ ID NO 177)
DIQMTQSPSSLSASVGDRVTITCRASESVDNYGISFMNWFQQKPG
KAPKLLIYAASNQGSGVPSRFSGSGSGTDFTLTISSLQPDDFATY
YCQQSKEVPWTFGQGTKVEIK,
which is encoded by DNA
(SEQ ID NO: 178)
GATATCCAGATGACCCAGAGCCCTAGCAGCCTGTCTGCCTCTGTT
GGCGACCGGGTGACCATCACCTGCAGAGCTTCTGAATCAGTTGAC
AACTACGGCATCAGCTTCATGAACTGGTTCCAGCAGAAACCTGGC
AAGGCCCCCAAGCTGCTGATCTATGCCGCTTCTAATCAGGGCAGC
GGCGTGCCTAGCAGATTTAGCGGCAGTGGATCGGGAACCGACTTC
ACCCTGACCATTTCTTCGCTTCAGCCCGACGATTTCGCCACATAC
TATTGTCAGCAGAGCAAGGAGGTGCCATGGACATTTGGCCAGGGC
ACTAAGGTGGAAATCAAG
Linker and Myc-TAG
(SEQ ID NO: 179)
GGSGEQKLISEEDLGG
(SEQ ID NO: 180)
GGCGGCAGCGGAGAGCAGAAGCTGATCTCCGAGGAAGACCTGGGT
GGA
MMP9:
(SEQ ID NO: 181)
PLGMTS
(SEQ ID NO: 182)
CCCCTGGGCATGACCTCT
CD63-EXTRACELLULAR
(SEQ ID NO: 183)
PKNNHTASILDRMQADFKCCGAANYTDWEKIPSMSKNRVPDSCCI
NVTVGCGINFNEKAIHKEGCVEKIGGWLRKNV
(SEQ ID NO: 184)
CCTAAGAACAACCACACAGCCAGCATCCTGGACAGAATGCAGGCC
GACTTCAAGTGCTGCGGCGCCGCTAATTACACCGACTGGGAGAAA
ATCCCCTCCATGAGCAAAAACCGGGTGCCCGACAGCTGTTGCATC
AATGTGACCGTGGGCTGTGGCATTAACTTCAACGAGAAGGCTATC
CATAAGGAGGGCTGCGTCGAGAAGATCGGCGGCTGGCTGAGAAAG
AACGTG
CD63-TM-HELICAL(outside-in)
(SEQ ID NO: 185)
LVVAAAALGIAFVEVLGIVFA
(SEQ ID NO: 186)
CTGGTGGTGGCCGCCGCCGCCCTGGGTATCGCCTTCGTGGAAGTG
CTGGGCATCGTGTTCGCC
CD63-CYTOPLASMIC
SEQ ID NO: 187)
CCLVKSIRSGYEVM
(SEQ ID NO: 189)
TGCTGCCTGGTGAAGTCTATCAGATCTGGCTATGAGGTGATG

### Example 4: Cell Surface Expressed BIKE

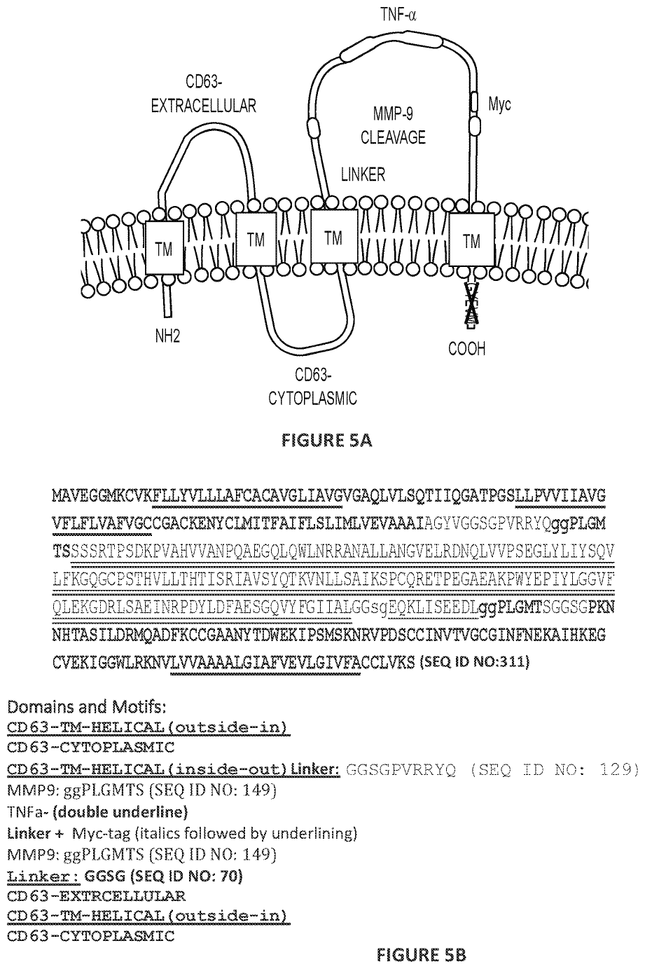

Referring to FIG. 5A-B, one embodiment of the invention is a BIKE with MMP/GrzB cleavage sequences embedded in the extracellular loop between the third and fourth transmembrane region of a tetraspanin. This molecule will be expressed on the cell surface due to the lack of signaling motif GYEVM (SEQ ID NO: 337) required for sorting to extracellular vesicles.

The construct has the amino acid sequence outlined in SEQ ID NO: 188 below:

(SEQ ID NO: 188)
MCGACKENYCLMITFAIFLSLIMLVEVAAAIAGYVGGSGPVRRYQGGPLGV
RGGMEVQLVESGGGVVRPGGSLRLSCAASGFTFDDYGMSWVRQAPGKGLEW
VSGINWNGGSTGYADSVKGRFTISRDNAKNSLYLQMNSLRAEDTAVYYCAR
GRSLLFDYWGQGTLVTVSRGGGGSGGGGSGGGGSSELTQDPAVSVALGQTV
RITCQGDSLRSYYASWYQQKPGQAPVLVIYGKNNRPSGIPDRFSGSSSGNT
ASLTITGAQAEDEADYYCNSRDSSGNHVVFGGGTKLTVLPSGQAGAAASES
LFVSNHAYEASGGPEQVQLVQSGAEVKKPGSSVKVSCKASGYTFTDYNMHW
VRQAPGQGLEWIGYIYPYNGGTGYNQKFKSKATITADESTNTAYMELSSLR
SEDTAVYYCARGRPAMDYWGQGTLVTVSSGGGGSGGGGSGGGGSDIQMTQS
PSSLSASVGDRVTITCRASESVDNYGISFMNWFQQKPGKAPKLLIYAASNQ
GSGVPSRFSGSGSGTDFTLTISSLQPDDFATYYCQQSKEVPWTFGQGTKVE
IKGGSGEQKLISEEDLGGPLGMTSGGSGPKNNHTASILDRMQADFKCCGAA
NYTDWEKIPSMSKNRVPDSCCINVTVGCGINFNEKAIHKEGCVEKIGGWLR
KNVLVVAAAALGIAFVEVLGIVFACCLVKS.

The corresponding cDNA is set forth below as (SEQ ID No: 189):

(SEQ ID No: 189)
ATGTGCGGCGCCTGTAAAGAAAACTACTGCCTGATGATCACATTCGCAATC
TTCCTGTCCCTCATCATGCTGGTGGAGGTGGCCGCCGCCATCGCTGGCTAC
GTGGGCGGCAGCGGCCCTGTGAGGAGATACCAGGGCGGTCCACTGGGCGTG
CGGGGCGGCATGGAAGTGCAGCTGGTGGAGAGCGGCGGCGGCGTGGTGAGA
CCTGGCGGGTCCCTGCGGCTGAGCTGTGCCGCCAGCGGATTCACATTCGAC
GATTACGGCATGAGCTGGGTCAGACAGGCCCCAGGAAAAGGCCTGGAATGG
GTGTCTGGCATTAACTGGAACGGCGGCAGCACCGGCTACGCCGATAGCGTT
AAGGGCAGATTCACCATCAGCAGAGATAACGCCAAGAACTCTCTGTACCTG
CAGATGAATAGCCTGCGGGCCGAAGATACCGCCGTCTACTACTGTGCCCGG
GGAAGAAGCTTGCTGTTTGACTACTGGGGACAGGGCACACTGGTCACCGTG
TCTAGGGGCGGCGGCGGCAGCGGAGGAGGCGGGTCGGGCGGCGGCGGATCC
AGCGAGCTGACACAAGATCCTGCTGTGAGCGTCGCCCTGGGCCAGACCGTG
CGGATCACTTGCCAGGGAGATAGCCTGAGGTCCTACTACGCCAGTTGGTAT
CAACAGAAGCCTGGACAGGCCCCTGTACTGGTGATCTACGGCAAGAACAAC
AGACCTTCTGGCATCCCTGATAGATTCAGCGGCTCCAGCAGCGGAAACACC
GCTAGCCTGACAATCACCGGAGCTCAGGCTGAGGACGAGGCCGACTACTAC
TGCAACAGCCGGGACTCTTCAGGAAATCACGTGGTGTTCGGCGGCGGCACC
AAGCTGACAGTGCTGCCTAGCGGCCAGGCCGGCGCCGCGGCCAGCGAAAGC
CTGTTCGTGTCTAACCACGCCTACGAGGCTTCCGGCGGCCCCGAGCAGGTG
CAGCTGGTGCAAAGCGGCGCCGAGGTGAAGAAGCCCGGCAGCAGCGTGAAA
GTGTCCTGCAAGGCCTCCGGCTACACCTTTACCGACTACAATATGCACTGG
GTTCGGCAGGCCCCTGGCCAAGGTCTCGAGTGGATCGGCTACATCTATCCT
TACAACGGCGGCACCGGCTACAACCAGAAGTTCAAAAGCAAGGCAACAATC
ACAGCCGACGAGAGCACTAACACCGCCTACATGGAACTGAGCAGCCTGAGA
AGCGAGGACACCGCTGTTTACTACTGCGCCAGAGGCAGACCAGCTATGGAC
TACTGGGGCCAAGGAACCCTGGTGACCGTGTCATCTGGGGGCGGCGGATCT
GGCGGCGGCGGGAGCGGCGGCGGAGGAAGTGATATCCAGATGACCCAGAGC
CCTAGCAGCCTGTCTGCCTCTGTTGGCGACCGGGTGACCATCACCTGCAGA
GCTTCTGAATCAGTTGACAACTACGGCATCAGCTTCATGAACTGGTTCCAG
CAGAAACCTGGCAAGGCCCCCAAGCTGCTGATCTATGCCGCTTCTAATCAG
GGCAGCGGCGTGCCTAGCAGATTTAGCGGCAGTGGATCGGGAACCGACTTC
ACCCTGACCATTTCTTCGCTTCAGCCCGACGATTTCGCCACATACTATTGT
CAGCAGAGCAAGGAGGTGCCATGGACATTTGGCCAGGGCACTAAGGTGGAA
ATCAAGGGCGGCAGCGGAGAGCAGAAGCTGATCTCCGAGGAAGACCTGGGT
GGACCCCTGGGCATGACCTCTGGCGGCAGCGGCCCTAAGAACAACCACACA
GCCAGCATCCTGGACAGAATGCAGGCCGACTTCAAGTGCTGCGGCGCCGCT
AATTACACCGACTGGGAGAAAATCCCCTCCATGAGCAAAAACCGGGTGCCC
GACAGCTGTTGCATCAATGTGACCGTGGGCTGTGGCATTAACTTCAACGAG
AAGGCTATCCATAAGGAGGGCTGCGTCGAGAAGATCGGCGGCTGGCTGAGA
AAGAACGTGCTGGTGGTGGCCGCCGCCGCCCTGGGTATCGCCTTCGTGGAA
GTGCTGGGCATCGTGTTCGCCTGCTGCCTGGTGAAGTCT

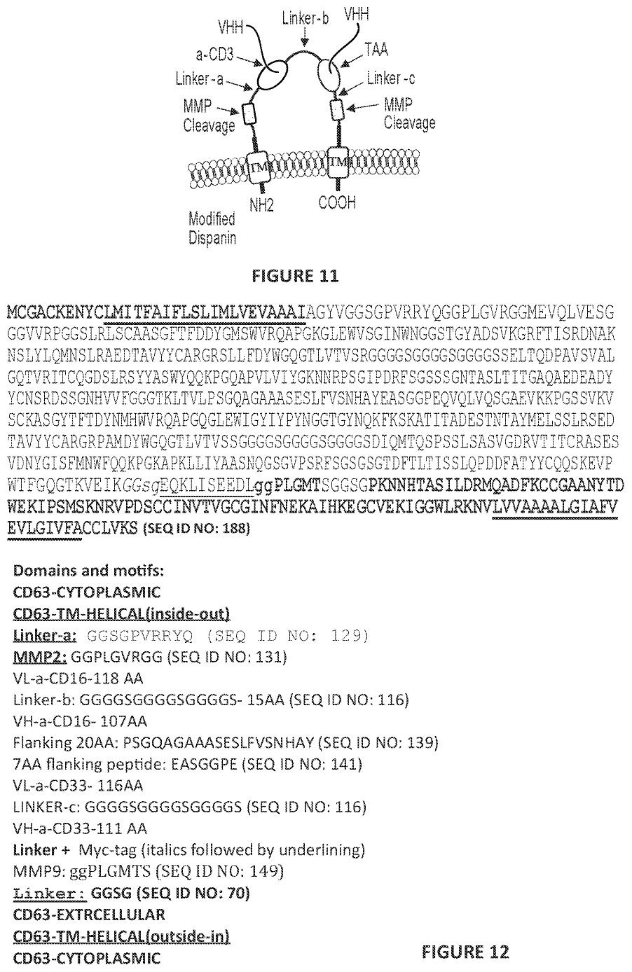

The above SEQ ID NO 188 is divided into functional domains and motifs as shown in FIG. 11 and as follows:
CD63-CYTOPLASMIC: MCGACKENYC (SEQ ID NO: 190) which is endcoded by cDNA:

(SEQ ID NO: 191)
ATGTGCGGCGCCTGTAAAGAAAACTACTGC

CD63-TM-HELICAL(inside-out) LMITFAIFLSLIMLVEVAAAI (SEQ ID NO: 192) which is encoded by cDNA:

(SEQ ID NO: 193)
CTGATGATCACATTCGCAATCTTCCTGTCCCTCATCATGCTGGTGGAGGT
GGCCGCCGCCATC

Linker-a: GGSGPVRRYQ (SEQ ID NO: 194) which is encoded by cDNA:

(SEQ ID NO: 195)
CGGCAGCGGCCCTGTGAGGAGATACCAG

MMP2: GGPLGVRGG (SEQ ID NO: 196) which is encoded by cDNA:

(SEQ ID NO: 197)
GGCGGTCCACTGGGCGTGCGGGGCGGC

VL-a-CD16-118AA

(SEQ ID NO: 198)
MEVQLVESGGGVVRPGGSLRLSCAASGFTFDDYGMSWVRQAPGKGLEWVS
GINWNGGSTGYADSVKGRFTISRDNAKNSLYLQMNSLRAEDTAVYYCARG
RSLLFDYWGQGTLVTVSR

Which is encoded by cDNA:

(SEQ ID NO: 199)
ATGGAAGTGCAGCTGGTGGAGAGCGGCGGCGGCGTGGTGAGACCTGGCGG
GTCCCTGCGGCTGAGCTGTGCCGCCAGCGGATTCACATTCGACGATTACG
GCATGAGCTGGGTCAGACAGGCCCCAGGAAAAGGCCTGGAATGGGTGTCT
GGCATTAACTGGAACGGCGGCAGCACCGGCTACGCCGATAGCGTTAAGGG
CAGATTCACCATCAGCAGAGATAACGCCAAGAACTCTCTGTACCTGCAGA
TGAATAGCCTGCGGGCCGAAGATACCGCCGTCTACTACTGTGCCCGGGGA
AGAAGCTTGCTGTTTGACTACTGGGGACAGGGCACACTGGTCACCGTGTC
TAGG

Linker-b and Linker-c: GGGGSGGGGSGGGGS 15AA (SEQ ID NO:200) which is encoded by cDNA:

(SEQ ID NO: 201)
GGCGGCGGCGGCAGCGGAGGAGGCGGGTCGGGCGGCGGCGGATCC

VH-a-CD16-107AA

(SEQ ID NO: 202)
SELTQDPAVSVALGQTVRITCQGDSLRSYYASWYQQKPGQAPVLVIYGKN
NRPSGIPDRFSGSSSGNTASLTITGAQAEDEADYYCNSRDSSGNHVVFGG
GTKLTVL

which is encoded by cDNA:

(SEQ ID NO: 203)
AGCGAGCTGACACAAGATCCTGCTGTGAGCGTCGCCCTGGGCCAGACCGT
GCGGATCACTTGCCAGGGAGATAGCCTGAGGTCCTACTACGCCAGTTGGT
ATCAACAGAAGCCTGGACAGGCCCCTGTACTGGTGATCTACGGCAAGAAC
AACAGACCTTCTGGCATCCCTGATAGATTCAGCGGCTCCAGCAGCGGAAA
CACCGCTAGCCTGACAATCACCGGAGCTCAGGCTGAGGACGAGGCCGACT
ACTACTGCAACAGCCGGGACTCTTCAGGAAATCACGTGGTGTTCGGCGGC
GGCACCAAGCTGACAGTGCTG

Flanking 20AA: PSGQAGAAASESLFVSNHAY (SEQ ID NO:204) which is encoded by cDNA:

(SEQ ID NO: 205)
CCTAGCGGCCAGGCCGGCGCCGCGGCCAGCGAAAGCCTGTTCGTGTCTAA
CCACGCCTAC

7AA flanking peptide: EASGGPE (SEQ ID NO:206) which is encoded by cDNA:

(SEQ ID NO: 207)
GAGGCTTCCGGCGGCCCCGAG

VL-a-CD33-116AA

(SEQ ID NO: 208)
QVQLVQSGAEVKKPGSSVKVSCKASGYTFTDYNMHWVRQAPGQGLEWIGY
IYPYNGGTGYNQKFKSKATITADESTNTAYMELSSLRSEDTAVYYCARGR
PAMDYWGQGTLVTVSS

Which is encoded by cDNA:

(SEQ ID NO: 209)
CAGGTGCAGCTGGTGCAAAGCGGCGCCGAGGTGAAGAAGCCCGGCAGCAG
CGTGAAAGTGTCCTGCAAGGCCTCCGGCTACACCTTTACCGACTACAATA
TGCACTGGGTTCGGCAGGCCCCTGGCCAAGGTCTCGAGTGGATCGGCTAC
ATCTATCCTTACAACGGCGGCACCGGCTACAACCAGAAGTTCAAAAGCAA
GGCAACAATCACAGCCGACGAGAGCACTAACACCGCCTACATGGAACTGA
GCAGCCTGAGAAGCGAGGACACCGCTGTTTACTACTGCGCCAGAGGCAGA
CCAGCTATGGACTACTGGGGCCAAGGAACCCTGGTGACCGTGTCATCT

VH-a-CD33

(SEQ ID NO: 210)
DIQMTQSPSSLSASVGDRVTITCRASESVDNYGISFMNWFQQKPGKAPKL
LIYAASNQGSGVPSRFSGSGSGTDFTLTISSLQPDDFATYYCQQSKEVPW
TFGQGTKVEIK

Which is encoded by cDNA:

(SEQ ID NO: 211)
GATATCCAGATGACCCAGAGCCCTAGCAGCCTGTCTGCCTCTGTTGGCGA
CCGGGTGACCATCACCTGCAGAGCTTCTGAATCAGTTGACAACTACGGCA
TCAGCTTCATGAACTGGTTCCAGCAGAAACCTGGCAAGGCCCCCAAGCTG
CTGATCTATGCCGCTTCTAATCAGGGCAGCGGCGTGCCTAGCAGATTTAG
CGGCAGTGGATCGGGAACCGACTTCACCCTGACCATTTCTTCGCTTCAGC
CCGACGATTTCGCCACATACTATTGTCAGCAGAGCAAGGAGGTGCCATGG
ACATTTGGCCAGGGCACTAAGGTGGAAATCAAG

Myc-tag ggsgeqkliseedlgg (SEQ ID NO: 380) which is encoded by cDNA:

(SEQ ID NO: 180)
GGCGGCAGCGGAGAGCAGAAGCTGATCTCCGAGGAAGACCTGGGTGGA

MMP9: ggPLGMTS (SEQ ID NO: 149) which is encoded by cDNA:

(SEQ ID NO: 182)
CCCCTGGGCATGACCTCT

CD63-EXTRACELLULAR

(SEQ ID NO: 151)
PKNNHTASILDRMQADFKCCGAANYTDWEKIPSMSKNRVPDSCCINVTVG
CGINFNEKAIHKEGCVEKIGGWLRKNV

Which is encoded by cDNA:

(SEQ ID NO: 184)
CCTAAGAACAACCACACAGCCAGCATCCTGGACAGAATGCAGGCCGACTT
CAAGTGCTGCGGCGCCGCTAATTACACCGACTGGGAGAAAATCCCCTCCA
TGAGCAAAAACCGGGTGCCCGACAGCTGTTGCATCAATGTGACCGTGGGC
TGTGGCATTAACTTCAACGAGAAGGCTATCCATAAGGAGGGCTGCGTCGA
GAAGATCGGCGGCTGGCTGAGAAAGAACGTG

CD63-TM-HELICAL(outside-in) LVVAAAALGIAFVEVLGIVFA (SEQ ID NO: 185) which is encoded

(SEQ ID NO: 186)
CTGGTGGTGGCCGCCGCCGCCCTGGGTATCGCCTTCGTGGAAGTGCTGGG
CATCGTGTTCGCC

CD63-CYTOPLASMIC CCLVKS (SEQ ID NO: 325) which is encoded by cDNA:

(SEQ ID NO: 379)
TGCTGCCTGGTGAAGTCT

### Example 5: Delivery of a TRIKE with ADAM 17

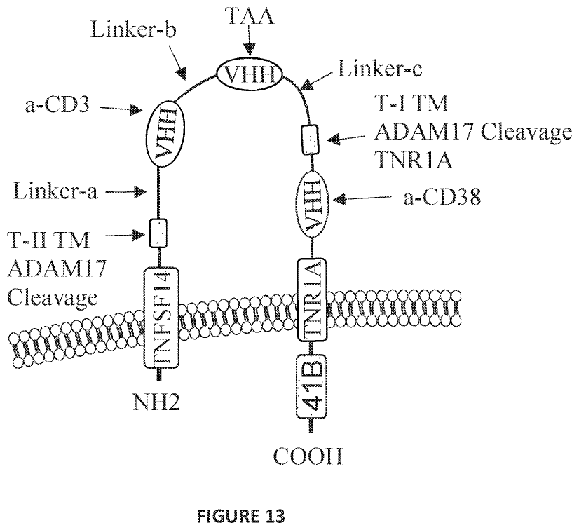

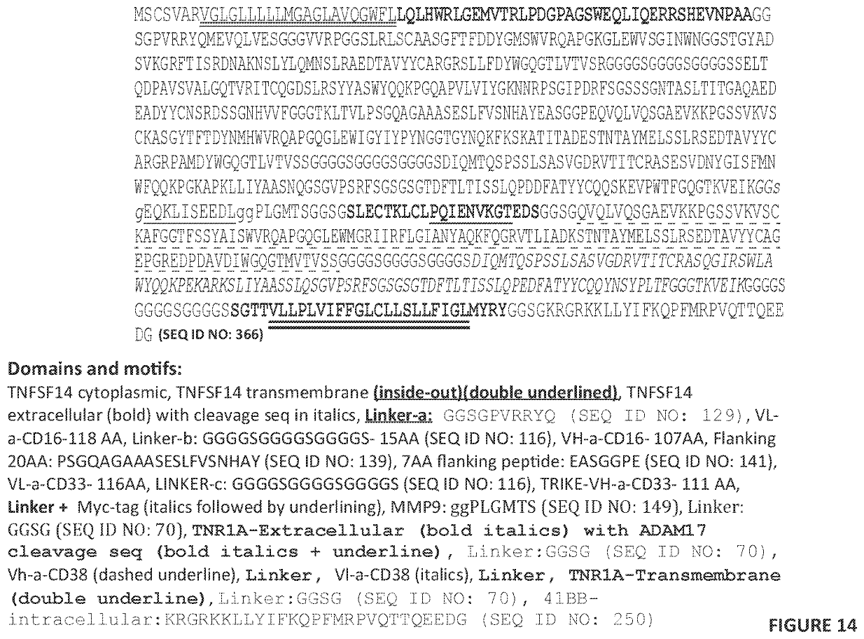

Referring to FIGS. 13-14, in one embodiment the invention comprises a TRIKE with ADAM17 target sequences embedded in the Type-II TM (TNFSF14) and Type-I TM (TNR1A) domains. This molecule will be expressed on the cell surface and cleaved upon NK cell activation.

This construct is outlined in SEQ ID NO: 212 below:

(SEQ ID NO: 212)
MSCSVARVGLGLLLLLMGAGLAVQGWFLLQLHWRLGEMVTRLPDGPAGSW
EQLIQERRSHEVNPAAGGSGPVRRYQMEVQLVESGGGVVRPGGSLRLSCA
ASGFTEDDYGMSWVRQAPGKGLEWVSGINWNGGSTGYADSVKGRFTISRD
NAKNSLYLQMNSLRAEDTAVYYCARGRSLLFDYWGQGTLVTVSRGGGGSG
GGGSGGGGSSELTQDPAVSVALGQTVRITCQGDSLRSYYASWYQQKPGQA
PVLVIYGKNNRPSGIPDRFSGSSSGNTASLTITGAQAEDEADYYCNSRDS
SGNHVVFGGGTKLTVLPSGQAGAAASESLFVSNHAYEASGGPEQVQLVQS
GAEVKKPGSSVKVSCKASGYTFTDYNMHWVRQAPGQGLEWIGYIYPYNGG
TGYNQKFKSKATITADESTNTAYMELSSLRSEDTAVYYCARGRPAMDYWG
QGTLVTVSSGGGGSGGGGSGGGGSDIQMTQSPSSLSASVGDRVTITCRAS
ESVDNYGISFMNWFQQKPGKAPKLLIYAASNQGSGVPSRFSGSGSGTDFT
LTISSLQPDDFATYYCQQSKEVPWTFGQGTKVEIKGGSGEQKLISEEDLS
GGSGSLECTKLCLPQIENVKGTEDSGGSGQVQLVQSGAEVKKPGSSVKVS
CKAFGGTFSSYAISWVRQAPGQGLEWMGRIIRFLGIANYAQKFQGRVTLI
ADKSTNTAYMELSSLRSEDTAVYYCAGEPGREDPDAVDIWGQGTMVTVSS
GGGGSGGGGSGGGGSDIQMTQSPSSLSASVGDRVTITCRASQGIRSWLAW
YQQKPEKARKSLIYAASSLQSGVPSRFSGSGSGTDFTLTISSLQPEDFAT
YYCQQYNSYPLTFGGGTKVEIKGGGGSGGGGSGGGGSSGTTVLLPLVIFF
GLCLLSLLFIGLMYRYGGSGKRGRKKLLYIFKQPFMRPVQTTQEEDG

The corresponding cDNA is set forth below:

(SEQ ID NO: 213)
ATGAGCTGCAGCGTGGCGAGAGTAGGGCTGGGCCTGCTCCTGCTGCTGAT
GGGCGCCGGCCTGGCCGTGCAAGGATGGTTCCTGCTGCAGCTTCATTGGA
GACTGGGAGAGATGGTGACTAGACTGCCTGACGGCCCTGCCGGCAGCTGG
GAGCAGCTGATCCAGGAGAGAAGGAGCCACGAGGTGAACCCTGCTGCAGG
TGGGAGCGGCCCTGTGCGGCGGTACCAAATGGAAGTGCAGCTGGTCGAGT
CTGGGGGCGGCGTGGTCCGGCCAGGCGGCAGCCTGCGTCTGTCTTGCGCC
GCTAGCGGCTTCACTTTCGATGACTACGGCATGAGCTGGGTGCGGCAGGC
CCCCGGCAAGGGCCTCGAGTGGGTTTCTGGCATCAACTGGAACGGCGGCT
CTACCGGCTACGCCGACAGCGTGAAGGGCCGCTTTACCATCTCCAGAGAT
AATGCCAAGAACAGCCTGTACCTGCAGATGAACAGCCTGCGGGCCGAGGA
TACGGCCGTGTACTATTGTGCCCGGGGCAGAAGCCTCCTCTTCGACTACT
GGGGCCAGGGCACCCTGGTTACCGTGAGCCGGGGCGGCGGCGGTTCTGGA
GGCGGAGGCAGCGGCGGCGGGGGCTCCTCTGAGCTGACCCAGGACCCCGC
CGTGAGCGTGGCCCTGGGCCAGACAGTCAGAATTACATGCCAGGGAGATA
GCCTGAGATCCTACTATGCCAGCTGGTACCAGCAGAAACCTGGCCAAGCT
CCTGTGCTGGTCATCTACGGCAAAAACAACAGACCTAGTGGAATCCCCGA
CAGATTTAGCGGATCCTCCAGCGGCAATACCGCCTCCTTGACAATCACCG
GCGCTCAGGCCGAGGATGAAGCCGACTACTACTGCAACAGCAGAGACAGC
TCTGGGAACCACGTGGTGTTCGGGGGGGGAACCAAGCTTACAGTGCTGCC
TTCTGGGCAGGCGGGAGCCGCCGCTAGCGAATCTCTGTTTGTGTCTAATC
ACGCCTACGAGGCCAGCGGCGGCCCCGAGCAGGTGCAGCTGGTGCAGAGT
GGGGCTGAAGTGAAGAAACCTGGCAGCTCGGTGAAGGTGAGCTGCAAGGC
CTCTGGCTACACCTTCACCGACTACAACATGCACTGGGTCAGACAGGCCC
CTGGCCAGGGACTGGAATGGATCGGCTACATCTACCCCTACAACGGCGGC
ACTGGCTATAACCAAAAATTCAAGTCTAAGGCAACCATCACAGCCGACGA
GAGCACCAACACCGCCTATATGGAGCTGTCTAGCCTGAGGAGCGAGGACA
CCGCCGTGTACTACTGCGCCCGGGGAAGACCTGCCATGGACTACTGGGGC
CAAGGAACACTGGTGACCGTCAGCTCTGGAGGCGGCGGATCTGGCGGAGG
AGGCAGCGGCGGCGGCGGTTCAGACATCCAGATGACCCAGTCACCTAGCT
CTCTGAGCGCCAGCGTGGGCGACCGGGTGACCATCACCTGTAGAGCCAGC
GAGAGCGTGGACAACTACGGAATCTCATTCATGAACTGGTTCCAGCAGAA
GCCCGGAAAGGCCCCGAAGCTGCTGATCTATGCTGCCAGCAACCAGGGTT
CAGGGGTGCCTAGCAGATTTTCTGGGAGCGGCTCTGGCACCGACTTTACC
CTGACAATCTCTAGCCTGCAGCCAGACGATTTCGCCACTTACTACTGTCA
GCAGAGCAAGGAAGTGCCATGGACCTTCGGACAAGGAACAAAGGTTGAAA
TTAAGGGAGGCAGCGGGGAACAAAAGCTGATCAGCGAAGAGGACCTGAGC
GGCGGAAGCGGCAGCCTGGAGTGCACCAAGCTGTGTCTGCCTCAGATCGA
GAATGTGAAGGGCACAGAGGATTCCGGCGGCAGCGGCCAAGTGCAGCTGG
TGCAGAGCGGCGCTGAGGTGAAGAAGCCCGGCAGCAGCGTGAAGGTGAGC
TGTAAAGCCTTCGGCGGAACCTTCAGCTCCTACGCTATCAGCTGGGTGAG
ACAGGCCCCTGGCCAGGGGCTAGAATGGATGGGCAGAATCATCCGGTTCC
TGGGCATCGCCAATTACGCCCAGAAGTTTCAGGGCCGGGTGACGCTGATC
GCCGATAAGAGCACCAACACCGCCTACATGGAACTGTCTTCCCTGCGATC
AGAAGACACCGCCGTGTACTACTGCGCCGGCGAACCTGGAAGAGAAGATC
CTGACGCCGTGGATATCTGGGGACAGGGCACAATGGTGACAGTGTCGAGC
GGCGGAGGCGGCTCTGGTGGCGGTGGCTCTGGCGGCGGCGGCTCCGACAT
TCAGATGACACAGTCCCCTTCCTCTCTGTCCGCCTCCGTGGGCGACCGGG
TGACCATCACCTGCAGAGCCAGTCAGGGCATCAGAAGCTGGCTGGCTTGG
TACCAGCAGAAGCCTGAGAAGGCCAGGAAAAGCCTGATCTACGCCGCGAG
CAGCCTGCAGTCTGGGGTGCCCTCTAGATTCAGCGGTTCTGGCAGCGGCA
CAGATTTCACACTGACAATAAGTTCGCTGCAGCCTGAAGACTTCGCCACC
TATTACTGCCAGCAATACAACAGCTACCCTCTGACCTTCGGCGGCGGAAC
CAAGGTTGAGATCAAGGGCGGCGGCGGTAGCGGCGGCGGCGGCTCCGGCG
GCGGAGGCAGCTCCGGCACCACCGTCCTGCTGCCTCTGGTGATCTTCTTC
GGCCTGTGCCTGCTGTCACTGCTGTTCATCGGACTCATGTACAGATACGG
CGGAAGCGGCC

### Domains and Motifs:

SEQ ID NO: 212 is divided into functional domains and motifs as set forth below and as shown in FIG. 14.
TNFSF14 cytoplasmic MSCSVAR (SEQ ID NO:214), which is encoded by cDNA:

(SEQ ID NO: 215)
ATGAGCTGCAGCGTGGCGAGA

TNFSF14 transmembrane (inside-out) VGLGLLLLLMGAGLAVQGWFL (SEQ ID NO: 216), which is encoded by cDNA:

(SEQ ID NO: 217)
GTGGCGAGAGTAGGGCTGGGCCTGCTCCTGCTGCTGATGGGCGCCGGCC
TGGCCGTGCAAGGATGGTTCCTG

TNFSF14 extracellular with cleavage seq:

(SEQ ID NO: 218)
GSWEQLLQLHWRLGEMVTRLPDGPAGSWEQLIQERRSHEVNPAA

which is encoded by cDNA:

(SEQ ID NO: 219)
CTGCAGCTTCATTGGAGACTGGGAGAGATGGTGACTAGACTGCCTGACG
GCCCTGCCGGCAGCTGGGAGCAGCTGATCCAGGAGAGAAGGAGCCACGA
GGTGAACCCTGCTGCA

Linker-a: GGSGPVRRYQ (SEQ ID NO:220) which is encoded by cDNA:

(SEQ ID NO: 221)
GGTGGGAGCGGCCCTGTGCGGCGGTACCAA

VL-a-CD16-118AA MEVQLVESGGGVVRPGGSLRLSCAASGFTFDDYGMSWVRQAPGKGLEWVSGINWNGGSTGY ADSVKGRFTISRDNAKNSLYLQMNSLRAEDTAVYYCARGRSLLFDYWGQGTLVTVSR (SEQ ID NO: 222), Which is encoded by cDNA:

(SEQ ID NO: 223)
ATGGAAGTGCAGCTGGTCGAGTCTGGGGGCGGCGTGGTCCGGCCAGGCG
GCAGCCTGCGTCTGTCTTGCGCCGCTAGCGGCTTCACTTTCGATGACTA
CGGCATGAGCTGGGTGCGGCAGGCCCCCGGCAAGGGCCTCGAGTGGGTT
TCTGGCATCAACTGGAACGGCGGCTCTACCGGCTACGCCGACAGCGTGA
AGGGCCGCTTTACCATCTCCAGAGATAATGCCAAGAACAGCCTGTACCT
GCAGATGAACAGCCTGCGGGCCGAGGATACGGCCGTGTACTATTGTGCC
CGGGGCAGAAGCCTCCTCTTCGACTACTGGGGCCAGGGCACCCTGGTTA
CCGTGAGCCGG

TRIKE-Linker: GGGGSGGGGSGGGGS (SEQ ID NO: 224) which is encoded by cDNA:

(SEQ ID NO: 225)
GGCGGCGGCGGTTCTGGAGGCGGAGGCAGCGGCGGCGGGGGCTCC

VH-a-CD16-107AA

(SEQ ID NO: 226)
SELTQDPAVSVALGQTVRITCQGDSLRSYYASWYQQKPGQAPVLVIYGK
NNRPSGIPDRFSGSSSGNTASLTITGAQAEDEADYYCNSRDSSGNHVVF
GGGTKLTVL

which is encoded by cDNA:

(SEQ ID NO: 227)
TCTGAGCTGACCCAGGACCCCGCCGTGAGCGTGGCCCTGGGCCAGACAG
TCAGAATTACATGCCAGGGAGATAGCCTGAGATCCTACTATGCCAGCTG
GTACCAGCAGAAACCTGGCCAAGCTCCTGTGCTGGTCATCTACGGCAAA
AACAACAGACCTAGTGGAATCCCCGACAGATTTAGCGGATCCTCCAGCG
GCAATACCGCCTCCTTGACAATCACCGGCGCTCAGGCCGAGGATGAAGC
CGACTACTACTGCAACAGCAGAGACAGCTCTGGGAACCACGTGGTGTTC
GGGGGGGGAACCAAGCTTACAGTGCTG

Flanking 20AA: PSGQAGAAASESLFVSNHAY (SEQ ID NO: 228)_which is encoded by cDNA:

(SEQ ID NO: 229)
CCTTCTGGGCAGGCGGGAGCCGCCGCTAGCGAATCTCTGTTTGTGTCTA
ATCACGCCTAC

7AA flanking peptide: EASGGPE (SEQ ID NO: 230) which is encoded by cDNA:

(SEQ ID NO: 231)
GAGGCCAGCGGCGGCCCCGAGCAGGTG

VL-a-CD33-116AA

(SEQ ID NO: 232)
QVQLVQSGAEVKKPGSSVKVSCKASGYTFTDYNMHWVRQAPGQGLEWIG
YIYPYNGGTGYNQKFKSKATITADESTNTAYMELSSLRSEDTAVYYCAR
GRPAMDYWGQGTLVTVSS

which is encoded by cDNA:

(SEQ ID NO: 233)
CAGCTGGTGCAGAGTGGGGCTGAAGTGAAGAAACCTGGCAGCTCGG
TGAAGGTGAGCTGCAAGGCCTCTGGCTACACCTTCACCGACTACAA
CATGCACTGGGTCAGACAGGCCCCTGGCCAGGGACTGGAATGGATC
GGCTACATCTACCCCTACAACGGCGGCACTGGCTATAACCAAAAAT
TCAAGTCTAAGGCAACCATCACAGCCGACGAGAGCACCAACACCGC
CTATATGGAGCTGTCTAGCCTGAGGAGCGAGGACACCGCCGTGTAC
TACTGCGCCCGGGGAAGACCTGCCATGGACTACTGGGGCCAAGGAA
CACTGGTGACCGTCAGC

TRIKE-LINKER: GGGGSGGGGSGGGGS (SEQ ID NO: 234) which is encoded by cDNA: TCTGGAGGCGGCGGATCTGGCGGAGGAGGCAGCGGCGGCGGCGGTTCA (SEQ ID NO: 235)
TRIKEVH-a-CD33

(SEQ ID NO: 236)
DIQMTQSPSSLSASVGDRVTITCRASESVDNYGISFMNWFQQKPGK
APKLLIYAASNQGSGVPSRFSGSGSGTDFTLTISSLQPDDFATYYC
QQSKEVPWTFGQGTKVEIK

Which is encoded by cDNA:

(SEQ ID NO: 237)
GACATCCAGATGACCCAGTCACCTAGCTCTCTGAGCGCCAGCGTGG
GCGACCGGGTGACCATCACCTGTAGAGCCAGCGAGAGCGTGGACAA
CTACGGAATCTCATTCATGAACTGGTTCCAGCAGAAGCCCGGAAAG
GCCCCGAAGCTGCTGATCTATGCTGCCAGCAACCAGGGTTCAGGGG
TGCCTAGCAGATTTTCTGGGAGCGGCTCTGGCACCGACTTTACCCT
GACAATCTCTAGCCTGCAGCCAGACGATTTCGCCACTTACTACTGT
CAGCAGAGCAAGGAAGTGCCATGGACCTTCGGACAAGGAACAAAGG
TTGAAATTAAG

Myc-tag GGSGEQKLISEEDLSGGSG (SEQ ID NO: 238) which is encoded by cDNA:

(SEQ ID NO: 239)
GGAGGCAGCGGGGAACAAAAGCTGATCAGCGAAGAGGACCTG

TNR 1A-Extracellular with ADAM17 cleavage seq SLECTKLCLPQIENVKGTEDS (SEQ ID NO: 240) which is encoded by cDNA:

(SEQ ID NO: 241)
AGCCTGGAGTGCACCAAGCTGTGTCTGCCTCAGATCGAGAATGTGA
AGGGCACAGAGGAT

Vh-a-CD38

(SEQ ID NO: 242)
QVQLVQSGAEVKKPGSSVKVSCKAFGGTFSSYAISWVRQAPGQGLE
WMGRIIRFLGIANYAQKFQGRVTLIADKSTNTAYMELSSLRSEDTA
VYYCAGEPGREDPDAVDIWGQGTMVTVSS

Which is encoded by cDNA:

(SEQ ID NO: 243)
CAAGTGCAGCTGGTGCAGAGCGGCGCTGAGGTGAAGAAGCCCGGCA
GCAGCGTGAAGGTGAGCTGTAAAGCCTTCGGCGGAACCTTCAGCTC
CTACGCTATCAGCTGGGTGAGACAGGCCCCTGGCCAGGGGCTAGAA
TGGATGGGCAGAATCATCCGGTTCCTGGGCATCGCCAATTACGCCC
AGAAGTTTCAGGGCCGGGTGACGCTGATCGCCGATAAGAGCACCAA
CACCGCCTACATGGAACTGTCTTCCCTGCGATCAGAAGACACCGCC
GTGTACTACTGCGCCGGCGAACCTGGAAGAGAAGATCCTGACGCCG
TGGATATCTGGGGACAGGGCACAATGGTGACAGTGTCGAGC

Linker GGGGSGGGGSGGGGS (SEQ ID NO: 244) which is encoded by cDNA:

(SEQ ID NO: 245)
GGCGGAGGCGGCTCTGGTGGCGGTGGCTCTGGCGGCGGCGGCTCC

Vl-a-CD38

(SEQ ID NO: 246)
DIQMTQSPSSLSASVGDRVTITCRASQGIRSWLAWYQQKPEKARKS
LIYAASSLQSGVPSRFSGSGSGTDFTLTISSLQPEDFATYYCQQYN
SYPLTFGGGTKVEIK

which is encoded by cDNA:

(SEQ ID NO: 247)
GACATTCAGATGACACAGTCCCCTTCCTCTCTGTCCGCCTCCGTGG
GCGACCGGGTGACCATCACCTGCAGAGCCAGTCAGGGCATCAGAAG
CTGGCTGGCTTGGTACCAGCAGAAGCCTGAGAAGGCCAGGAAAAGC
CTGATCTACGCCGCGAGCAGCCTGCAGTCTGGGGTGCCCTCTAGAT
TCAGCGGTTCTGGCAGCGGCACAGATTTCACACTGACAATAAGTTC
GCTGCAGCCTGAAGACTTCGCCACCTATTACTGCCAGCAATACAAC
AGCTACCCTCTGACCTTCGGCGGCGGAACCAAGGTTGAGATCAAG

TNR1A-Transmembrane SGTTVLLPLVIFFGLCLLSLLFIGLMYRY (SEQ ID NO: 248) which is encoded by cDNA:

(SEQ ID NO: 249)
TCCGGCACCACCGTCCTGCTGCCTCTGGTGATCTTCTTCGGCCTGT
GCCTGCTGTCACTGCTGTTCATCGGACTCATGTACAGATAC

41BB-intracellular: KRGRKKLLYIFKQPFMRPVQTTQEEDG (SEQ ID NO: 250) which is encoded by cDNA:

(SEQ ID NO: 251)
AAAAGAGGCCGAAAGAAGCTGCTGTACATCTTTAAACAGCCCTTCA
TGAGACCCGTGCAAACAACCCAGGAGGAAGATGGC

### Example 6: BIKE with Anti-CD38 with Granzyme B Cleavage Sequences

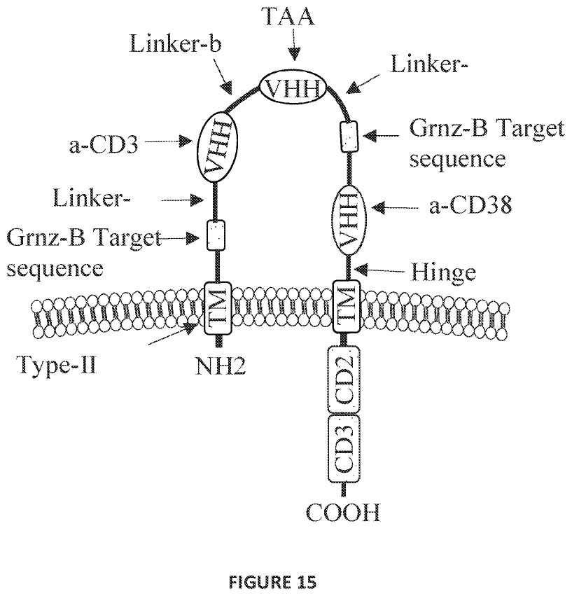

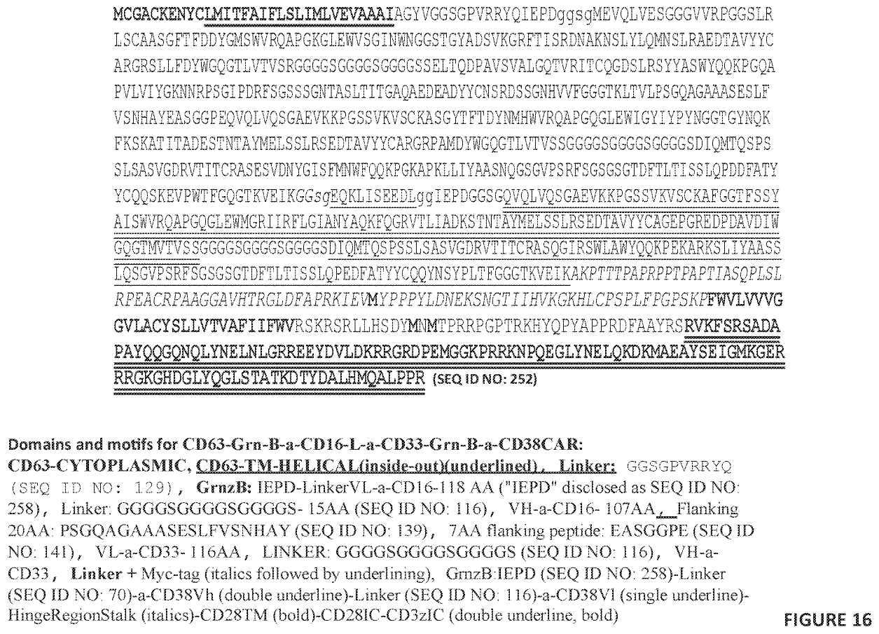

Referring to FIGS. 15 and 16, in some embodiments the invention comprises a BIKE+anti-CD38 with Granzyme-B cleavage sequences with activation domains having SEQ ID NO 71 of 252. This molecule will be membrane-bound and released upon Granzyme-B release following NK cell degranulation. Anti-CD38 will stay on NK cells following the release of the rest of the cargo and will have a function similar to a chimeric antigen receptor (CAR).

(SEQ ID NO: 252)
MCGACKENYCLMITFAIFLSLIMLVEVAAAIAGYVGGSGPVRRYQI
EPDGGSGMEVQLVESGGGVVRPGGSLRLSCAASGFTFDDYGMSWVR
QAPGKGLEWVSGINWNGGSTGYADSVKGRFTISRDNAKNSLYLQMN
SLRAEDTAVYYCARGRSLLFDYWGQGTLVTVSRGGGGSGGGGSGGG
GSSELTQDPAVSVALGQTVRITCQGDSLRSYYASWYQQKPGQAPVL
VIYGKNNRPSGIPDRFSGSSSGNTASLTITGAQAEDEADYYCNSRD
SSGNHVVFGGGTKLTVLPSGQAGAAASESLFVSNHAYEASGGPEQV
QLVQSGAEVKKPGSSVKVSCKASGYTFTDYNMHWVRQAPGQGLEWI
GYIYPYNGGTGYNQKFKSKATITADESTNTAYMELSSLRSEDTAVY
YCARGRPAMDYWGQGTLVTVSSGGGGSGGGGSGGGGSDIQMTQSPS
SLSASVGDRVTITCRASESVDNYGISFMNWFQQKPGKAPKLLIYAA
SNQGSGVPSRFSGSGSGTDFTLTISSLQPDDFATYYCQQSKEVPWT
FGQGTKVEIKGGSGEQKLISEEDLGGIEPDGGSGQVQLVQSGAEVK
KPGSSVKVSCKAFGGTFSSYAISWVRQAPGQGLEWMGRIIRFLGIA
NYAQKFQGRVTLIADKSTNTAYMELSSLRSEDTAVYYCAGEPGRED
PDAVDIWGQGTMVTVSSGGGGSGGGGSGGGGSDIQMTQSPSSLSAS
VGDRVTITCRASQGIRSWLAWYQQKPEKARKSLIYAASSLQSGVPS
RFSGSGSGTDFTLTISSLQPEDFATYYCQQYNSYPLTFGGGTKVEI
KAKPTTTPAPRPPTPAPTIASQPLSLRPEACRPAAGGAVHTRGLDF
APRKIEVMYPPPYLDNEKSNGTIIHVKGKHLCPSPLFPGPSKPFWV
LVVVGGVLACYSLLVTVAFIIFWVRSKRSRLLHSDYMNMTPRRPGP
TRKHYQPYAPPRDFAAYRSRVKFSRSADAPAYQQGQNQLYNELNLG
RREEYDVLDKRRGRDPEMGGKPRRKNPQEGLYNELQKDKMAEAYSE
IGMKGERRRGKGHDGLYQGLSTATKDTYDALHMQALPPR

The corresponding cDNA is set forth below:

(SEQ ID NO: 253)
ATGTGCGGCGCCTGCAAGGAAAACTACTGTCTGATGATCACTTTCG
CCATCTTTCTGTCTCTGATCATGCTGGTGGAAGTGGCCGCTGCCAT
TGCCGGATACGTGGGCGGCTCTGGCCCTGTGCGAAGATACCAAATC
GAGCCTGACGGCGGCAGCGGCATGGAAGTGCAGCTGGTTGAGTCCG
GCGGCGGAGTGGTGCGGCCTGGCGGCTCTCTGAGACTGTCTTGCGC
TGCCAGCGGTTTCACCTTCGACGATTACGGAATGAGCTGGGTGCGG
CAGGCCCCAGGCAAGGGCCTGGAGTGGGTGTCCGGCATCAACTGGA
ACGGCGGAAGCACCGGCTACGCAGACAGCGTGAAGGGCAGATTCAC
CATCTCCAGAGATAACGCCAAGAATTCACTGTACCTGCAGATGAAC
AGCCTGAGAGCTGAGGACACTGCAGTTTACTACTGCGCCAGAGGCC
GGAGCCTGCTGTTCGACTACTGGGGACAGGGAACACTGGTGACCGT
GAGCAGAGGCGGCGGCGGCTCTGGCGGCGGCGGAAGCGGCGGTGGA
GGCAGCTCCGAGCTGACCCAGGACCCCGCCGTGTCCGTGGCCCTGG
GCCAAACAGTGAGAATCACCTGTCAGGGCGACTCTCTGCGCTCTTA
CTACGCCAGCTGGTACCAACAGAAGCCCGGCCAGGCCCCTGTGCTG
GTGATCTACGGCAAGAACAACAGACCTTCTGGTATCCCCGACAGAT
TTAGCGGCTCTTCTAGCGGCAACACCGCCTCCCTGACCATCACAGG
CGCCCAGGCCGAGGACGAGGCTGACTACTATTGCAATAGTAGAGAC
AGCAGCGGAAACCATGTGGTGTTCGGCGGTGGCACAAAGCTGACAG
TGCTTCCCAGCGGACAGGCCGGAGCCGCCGCCTCTGAAAGCCTGTT
CGTGAGTAATCACGCCTACGAGGCCTCCGGAGGCCCTGAGCAGGTG
CAGCTGGTGCAGTCCGGCGCCGAGGTGAAGAAGCCAGGCAGCAGCG
TTAAGGTGAGTTGTAAAGCCTCCGGATACACCTTCACCGACTACAA
CATGCACTGGGTTAGACAGGCGCCTGGCCAGGGCCTGGAATGGATC
GGCTACATCTACCCTTACAACGGCGGGACCGGCTATAATCAGAAGT
TCAAGAGCAAGGCCACGATCACAGCCGACGAGAGCACCAACACAGC
ATACATGGAACTGAGCTCCCTGAGATCTGAGGACACCGCCGTGTAC
TACTGCGCCAGGGGTCGGCCGGCCATGGACTATTGGGGCCAGGGCA
CCCTGGTCACAGTGTCTAGCGGCGGCGGCGGCTCTGGCGGCGGCGG
CTCAGGCGGCGGCGGCAGCGATATTCAGATGACACAGAGCCCTAGC
AGCCTGTCTGCCTCTGTTGGAGATCGGGTGACAATCACATGTCGGG
CCTCCGAGTCTGTGGACAACTACGGCATCAGCTTCATGAACTGGTT
CCAGCAGAAACCAGGCAAGGCCCCTAAGCTGCTGATCTACGCCGCT
AGCAACCAGGGCAGCGGCGTGCCTTCCCGCTTCAGCGGCAGCGGCA
GCGGAACAGATTTCACCCTGACCATCTCTTCTCTCCAACCTGATGA
TTTCGCTACCTACTACTGCCAGCAGAGCAAGGAGGTGCCTTGGACC
TTCGGACAGGGCACCAAAGTGGAGATCAAGGGCGGCTCTGGGGAAC
AGAAGCTGATCAGCGAGGAAGACCTGGGAGGCATCGAGCCTGATGG
AGGGAGCGGACAAGTGCAGCTGGTTCAAAGCGGCGCTGAGGTGAAA
AAACCTGGGAGCAGCGTGAAGGTGTCCTGCAAGGCTTTCGGGGGCA
CCTTCAGCAGCTACGCAATCAGCTGGGTGCGGCAGGCTCCCGGCCA
GGGCCTGGAATGGATGGGCAGAATCATCCGGTTCCTCGGAATCGCC
AACTACGCCCAGAAGTTCCAGGGTAGAGTGACCCTGATTGCCGACA
AGAGCACAAACACCGCTTATATGGAACTGAGCTCTCTGCGCAGCGA
AGATACCGCCGTGTACTACTGCGCCGGCGAACCCGGCCGGGAAGAT
CCTGATGCCGTGGATATCTGGGGCCAGGGCACCATGGTGACAGTGT
CATCCGGTGGCGGCGGAAGCGGCGGCGGCGGTAGTGGGGGAGGCGG
CAGCGACATCCAGATGACCCAGTCACCTAGCTCTTTGTCGGCCAGC
GTCGGAGATAGAGTGACCATTACCTGTCGGGCCTCTCAGGGAATCA
GAAGCTGGCTCGCCTGGTACCAGCAAAAGCCTGAGAAGGCCAGAAA
ATCCCTAATCTATGCCGCTAGCAGCCTGCAGTCTGGCGTGCCCTCC
AGATTTAGCGGAAGCGGAAGCGGCACAGACTTCACACTGACAATCA
GCTCTCTGCAACCTGAGGATTTCGCCACCTACTACTGCCAACAGTA
CAACAGCTACCCACTGACATTTGGAGGCGGTACAAAGGTGGAAATC
AAGGCCAAGCCCACCACAACCCCTGCCCCTAGACCCCCCACCCCAG
CTCCCACCATCGCCAGCCAGCCTCTGTCCCTGCGGCCTGAAGCCTG
CAGACCCGCTGCCGGCGGCGCCGTCCACACCCGGGGACTGGACTTC
GCCCCTAGAAAGATCGAGGTGATGTACCCCCCTCCCTACCTGGATA
ATGAGAAGAGCAATGGCACAATCATCCACGTGAAAGGCAAACACCT
GTGCCCTTCTCCTCTGTTTCCTGGACCTAGCAAGCCCTTCTGGGTC
CTGGTCGTGGTCGGCGGCGTGCTGGCTTGCTACAGCCTCCTGGTGA
CCGTGGCCTTCATCATTTTTTGGGTGCGCAGCAAGAGATCCAGGCT
GCTGCACTCTGACTACATGAACATGACCCCTAGACGGCCTGGTCCT
ACAAGAAAGCACTACCAGCCTTACGCCCCTCCCAGAGATTTCGCTG
CCTACCGTAGCCGGGTGAAGTTCAGCAGATCTGCCGATGCCCCTGC
TTACCAACAGGGCCAGAACCAGCTGTACAACGAGCTGAACCTGGGC
CGGCGGGAAGAGTACGACGTGCTGGACAAAAGAAGAGGCAGAGACC
CTGAGATGGGGGGGAAGCCTCGGAGAAAGAACCCCCAGGAGGGCCT
GTACAATGAGCTGCAGAAGGACAAGATGGCCGAGGCCTATTCTGAG
ATCGGAATGAAGGGAGAAAGACGGAGAGGCAAAGGCCACGACGGCC
TGTACCAGGGTCTGTCGACCGCTACCAAGGACACCTACGACGCCCT
GCACATGCAGGCTCTGCCTCCAAGA

SEQ ID NO: 252 is divided into functional domains and motifs as set forth in FIG. 15 and as set out below.

CD63-CYTOPLASMIC MCGACKENYC (SEQ ID NO: 254) which is encoded by cDNA:

(SEQ ID NO: 255)
ATGTGCGGCGCCTGCAAGGAAAACTACTGT

CD63-TM-HELICAL(inside-out) LMITFAIFLSLIMLVEVAAAI (SEQ ID NO: 256) which is encoded by cDNA:

(SEQ ID NO: 257)
CTGATGATCACTTTCGCCATCTTTCTGTCTCTGATCATGCTGGTGGAAG
TGGCCGCT

Granzyme B cleavage site: IEPD (SEQ ID NO: 258) which is encoded by cDNA: ATCGAGCCTGAC (SEQ ID NO: 259). Alternatively, Granzyme B cleavage site EEEEEVEADSEEEEEEE (SEQ ID NO: 260), Granzyme B cleavage site: AQGVISADASNLDDFY (SEQ ID NO: 261), or Granzyme B cleavage site: LEADKGKLEYD (SEQ ID NO: 262) may be used.
Linker-a: GGSG (SEQ ID NO: 263) GGCGGCAGCGGC (SEQ ID NO: 264)
   VL-a-CD16-118AA

(SEQ ID NO: 265)
MEVQLVESGGGVVRPGGSLRLSCAASGFTFDDYGMSWVRQAPGKGLEWV
SGINWNGGSTGYADSVKGRFTISRDNAKNSLYLQMNSLRAEDTAVYYCA
RGRSLLFDYWGQGTLVTVSR

Which is encoded by cDNA:

(SEQ ID NO: 266)
ATGGAAGTGCAGCTGGTTGAGTCCGGCGGCGGAGTGGTGCGGCCTGGCGG
CTCTCTGAGACTGTCTTGCGCTGCCAGCGGTTTCACCTTCGACGATTACG
GAATGAGCTGGGTGCGGCAGGCCCCAGGCAAGGGCCTGGAGTGGGTGTCC
GGCATCAACTGGAACGGCGGAAGCACCGGCTACGCAGACAGCGTGAAGGG
CAGATTCACCATCTCCAGAGATAACGCCAAGAATTCACTGTACCTGCAGA
TGAACAGCCTGAGAGCTGAGGACACTGCAGTTTACTACTGCGCCAGAGGC
CGGAGCCTGCTGTTCGACTACTGGGGACAGGGAACACTGGTGACCGTGAG
CAGA

VH-a-CD16-107AA

(SEQ ID NO: 267)
SELTQDPAVSVALGQTVRITCQGDSLRSYYASWYQQKPGQAPVLVIYGK
NNRPSGIPDRFSGSSSGNTASLTITGAQAEDEADYYCNSRDSSGNHVVF
GGGTKLTVL

Which is encoded by cDNA:

(SEQ ID NO: 268)
TCCGAGCTGACCCAGGACCCCGCCGTGTCCGTGGCCCTGGGCCAAACAGT
GAGAATCACCTGTCAGGGCGACTCTCTGCGCTCTTACTACGCCAGCTGGT
ACCAACAGAAGCCCGGCCAGGCCCCTGTGCTGGTGATCTACGGCAAGAAC
AACAGACCTTCTGGTATCCCCGACAGATTTAGCGGCTCTTCTAGCGGCAA
CACCGCCTCCCTGACCATCACAGGCGCCCAGGCCGAGGACGAGGCTGACT
ACTATTGCAATAGTAGAGACAGCAGCGGAAACCATGTGGTGTTCGGCGGT
GGCACAAAGCTGACAGTGCTT

Flanking 20AA: PSGQAGAAASESLFVSNHAY (SEQ ID NO: 269) which is encoded by cDNA:

(SEQ ID NO: 270)
CCCAGCGGACAGGCCGGAGCCGCCGCCTCTGAAAGCCTGTTCGTGAGTA
ATCACGCCTAC

7AA flanking peptide: EASGGPE (SEQ ID NO: 271) which is encoded by cDNA:

(SEQ ID NO: 272)
GAGGCCTCCGGAGGCCCTGAG

VL-a-CD33-116AA

(SEQ ID NO: 273)
QVQLVQSGAEVKKPGSSVKVSCKASGYTFTDYNMHWVRQAPGQGLEWIG
YIYPYNGGTGYNQKFKSKATITADESTNTAYMELSSLRSEDTAVYYCAR
GRPAMDYWGQGTLVTVSS

Which is encoded by cDNA:

(SEQ ID NO: 274)
CAGGTGCAGCTGGTGCAGTCCGGCGCCGAGGTGAAGAAGCCAGGCAGCAG
CGTTAAGGTGAGTTGTAAAGCCTCCGGATACACCTTCACCGACTACAACA
TGCACTGGGTTAGACAGGCGCCTGGCCAGGGCCTGGAATGGATCGGCTAC
ATCTACCCTTACAACGGCGGGACCGGCTATAATCAGAAGTTCAAGAGCAA
GGCCACGATCACAGCCGACGAGAGCACCAACACAGCATACATGGAACTGA
GCTCCCTGAGATCTGAGGACACCGCCGTGTACTACTGCGCCAGGGGTCGG
CCGGCCATGGACTATTGGGGCCAGGGCACCCTGGTCACAGTGTCTAGC

TRIKE VH-a-CD33

(SEQ ID NO: 275)
DIQMTQSPSSLSASVGDRVTITCRASESVDNYGISFMNWFQQKPGKAPK
LLIYAASNQGSGVPSRFSGSGSGTDFTLTISSLQPDDFATYYCQQSKEV
PWTFGQGTKVEIK

Which is encoded by cDNA:

(SEQ ID NO: 276)
GATATTCAGATGACACAGAGCCCTAGCAGCCTGTCTGCCTCTGTTGGAGA
TCGGGTGACAATCACATGTCGGGCCTCCGAGTCTGTGGACAACTACGGCA
TCAGCTTCATGAACTGGTTCCAGCAGAAACCAGGCAAGGCCCCTAAGCTG
CTGATCTACGCCGCTAGCAACCAGGGCAGCGGCGTGCCTTCCCGCTTCAG
CGGCAGCGGCAGCGGAACAGATTTCACCCTGACCATCTCTTCTCTCCAAC
CTGATGATTTCGCTACCTACTACTGCCAGCAGAGCAAGGAGGTGCCTTGG
ACCTTCGGACAGGGCACCAAAGTGGAGATCAAG

Myc-tag: GGSGEQKLISEEDLGG (SEQ ID NO: 277), which is encoded by cDNA:

(SEQ ID NO: 278)
GGCGGCTCTGGGGAACAGAAGCTGATCAGCGAGGAAGACCTGGGAGGC

Granzyme B cleavage site. IEPD (SEQ ID NO: 279)_ATCGAGCCTGAT (SEQ ID NO: 280)
   CD38Vh:

(SEQ ID NO: 281)
QVQLVQSGAEVKKPGSSVKVSCKAFGGTFSSYAISWVRQAPGQGLEWMG
RIIRFLGIANYAQKFQGRVTLIADKSTNTAYMELSSLRSEDTAVYYCAG
EPGREDPDAVDIWGQGTMVTVSS

Which is encoded by cDNA:

(SEQ ID NO: 282)
CAAGTGCAGCTGGTTCAAAGCGGCGCTGAGGTGAAAAAACCTGGGAGCAG
CGTGAAGGTGTCCTGCAAGGCTTTCGGGGGCACCTTCAGCAGCTACGCAA
TCAGCTGGGTGCGGCAGGCTCCCGGCCAGGGCCTGGAATGGATGGGCAGA
ATCATCCGGTTCCTCGGAATCGCCAACTACGCCCAGAAGTTCCAGGGTAG
AGTGACCCTGATTGCCGACAAGAGCACAAACACCGCTTATATGGAACTGA
GCTCTCTGCGCAGCGAAGATACCGCCGTGTACTACTGCGCCGGCGAACCC
GGCCGGGAAGATCCTGATGCCGTGGATATCTGGGGCCAGGGCACCATGGT
GACAGTGTCATCC

Linker: GGGGSGGGGSGGGGS (SEQ ID NO: 283) which is encoded by cDNA:

(SEQ ID NO: 284)
GGTGGCGGCGGAAGCGGCGGCGGCGGTAGTGGGGGAGGCGGCAGC

CD38Vl:

(SEQ ID NO: 285)
DIQMTQSPSSLSASVGDRVTITCRASQGIRSWLAWYQQKPEKARKSLIY
AASSLQSGVPSRFSGSGSGTDFTLTISSLQPEDFATYYCQQYNSYPLTF
GGGTKVEIK

Which is encoded by cDNA:

(SEQ ID NO: 286)
GACATCCAGATGACCCAGTCACCTAGCTCTTTGTCGGCCAGCGTCGGAGA
TAGAGTGACCATTACCTGTCGGGCCTCTCAGGGAATCAGAAGCTGGCTCG
CCTGGTACCAGCAAAAGCCTGAGAAGGCCAGAAAATCCCTAATCTATGCC
GCTAGCAGCCTGCAGTCTGGCGTGCCCTCCAGATTTAGCGGAAGCGGAAG
CGGCACAGACTTCACACTGACAATCAGCTCTCTGCAACCTGAGGATTTCG
CCACCTACTACTGCCAACAGTACAACAGCTACCCACTGACATTTGGAGGC
GGTACAAAGGTGGAAATCAAG

Hinge stalk region: AKPTTTPAPRPPTPAPTIASQPL SLRPEACRPAAGGAVHTRGLDFAPRKIEVMYPPPYLDN EKSNGTIIHVKGKHLCPSPLFPGPSKP (SEQ ID NO: 287) which is encoded by cDNA:

(SEQ ID NO: 288)
GCCAAGCCCACCACAACCCCTGCCCCTAGACCCCCCACCCCAGCTCCCAC
CATCGCCAGCCAGCCTCTGTCCCTGCGGCCTGAAGCCTGCAGACCCGCTG
CCGGCGGCGCCGTCCACACCCGGGGACTGGACTTCGCCCCTAGAAAGATC
GAGGTGATGTACCCCCCTCCCTACCTGGATAATGAGAAGAGCAATGGCAC
AATCATCCACGTGAAAGGCAAACACCTGTGCCCTTCTCCTCTGTTTCCTG
GACCTAGCAAGCCC

CD28 transmembrane: FWVLVVVGGVLACYSLLVTVAFIIFWV (SEQ ID NO: 289) which is encoded by cDNA:

(SEQ ID NO: 290)
TTCTGGGTCCTGGTCGTGGTCGGCGGCGTGCTGGCTTGCTACAGCCTCCT
GGTGACCGTGGCCTTCATCATTTTTTGGGTG

CD28 intracellular: RSKRSRLLHSDYMNMTPRRPGPTRKHYQPYAPPRDFAAYRS (SEQ ID NO:291) which is encoded by cDNA:

(SEQ ID NO: 292)
CGCAGCAAGAGATCCAGGCTGCTGCACTCTGACTACATGAACATGACCCC
TAGACGGCCTGGTCCTACAAGAAAGCACTACCAGCCTTACGCCCCTCCCA
GAGATTTCGCTGCCTACCGTAGC

CD3z intracellular: RVKFSRSADAPAYQQGQNQLYNELNLGRREEYDVLDKRRGRDPEMGGKPRRKNPQEG LYNELQKDKMAEAYSEIGMKGERRRGKGHDGLYQGL STATKDTYDALHMQALPPR (SEQ ID NO:293) which is encoded by cDNA:

(SEQ ID NO: 294)
CGGGTGAAGTTCAGCAGATCTGCCGATGCCCCTGCTTACCAACAGGGCCA
GAACCAGCTGTACAACGAGCTGAACCTGGGCCGGCGGGAAGAGTACGACG
TGCTGGACAAAAGAAGAGGCAGAGACCCTGAGATGGGGGGGAAGCCTCGG
AGAAAGAACCCCCAGGAGGGCCTGTACAATGAGCTGCAGAAGGACAAGAT
GGCCGAGGCCTATTCTGAGATCGGAATGAAGGGAGAAAGACGGAGAGGCA
AAGGCCACGACGGCCTGTACCAGGGTCTGTCGACCGCTACCAAGGACACC
TACGACGCCCTGCACATGCAGGCTCTGCCTCCAAGA.

### Example 7: HEK293 Cells Harboring TNF-a and MMP2 or MMP9 Cleavage Sites

Referring to FIGS. 4A-B, 5A-B, and 9-12, in some embodiments, following the teachings above, we created the constructs CD63-9-2L-TNFa-Myc-9-CD63-cyt (contains EV sorting motif) shown in FIGS. 4A-B, 9 and 10, and construct CD63-9-2L-TNFa-Myc-9-CD63-D-Cyt (Lacks EVs sorting motif) shown in FIGS. 5A-B, 11 and 12. These constructs differ only by the extracellular vesical sorting motif.
Construct CD63-9-2L-TNFa-Myc-9-CD63-cyt (EVs sorting motif) LeGo-CD63-9-2L-TNFa-Myc-9-CD63-cyt
is set out below as SEQ ID NO: 295

(SEQ ID NO: 295)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGMTSSSSRTPSDKPVAHVVANPQAEGQLQW
LNRRANALLANGVELRDNQLVVPSEGLYLIYSQVLFKGQGCPSTHVLLTH
TISRIAVSYQTKVNLLSAIKSPCQRETPEGAEAKPWYEPIYLGGVFQLEK
GDRLSAEINRPDYLDFAESGQVYFGIIALGGSGEQKLISEEDLGGPLGMT
SGGSGPKNNHTASILDRMQADFKCCGAANYTDWEKIPSMSKNRVPDSCCI
NVTVGCGINFNEKAIHKEGCVEKIGGWLRKNVLVVAAAALGIAFVEVLGI
VFACCLVKSIRSGYEVM

Referring to FIGS. 4A-4B, 9 and 10, the domains and motifs are encoded by the following amino acid sequences.

CD63-CYTOPLASMIC:
(SEQ ID NO: 297)
MAVEGGMKCVK
CD63-TM-HELICAL(inside-out)
(SEQ ID NO: 298)
FLLYVLLLAFCACAVGLIAVG_
CD63-EXTRACELLULAR
(SEQ ID NO: 299)
VGAQLVLSQTIIQGATPGS
CD63-TM-HELICAL(outside-in)
(SEQ ID NO: 300)
LLPVVIIAVGVFLFLVAFVGC
CD63-CYTOPLASMIC
(SEQ ID NO: 301)
CGACKENYC
CD63-TM-HELICAL(inside-out)
((SEQ ID NO: 302)
LMITFAIFLSLIMLVEVAAAI
Linker:
(SEQ ID NO: 303)
GGSGPVRRYQ
MMP9:
(SEQ ID NO: 304)
GGPLGMTS

TNFa:

(SEQ ID NO: 305)
SSSRTPSDKPVAHVVANPQAEGQLQWLNRRANALLANGVELRDNQLVVPS
EGLYLIYSQVLFKGQGCPSTHVLLTHTISRIAVSYQTKVNLLSAIKSPCQ
RETPEGAEAKPWYEPIYLGGVFQLEKGDRLSAEINRPDYLDFAESGQVYF
GIIA
Linker + Myc-tag:
(SEQ ID NO: 306)
GGSGEQKLISEEDL
MMP9
(SEQ ID NO: 307)
GGPLGMTS

CD63-EXTRACELLULAR

(SEQ ID NO: 308)
PKNNHTASILDRMQADFKCCGAANYTDWEKIPSMSKNRVPDSCCINVTVG
CGINFNEKAIHKEGCVEKIGGWLRKNVLVVAAAALGIAFVEVLGIVFACC
LVKSIRSGYEVM
CD63-TM-HELICAL(outside-in)
(SEQ ID NO: 309)
LVVAAAALGIAFVEVLGIVFA
CD63-CYTOPLASMIC
(SEQ ID NO: 310)
LVKSIRSGYEVM

Construct CD63-9-2L-TNFa-Myc-9-CD63-D-Cyt (Lacks EVs sorting motif) is outlined in SEQ ID NO: 311 below.

(SEQ ID NO: 311)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGMTSSSSRTPSDKPVAHVVANPQAEGQLQW
LNRRANALLANGVELRDNQLVVPSEGLYLIYSQVLFKGQGCPSTHVLLTH
TISRIAVSYQTKVNLLSAIKSPCQRETPEGAEAKPWYEPIYLGGVFQLEK
GDRLSAEINRPDYLDFAESGQVYFGIIALGGSGEQKLISEEDLGGPLGMT
SGGSGPKNNHTASILDRMQADFKCCGAANYTDWEKIPSMSKNRVPDSCCI
NVTVGCGINFNEKAIHKEGCVEKIGGWLRKNVLVVAAAALGIAFVEVLGI
VFACCLVKS

Referring to FIGS. 5A-B, 11 and 12, the domains and motifs of this construct are encoded by the following amino acid sequences.

CD63-CYTOPLASMIC:
(SEQ ID NO: 313)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYV
CD63-TM-HELICAL(inside-out)
(SEQ ID NO: 314)
FLLYVLLLAFCACAVGLIAVG
CD63-EXTRACELLULAR
(SEQ ID NO: 315)
VGAQLVLSQTIIQGATPGS
CD63-TM-HELICAL(outside-in)
(SEQ ID NO: 316)
LLPVVIIAVGVFLFLVAFVGC_
CD63-CYTOPLASMIC
(SEQ ID NO: 317)
CGACKENYC
CD63-TM-HELICAL(inside-out)
(SEQ ID NO: 318)
LMITFAIFLSLIMLVEVAAAI
Linker:
(SEQ ID NO: 319)
GGSGPVRRYQ
MMP9:
(SEQ ID NO: 320)
GGPLGMTS
TNFa-
(SEQ ID NO: 321)
SSSRTPSDKPVAHVVANPQAEGQLQWLNRRANALLANGVELRDNQLVVPS
EGLYLIYSQVLFKGQGCPSTHVLLTHTISRIAVSYQTKVNLLSAIKSPCQ
RETPEGAEAKPWYEPIYLGGVFQLEKGDRLSAEINRPDYLDFAESGQVYF
GIIA
Linker + Myc-tag:
(SEQ ID NO: 322)
GGSGEQKLISEEDL
MMP9:
(SEQ ID NO: 323)
GGPLGMTS
CD63-EXTRCELLULAR
(SEQ ID NO: 324)
PKNNHTASILDRMQADFKCCGAANYTDWEKIPSMSKNRVPDSCCINVTVG
CGINFNEKAIHKEGCVEKIGGWLRKNVLVVAAAALGIAFVEVLGIVFACC
LVKSIRSGYEVM
CD63-TM-HELICAL(outside-in)
(SEQ ID NO: 185)
LVVAAAALGIAFVEVLGIVFA
CD63-CYTOPLASMIC
(SEQ ID NO: 325)
CCLVKS

### Functional Assays

### Example 8: Assay for TNFa Using HEK 293 Cells

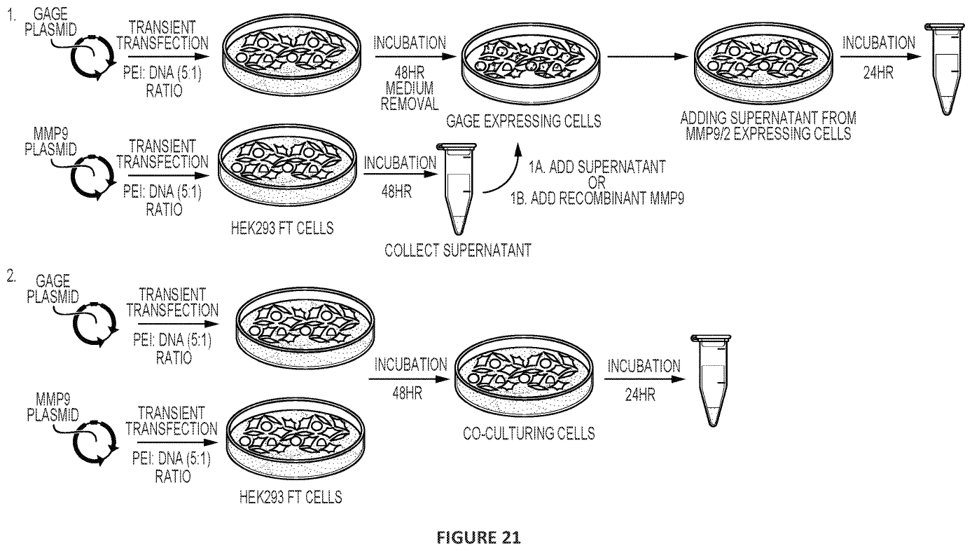

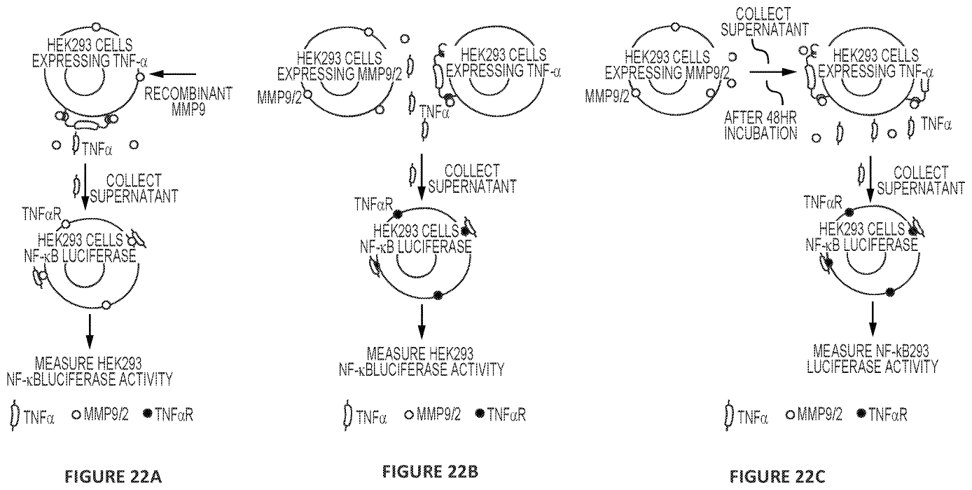

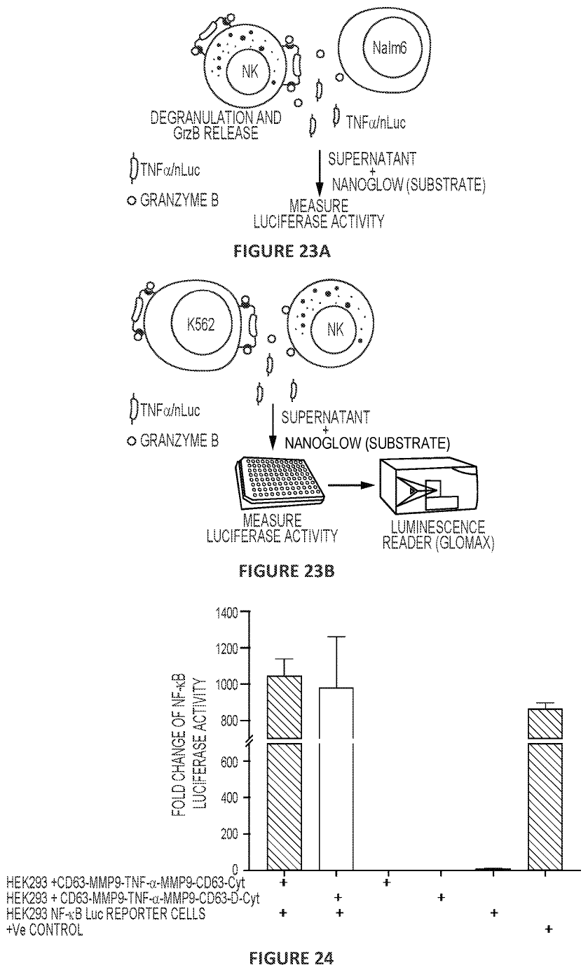

Referring to FIGS. 21 and 22, we created a functional assay to measure the presence of TNFa using HEK293 NF-κB luciferase reporter cells.

Referring to FIGS. 22 and 24, to test whether the GAGE payload in this system is functional, HEK293 cells were transfected with GAGE plasmids using polyethyleneimine (PEI) in a transient transfection as follows:
Day 0: 400,000 cells were seeded in 6 well plates.
Day 1: 2.5 ug of the GAGE plasmids were transfected into HEK293 wt cells.
Day 2: HEK293+GAGE and HEK293 NF-κB Luciferase reporter cells were co-cultured for 24 hr or kept separately.
Day 3: The reporter cells were collected, lysed and we assayed for the functional activity of TNFa presence on HEK293 cells using NF-κB-Luciferase reporter assay. Referring to FIG. 26, it is readily observed that TNFa expressed on the HEK293 cells was functional regardless of the presence or absence of the EVs sorting motif.

### Example 9: Assay for Payload Release by MMP 9 or MMP2

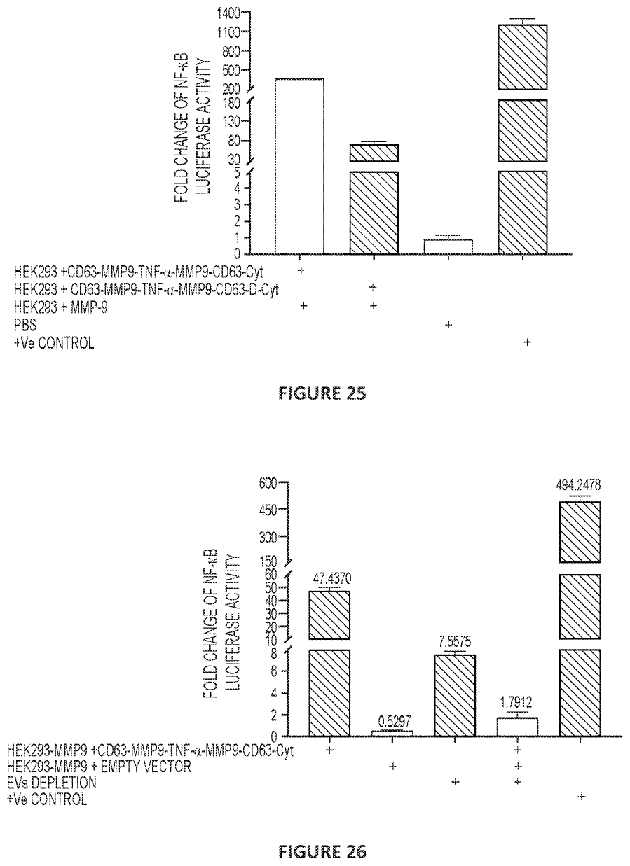

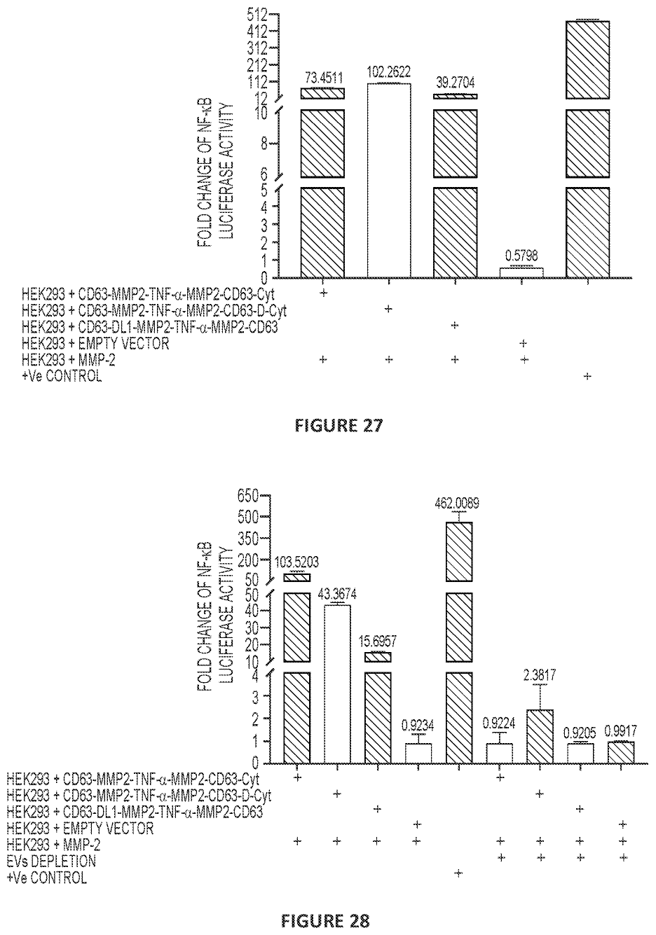

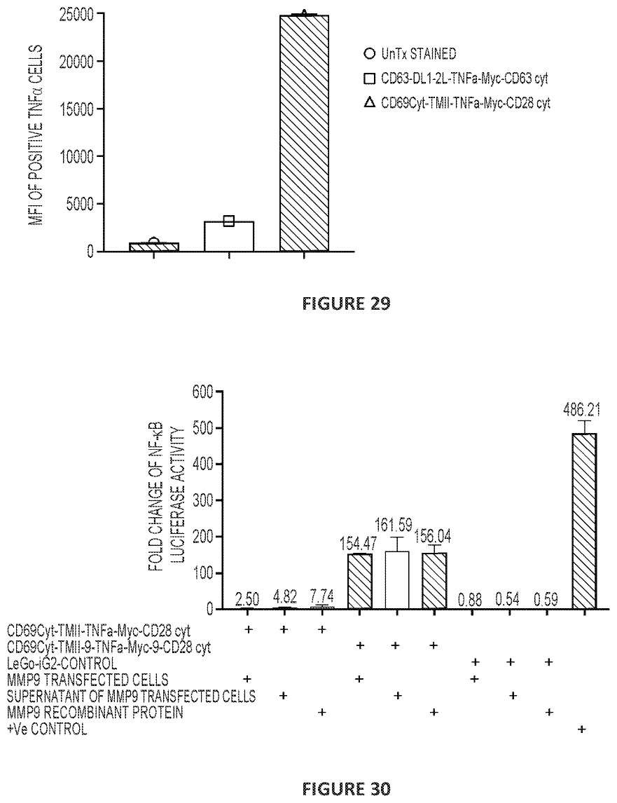

Next, referring to FIGS. 25 and 27, to test whether the GAGE-expressing cells are released in the presence of MMP9 or MMP2. HEK293 cells were transfected with GAGE and MMP9 or MMP2 plasmids using polyethyleneimine (PEI) in a transient transfection as follows:
Day 0: 400,000 cells were seeded in 6 well plates.
Day 1: 2.5 ug of the GAGE and MMP9 or MMP2 plasmids were transfected separately into HEK293 wt cells.
Day 2: HEK293+GAGE and HEK293+MMP9 or HEK293+MMP2 cells were co-cultured for 24 hr.
Day 3: 200 μL of the supernatant was collected and given to HEK293 NF-κB reporter cells.

After 6 hr incubation, the reporter cells were collected, lysed with 1% Triton, and we assayed for the functional activity of TNFa released from HEK293 cells using NF-κB-Luciferase reporter assay. The substrate (D-luciferin) was automatically added to the samples, and the luminescence was measured.

Referring to FIGS. 25 and 27, it is readily observed that TNFa expressed on the HEK293 cells was higher for the EVs sorted plasmid, indicating that most of the GAGE payloads enriched into EVs than non-EVs sorting plasmid.

### Example 10: Delivery of Payload to Extracellular Vesicles

To verify the delivery of the GAGE payloads mainly into EVs, the experiment of Example 9 was repeated with supernatant depletion of EVs by running it through Amicon® Ultra 0.5 mL Centrifugal Filters. The luciferase reporter test was performed with HEK293 NF-κB Luciferase reporter cells. Supernatant from co-culture was either depleted from EVs or added as it is to HEK293 reporter cells expressing NF-kB in response to TNFa-payload. FIGS. 26 and 28 show that the Luciferase activity was remarkably reduced after EVs depletion, suggesting delivery of the GAGE presence in the supernatant containing extracellular vesicles.

### Example 11: Selection of TMD for Better Cell Surface Expression

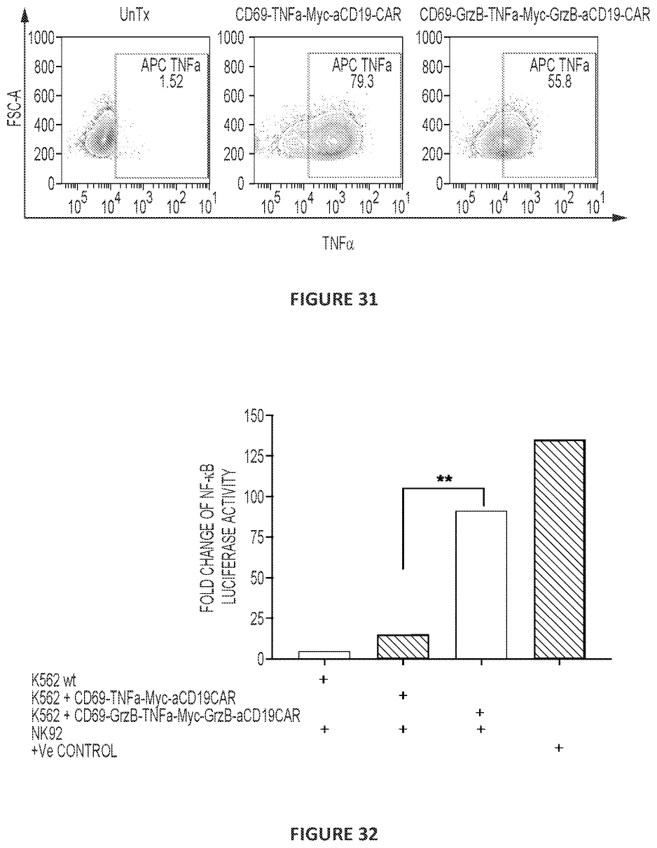

To evaluate alternative transmembrane (TM) protein that can mainly express on the cell surface, we exchanged the CD63 TM with C-type Lectin transmembrane CD69. FIG. 31 shows higher percentage of TNFa positive cells where CD69 was used as TM protein compared to CD63 TM.

Next, to evaluate whether the GAGE TNFa expressed on the HEK293 cells can be released in the presence of MMP9, the MMP9 cleavage was carried out by direct co-culturing of GAGE TNFa expressing cells with MMP expressing cells, supernatant from MMP9 transfected cells or providing MMP9 recombinant protein. The successful MMP9 cleavage of the MMP9 protease substrate linked to the TNFa GAGE was verified by luciferase assay using HEK293 NF-κB Luciferase reporter cells. As shown in FIGS. 17A-B, 18A-B, 29 and 30, the luciferase activity was only observed in the clone with the presence of MMP9 cleavage substrate, indicating that the TNFa can be released by specific protease cleavage.

### Example 12: Payload Release with Granzyme B

We used another sheddase cleavage substrate for Granzyme B (GrzB) protein connected to TNFa GAGE as a payload to further evaluate our invention. First, both clones CD69-GrzB-TNFa-Myc-GrzB-aCD19-CAR and CD69-TNFa-Myc-aCD19-CAR were generated and transduced in K562 cells (FIG. 31).

Next, to evaluate whether the GAGE TNFa with GrzB cleavage substrate expressed on the K562 cells can be released upon co-culturing with NK92. Encountering NK92 cells against target cells usually leads to induced degranulation of NK92 with granules containing Granzyme B. The GrzB cleavage with TNFa release was carried out via co-culturing GAGE TNFa expressing K562 cells with NK92 cells. After 4 hrs incubation, the cells were centrifuged, and the supernatant was collected. The successful cleavage of the GrzB protease substrate linked to the TNFa GAGE was verified by luciferase assay using HEK293 NF-κB Luciferase reporter cells. As shown in FIG. 32, the luciferase activity was higher in the clone with the presence of GrzB cleavage substrate, indicating that the TNFa was specifically released by specific protease cleavage.

### Example 13: Delivery of NanoLuciferase Functional Assay as a Proof of Principle

To increase the sensitivity of the readout, we repeated the experiment of Example 12 with another GAGE payload, in this case, we used NanoLuciferase as a payload.

We generated three clones as follows:
CD69-GrzB-nLuc-Myc-GrzB-aCD19-CAR (GAGE 1 or GAGE original)
CD69-nLuc-Myc-aCD19-CAR (GAGE A)
CD69-GrzB-nLuc-Myc-GrzB-CAR (GAGE B)

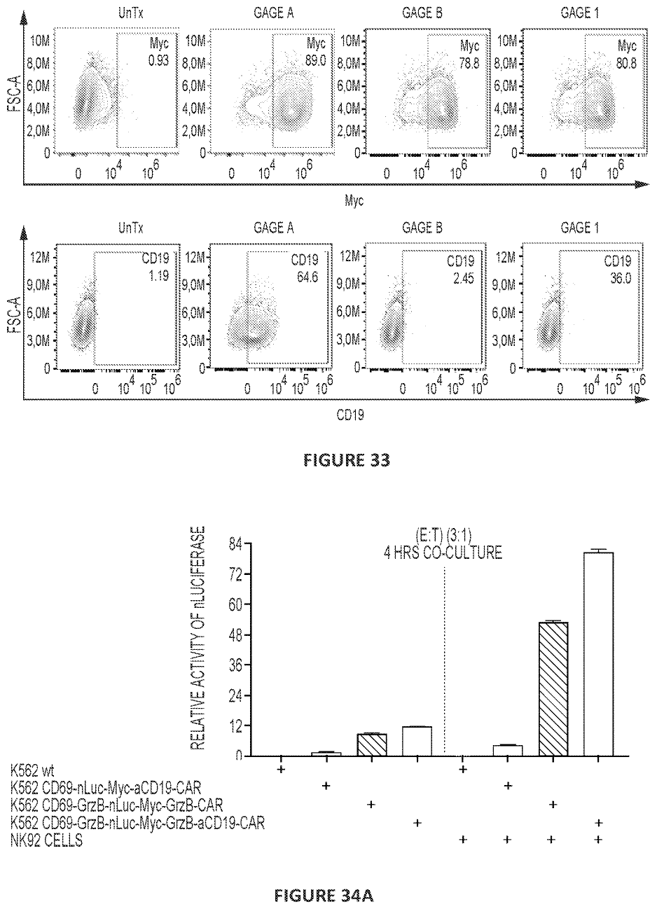

These plasmids were transduced in K562 cells with MOI6 and then assayed with the Luciferase assay. Referring to FIG. 33, the surface expression of GAGE nLuc in K562 cells was observed.

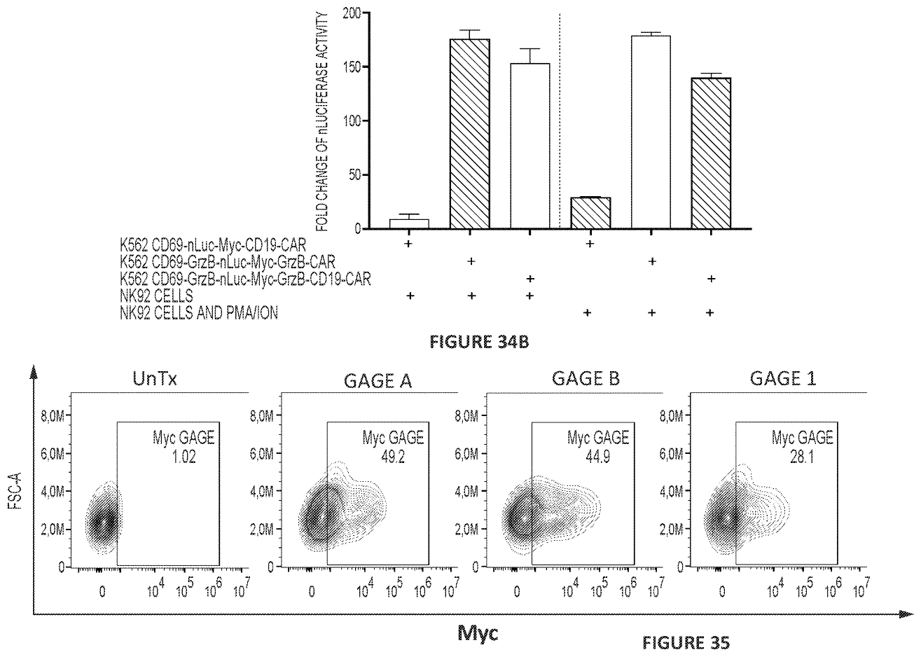

Next, to evaluate whether the GAGE nLuc with GrzB cleavage substrate expressed on the K562 cells can be released upon co-culturing with NK92. The GrzB cleavage with nLuc release was carried out via co-culturing GAGE nLuc expressing K562 cells with NK92 cells. After 4 hrs incubation, the cells were centrifuged, and the supernatant was collected. The successful cleavage of the GrzB protease substrate linked to the nLuc GAGE was verified by direct luciferase assay. As shown in FIG. 34A, the luciferase activity was higher in the clone with the presence of GrzB cleavage substrate, indicating that the nLuc was particularly released by specific protease cleavage. Further activation of NK92 cells using PAM/Ion had no significant effect on additional cleavage of the substrate, indicated by no differences in luciferase activity assay FIG. 34B.

### Example 14: Additional Clones to Evaluate Payload Cleavage and Release

We generated the following clones CD69-GrzB-nLuc-Myc-GrzB-aCD19-CAR (GAGE 1 or GAGE original), CD69-nLuc-Myc-aCD19-CAR (GAGE A), and CD69-GrzB-nLuc-Myc-GrzB-CAR (GAGE B) in KHYG-1. To further evaluate the specific cleavage and payload release. We transduced the three clones mentioned above in KHYG-1 with MOI30. Referring to FIG. 35, the surface expression of GAGE nLuc in KHYG1 cells was observed.

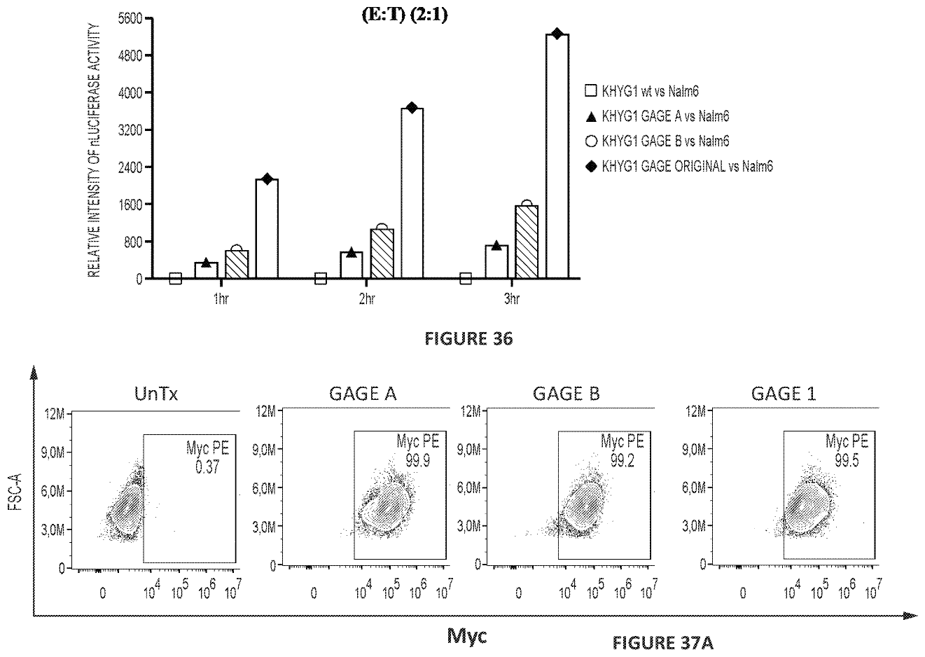

Following the co-culturing of KHYG-1 cells expressing GAGE nLuc with Nalm-6 cells (highly expressed CD19) for three different time points indicated in FIG. 36, the luciferase activity was higher in the clone with the presence of GrzB cleavage substrate with nLuc connected to sc-aCD19CAR, indicating that the nLuc was particularly released by specific protease cleavage during the period of co-culturing with highest cleavage at three hours incubation.

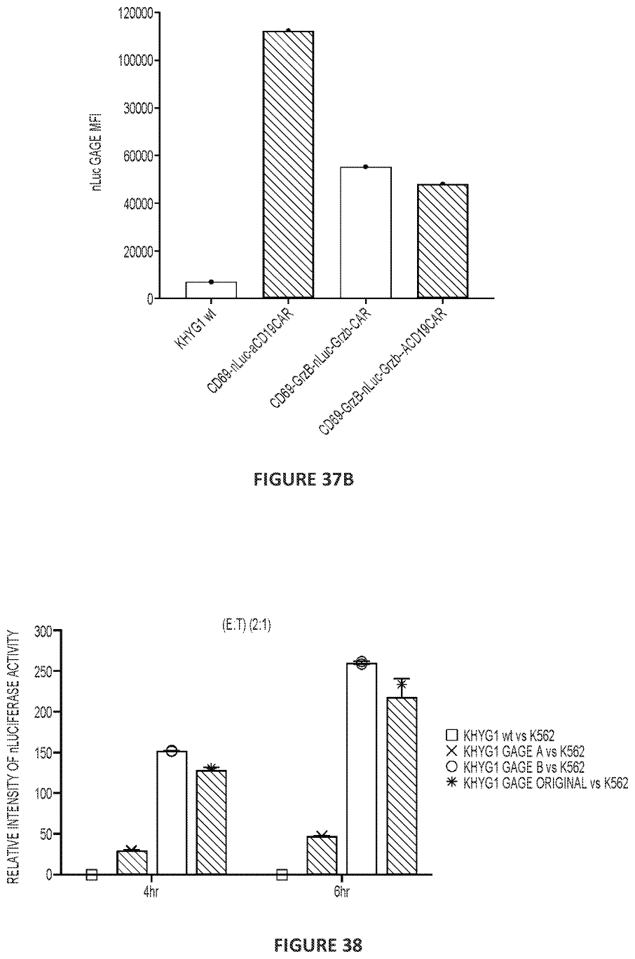

Next, we sorted the cells to reduce the background activity of the NanoLuciferase. As shown in FIG. 37A, KHYG-1 cells were more than 99% positive for the three clones. Interestingly, the MFI of nLuc GAGE positive cells was higher in the clone without the cleavage substrate site compared to the other two clones (FIG. 37B).

We repeated the experiment on sorted cells, and two different time points (4 hr and 6 hr) were selected for co-culturing sorted KHYG-1 cells expressing GAGE nLuc with K562 cells. As shown in FIG. 38, the luciferase activity was higher in the clone with the presence of GrzB cleavage substrate with nLuc, indicating that the nLuc was particularly released by specific protease cleavage during the period of co-culturing with the highest cleavage at 6 hours incubation.

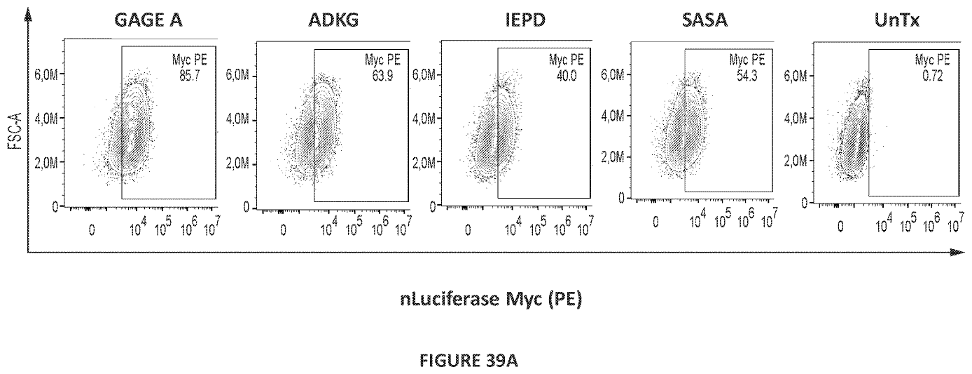

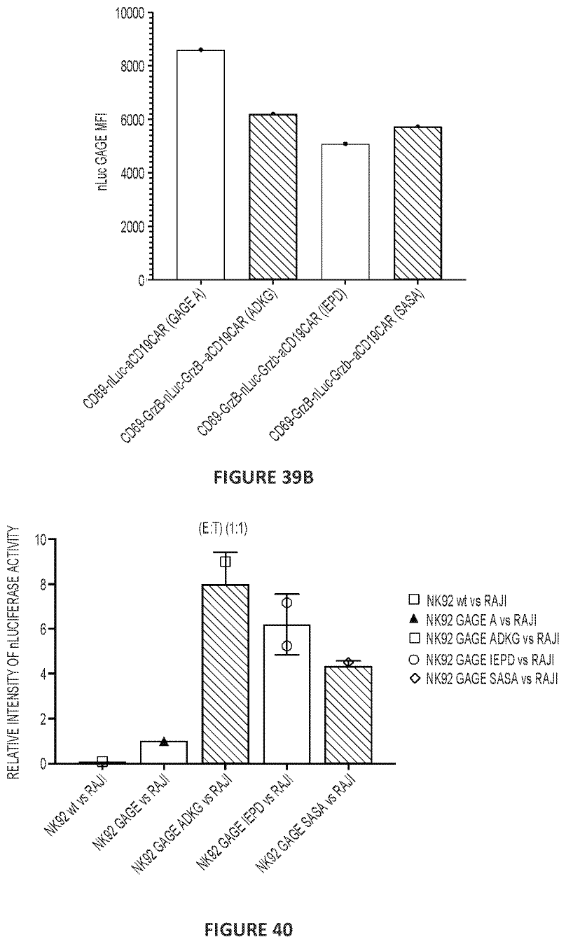

To evaluate the specificity and sensitivity of the cleavage by Granzyme B, we generated three clones of CD69-GrzB-nLuc-Myc-GrzB-aCD19-CAR with different GrzB cleavage substrates, as follows (GrzB (IEPD (SEQ ID NO: 258)), GrzB (AQGVISADASNLDDFY (SEQ ID NO: 261)) and GrzB (LEADKGKLEYD (SEQ ID NO: 262) in NK92 cells. We transduced the clones mentioned above in NK92 cells with MOI30. Referring to FIGS. 39A and 39B, the surface expression of GAGE nLuc in NK92 cells was observed.

Following the co-culturing of NK92 cells expressing GAGE nLuc with Raji cells for four hours, the luciferase activity was higher, with a nine-fold increase in the clone with the presence of GrzB (LEADKGKLEYD (SEQ ID NO: 262)) as cleavage substrate with nLuc connected compared to the control clone (GAGE A), indicating that this particular substrate has the higher affinity to the GrzB cleavage during the period of co-culturing (FIG. 40).

### Example 15: Payload Release Via Caspase 3

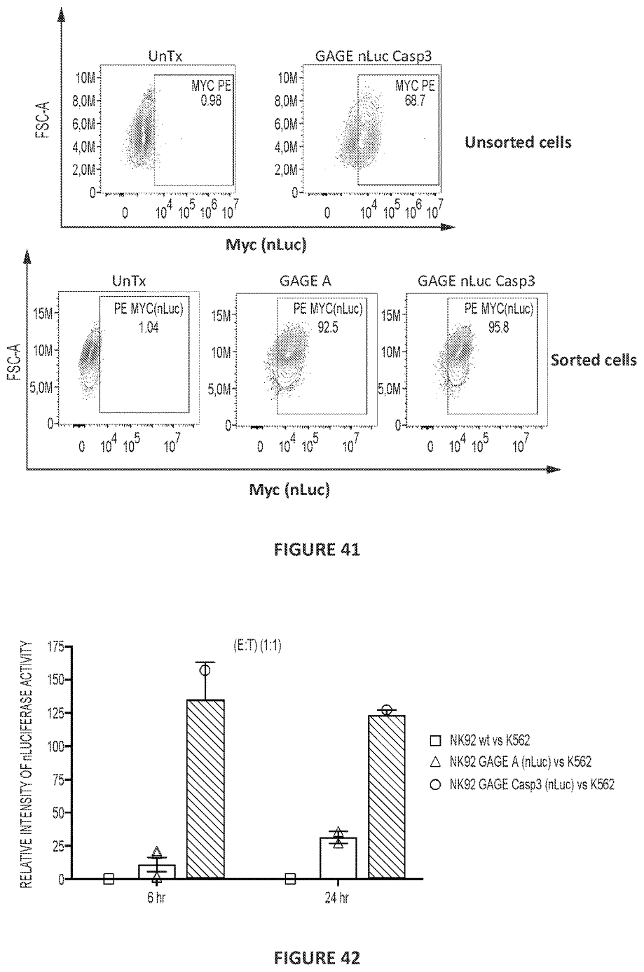

We generated the following clones CD69-Casp3-nLuc-Myc-Casp3-aCD19-CAR (GAGE Casp3) and CD69-nLuc-Myc-aCD19-CAR (GAGE A), in NK92. To further evaluate the specific cleavage and payload release. We transduced the two clones mentioned above in NK92 with MOI30. Referring to FIG. 41, the surface expression of GAGE nLuc in NK92 cells was observed.

Following the co-culturing of NK92 cells expressing GAGE Casp3-nLuc against K562 cells for two different time points indicated in FIG. 42, the luciferase activity was higher in the clone with the presence of Casp3 cleavage substrate with nLuc, indicating that the nLuc was particularly released by specific protease cleavage during the period of co-culturing.
CD69cyt-TMII-TNFa-Myctag-CD28TM-cyt

The cDNA of CD69Cyt-TM-II-TNFa-Myc-CD28TM-Cyt is set forth below as SEQ ID NO: 326

(SEQ ID NO: 326)
GATCCGCCGCCACCATGAGTTCCGAAAACTGCTTTGTCGCCGAGAATAGT
AGTCTGCACCCCGAATCAGGACAGGAGAATGACGCAACCAGCCCCCACTT
TTCAACAAGGCACGAAGGGAGCTTCCAGGTCCCTGTGCTGTGCGCAGTGA
TGAACGTGGTCTTTATCACTATTCTGATCATTGCTCTGATCGCACTGAGT
GTCGGACAGTACAATGGCGGGTCAGGCCCCGTGCGGAGATATCAGAGCTC
CTCTCGCACCCCTTCCGACAAGCCAGTGGCCCATGTGGTCGCTAACCCTC
AGGCAGAGGGACAGCTGCAGTGGCTGAACAGGCGAGCCAATGCTCTGCTG
GCTAACGGCGTGGAACTGCGAGATAATCAGCTGGTGGTCCCTAGCGAGGG
GCTGTACCTGATCTATTCCCAGGTCCTGTTCAAAGGGCAGGGATGCCCAT
CTACACACGTGCTGCTGACCCATACAATCTCTAGAATTGCCGTCAGTTAC
CAGACTAAGGTGAACCTGCTGAGCGCCATCAAGTCACCATGTCAGAGGGA
GACACCCGAAGGAGCAGAGGCCAAGCCCTGGTACGAGCCTATCTATCTGG
GAGGCGTGTTTCAGCTGGAAAAAGGCGACCGACTGTCTGCCGAGATTAAT
CGGCCAGACTACCTGGATTTCGCTGAAAGTGGCCAGGTGTATTTTGGGAT
CATTGCACTGGGAGGCAGCGGAGAGCAGAAGCTGATCTCTGAGGAAGATC
TGGGAGGGAGCGGATGCCCATCCCCTCTGTTCCCAGGACCCAGCAAACCC
TTTTGGGTCCTGGTGGTCGTGGGAGGCGTGCTGGCATGTTATAGCCTGCT
GGTCACAGTGGCTTTCATTATCTTTTGGGTCCGCTCTAAACGGTCTCGGC
TGCTGTAATGTACAA

The corresponding amino acid sequence is set forth as SE ID NO: 327.

(SEQ ID NO: 327)
MSSENCFVAENSSLHPESGQENDATSPHFSTRHEGSFQVPVLCAVMNVVF
ITILIIALIALSVGQYNGGSGPVRRYQSSSRTPSDKPVAHVVANPQAEGQ
LQWLNRRANALLANGVELRDNQLVVPSEGLYLIYSQVLFKGQGCPSTHVL
LTHTISRIAVSYQTKVNLLSAIKSPCQRETPEGAEAKPWYEPIYLGGVFQ
LEKGDRLSAEINRPDYLDFAESGQVYFGIIALGGSGEQKLISEEDLGGSG
CPSPLFPGPSKPFWVLVVVGGVLACYSLLVTVAFIIFWVRSKRSRLL

SEQ ID NO: 327 is also shown in FIG. 17A-B, together with a legend of domains and motifs

CD69-CYTOPLASMIC
(SEQ ID NO: 328)
MSSENCFVAENSSLHPESGQENDATSPHFSTRHEGSFQVP
CD69-TM-type-II(inside-out)
(SEQ ID NO 329)
VLCAVMNVVFITILIIALIAL
CD69-Extracellular
(SEQ ID NO 330)
SVGQYNGGSGPVRRYQ
TNFa
(SEQ ID NO 331)
RTPSDKPVAHVVANPQAEGQLQWLNRRANALLANGVELRDNQLVVPSEGL
YLIYSQVLFKGQGCPSTHVLLTHTISRIAVSYQTKVNLLSAIKSPCQRET
PEGAEAKPWYEPIYLGGVFQLEKGDRLSAEINRPDYLDFAESGQVYFGII
AL
linker
(SEQ ID NO 332)
GGSG
Myc-tag
(SEQ ID NO: 333)
EQKLISEEDL
CD28TM
(SEQ ID NO 334)
FWVLVVVGGVLACYSLLVTVAFIIFWV

CD69cyt-TMII-9-TNFa-Myctag-9-CD28TM-cyt

The cDNA of CD69cyt-TMII-9-TNFa-Myctag-9-CD28TM-cyt is set forth below as SEQ ID NO: 335).

(SEQ ID NO: 335)
ATCCGCCGCCACCATGAGTTCCGAAAACTGCTTTGTCGCCGAGAATAGTA
GTCTGCACCCCGAATCAGGACAGGAGAATGACGCAACCAGCCCCCACTTT
TCAACAAGGCACGAAGGGAGCTTCCAGGTCCCTGTGCTGTGCGCAGTGAT
GAACGTGGTCTTTATCACTATTCTGATCATTGCTCTGATCGCACTGAGTG
TCGGACAGTACAATGGCGGGTCAGGCCCCGTGCGGAGATATCAGGGAGGG
TCTGGGGGAGGACCACTGGGAATGACCTCCGGAGGGTCTGGGAGCTCCTC
TCGCACCCCTTCCGACAAGCCAGTGGCCCATGTGGTCGCTAACCCTCAGG
CAGAGGGACAGCTGCAGTGGCTGAACAGGCGAGCCAATGCTCTGCTGGCT
AACGGCGTGGAACTGCGAGATAATCAGCTGGTGGTCCCTAGCGAGGGGCT
GTACCTGATCTATTCCCAGGTCCTGTTCAAAGGGCAGGGATGCCCATCTA
CACACGTGCTGCTGACCCATACAATCTCTAGAATTGCCGTCAGTTACCAG
ACTAAGGTGAACCTGCTGAGCGCCATCAAGTCACCATGTCAGAGGGAGAC
ACCCGAAGGAGCAGAGGCCAAGCCCTGGTACGAGCCTATCTATCTGGGAG
GCGTGTTTCAGCTGGAAAAAGGCGACCGACTGTCTGCCGAGATTAATCGG
CCAGACTACCTGGATTTCGCTGAAAGTGGCCAGGTGTATTTTGGGATCAT
TGCACTGGGAGGCAGCGGAGAGCAGAAGCTGATCTCTGAGGAAGATCTGG
GAGGACCACTGGGAATGACCTCCGGAGGGAGCGGATGCCCATCCCCTCTG
TTCCCAGGACCCAGCAAACCCTTTTGGGTCCTGGTGGTCGTGGGAGGCGT
GCTGGCATGTTATAGCCTGCTGGTCACAGTGGCTTTCATTATCTTTTGGG
TCCGCTCTAAACGGTCTCGGCTGCTGTAATGTACA

The corresponding amino acid sequence is set out below as SEQ ID NO: 336 and is also set out in FIG. 18A-B together with a legend of domains and motifs.

(SEQ ID NO: 336)
MSSENCFVAENSSLHPESGQENDATSPHFSTRHEGSFQVPVLCAVMNVVF
ITILIIALIALSVGQYNGGSGPVRRYQGGSGGGPLGMTSGGSGSSSRTPS
DKPVAHVVANPQAEGQLQWLNRRANALLANGVELRDNQLVVPSEGLYLIY
SQVLFKGQGCPSTHVLLTHTISRIAVSYQTKVNLLSAIKSPCQRETPEGA
EAKPWYEPIYLGGVFQLEKGDRLSAEINRPDYLDFAESGQVYFGIIALGG
SGEQKLISEEDLGGPLGMTSGGSGCPSPLFPGPSKPFWVLVVVGGVLACY
SLLVTVAFIIFWVRSKRSRLL

### Domains and Motifs

CD69-CYTOPLASMIC
(SEQ ID NO: 338)
MSSENCFVAENSSLHPESGQENDATSPHFSTRHEGSFQVP
CD69-TM-type-II(inside-out)_
(SEQ ID NO: 339)
VLCAVMNVVFITILIIALIAL
CD69-Extracellular_
(SEQ ID NO: 340)
SVGQYNGGSGPVRRYQ
Linker
(SEQ ID NO: 341)
GGSG
MMP9
(SEQ ID NO: 342)
GGPLGMTSG
Linker
(SEQ ID NO: 343)
GSGSSS

### TNFa

(SEQ ID NO 344)
RTPSDKPVAHVVANPQAEGQLQWLNRRANALLANGVELRDNQLVVPSEG
LYLIYSQVLFKGQGCPSTHVLLTHTISRIAVSYQTKVNLLSAIKSPCQR
ETPEGAEAKPWYEPIYLGGVFQLEKGDRLSAEINRPDYLDFAESGQVYF
GIIAL
Myc-tag
(SEQ ID NO 345)
EQKLISEEDL
CD28TM
(SEQ ID NO: 346)
FWVLVVVGGVLACYSLLVTVAFIIFW

Referring to FIG. 20, some of the GAGE DNA SEQs are shown as follows:

LeGo-CD63-9-2L-TNFa-Myc-9-CD63-cyt
(SEQ ID NO: 295)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGMTSSSSRTPSDKPVAHVVANPQAEGQLQW
LNRRANALLANGVELRDNQLVVPSEGLYLIYSQVLFKGQGCPSTHVLLTH
TISRIAVSYQTKVNLLSAIKSPCQRETPEGAEAKPWYEPIYLGGVFQLEK
GDRLSAEINRPDYLDFAESGQVYFGIIALGGSGEQKLISEEDLGGPLGMT
SGGSGPKNNHTASILDRMQADFKCCGAANYTDWEKIPSMSKNRVPDSCCI
NVTVGCGINFNEKAIHKEGCVEKIGGWLRKNVLVVAAAALGIAFVEVLGI
VFACCLVKSIRSGYEVM

Which is encoded as cDNA:

(SEQ ID NO: 296)
GATCCGCCGCCACCATGGCCGTGGAGGGCGGGATGAAGTGCGTGAAATTC
CTGCTGTACGTCCTGCTGCTGGCCTTTTGCGCATGTGCTGTGGGACTGAT
CGCAGTGGGAGTCGGAGCTCAGCTGGTGCTGAGTCAGACTATCATTCAGG
GCGCAACCCCTGGGTCACTGCTGCCAGTGGTCATCATTGCCGTGGGCGTC
TTCCTGTTTCTGGTGGCATTCGTCGGGTGCTGTGGAGCCTGCAAGGAAAA
CTATTGTCTGATGATCACCTTCGCTATTTTTCTGTCTCTGATCATGCTGG
TGGAGGTCGCCGCTGCAATTGCCGGCTACGTGGGAGGCAGTGGACCAGTC
CGGAGATATCAGGGGGGACCCCTGGGAATGACAAGCTCCTCTAGTCGGAC
TCCCTCTGACAAGCCTGTGGCCCACGTGGTCGCTAATCCTCAGGCAGAAG
GCCAGCTGCAGTGGCTGAACAGGCGAGCAAATGCACTGCTGGCCAACGGA
GTGGAACTGAGAGATAATCAGCTGGTGGTCCCATCCGAGGGACTGTACCT
GATCTATTCTCAGGTGCTGTTCAAAGGACAGGGATGCCCAAGCACACACG
TGCTGCTGACCCATACAATCTCTAGAATTGCTGTGAGTTACCAGACTAAG
GTCAACCTGCTGAGCGCCATCAAATCCCCCTGTCAGAGGGAGACCCCTGA
AGGCGCTGAGGCAAAGCCATGGTACGAGCCCATCTATCTGGGCGGGGTGT
TTCAGCTGGAAAAAGGGGACCGACTGAGCGCAGAGATTAATCGGCCCGAC
TACCTGGATTTCGCCGAATCCGGACAGGTGTATTTTGGCATCATTGCTCT
GGGAGGCTCAGGCGAGCAGAAGCTGATCAGCGAGGAAGATCTGGGAGGAC
CACTGGGAATGACCTCCGGAGGGTCTGGGCCTAAGAACAATCACACAGCC
AGCATTCTGGACCGCATGCAGGCAGATTTCAAATGCTGTGGCGCCGCTAA
CTACACAGACTGGGAAAAGATCCCTTCAATGAGCAAAAACCGAGTGCCAG
ATTCCTGCTGTATTAATGTGACTGTCGGATGCGGCATCAACTTTAATGAG
AAGGCCATTCATAAAGAAGGCTGCGTGGAGAAGATCGGAGGCTGGCTGAG
GAAAAATGTGCTGGTGGTCGCAGCCGCTGCACTGGGAATCGCCTTCGTGG
AAGTCCTGGGCATCGTGTTCGCTTGCTGTCTGGTCAAAAGTATTCGCTCA
GGCTATGAAGTGATGTAATGTACA
LeGo-CD63-9-2L-TNFa-Myc-9-CD63-D-Cyt
(SEQ ID NO: 311)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGMTSSSSRTPSDKPVAHVVANPQAEGQLQW
LNRRANALLANGVELRDNQLVVPSEGLYLIYSQVLFKGQGCPSTHVLLTH
TISRIAVSYQTKVNLLSAIKSPCQRETPEGAEAKPWYEPIYLGGVFQLEK
GDRLSAEINRPDYLDFAESGQVYFGIIALGGSGEQKLISEEDLGGPLGMT
SGGSGPKNNHTASILDRMQADFKCCGAANYTDWEKIPSMSKNRVPDSCCI
NVTVGCGINFNEKAIHKEGCVEKIGGWLRKNVLVVAAAALGIAFVEVLGI
VFACCLVKS

Which is encoded as cDNA:

(SEQ ID NO: 347)
GGATCCGCCGCCACCATGGCCGTGGAGGGCGGGATGAAGTGCGTGAAATT
CCTGCTGTACGTCCTGCTGCTGGCCTTTTGCGCATGTGCTGTGGGACTGA
TCGCAGTGGGAGTCGGAGCTCAGCTGGTGCTGTCCCAGACTATCATTCAG
GGCGCAACCCCTGGGTCTCTGCTGCCAGTGGTCATCATTGCCGTGGGCGT
CTTCCTGTTTCTGGTGGCATTCGTCGGGTGCTGTGGAGCCTGCAAGGAAA
ACTATTGTCTGATGATCACCTTCGCTATTTTTCTGAGCCTGATCATGCTG
GTGGAGGTCGCCGCTGCAATTGCAGGATACGTGGGAGGCTCCGGACCAGT
CCGGAGATATCAGGGGGGACCCCTGGGAATGACAAGCTCCTCTAGTAGGA
CTCCCAGCGACAAGCCTGTGGCCCACGTGGTCGCTAATCCTCAGGCAGAA
GGCCAGCTGCAGTGGCTGAACAGGCGAGCAAATGCACTGCTGGCCAACGG
AGTGGAACTGCGAGATAATCAGCTGGTGGTCCCATCAGAGGGACTGTACC
TGATCTATAGCCAGGTGCTGTTCAAAGGACAGGGATGCCCATCCACACAC
GTGCTGCTGACCCATACAATCAGCAGAATTGCCGTGAGCTACCAGACTAA
GGTCAACCTGCTGAGCGCCATCAAGTCACCCTGTCAGAGGGAGACCCCTG
AAGGCGCTGAGGCAAAGCCATGGTACGAGCCCATCTATCTGGGCGGGGTG
TTTCAGCTGGAAAAAGGGGACCGACTGAGTGCAGAGATTAATCGGCCCGA
CTACCTGGATTTCGCCGAATCAGGACAGGTGTATTTTGGCATCATTGCTC
TGGGAGGCTCTGGCGAGCAGAAGCTGATCAGTGAGGAAGATCTGGGAGGA
CCACTGGGAATGACCTCCGGAGGGTCTGGGCCTAAGAACAATCACACAGC
TTCTATTCTGGACCGCATGCAGGCAGATTTCAAATGCTGTGGCGCCGCTA
ACTATACAGACTGGGAAAAGATCCCTTCAATGAGCAAAAACCGAGTGCCA
GATAGTTGCTGTATTAATGTGACTGTCGGATGCGGCATCAACTTTAATGA
GAAGGCCATTCATAAAGAAGGCTGCGTGGAGAAGATCGGAGGCTGGCTGC
GGAAAAATGTGCTGGTGGTCGCAGCCGCTGCACTGGGAATCGCCTTCGTG
GAGGTCCTGGGCATCGTGTTCGCTTGCTGTCTGGTCAAATCTTAATGTAC
A
LeGo-CD63-DL1-2-2L-TNFa-Myc-2-CD63-Cyt
(SEQ ID NO: 368)
MAVEGGMKCVKCGACKENYCLMITFAIFLSLIMLVEVAAAIAGYVGGSGP
VRRYQGGPLGVRGGSSSRTPSDKPVAHVVANPQAEGQLQWLNRRANALLA
NGVELRDNQLVVPSEGLYLIYSQVLFKGQGCPSTHVLLTHTISRIAVSYQ
TKVNLLSAIKSPCQRETPEGAEAKPWYEPIYLGGVFQLEKGDRLSAEINR
PDYLDFAESGQVYFGIIALGGSGEQKLISEEDLGGPLGVRGGGGSGPKNN
HTASILDRMQADFKCCGAANYTDWEKIPSMSKNRVPDSCCINVTVGCGIN
FNEKAIHKEGCVEKIGGWLRKNVLVVAAAALGIAFVEVLGIVFACCLVKS
IRSGYEVM

Which is encoded by cDNA:

(SEQ ID NO: 348)
GGATCCGCCGCCACCATGGCAGTGGAAGGCGGGATGAAGTGCGTCAAATG
TGGCGCCTGCAAGGAGAACTACTGTCTGATGATCACCTTCGCTATTTTTC
TGAGCCTGATCATGCTGGTGGAAGTCGCCGCTGCAATTGCAGGATACGTG
GGAGGCAGCGGACCCGTCCGGAGATATCAGGGAGGACCTCTGGGCGTGCG
AGGAGGGAGCTCCTCTCGAACACCATCCGACAAGCCTGTGGCTCACGTGG
TCGCAAATCCACAGGCAGAGGGACAGCTGCAGTGGCTGAACAGGCGAGCC
AATGCTCTGCTGGCCAACGGAGTGGAACTGAGGGATAATCAGCTGGTGGT
CCCCAGTGAGGGCCTGTACCTGATCTATTCACAGGTGCTGTTCAAAGGAC
AGGGCTGTCCTTCTACTCACGTGCTGCTGACCCATACAATCTCAAGGATT
GCCGTGAGCTATCAGACTAAGGTCAACCTGCTGTCTGCTATCAAAAGTCC
TTGCCAGCGAGAGACCCCAGAAGGAGCAGAGGCCAAGCCATGGTACGAGC
CCATCTATCTGGGAGGCGTGTTTCAGCTGGAAAAAGGAGACAGACTGTCT
GCAGAGATTAATAGGCCTGACTACCTGGATTTCGCCGAAAGTGGCCAGGT
GTATTTTGGGATCATTGCTCTGGGAGGCAGCGGAGAGCAGAAGCTGATCT
CTGAGGAAGATCTGGGAGGACCTCTGGGGGTGCGCGGAGGAGGGGGAAGC
GGACCAAAGAACAATCACACAGCTTCCATTCTGGACCGAATGCAGGCAGA
TTTCAAATGCTGTGGCGCCGCTAACTACACTGACTGGGAAAAGATCCCAA
GTATGTCAAAAAACCGGGTGCCCGATAGCTGCTGTATTAATGTGACAGTC
GGGTGTGGAATCAACTTTAATGAGAAGGCCATTCATAAAGAAGGCTGCGT
GGAGAAGATCGGCGGGTGGCTGCGGAAAAATGTCCTGGTGGTCGCAGCAG
CTGCACTGGGAATCGCCTTCGTGGAAGTCCTGGGGATCGTGTTCGCTTGC
TGTCTGGTCAAAAGCATTAGATCCGGCTATGAAGTGATGTAATGTACAA
LeGo-CD63-9-2L-TNFa-Myc-9-CD19CAR
(SEQ ID NO: 369)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGMTSSSSRTPSDKPVAHVVANPQAEGQLQW
LNRRANALLANGVELRDNQLVVPSEGLYLIYSQVLFKGQGCPSTHVLLTH
TISRIAVSYQTKVNLLSAIKSPCQRETPEGAEAKPWYEPIYLGGVFQLEK
GDRLSAEINRPDYLDFAESGQVYFGIIALGGSGEQKLISEEDLGGPLGMT
SGGSGGGSGPDIQMTQTTSSLSASLGDRVTISCRASQDISKYLNWYQQKP
DGTVKLLIYHTSRLHSGVPSRFSGSGSGTDYSLTISNLEQEDIATYFCQQ
GNTLPYTFGGGTKLEITGGGGSGGGGSGGGGSEVKLQESGPGLVAPSQSL
SVTCTVSGVSLPDYGVSWIRQPPRKGLEWLGVIWGSETTYYNSALKSRLT
IIKDNSKSQVFLKMNSLQTDDTAIYYCAKHYYYGGSYAMDYWGQGTSVTV
SSAKPTTTPAPRPPTPAPTIASQPLSLRPEACRPAAGGAVHTRGLDFAPR
KIEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPR

Which is encoded by cDNA:

(SEQ ID NO: 349)
GATCCGCCGCCACCATGGCCGTGGAGGGCGGGATGAAGTGCGTCAAATTC
CTGCTGTACGTGCTGCTGCTGGCCTTTTGCGCATGTGCAGTCGGACTGAT
CGCAGTCGGAGTGGGAGCCCAGCTGGTGCTGTCCCAGACTATCATTCAGG
GGGCAACCCCTGGATCTCTGCTGCCAGTGGTCATCATTGCTGTCGGCGTG
TTCCTGTTTCTGGTCGCTTTCGTGGGCTGCTGTGGGGCATGCAAGGAGAA
CTACTGTCTGATGATCACATTCGCCATTTTTCTGTCCCTGATCATGCTGG
TCGAAGTGGCCGCTGCAATTGCTGGCTACGTCGGAGGCTCTGGACCTGTG
CGGAGATATCAGGGGGGACCACTGGGCATGACAAGCTCCTCTAGTAGAAC
TCCCAGCGACAAGCCTGTGGCCCACGTGGTCGCTAATCCACAGGCAGAGG
GACAGCTGCAGTGGCTGAACAGGCGAGCTAATGCACTGCTGGCCAACGGG
GTGGAGCTGAGGGATAATCAGCTGGTGGTCCCTTCCGAAGGACTGTACCT
GATCTATTCTCAGGTCCTGTTCAAAGGACAGGGCTGCCCAAGCACCCACG
TGCTGCTGACCCATACAATCTCCCGCATTGCCGTCTCTTACCAGACCAAG
GTGAACCTGCTGAGCGCCATCAAGTCCCCATGTCAGCGGGAAACACCCGA
GGGCGCCGAAGCTAAGCCATGGTACGAGCCCATCTATCTGGGCGGGGTGT
TTCAGCTGGAGAAAGGCGACCGGCTGAGTGCAGAAATTAATAGACCTGAC
TACCTGGATTTCGCCGAGAGCGGACAGGTGTATTTTGGCATCATTGCTCT
GGGAGGCTCTGGCGAACAGAAGCTGATCAGTGAGGAAGATCTGGGAGGAC
CACTGGGAATGACATCTGGAGGGAGTGGAGGAGGGAGTGGACAGGTCCAG
CTGGTGGAATCAGGAGGAGGACTGGTGCAGCCTGGAGGCTCTCTGAAGCT
GAGTTGCGCCGCTTCAGGCTTCGACTTTAGTAGGTACTGGATGTCATGGG
TGCGCCAGGCACCAGGGAAAGGACTGGAGTGGATCGGCGAAATTAACCCC
ACATCAAGCACTATCAATTTCACTCCTAGCCTGAAGGACAAAGTGTTTAT
TTCCAGAGATAACGCCAAGAATACACTGTACCTGCAGATGAGCAAAGTGA
GGTCCGAGGACACTGCTCTGTACTATTGTGCACGAGGCAACTACTATCGG
TACGGGGACGCCATGGATTATTGGGGCCAGGGGACCTCCGTCACAGTGTC
AAAGATCAGCGGGGGAGGAGGGAGCGGAGGAGGAGGCAGCGGAGGAGGAG
GCTCTGGGGGAGGCGGGAGTGGAGGCGGGGGCAGCTCTGATATCGTCCTG
ACACAGTCCCCAGCTTCTCTGGCAGTGTCTCTGGGCCAGCGCGCAACTAT
TAGTTGTCGAGCCAGTAAATCAGTGAGCACCTCCGGATACAGCTATCTGC
ACTGGTATCAGCAGAAGCCCGGCCAGCCCCCTAAACTGCTGATCTATCTG
GCTTCTAACCTGGAGAGTGGCGTGCCTGCAAGATTCTCTGGAAGTGGCTC
AGGGACTGACTTTACCCTGAATATTCACCCAGTGGAGGAAGAGGATGCAG
CCACCTACTATTGCCAGCATTCAAGGGAGCTGCCCTTCACCTTTGGAAGC
GGCACAAAGCTGGAAATCAAAGGCGGGGGAGGCTCAGGAGGCGGCGGGAG
CGGCGGCGGAGGCAGCGCCAAGCCAACCACAACTCCAGCTCCACGACCAC
CAACACCTGCACCAACTATCGCTTCACAGCCCCTGAGCCTGAGACCTGAG
GCATGTAGGCCAGCTGCAGGAGGAGCAGTCCATACTCGAGGACTGGACTT
CGCCCCTAGGAAGATTGAAGTGATGTACCCTCCACCCTATCTGGATAACG
AAAAAAGCAATGGAACCATCATTCACGTGAAGGGCAAACATCTGTGCCCC
TCACCTCTGTTCCCAGGCCCCAGCAAGCCATTTTGGGTCCTGGTGGTCGT
GGGAGGCGTGCTGGCCTGTTACAGCCTGCTGGTCACCGTGGCTTTCATCA
TCTTCTGGGTGCGGAGCAAACGGTCAAGACTGCTGCACAGCGACTATATG
AACATGACCCCACGACGGCCTGGACCAACACGAAAGCATTACCAGCCATA
TGCACCTCCACGAGACTTCGCAGCTTACAGGTCCCGCGTGAAGTTCAGCC
GCTCCGCAGATGCACCAGCTTACCAGCAGGGACAGAATCAGCTGTATAAC
GAGCTGAATCTGGGGAGAAGGGAAGAGTATGACGTGCTGGATAAGCGACG
AGGACGGGACCCCGAAATGGGAGGAAAGCCACGGAGAAAAAACCCTCAGG
AGGGGCTTTACAATGAACTGCAGAAGGACAAAATGGCAGAGGCCTATTCC
GAAATCGGAATGAAGGGCGAGAGGCGCCGAGGGAAAGGACACGATGGCCT
GTACCAGGGGCTGTCTACTGCCACCAAGGACACCTATGATGCCCTGCATA
TGCAGGCTCTGCCCCCTAGATAATGTACA
LeGo-CD63-2-2L-TNFa-Myc-2-CD63-D-Cyt
(SEQ ID NO: 371)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGVRGGSSSRTPSDKPVAHVVANPQAEGQLQ
WLNRRANALLANGVELRDNQLVVPSEGLYLIYSQVLFKGQGCPSTHVLLT
HTISRIAVSYQTKVNLLSAIKSPCQRETPEGAEAKPWYEPIYLGGVFQLE
KGDRLSAEINRPDYLDFAESGQVYFGIIALGGSGEQKLISEEDLGGPLGV
RGGGGSGPKNNHTASILDRMQADFKCCGAANYTDWEKIPSMSKNRVPDSC
CINVTVGCGINFNEKAIHKEGCVEKIGGWLRKNVLVVAAAALGIAFVEVL
GIVFACCLVKS

Which is encoded by cDNA:

(SEQ ID NO: 350)
GATCCGCCGCCACCATGGCCGTGGAGGGCGGGATGAAGTGCGTGAAATTC
CTGCTGTACGTCCTGCTGCTGGCCTTTTGCGCATGTGCTGTGGGACTGAT
CGCAGTGGGAGTCGGAGCTCAGCTGGTGCTGAGCCAGACTATCATTCAGG
GAGCAACCCCTGGCAGCCTGCTGCCAGTGGTCATCATTGCCGTGGGCGTC
TTCCTGTTTCTGGTGGCATTCGTCGGATGCTGTGGCGCCTGCAAGGAAAA
CTATTGTCTGATGATCACTTTCGCTATTTTTCTGAGCCTGATCATGCTGG
TGGAGGTCGCCGCTGCAATTGCAGGATACGTGGGAGGCAGCGGACCAGTC
CGGAGATATCAGGGAGGACCACTGGGCGTGCGAGGAGGGAGCTCCTCTCG
AACCCCATCCGACAAGCCTGTGGCACACGTGGTCGCTAATCCTCAGGCAG
AAGGGCAGCTGCAGTGGCTGAACAGGCGCGCAAATGCCCTGCTGGCCAAC
GGAGTGGAACTGAGGGATAATCAGCTGGTGGTCCCATCCGAGGGCCTGTA
CCTGATCTATTCTCAGGTGCTGTTCAAAGGGCAGGGATGCCCCTCTACAC
ACGTGCTGCTGACCCATACAATCTCTAGGATTGCTGTGAGTTACCAGACA
AAGGTCAACCTGCTGAGCGCCATCAAGTCCCCCTGTCAGCGCGAGACTCC
TGAAGGGGCTGAGGCAAAGCCATGGTACGAGCCCATCTATCTGGGAGGCG
TGTTTCAGCTGGAAAAAGGAGACAGACTGTCTGCAGAGATTAATAGGCCC
GACTACCTGGATTTCGCCGAAAGTGGCCAGGTGTATTTTGGGATCATTGC
TCTGGGGGGATCAGGCGAGCAGAAGCTGATCAGCGAGGAAGATCTGGGAG
GACCACTGGGGGTGCGCGGAGGAGGGGGAAGTGGACCTAAGAACAATCAC
ACCGCTTCAATTCTGGACCGAATGCAGGCAGATTTCAAATGCTGTGGCGC
CGCTAACTATACAGACTGGGAAAAGATCCCTAGTATGTCAAAAAACCGGG
TGCCAGATAGCTGCTGTATTAATGTGACCGTCGGCTGCGGGATCAACTTT
AATGAGAAGGCCATTCATAAAGAAGGCTGCGTGGAGAAGATCGGCGGGTG
GCTGAGAAAAAATGTCCTGGTGGTCGCAGCAGCTGCACTGGGAATCGCCT
TCGTGGAGGTCCTGGGGATCGTGTTCGCTTGCTGTCTGGTCAAAAGCTAA
TGTA
LeGo_CD63-2-2L-TNFa-Myc-2-CD19CAR
(SEQ ID NO: 372)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGVRGGSSSRTPSDKPVAHVVANPQAEGQLQ
WLNRRANALLANGVELRDNQLVVPSEGLYLIYSQVLFKGQGCPSTHVLLT
HTISRIAVSYQTKVNLLSAIKSPCQRETPEGAEAKPWYEPIYLGGVFQLE
KGDRLSAEINRPDYLDFAESGQVYFGIIALGGSGEQKLISEEDLGGPLGV
RGGGGSGGGSGPDIQMTQTTSSLSASLGDRVTISCRASQDISKYLNWYQQ
KPDGTVKLLIYHTSRLHSGVPSRFSGSGSGTDYSLTISNLEQEDIATYFC
QQGNTLPYTFGGGTKLEITGGGGSGGGGSGGGGSEVKLQESGPGLVAPSQ
SLSVTCTVSGVSLPDYGVSWIRQPPRKGLEWLGVIWGSETTYYNSALKSR
LTIIKDNSKSQVFLKMNSLQTDDTAIYYCAKHYYYGGSYAMDYWGQGTSV
TVSSAKPTTTPAPRPPTPAPTIASQPLSLRPEACRPAAGGAVHTRGLDFA
PRKIEVMYPPPYLDNEKSNGTIIHVKGKHLCPSPLFPGPSKPFWVLVVVG
GVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLY
QGLSTATKDTYDALHMQALPPR

Which is encoded by cDNA:

(SEQ ID NO: 351)
GGATCCGCCGCCACCATGGCCGTGGAGGGCGGGATGAAGTGCGTCAAATT
CCTGCTGTACGTGCTGCTGCTGGCCTTTTGCGCATGTGCAGTCGGACTGA
TCGCAGTCGGAGTGGGAGCACAGCTGGTGCTGAGTCAGACTATCATTCAG
GGAGCAACCCCTGGCAGCCTGCTGCCAGTGGTCATCATTGCTGTCGGCGT
GTTCCTGTTTCTGGTCGCTTTCGTGGGGTGCTGTGGAGCATGCAAGGAGA
ACTACTGTCTGATGATCACTTTCGCCATTTTTCTGTCTCTGATCATGCTG
GTCGAAGTGGCCGCTGCAATTGCTGGATACGTCGGAGGCAGCGGACCTGT
GCGGAGATATCAGGGAGGACCACTGGGCGTCCGCGGAGGGAGCTCCTCTC
GAACCCCAAGCGACAAGCCTGTGGCACACGTGGTCGCTAATCCTCAGGCA
GAGGGACAGCTGCAGTGGCTGAACAGGCGAGCTAATGCACTGCTGGCCAA
CGGAGTGGAGCTGAGAGATAATCAGCTGGTGGTCCCTAGCGAAGGCCTGT
ACCTGATCTATTCCCAGGTCCTGTTCAAAGGACAGGGATGCCCATCCACC
CACGTGCTGCTGACCCATACAATCAGCAGAATTGCCGTCTCCTACCAGAC
AAAGGTGAACCTGCTGAGCGCCATCAAGAGTCCATGTCAGAGGGAAACTC
CCGAGGGGGCCGAAGCTAAGCCATGGTACGAGCCCATCTATCTGGGAGGC
GTGTTTCAGCTGGAGAAAGGCGACAGGCTGAGCGCAGAAATTAATCGCCC
TGACTACCTGGATTTCGCCGAGTCCGGCCAGGTGTATTTTGGGATCATTG
CTCTGGGGGGCAGCGGCGAACAGAAGCTGATCTCTGAGGAAGATCTGGGA
GGACCACTGGGGGTGAGGGGAGGAGGGGGATCAGGAGGAGGAAGCGGACA
GGTCCAGCTGGTGGAAAGCGGAGGAGGACTGGTGCAGCCAGGAGGGTCCC
TGAAGCTGTCTTGCGCCGCTAGTGGCTTCGACTTTTCTCGATACTGGATG
AGTTGGGTGAGGCAGGCCCCTGGAAAAGGCCTGGAGTGGATCGGCGAAAT
TAACCCCACAAGTTCAACTATCAATTTCACCCCTTCTCTGAAGGACAAAG
TGTTTATTAGTCGCGATAACGCCAAGAATACTCTGTACCTGCAGATGTCA
AAAGTGCGGAGCGAGGACACCGCTCTGTACTATTGTGCACGAGGGAACTA
CTATCGGTACGGAGACGCCATGGATTATTGGGGGCAGGGAACCTCTGTCA
CAGTGTCCAAGATCTCTGGCGGAGGAGGATCAGGAGGAGGAGGCAGCGGC
GGAGGAGGGTCCGGAGGCGGGGGATCTGGCGGGGGAGGCAGCTCCGATAT
CGTCCTGACACAGAGTCCCGCTTCACTGGCAGTGTCACTGGGCCAGCGGG
CAACCATTAGCTGTAGAGCCTCTAAAAGCGTGAGCACCAGCGGCTACAGC
TATCTGCACTGGTATCAGCAGAAGCCTGGGCAGCCCCCTAAACTGCTGAT
CTATCTGGCTTCCAACCTGGAGTCTGGCGTGCCTGCACGCTTCTCCGGCT
CTGGGAGTGGAACTGACTTTACCCTGAATATTCACCCAGTGGAGGAAGAG
GATGCAGCCACTTACTATTGCCAGCATTCCCGGGAGCTGCCCTTCACCTT
TGGCTCTGGGACAAAGCTGGAAATCAAAGGGGGAGGCGGGAGTGGCGGCG
GCGGATCAGGCGGAGGAGGCAGCGCCAAGCCAACCACAACTCCAGCTCCA
CGACCACCAACACCTGCACCAACTATCGCTAGTCAGCCCCTGTCACTGAG
ACCTGAGGCATGTAGGCCAGCTGCAGGAGGAGCAGTCCATACCCGAGGAC
TGGACTTCGCACCACGAAAGATTGAAGTGATGTACCCTCCACCCTATCTG
GATAACGAAAAAAGCAATGGCACAATCATTCACGTGAAGGGGAAACATCT
GTGCCCCAGTCCTCTGTTCCCAGGCCCCTCAAAGCCCTTTTGGGTCCTGG
TGGTCGTGGGAGGGGTGCTGGCCTGTTACAGCCTGCTGGTCACAGTGGCT
TTCATCATCTTCTGGGTGCGGAGCAAACGGTCCAGACTGCTGCACTCCGA
CTATATGAACATGACACCACGACGGCCTGGCCCAACTAGAAAGCATTACC
AGCCCTATGCCCCTCCAAGGGACTTCGCCGCTTACAGGTCCCGCGTGAAA
TTTTCAAGAAGCGCAGATGCCCCAGCTTACCAGCAGGGACAGAATCAGCT
GTATAACGAGCTGAATCTGGGCAGAAGGGAAGAGTATGACGTGCTGGATA
AGCGACGAGGACGGGACCCCGAAATGGGAGGCAAGCCACGGAGAAAAAAC
CCTCAGGAGGGACTTTACAATGAACTGCAGAAGGACAAAATGGCAGAGGC
CTATTCTGAAATCGGCATGAAGGGGGAGAGGCGCCGAGGAAAAGGCCACG
ATGGGCTGTACCAGGGACTGAGCACTGCCACCAAGGACACCTATGATGCC
CTGCATATGCAGGCTCTGCCCCCTCGGTAATGTACA
LeGo-CD63-2L-TNFa-Myc-CD63-Cyt
(SEQ ID NO: 364)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVGGSGPVRRYQGGPLGVRGGSSSRTPSDKPVAHVVANPQAEGQLQ
WLNRRANALLANGVELRDNQLVVPSEGLYLIYSQVLFKGQGCPSTHVLLT
HTISRIAVSYQTKVNLLSAIKSPCQRETPEGAEAKPWYEPIYLGGVFQLE
KGDRLSAEINRPDYLDFAESGQVYFGIIALGGSGEQKLISEEDLGGPLGV
RGGGGSGPKNNHTASILDRMQADFKCCGAANYTDWEKIPSMSKNRVPDSC
CINVTVGCGINFNEKAIHKEGCVEKIGGWLRKNVLVVAAAALGIAFVEVL
GIVFACCLVKSIRSGYEVM

Which is encoded by cDNA:

(SEQ ID NO: 352)
GGATCCGCCGCCACCATGGCCGTGGAGGGCGGGATGAAGTGCGTGAAATT
CCTGCTGTACGTCCTGCTGCTGGCCTTTTGCGCATGTGCTGTGGGACTGA
TCGCAGTGGGAGTCGGAGCTCAGCTGGTGCTGTCTCAGACTATCATTCAG
GGGGCAACCCCTGGAAGTCTGCTGCCAGTGGTCATCATTGCCGTGGGCGT
CTTCCTGTTTCTGGTGGCATTCGTCGGATGCTGTGGCGCCTGCAAGGAAA
ACTATTGTCTGATGATCACTTTCGCTATTTTTCTGTCCCTGATCATGCTG
GTGGAGGTCGCCGCTGCAATTGCCGGATACGTGGGAGGCTCTGGCCCAGT
CCGGAGATATCAGGGAGGACCACTGGGCGTGCGAGGAGGGAGCTCCTCTC
GAACCCCAAGTGACAAGCCTGTGGCCCACGTGGTCGCTAATCCTCAGGCA
GAAGGGCAGCTGCAGTGGCTGAACAGGCGCGCAAATGCCCTGCTGGCCAA
CGGAGTGGAACTGAGGGATAATCAGCTGGTGGTCCCAAGTGAGGGCCTGT
ACCTGATCTATTCACAGGTGCTGTTCAAAGGGCAGGGATGCCCCAGCACA
CACGTGCTGCTGACCCATACAATCTCAAGGATTGCTGTGAGCTACCAGAC
AAAGGTCAACCTGCTGTCTGCCATCAAAAGTCCCTGTCAGCGCGAGACTC
CTGAAGGGGCTGAGGCAAAGCCATGGTACGAGCCCATCTATCTGGGAGGC
GTGTTTCAGCTGGAAAAAGGAGACAGACTGTCAGCAGAGATTAATAGGCC
CGACTACCTGGATTTCGCCGAAAGCGGCCAGGTGTATTTTGGGATCATTG
CTCTGGGAGGCAGCGGAGAGCAGAAGCTGATCTCTGAGGAAGATCTGGGA
GGACCACTGGGGGTGCGCGGAGGAGGGGGAAGCGGACCTAAGAACAATCA
CACCGCTTCCATTCTGGACCGAATGCAGGCAGATTTCAAATGCTGTGGCG
CCGCTAACTACACAGACTGGGAAAAGATCCCTAGTATGTCAAAAAACCGG
GTGCCAGATTCCTGCTGTATTAATGTGACCGTCGGCTGCGGGATCAACTT
TAATGAGAAGGCCATTCATAAAGAAGGCTGCGTGGAGAAGATCGGCGGGT
GGCTGCGGAAAAATGTCCTGGTGGTCGCAGCAGCTGCACTGGGAATCGCC
TTCGTGGAAGTCCTGGGGATCGTGTTCGCTTGCTGTCTGGTCAAAAGCAT
TAGATCCGGCTATGAAGTGATGTAATGTACAA
LeGo-CD63-DL1-2L-TNFa-Myc-CD63-Cyt
(SEQ ID NO: 368)
MAVEGGMKCVKCGACKENYCLMITFAIFLSLIMLVEVAAAIAGYVGGSGP
VRRYQGGPLGVRGGSSSRTPSDKPVAHVVANPQAEGQLQWLNRRANALLA
NGVELRDNQLVVPSEGLYLIYSQVLFKGQGCPSTHVLLTHTISRIAVSYQ
TKVNLLSAIKSPCQRETPEGAEAKPWYEPIYLGGVFQLEKGDRLSAEINR
PDYLDFAESGQVYFGIIALGGSGEQKLISEEDLGGPLGVRGGGGSGPKNN
HTASILDRMQADFKCCGAANYTDWEKIPSMSKNRVPDSCCINVTVGCGIN
FNEKAIHKEGCVEKIGGWLRKNVLVVAAAALGIAFVEVLGIVFACCLVKS
IRSGYEVM

Which is encoded by cDNA:

(SEQ ID NO: 353)
GGATCCGCCGCCACCATGGCAGTGGAAGGCGGGATGAAGTGCGTCAAATG
TGGCGCCTGCAAGGAGAACTACTGTCTGATGATCACCTTCGCTATTTTTC
TGAGCCTGATCATGCTGGTGGAAGTCGCCGCTGCAATTGCAGGATACGTG
GGAGGCAGCGGACCCGTCCGGAGATATCAGAGCTCCTCTCGAACACCATC
CGACAAGCCTGTGGCTCACGTGGTCGCAAATCCACAGGCAGAGGGACAGC
TGCAGTGGCTGAACAGGCGAGCCAATGCTCTGCTGGCCAACGGAGTGGAA
CTGAGGGATAATCAGCTGGTGGTCCCCAGTGAGGGCCTGTACCTGATCTA
TTCACAGGTGCTGTTCAAAGGACAGGGCTGTCCTTCTACTCACGTGCTGC
TGACCCATACAATCTCAAGGATTGCCGTGAGCTATCAGACTAAGGTCAAC
CTGCTGTCTGCTATCAAAAGTCCTTGCCAGCGAGAGACCCCAGAAGGAGC
AGAGGCCAAGCCATGGTACGAGCCCATCTATCTGGGAGGCGTGTTTCAGC
TGGAAAAAGGAGACAGACTGTCTGCAGAGATTAATAGGCCTGACTACCTG
GATTTCGCCGAAAGTGGCCAGGTGTATTTTGGGATCATTGCTCTGGGAGG
CAGCGGAGAGCAGAAGCTGATCTCTGAGGAAGATCTGGGGGGAAGCGGAC
CAAAGAACAATCACACAGCTTCCATTCTGGACCGAATGCAGGCAGATTTC
AAATGCTGTGGCGCCGCTAACTACACTGACTGGGAAAAGATCCCAAGTAT
GTCAAAAAACCGGGTGCCCGATAGCTGCTGTATTAATGTGACAGTCGGGT
GTGGAATCAACTTTAATGAGAAGGCCATTCATAAAGAAGGCTGCGTGGAG
AAGATCGGCGGGTGGCTGCGGAAAAATGTCCTGGTGGTCGCAGCAGCTGC
ACTGGGAATCGCCTTCGTGGAAGTCCTGGGGATCGTGTTCGCTTGCTGTC
TGGTCAAAAGCATTAGATCCGGCTATGAAGTGATGTAATGTACAA
LeGO-CD69Cyt-TM-GrnzB-TNFa-Myc-GrnzB-a-CD19sc-
Stalk-CD28-CD3Z
(SEQ ID NO: 373)
MSSENCFVAENSSLHPESGQENDATSPHFSTRHEGSFQVPVLCAVMNVVF
ITILIIALIALSVGQYNGGSGPVRRYQGGSGGGEEEEEVEADSEEEEEEE
SGGSGSSSRTPSDKPVAHVVANPQAEGQLQWLNRRANALLANGVELRDNQ
LVVPSEGLYLIYSQVLFKGQGCPSTHVLLTHTISRIAVSYQTKVNLLSAI
KSPCQRETPEGAEAKPWYEPIYLGGVFQLEKGDRLSAEINRPDYLDFAES
GQVYFGIIALEQKLISEEDLGGSGEEEEEVEADSEEEEEEEGGSGGGSGG
GSGPDIQMTQTTSSLSASLGDRVTISCRASQDISKYLNWYQQKPDGTVKL
LIYHTSRLHSGVPSRFSGSGSGTDYSLTISNLEQEDIATYFCQQGNTLPY
TFGGGTKLEITGGGGSGGGGSGGGGSEVKLQESGPGLVAPSQSLSVTCTV
SGVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSSGGGG
SGGGGSAKPTTTPAPRPPTPAPTIASQPLSLRPEACRPAAGGAVHTRGLD
FAPRKIEVMYPPPYLDNEKSNGTIIHVKGKHLCPSPLFPGPSKPFWVLVV
VGGVLACYSLLVTVAFIIFWVRSKRSRLLHSDYMNMTPRRPGPTRKHYQP
YAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPR

Which is encoded by cDNA:

(SEQ ID NO: 354)
GGATCCATGTCATCCGAGAACTGCTTTGTCGCCGAAAACTCATCACTGCA
CCCCGAATCAGGCCAGGAAAACGACGCAACCTCCCCCCACTTCTCAACTA
GACACGAGGGCAGCTTCCAGGTGCCAGTGCTGTGCGCCGTGATGAACGTG
ATTGCTCTGATCGCCCTGAGCGTGGGGCAGTACAATGGCGGGTCCGGACC
GTGTTTATCACCATTCTGATCCGTGCGGAGATATCAGGGAGGCTCCGGAG
GAGGAGAGGAAGAGGAAGAGGTGGAGGCCGACTCTGAAGAGGAAGAGGAA
GAGGAATCCGGGGGATCTGGCAGCTCCTCTAGAACCCCTTCCGATAAGCC
AGTGGCTCATGTGGTGGCCAACCCTCAGGCTGAGGGACAGCTGCAGTGGC
TGAACAGGAGGGCTAATGCTCTGCTGGCTAACGGAGTGGAGCTGAGGGAC
AATCAGCTGGTGGTGCCTTCTGAAGGGCTGTACCTGATCTATAGTCAGGT
GCTGTTCAAAGGGCAGGGATGCCCATCTACACACGTGCTGCTGACCCATA
CAATCAGTCGCATTGCCGTGAGCTACCAGACCAAGGTGAACCTGCTGAGC
GCCATCAAGAGCCCTTGTCAGCGGGAAACACCAGAGGGGGCCGAAGCTAA
GCCCTGGTACGAGCCTATCTATCTGGGCGGGGTGTTTCAGCTGGAGAAAG
GCGATAGACTGAGCGCCGAAATTAATAGGCCCGACTACCTGGATTTCGCT
GAGAGCGGCCAGGTGTATTTTGGGATCATTGCCCTGGAACAGAAGCTGAT
CTCCGAGGAAGACCTGGGAGGCTCTGGCGAAGAGGAAGAGGAGGTGGAGG
CCGATAGCGAGGAAGAAGAGGAAGAGGAAGGAGGCAGCGGAGGAGGATCT
GGAGGAGGAAGTGGACAGGTGCAGCTGGTGGAGAGCGGCGGAGGACTGGT
GCAGCCAGGAGGGTCACTGAAGCTGAGCTGCGCCGCTTCCGGCTTCGACT
TTAGCCGCTACTGGATGTCCTGGGTGCGGCAGGCTCCTGGCAAAGGGCTG
GAGTGGATCGGCGAAATTAACCCCACTAGTTCAACCATCAATTTCACACC
TTCACTGAAGGACAAAGTGTTTATTAGCAGAGATAACGCCAAGAATACAC
TGTACCTGCAGATGTCTAAAGTGAGGAGTGAGGACACTGCCCTGTACTAT
TGTGCTCGCGGAAATTACTATCGGTACGGCGACGCCATGGATTATTGGGG
ACAGGGCACAAGCGTGACTGTGAGTAAGATCTCAGGAGGCGGCGGATCTG
GAGGAGGAGGCAGTGGGGGAGGAGGGTCAGGAGGAGGAGGAAGCGGCGGA
GGAGGCAGCTCCGATATCGTGCTGACTCAGAGCCCAGCTTCCCTGGCCGT
GTCCCTGGGACAGCGCGCCACTATTTCTTGTCGGGCTTCTAAGAGCGTGA
GCACCAGCGGGTACTCCTATCTGCACTGGTACCAGCAGAAGCCTGGACAG
CCCCCTAAACTGCTGATCTATCTGGCCTCAAACCTGGAGAGCGGCGTGCC
TGCTAGATTCTCCGGGTCTGGAAGTGGCACCGACTTTACACTGAATATTC
ACCCAGTGGAGGAAGAGGATGCCGCTACCTACTATTGCCAGCATAGTAGG
GAGCTGCCCTTCACATTTGGGAGCGGAACTAAGCTGGAGATCAAGGGAGG
AGGAGGGTCCGGAGGCGGGGGATCTGGCGGCGGAGGCAGTGCCAAGCCAA
CCACAACTCCAGCTCCTCGCCCACCAACTCCAGCTCCAACCATCGCTTCC
CAGCCACTGTCTCTGCGGCCCGAGGCTTGTAGGCCTGCTGCTGGGGGAGC
TGTGCATACAAGAGGCCTGGACTTCGCCCCAAGGAAGATTGAAGTGATGT
ACCCTCCACCCTATCTGGATAACGAAAAATCCAATGGCACTATCATTCAC
GTGAAGGGGAAACATCTGTGCCCTAGTCCACTGTTCCCCGGCCCTTCAAA
GCCCTTTTGGGTGCTGGTGGTGGTGGGAGGGGTGCTGGCCTGTTACTCCC
TGCTGGTGACCGTGGCTTTCATCATCTTCTGGGTGCGGAGCAAACGCAGT
CGGCTGCTGCACTCTGACTATATGAACATGACCCCAAGACGGCCCGGACC
AACAAGGAAGCATTACCAGCCATATGCTCCTCCAAGGGACTTCGCTGCTT
ACAGAAGCAGGGTGAAATTTTCACGCAGCGCCGATGCTCCTGCCTACCAG
CAGGGACAGAATCAGCTGTATAACGAGCTGAATCTGGGCAGGCGCGAAGA
GTATGACGTGCTGGATAAGCGGAGAGGACGGGACCCCGAAATGGGAGGCA
AGCCAAGGCGCAAAAACCCCCAGGAGGGCCTGTATAACGAACTGCAGAAG
GACAAAATGGCTGAGGCCTATAGCGAAATCGGAATGAAGGGCGAGCGGAG
AAGGGGCAAAGGGCACGATGGGCTGTACCAGGGACTGTCCACCGCCACTA
AAGATACCTATGATGCTCTGCACATGCAGGCTCTGCCACCCCGATAATGT
ACAA
LeGO-CD69Cyt-TM-II-9-TNFa-Myc-9-CD28TM-Cyt
(SEQ ID NO: 377)
MSSENCFVAENSSLHPESGQENDATSPHFSTRHEGSFQVPVLCAVMNVVF
ITILIIALIALSVGQYNGGSGPVRRYQGGSGGGPLGMTSGGSGSSSRTPS
DKPVAHVVANPQAEGQLQWLNRRANALLANGVELRDNQLVVPSEGLYLIY
SQVLFKGQGCPSTHVLLTHTISRIAVSYQTKVNLLSAIKSPCQRETPEGA
EAKPWYEPIYLGGVFQLEKGDRLSAEINRPDYLDFAESGQVYFGIIALGG
SGEQKLISEEDLGGPLGMTSGGSGCPSPLFPGPSKPFWVLVVVGGVLACY
SLLVTVAFIIFWVRSKRSRLL

Which is encoded by cDNA:

(SEQ ID NO: 355)
GGATCCGCCGCCACCATGAGTTCCGAAAACTGCTTTGTCGCCGAGAATAG
TAGTCTGCACCCCGAATCAGGACAGGAGAATGACGCAACCAGCCCCCACT
TTTCAACAAGGCACGAAGGGAGCTTCCAGGTCCCTGTGCTGTGCGCAGTG
ATGAACGTGGTCTTTATCACTATTCTGATCATTGCTCTGATCGCACTGAG
TGTCGGACAGTACAATGGCGGGTCAGGCCCCGTGCGGAGATATCAGGGAG
GGTCTGGGGGAGGACCACTGGGAATGACCTCCGGAGGGTCTGGGAGCTCC
TCTCGCACCCCTTCCGACAAGCCAGTGGCCCATGTGGTCGCTAACCCTCA
GGCAGAGGGACAGCTGCAGTGGCTGAACAGGCGAGCCAATGCTCTGCTGG
CTAACGGCGTGGAACTGCGAGATAATCAGCTGGTGGTCCCTAGCGAGGGG
CTGTACCTGATCTATTCCCAGGTCCTGTTCAAAGGGCAGGGATGCCCATC
TACACACGTGCTGCTGACCCATACAATCTCTAGAATTGCCGTCAGTTACC
AGACTAAGGTGAACCTGCTGAGCGCCATCAAGTCACCATGTCAGAGGGAG
ACACCCGAAGGAGCAGAGGCCAAGCCCTGGTACGAGCCTATCTATCTGGG
AGGCGTGTTTCAGCTGGAAAAAGGCGACCGACTGTCTGCCGAGATTAATC
GGCCAGACTACCTGGATTTCGCTGAAAGTGGCCAGGTGTATTTTGGGATC
ATTGCACTGGGAGGCAGCGGAGAGCAGAAGCTGATCTCTGAGGAAGATCT
GGGAGGACCACTGGGAATGACCTCCGGAGGGAGCGGATGCCCATCCCCTC
TGTTCCCAGGACCCAGCAAACCCTTTTGGGTCCTGGTGGTCGTGGGAGGC
GTGCTGGCATGTTATAGCCTGCTGGTCACAGTGGCTTTCATTATCTTTTG
GGTCCGCTCTAAACGGTCTCGGCTGCTGTAATGTACAA
LeGO-CD69Cyt-TM-II-TNFa-Myc-CD28TM-Cyt
(SEQ ID NO: 370)
MSSENCFVAENSSLHPESGQENDATSPHFSTRHEGSFQVPVLCAVMNVVF
ITILIIALIALSVGQYNGGSGPVRRYQSSSRTPSDKPVAHVVANPQAEGQ
LQWLNRRANALLANGVELRDNQLVVPSEGLYLIYSQVLFKGQGCPSTHVL
LTHTISRIAVSYQTKVNLLSAIKSPCQRETPEGAEAKPWYEPIYLGGVFQ
LEKGDRLSAEINRPDYLDFAESGQVYFGIIALGGSGEQKLISEEDLGGSG
CPSPLFPGPSKPFWVLVVVGGVLACYSLLVTVAFIIFWVRSKRSRLL

Which is encoded by cDNA:

(SEQ ID NO: 356)
GGATCCGCCGCCACCATGAGTTCCGAAAACTGCTTTGTCGCCGAGAATAG
TAGTCTGCACCCCGAATCAGGACAGGAGAATGACGCAACCAGCCCCCACT
TTTCAACAAGGCACGAAGGGAGCTTCCAGGTCCCTGTGCTGTGCGCAGTG
ATGAACGTGGTCTTTATCACTATTCTGATCATTGCTCTGATCGCACTGAG
TGTCGGACAGTACAATGGCGGGTCAGGCCCCGTGCGGAGATATCAGAGCT
CCTCTCGCACCCCTTCCGACAAGCCAGTGGCCCATGTGGTCGCTAACCCT
CAGGCAGAGGGACAGCTGCAGTGGCTGAACAGGCGAGCCAATGCTCTGCT
GGCTAACGGCGTGGAACTGCGAGATAATCAGCTGGTGGTCCCTAGCGAGG
GGCTGTACCTGATCTATTCCCAGGTCCTGTTCAAAGGGCAGGGATGCCCA
TCTACACACGTGCTGCTGACCCATACAATCTCTAGAATTGCCGTCAGTTA
CCAGACTAAGGTGAACCTGCTGAGCGCCATCAAGTCACCATGTCAGAGGG
AGACACCCGAAGGAGCAGAGGCCAAGCCCTGGTACGAGCCTATCTATCTG
GGAGGCGTGTTTCAGCTGGAAAAAGGCGACCGACTGTCTGCCGAGATTAA
TCGGCCAGACTACCTGGATTTCGCTGAAAGTGGCCAGGTGTATTTTGGGA
TCATTGCACTGGGAGGCAGCGGAGAGCAGAAGCTGATCTCTGAGGAAGAT
CTGGGAGGGAGCGGATGCCCATCCCCTCTGTTCCCAGGACCCAGCAAACC
CTTTTGGGTCCTGGTGGTCGTGGGAGGCGTGCTGGCATGTTATAGCCTGC
TGGTCACAGTGGCTTTCATTATCTTTTGGGTCCGCTCTAAACGGTCTCGG
CTGCTGTAATGTACAA
LeGO-CD69Cyt-TM-GrzB-TNFa-Myc-GrzB-a-CD19sc-
Stalk-CD28-CD3z
(SEQ ID NO: 373)
MSSENCFVAENSSLHPESGQENDATSPHFSTRHEGSFQVPVLCAVMNVVF
ITILIIALIALSVGQYNGGSGPVRRYQGGSGGGEEEEEVEADSEEEEEEE
SGGSGSSSRTPSDKPVAHVVANPQAEGQLQWLNRRANALLANGVELRDNQ
LVVPSEGLYLIYSQVLFKGQGCPSTHVLLTHTISRIAVSYQTKVNLLSAI
KSPCQRETPEGAEAKPWYEPIYLGGVFQLEKGDRLSAEINRPDYLDFAES
GQVYFGIIALEQKLISEEDLGGSGEEEEEVEADSEEEEEEEGGSGGGSGG
GSGPDIQMTQTTSSLSASLGDRVTISCRASQDISKYLNWYQQKPDGTVKL
LIYHTSRLHSGVPSRFSGSGSGTDYSLTISNLEQEDIATYFCQQGNTLPY
TFGGGTKLEITGGGGSGGGGSGGGGSEVKLQESGPGLVAPSQSLSVTCTV
SGVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSSGGGG
SGGGGSAKPTTTPAPRPPTPAPTIASQPLSLRPEACRPAAGGAVHTRGLD
FAPRKIEVMYPPPYLDNEKSNGTIIHVKGKHLCPSPLFPGPSKPFWVLVV
VGGVLACYSLLVTVAFIIFWVRSKRSRLLHSDYMNMTPRRPGPTRKHYQP
YAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPR

Which is encoded by cDNA:

(SEQ ID NO: 357)
GGATCCATGTCATCCGAGAACTGCTTTGTCGCCGAAAACTCATCACTGCACCCCGAA
TCAGGCCAGGAAAACGACGCAACCTCCCCCCACTTCTCAACTAGACACGAGGGCAG
CTTCCAGGTGCCAGTGCTGTGCGCCGTGATGAACGTGGTGTTTATCACCATTCTGATC
ATTGCTCTGATCGCCCTGAGCGTGGGGCAGTACAATGGCGGGTCCGGACCCGTGCGG
AGATATCAGGGAGGCTCCGGAGGAGGAGAGGAAGAGGAAGAGGTGGAGGCCGACT
CTGAAGAGGAAGAGGAAGAGGAATCCGGGGGATCTGGCAGCTCCTCTAGAACCCCT
TCCGATAAGCCAGTGGCTCATGTGGTGGCCAACCCTCAGGCTGAGGGACAGCTGCA
GTGGCTGAACAGGAGGGCTAATGCTCTGCTGGCTAACGGAGTGGAGCTGAGGGACA
ATCAGCTGGTGGTGCCTTCTGAAGGGCTGTACCTGATCTATAGTCAGGTGCTGTTCA
AAGGGCAGGGATGCCCATCTACACACGTGCTGCTGACCCATACAATCAGTCGCATTG
CCGTGAGCTACCAGACCAAGGTGAACCTGCTGAGCGCCATCAAGAGCCCTTGTCAG
CGGGAAACACCAGAGGGGGCCGAAGCTAAGCCCTGGTACGAGCCTATCTATCTGGG
CGGGGTGTTTCAGCTGGAGAAAGGCGATAGACTGAGCGCCGAAATTAATAGGCCCG
ACTACCTGGATTTCGCTGAGAGCGGCCAGGTGTATTTTGGGATCATTGCCCTGGAAC
AGAAGCTGATCTCCGAGGAAGACCTGGGAGGCTCTGGCGAAGAGGAAGAGGAGGT
GGAGGCCGATAGCGAGGAAGAAGAGGAAGAGGAAGGAGGCAGCGGAGGAGGATCT
GGAGGAGGAAGTGGACAGGTGCAGCTGGTGGAGAGCGGCGGAGGACTGGTGCAGC
CAGGAGGGTCACTGAAGCTGAGCTGCGCCGCTTCCGGCTTCGACTTTAGCCGCTACT
GGATGTCCTGGGTGCGGCAGGCTCCTGGCAAAGGGCTGGAGTGGATCGGCGAAATT
AACCCCACTAGTTCAACCATCAATTTCACACCTTCACTGAAGGACAAAGTGTTTATT
AGCAGAGATAACGCCAAGAATACACTGTACCTGCAGATGTCTAAAGTGAGGAGTGA
GGACACTGCCCTGTACTATTGTGCTCGCGGAAATTACTATCGGTACGGCGACGCCAT
GGATTATTGGGGACAGGGCACAAGCGTGACTGTGAGTAAGATCTCAGGAGGCGGCG
GATCTGGAGGAGGAGGCAGTGGGGGAGGAGGGTCAGGAGGAGGAGGAAGCGGCGG
AGGAGGCAGCTCCGATATCGTGCTGACTCAGAGCCCAGCTTCCCTGGCCGTGTCCCT
GGGACAGCGCGCCACTATTTCTTGTCGGGCTTCTAAGAGCGTGAGCACCAGCGGGTA
CTCCTATCTGCACTGGTACCAGCAGAAGCCTGGACAGCCCCCTAAACTGCTGATCTA
TCTGGCCTCAAACCTGGAGAGCGGCGTGCCTGCTAGATTCTCCGGGTCTGGAAGTGG
CACCGACTTTACACTGAATATTCACCCAGTGGAGGAAGAGGATGCCGCTACCTACTA
TTGCCAGCATAGTAGGGAGCTGCCCTTCACATTTGGGAGCGGAACTAAGCTGGAGAT
CAAGGGAGGAGGAGGGTCCGGAGGCGGGGGATCTGGCGGCGGAGGCAGTGCCAAG
CCAACCACAACTCCAGCTCCTCGCCCACCAACTCCAGCTCCAACCATCGCTTCCCAG
CCACTGTCTCTGCGGCCCGAGGCTTGTAGGCCTGCTGCTGGGGGAGCTGTGCATACA
AGAGGCCTGGACTTCGCCCCAAGGAAGATTGAAGTGATGTACCCTCCACCCTATCTG
GATAACGAAAAATCCAATGGCACTATCATTCACGTGAAGGGGAAACATCTGTGCCC
TAGTCCACTGTTCCCCGGCCCTTCAAAGCCCTTTTGGGTGCTGGTGGTGGTGGGAGG
GGTGCTGGCCTGTTACTCCCTGCTGGTGACCGTGGCTTTCATCATCTTCTGGGTGCGG
AGCAAACGCAGTCGGCTGCTGCACTCTGACTATATGAACATGACCCCAAGACGGCC
CGGACCAACAAGGAAGCATTACCAGCCATATGCTCCTCCAAGGGACTTCGCTGCTTA
CAGAAGCAGGGTGAAATTTTCACGCAGCGCCGATGCTCCTGCCTACCAGCAGGGAC
AGAATCAGCTGTATAACGAGCTGAATCTGGGCAGGCGCGAAGAGTATGACGTGCTG
GATAAGCGGAGAGGACGGGACCCCGAAATGGGAGGCAAGCCAAGGCGCAAAAACC
CCCAGGAGGGCCTGTATAACGAACTGCAGAAGGACAAAATGGCTGAGGCCTATAGC
GAAATCGGAATGAAGGGCGAGCGGAGAAGGGGCAAAGGGCACGATGGGCTGTACC
AGGGACTGTCCACCGCCACTAAAGATACCTATGATGCTCTGCACATGCAGGCTCTGC
CACCCCGATAATGTACA
LeGO-CD69Cyt-TM-GrzB-nLuc-Myc-GrzB-a-CD19sc-Stalk CD28-CD3z
(SEQ ID NO: 358)
MSSENCFVAENSSLHPESGQENDATSPHFSTRHEGSFQVPVLCAVMNVVFITILIIALI
ALSVGQYNGGSGPVRRYSRQGGSGGGEEEEEVEADSEEEEEEESGGSGSSSSRVFTL
EDFVGDWRQTAGYNLDQVLEQGGVSSLFQNLGVSVTPIQRIVLSGENGLKIDIHVIIP
YEGLSGDQMGQIEKIFKVVYPVDDHHFKVILHYGTLVIDGVTPNMIDYFGRPYEGIA
VFDGKKITVTGTLWNGNKIIDERLINPDGSLLFRVTINGVTGWRLCERILAEQKLISEE
DLLEGGSGEEEEEVEADSEEEEEEEGGSGGGSGLEPRGGSGVNPDIQMTQTTSSLSAS
LGDRVTISCRASQDISKYLNWYQQKPDGTVKLLIYHTSRLHSGVPSRFSGSGSGTDYS
LTISNLEQEDIATYFCQQGNTLPYTFGGGTKLEITGGGGSGGGGSGGGGSEVKLQESG
PGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSSV
NSGGGGSGGGGSAKPTTTPAPRPPTPAPTIASQPLSLRPEACRPAAGGAVHTRGLDFA
PRKIEVMYPPPYLDNEKSNGTIIHVKGKHLCPSPLFPGPSKPFWVLVVVGGVLACYSL
LVTVAFIIFWVRSKRSRLLHSDYMNMTPRRPGPTRKHYQPYAPPRDFAAYRSRVKFS
RSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR

Which is encoded by cDNA:

(SEQ ID NO: 382)
GCCAGTCCTCCGATTGACTGAGTCGCCCGGATCCCGCCACCATGAGCTCTGAGAA
CTGCTTCGTGGCCGAGAACAGCAGCCTCCACCCCGAGAGTGGTCAGGAGAACGA
CGCCACCAGCCCCCACTTCAGCACAAGACACGAGGGCTCATTCCAGGTGCCTGT
GCTGTGCGCCGTGATGAACGTGGTGTTCATCACCATCCTGATCATCGCCCTGATT
GCCCTTTCTGTGGGCCAATACAATGGCGGCTCCGGTCCTGTGCGCAGATACTCTA
GACAGGGCGGCTCTGGCGGAGGCGAAGAGGAAGAAGAGGTGGAAGCCGATAGC
GAGGAAGAAGAAGAAGAGGAGAGCGGCGGCTCCGGCAGCAGCAGCTCTAGAGT
GTTCACCCTCGAAGATTTCGTGGGCGACTGGAGACAGACCGCCGGCTACAACCT
GGACCAGGTGCTGGAACAGGGCGGCGTGAGCTCTCTGTTTCAGAATCTGGGGGT
TAGCGTGACACCAATCCAGAGGATCGTGCTGTCCGGCGAGAACGGGCTGAAGAT
CGACATCCACGTGATCATTCCATACGAGGGCCTGTCTGGCGATCAGATGGGCCA
GATCGAGAAGATCTTCAAGGTGGTGTACCCTGTAGACGACCACCATTTTAAGGTG
ATCCTGCACTACGGCACCCTGGTCATCGACGGCGTGACACCCAACATGATCGACT
ACTTCGGCAGACCATATGAGGGTATTGCCGTGTTTGATGGAAAGAAGATCACAG
TCACCGGCACCCTGTGGAACGGCAACAAGATTATCGATGAACGGCTGATCAACC
CCGACGGCTCTCTGCTGTTTAGAGTGACCATCAATGGCGTGACAGGCTGGAGACT
GTGCGAGAGAATCCTGGCTGAGCAGAAACTGATCAGCGAGGAAGACCTGCTCGA
GGGCGGTAGCGGCGAGGAGGAAGAAGAAGTGGAAGCTGACAGCGAAGAAGAG
GAAGAGGAGGAGGGCGGGAGCGGAGGCGGCAGTGGCCTCGAGCCTAGGGGCGG
CAGCGGAGTTAACCCCGATATTCAGATGACTCAGACTACCAGCTCCCTGAGCGC
ATCCCTGGGCGACCGAGTGACAATCAGTTGCCGAGCCAGCCAGGACATCAGCAA
GTATCTGAACTGGTACCAGCAGAAGCCTGACGGAACCGTCAAACTGCTGATCTA
TCACACATCCCGCCTGCATTCTGGCGTGCCAAGTCGGTTCTCTGGAAGTGGCTCA
GGGACCGACTACTCACTGACAATCAGCAACCTGGAGCAGGAAGATATTGCCACC
TATTTCTGCCAGCAGGGCAATACTCTGCCTTACACCTTTGGCGGGGGAACTAAGC
TGGAGATTACCGGCGGAGGAGGCAGCGGAGGAGGAGGGTCCGGAGGCGGGGGA
TCTGAGGTGAAACTGCAGGAAAGCGGACCTGGCCTGGTCGCTCCAAGCCAGTCC
CTGTCTGTGACTTGTACCGTCAGCGGAGTGTCCCTGCCCGATTACGGCGTCTCTT
GGATCAGACAGCCCCCTAGAAAGGGCCTGGAGTGGCTGGGCGTGATCTGGGGAA
GTGAAACCACATACTATAATTCCGCCCTGAAAAGCCGGCTGACAATCATTAAGG
ACAACAGTAAATCACAGGTGTTCCTGAAGATGAATTCCCTGCAGACAGACGATA
CTGCAATCTACTATTGCGCCAAACACTACTATTACGGCGGGAGCTATGCTATGGA
TTACTGGGGGCAGGGAACCTCCGTCACAGTGTCTAGTGTTAACTCAGGCGGCGG
CGGCAGCGGAGGCGGAGGCAGTGCCAAGCCTACAACCACCCCCGCCCCTAGACC
TCCTACACCTGCCCCTACCATCGCTAGCCAGCCACTCTCTCTCAGACCTGAAGCC
TGTAGACCCGCCGCTGGCGGCGCCGTGCACACCAGAGGCCTGGACTTCGCGCCT
CGGAAGATCGAGGTGATGTACCCCCCGCCTTACCTGGACAACGAGAAGTCTAAT
GGAACAATCATCCACGTGAAGGGAAAACACCTGTGTCCTTCTCCTCTGTTCCCTG
GCCCCAGCAAGCCTTTCTGGGTCCTGGTGGTGGTCGGCGGCGTGCTGGCGTGCTA
CAGCCTGCTCGTGACCGTGGCCTTCATCATTTTCTGGGTGCGGAGCAAACGGAGC
AGACTGCTGCATAGCGATTACATGAACATGACCCCAAGAAGACCTGGGCCAACC
AGAAAGCACTACCAGCCTTACGCCCCCCCCAGAGATTTCGCCGCTTATAGAAGCC
GGGTGAAGTTCTCCAGATCCGCCGACGCCCCTGCTTACCAACAGGGCCAGAACC
AGCTGTATAACGAGCTGAATCTGGGCAGAAGAGAGGAGTACGACGTGCTGGATA
AGCGGAGAGGAAGAGACCCTGAGATGGGCGGCAAGCCCCGGCGGAAGAACCCT
CAGGAGGGCCTGTATAACGAGCTGCAAAAAGATAAGATGGCCGAGGCTTATAGC
GAGATCGGAATGAAGGGCGAGCGGCGGAGGGGCAAAGGCCACGACGGCCTGTA
CCAGGGACTGAGCACAGCCACAAAGGACACCTACGACGCTCTGCACATGCAGGC
CCTGCCTCCTAGATAATGTACAAGTAAAGCGGCCGGCC
LeGO-CD69Cyt-TM-GrzB-nLuc-Myc-GrzB-CD28-CD3z, (Delta-sc, GAGE B)
(SEQ ID NO: 374)
MSSENCFVAENSSLHPESGQENDATSPHFSTRHEGSFQVPVLCAVMNVVFITILIIALIALS
VGQYNGGSGPVRRYSRQGGSGGGEEEEEVEADSEEEEEEESGGSGSSSSRVFTLEDFVG
DWRQTAGYNLDQVLEQGGVSSLFQNLGVSVTPIQRIVLSGENGLKIDIHVIIPYEGLSGD
QMGQIEKIFKVVYPVDDHHFKVILHYGTLVIDGVTPNMIDYFGRPYEGIAVFDGKKITVT
GTLWNGNKIIDERLINPDGSLLFRVTINGVTGWRLCERILAEQKLISEEDLLEGGSGEEEE
EVEADSEEEEEEEGGSGGGSGLEPRGGSGVNSGGGGSGGGGSAKPTTTPAPRPPTPAPTI
ASQPLSLRPEACRPAAGGAVHTRGLDFAPRKIEVMYPPPYLDNEKSNGTIIHVKGKHLCP
SPLFPGPSKPFWVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTA
TKDTYDALHMQALPPR

Which is encoded by cDNA:

(SEQ ID NO: 359)
ATGAGCTCTGAGAACTGCTTCGTGGCCGAGAACAGCAGCCTCCACCCCGAGAGT
GGTCAGGAGAACGACGCCACCAGCCCCCACTTCAGCACAAGACACGAGGGCTCA
TTCCAGGTGCCTGTGCTGTGCGCCGTGATGAACGTGGTGTTCATCACCATCCTGA
TCATCGCCCTGATTGCCCTTTCTGTGGGCCAATACAATGGCGGCTCCGGTCCTGT
GCGCAGATACTCTAGACAGGGCGGCTCTGGCGGAGGCGAAGAGGAAGAAGAGG
TGGAAGCCGATAGCGAGGAAGAAGAAGAAGAGGAGAGCGGCGGCTCCGGCAGC
AGCAGCTCTAGAGTGTTCACCCTCGAAGATTTCGTGGGCGACTGGAGACAGACC
GCCGGCTACAACCTGGACCAGGTGCTGGAACAGGGCGGCGTGAGCTCTCTGTTT
CAGAATCTGGGGGTTAGCGTGACACCAATCCAGAGGATCGTGCTGTCCGGCGAG
AACGGGCTGAAGATCGACATCCACGTGATCATTCCATACGAGGGCCTGTCTGGC
GATCAGATGGGCCAGATCGAGAAGATCTTCAAGGTGGTGTACCCTGTAGACGAC
CACCATTTTAAGGTGATCCTGCACTACGGCACCCTGGTCATCGACGGCGTGACAC
CCAACATGATCGACTACTTCGGCAGACCATATGAGGGTATTGCCGTGTTTGATGG
AAAGAAGATCACAGTCACCGGCACCCTGTGGAACGGCAACAAGATTATCGATGA
ACGGCTGATCAACCCCGACGGCTCTCTGCTGTTTAGAGTGACCATCAATGGCGTG
ACAGGCTGGAGACTGTGCGAGAGAATCCTGGCTGAGCAGAAACTGATCAGCGAG
GAAGACCTGCTCGAGGGCGGTAGCGGCGAGGAGGAAGAAGAAGTGGAAGCTGA
CAGCGAAGAAGAGGAAGAGGAGGAGGGCGGGAGCGGAGGCGGCAGTGGCCTCG
AGCCTAGGGGCGGCAGCGGAGTTAACTCAGGCGGCGGCGGCAGCGGAGGCGGA
GGCAGTGCCAAGCCTACAACCACCCCCGCCCCTAGACCTCCTACACCTGCCCCTA
CCATCGCTAGCCAGCCACTCTCTCTCAGACCTGAAGCCTGTAGACCCGCCGCTGG
CGGCGCCGTGCACACCAGAGGCCTGGACTTCGCGCCTCGGAAGATCGAGGTGAT
GTACCCCCCGCCTTACCTGGACAACGAGAAGTCTAATGGAACAATCATCCACGT
GAAGGGAAAACACCTGTGTCCTTCTCCTCTGTTCCCTGGCCCCAGCAAGCCTTTC
TGGGTCCTGGTGGTGGTCGGCGGCGTGCTGGCGTGCTACAGCCTGCTCGTGACCG
TGGCCTTCATCATTTTCTGGGTGCGGAGCAAACGGAGCAGACTGCTGCATAGCGA
TTACATGAACATGACCCCAAGAAGACCTGGGCCAACCAGAAAGCACTACCAGCC
TTACGCCCCCCCCAGAGATTTCGCCGCTTATAGAAGCCGGGTGAAGTTCTCCAGA
TCCGCCGACGCCCCTGCTTACCAACAGGGCCAGAACCAGCTGTATAACGAGCTG
AATCTGGGCAGAAGAGAGGAGTACGACGTGCTGGATAAGCGGAGAGGAAGAGA
CCCTGAGATGGGCGGCAAGCCCCGGCGGAAGAACCCTCAGGAGGGCCTGTATAA
CGAGCTGCAAAAAGATAAGATGGCCGAGGCTTATAGCGAGATCGGAATGAAGG
GCGAGCGGCGGAGGGGCAAAGGCCACGACGGCCTGTACCAGGGACTGAGCACA
GCCACAAAGGACACCTACGACGCTCTGCACATGCAGGCCCTGCCTCCTAGAtaaTG
TACA
CD69Cyt-TM-nLuc-Myc-a-CD19sc-Stalk-CD28-CD3z,, (Delta-GrzB, GAGE A)
(SEQ ID NO: 375)
MSSENCFVAENSSLHPESGQENDATSPHFSTRHEGSFQVPVLCAVMNVVFITILIIALIALS
VGQYNGGSGPVRRYSRVFTLEDFVGDWRQTAGYNLDQVLEQGGVSSLFQNLGVSVTPI
QRIVLSGENGLKIDIHVIIPYEGLSGDQMGQIEKIFKVVYPVDDHHFKVILHYGTLVIDGV
TPNMIDYFGRPYEGIAVFDGKKITVTGTLWNGNKIIDERLINPDGSLLFRVTINGVTGWR
LCERILAEQKLISEEDLLEPRGGSGVNPDIQMTQTTSSLSASLGDRVTISCRASQDISKYLN
WYQQKPDGTVKLLIYHTSRLHSGVPSRFSGSGSGTDYSLTISNLEQEDIATYFCQQGNTL
PYTFGGGTKLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKMNSLQTDDT
AIYYCAKHYYYGGSYAMDYWGQGTSVTVSSVNSGGGGSGGGGSAKPTTTPAPRPPTPA
PTIASQPLSLRPEACRPAAGGAVHTRGLDFAPRKIEVMYPPPYLDNEKSNGTIIHVKGKH
LCPSPLFPGPSKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLS
TATKDTYDALHMQALPPR

Which is encoded by cDNA:

(SEQ ID NO: 360)
ATGAGCTCTGAGAACTGCTTCGTGGCCGAGAACAGCAGCCTCCACCCCGAGAGTGG
TCAGGAGAACGACGCCACCAGCCCCCACTTCAGCACAAGACACGAGGGCTCATTCC
AGGTGCCTGTGCTGTGCGCCGTGATGAACGTGGTGTTCATCACCATCCTGATCATCG
CCCTGATTGCCCTTTCTGTGGGCCAATACAATGGCGGCTCCGGTCCTGTGCGCAGAT
ACTCTAGAGTGTTCACCCTCGAAGATTTCGTGGGCGACTGGAGACAGACCGCCGGCT
ACAACCTGGACCAGGTGCTGGAACAGGGCGGCGTGAGCTCTCTGTTTCAGAATCTGG
GGGTTAGCGTGACACCAATCCAGAGGATCGTGCTGTCCGGCGAGAACGGGCTGAAG
ATCGACATCCACGTGATCATTCCATACGAGGGCCTGTCTGGCGATCAGATGGGCCAG
ATCGAGAAGATCTTCAAGGTGGTGTACCCTGTAGACGACCACCATTTTAAGGTGATC
CTGCACTACGGCACCCTGGTCATCGACGGCGTGACACCCAACATGATCGACTACTTC
GGCAGACCATATGAGGGTATTGCCGTGTTTGATGGAAAGAAGATCACAGTCACCGG
CACCCTGTGGAACGGCAACAAGATTATCGATGAACGGCTGATCAACCCCGACGGCT
CTCTGCTGTTTAGAGTGACCATCAATGGCGTGACAGGCTGGAGACTGTGCGAGAGA
ATCCTGGCTGAGCAGAAACTGATCAGCGAGGAAGACCTGCTCGAGCCTAGGGGCGG
CAGCGGAGTTAACCCCGATATTCAGATGACTCAGACTACCAGCTCCCTGAGCGCATC
CCTGGGCGACCGAGTGACAATCAGTTGCCGAGCCAGCCAGGACATCAGCAAGTATC
TGAACTGGTACCAGCAGAAGCCTGACGGAACCGTCAAACTGCTGATCTATCACACAT
CCCGCCTGCATTCTGGCGTGCCAAGTCGGTTCTCTGGAAGTGGCTCAGGGACCGACT
ACTCACTGACAATCAGCAACCTGGAGCAGGAAGATATTGCCACCTATTTCTGCCAGC
AGGGCAATACTCTGCCTTACACCTTTGGCGGGGGAACTAAGCTGGAGATTACCGGCG
GAGGAGGCAGCGGAGGAGGAGGGTCCGGAGGCGGGGGATCTGAGGTGAAACTGCA
GGAAAGCGGACCTGGCCTGGTCGCTCCAAGCCAGTCCCTGTCTGTGACTTGTACCGT
CAGCGGAGTGTCCCTGCCCGATTACGGCGTCTCTTGGATCAGACAGCCCCCTAGAAA
GGGCCTGGAGTGGCTGGGCGTGATCTGGGGAAGTGAAACCACATACTATAATTCCG
CCCTGAAAAGCCGGCTGACAATCATTAAGGACAACAGTAAATCACAGGTGTTCCTG
AAGATGAATTCCCTGCAGACAGACGATACTGCAATCTACTATTGCGCCAAACACTAC
TATTACGGCGGGAGCTATGCTATGGATTACTGGGGGCAGGGAACCTCCGTCACAGTG
TCTAGTGTTAACTCAGGCGGCGGCGGCAGCGGAGGCGGAGGCAGTGCCAAGCCTAC
AACCACCCCCGCCCCTAGACCTCCTACACCTGCCCCTACCATCGCTAGCCAGCCACT
CTCTCTCAGACCTGAAGCCTGTAGACCCGCCGCTGGCGGCGCCGTGCACACCAGAGG
CCTGGACTTCGCGCCTCGGAAGATCGAGGTGATGTACCCCCCGCCTTACCTGGACAA
CGAGAAGTCTAATGGAACAATCATCCACGTGAAGGGAAAACACCTGTGTCCTTCTCC
TCTGTTCCCTGGCCCCAGCAAGCCTTTCTGGGTCCTGGTGGTGGTCGGCGGCGTGCT
GGCGTGCTACAGCCTGCTCGTGACCGTGGCCTTCATCATTTTCTGGGTGCGGAGCAA
ACGGAGCAGACTGCTGCATAGCGATTACATGAACATGACCCCAAGAAGACCTGGGC
CAACCAGAAAGCACTACCAGCCTTACGCCCCCCCCAGAGATTTCGCCGCTTATAGAA
GCCGGGTGAAGTTCTCCAGATCCGCCGACGCCCCTGCTTACCAACAGGGCCAGAACC
AGCTGTATAACGAGCTGAATCTGGGCAGAAGAGAGGAGTACGACGTGCTGGATAAG
CGGAGAGGAAGAGACCCTGAGATGGGCGGCAAGCCCCGGCGGAAGAACCCTCAGG
AGGGCCTGTATAACGAGCTGCAAAAAGATAAGATGGCCGAGGCTTATAGCGAGATC
GGAATGAAGGGCGAGCGGCGGAGGGGCAAAGGCCACGACGGCCTGTACCAGGGAC
TGAGCACAGCCACAAAGGACACCTACGACGCTCTGCACATGCAGGCCCTGCCTCCTA
GAtaaTGTACA
CD69Cyt-TM-GrzB(IEPD)-nLuc-Myc-GrzB(IEPD)-a-CD19sc-Stalk-CD28-CD3z
(SEQ ID NO: 383)
MSSENCFVAENSSLHPESGQENDATSPHFSTRHEGSFQVPVLCAVMNVVFITILIIALIALS
VGQYNGGSGPVRRYSRQGGSGGGIEPDSGGSGSRVFTLEDFVGDWRQTAGYNLDQVLE
QGGVSSLFQNLGVSVTPIQRIVLSGENGLKIDIHVIIPYEGLSGDQMGQIEKIFKVVYPVD
DHHFKVILHYGTLVIDGVTPNMIDYFGRPYEGIAVFDGKKITVTGTLWNGNKIIDERLINP
DGSLLFRVTINGVTGWRLCERILAEQKLISEEDLLEGGSGIEPDGGSGGGSGLEPRGGSGV
NPDIQMTQTTSSLSASLGDRVTISCRASQDISKYLNWYQQKPDGTVKLLIYHTSRLHSGV
PSRFSGSGSGTDYSLTISNLEQEDIATYFCQQGNTLPYTFGGGTKLEITGGGGSGGGGSGG
GGSEVKLQESGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYAMDYWGQGT
SVTVSSVNSGGGGSGGGGSAKPTTTPAPRPPTPAPTIASQPLSLRPEACRPAAGGAVHTR
GLDFAPRKIEVMYPPPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAPPRDFAAYRSRVKF
SRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR

Which is encoded by cDNA:

(SEQ ID NO: 361)
GCCAGTCCTCCGATTGACTGAGTCGCCCGGATCCCGCCACCATGAGCTCTGAGAA
CTGCTTCGTGGCCGAGAACAGCAGCCTCCACCCCGAGAGTGGTCAGGAGAACGA
CGCCACCAGCCCCCACTTCAGCACAAGACACGAGGGCTCATTCCAGGTGCCTGT
GCTGTGCGCCGTGATGAACGTGGTGTTCATCACCATCCTGATCATCGCCCTGATT
GCCCTTTCTGTGGGCCAATACAATGGCGGCTCCGGTCCTGTGCGCAGATACTCTA
GACAGGGCGGCTCTGGCGGAGGCATCGAGCCTGACAGCGGCGGCTCCGGCTCTA
GAGTGTTCACCCTCGAAGATTTCGTGGGCGACTGGAGACAGACCGCCGGCTACA
ACCTGGACCAGGTGCTGGAACAGGGCGGCGTGAGCTCTCTGTTTCAGAATCTGG
GGGTTAGCGTGACACCAATCCAGAGGATCGTGCTGTCCGGCGAGAACGGGCTGA
AGATCGACATCCACGTGATCATTCCATACGAGGGCCTGTCTGGCGATCAGATGG
GCCAGATCGAGAAGATCTTCAAGGTGGTGTACCCTGTAGACGACCACCATTTTAA
GGTGATCCTGCACTACGGCACCCTGGTCATCGACGGCGTGACACCCAACATGATC
GACTACTTCGGCAGACCATATGAGGGTATTGCCGTGTTTGATGGAAAGAAGATC
ACAGTCACCGGCACCCTGTGGAACGGCAACAAGATTATCGATGAACGGCTGATC
AACCCCGACGGCTCTCTGCTGTTTAGAGTGACCATCAATGGCGTGACAGGCTGGA
GACTGTGCGAGAGAATCCTGGCTGAGCAGAAACTGATCAGCGAGGAAGACCTGC
TCGAGGGCGGTAGCGGCATCGAGCCTGACGGCGGGAGCGGAGGCGGCAGTGGC
CTCGAGCCTAGGGGCGGCAGCGGAGTTAACCCCGATATTCAGATGACTCAGACT
ACCAGCTCCCTGAGCGCATCCCTGGGCGACCGAGTGACAATCAGTTGCCGAGCC
AGCCAGGACATCAGCAAGTATCTGAACTGGTACCAGCAGAAGCCTGACGGAACC
GTCAAACTGCTGATCTATCACACATCCCGCCTGCATTCTGGCGTGCCAAGTCGGT
TCTCTGGAAGTGGCTCAGGGACCGACTACTCACTGACAATCAGCAACCTGGAGC
AGGAAGATATTGCCACCTATTTCTGCCAGCAGGGCAATACTCTGCCTTACACCTT
TGGCGGGGGAACTAAGCTGGAGATTACCGGCGGAGGAGGCAGCGGAGGAGGAG
GGTCCGGAGGCGGGGGATCTGAGGTGAAACTGCAGGAAAGCGGACCTGGCCTG
GTCGCTCCAAGCCAGTCCCTGTCTGTGACTTGTACCGTCAGCGGAGTGTCCCTGC
CCGATTACGGCGTCTCTTGGATCAGACAGCCCCCTAGAAAGGGCCTGGAGTGGC
TGGGCGTGATCTGGGGAAGTGAAACCACATACTATAATTCCGCCCTGAAAAGCC
GGCTGACAATCATTAAGGACAACAGTAAATCACAGGTGTTCCTGAAGATGAATT
CCCTGCAGACAGACGATACTGCAATCTACTATTGCGCCAAACACTACTATTACGG
CGGGAGCTATGCTATGGATTACTGGGGGCAGGGAACCTCCGTCACAGTGTCTAG
TGTTAACTCAGGCGGCGGCGGCAGCGGAGGCGGAGGCAGTGCCAAGCCTACAAC
CACCCCCGCCCCTAGACCTCCTACACCTGCCCCTACCATCGCTAGCCAGCCACTC
TCTCTCAGACCTGAAGCCTGTAGACCCGCCGCTGGCGGCGCCGTGCACACCAGA
GGCCTGGACTTCGCGCCTCGGAAGATCGAGGTGATGTACCCCCCGCCTTACCTGG
ACAACGAGAAGTCTAATGGAACAATCATCCACGTGAAGGGAAAACACCTGTGTC
CTTCTCCTCTGTTCCCTGGCCCCAGCAAGCCTTTCTGGGTCCTGGTGGTGGTCGGC
GGCGTGCTGGCGTGCTACAGCCTGCTCGTGACCGTGGCCTTCATCATTTTCTGGG
TGCGGAGCAAACGGAGCAGACTGCTGCATAGCGATTACATGAACATGACCCCAA
GAAGACCTGGGCCAACCAGAAAGCACTACCAGCCTTACGCCCCCCCCAGAGATT
TCGCCGCTTATAGAAGCCGGGTGAAGTTCTCCAGATCCGCCGACGCCCCTGCTTA
CCAACAGGGCCAGAACCAGCTGTATAACGAGCTGAATCTGGGCAGAAGAGAGG
AGTACGACGTGCTGGATAAGCGGAGAGGAAGAGACCCTGAGATGGGCGGCAAG
CCCCGGCGGAAGAACCCTCAGGAGGGCCTGTATAACGAGCTGCAAAAAGATAAG
ATGGCCGAGGCTTATAGCGAGATCGGAATGAAGGGCGAGCGGCGGAGGGGCAA
AGGCCACGACGGCCTGTACCAGGGACTGAGCACAGCCACAAAGGACACCTACGA
CGCTCTGCACATGCAGGCCCTGCCTCCTAGATAATGTACAAGTAAAGCGGCCGG
CC
CD69Cyt-TM-GrzB(SASA)-nLuc-Myc-GrzB(SASA)-a-CD19sc-Stalk-CD28-CD3z
(SEQ ID NO: 376)
MSSENCFVAENSSLHPESGQENDATSPHFSTRHEGSFQVPVLCAVMNVVFITILIIALIALS
VGQYNGGSGPVRRYSRQGGSGGGAQGVISASASNLDDFYSGGSGSRVFTLEDFVGDWR
QTAGYNLDQVLEQGGVSSLFQNLGVSVTPIQRIVLSGENGLKIDIHVIIPYEGLSGDQMG
QIEKIFKVVYPVDDHHFKVILHYGTLVIDGVTPNMIDYFGRPYEGIAVFDGKKITVTGTL
WNGNKIIDERLINPDGSLLFRVTINGVTGWRLCERILAEQKLISEEDLLEGGSGAQGVISA
SASNLDDFYGGSGGGSGLEPRGGSGVNPDIQMTQTTSSLSASLGDRVTISCRASQDISKY
LNWYQQKPDGTVKLLIYHTSRLHSGVPSRFSGSGSGTDYSLTISNLEQEDIATYFCQQGN
TLPYTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQTDD
TAIYYCAKHYYYGGSYAMDYWGQGTSVTVSSVNSGGGGSGGGGSAKPTTTPAPRPPTP
APTIASQPLSLRPEACRPAAGGAVHTRGLDFAPRKIEVMYPPPYLDNEKSNGTIIHVKGK
HLCPSPLFPGPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKGHDGLYQ
GLSTATKDTYDALHMQALPPR

Which is encoded by cDNA:

(SEQ ID NO: 362)
GCCAGTCCTCCGATTGACTGAGTCGCCCGGATCCCGCCACCATGAGCTCTGAGAACT
GCTTCGTGGCCGAGAACAGCAGCCTCCACCCCGAGAGTGGTCAGGAGAACGACGCC
ACCAGCCCCCACTTCAGCACAAGACACGAGGGCTCATTCCAGGTGCCTGTGCTGTGC
GCCGTGATGAACGTGGTGTTCATCACCATCCTGATCATCGCCCTGATTGCCCTTTCTG
TGGGCCAATACAATGGCGGCTCCGGTCCTGTGCGCAGATACTCTAGACAGGGCGGCT
CTGGCGGAGGCGCCCAGGGCGTGATTAGCGCCAGCGCCAGCAATCTGGACGACTTT
TACAGCGGCGGCTCCGGCTCTAGAGTGTTCACCCTCGAAGATTTCGTGGGCGACTGG
AGACAGACCGCCGGCTACAACCTGGACCAGGTGCTGGAACAGGGCGGCGTGAGCTC
TCTGTTTCAGAATCTGGGGGTTAGCGTGACACCAATCCAGAGGATCGTGCTGTCCGG
CGAGAACGGGCTGAAGATCGACATCCACGTGATCATTCCATACGAGGGCCTGTCTG
GCGATCAGATGGGCCAGATCGAGAAGATCTTCAAGGTGGTGTACCCTGTAGACGAC
CACCATTTTAAGGTGATCCTGCACTACGGCACCCTGGTCATCGACGGCGTGACACCC
AACATGATCGACTACTTCGGCAGACCATATGAGGGTATTGCCGTGTTTGATGGAAAG
AAGATCACAGTCACCGGCACCCTGTGGAACGGCAACAAGATTATCGATGAACGGCT
GATCAACCCCGACGGCTCTCTGCTGTTTAGAGTGACCATCAATGGCGTGACAGGCTG
GAGACTGTGCGAGAGAATCCTGGCTGAGCAGAAACTGATCAGCGAGGAAGACCTGC
TCGAGGGCGGTAGCGGCGCCCAGGGCGTGATTAGCGCCAGCGCCAGCAATCTGGAC
GACTTTTACGGCGGGAGCGGAGGCGGCAGTGGCCTCGAGCCTAGGGGCGGCAGCGG
AGTTAACCCCGATATTCAGATGACTCAGACTACCAGCTCCCTGAGCGCATCCCTGGG
CGACCGAGTGACAATCAGTTGCCGAGCCAGCCAGGACATCAGCAAGTATCTGAACT
GGTACCAGCAGAAGCCTGACGGAACCGTCAAACTGCTGATCTATCACACATCCCGCC
TGCATTCTGGCGTGCCAAGTCGGTTCTCTGGAAGTGGCTCAGGGACCGACTACTCAC
TGACAATCAGCAACCTGGAGCAGGAAGATATTGCCACCTATTTCTGCCAGCAGGGC
AATACTCTGCCTTACACCTTTGGCGGGGGAACTAAGCTGGAGATTACCGGCGGAGG
AGGCAGCGGAGGAGGAGGGTCCGGAGGCGGGGGATCTGAGGTGAAACTGCAGGAA
AGCGGACCTGGCCTGGTCGCTCCAAGCCAGTCCCTGTCTGTGACTTGTACCGTCAGC
GGAGTGTCCCTGCCCGATTACGGCGTCTCTTGGATCAGACAGCCCCCTAGAAAGGGC
CTGGAGTGGCTGGGCGTGATCTGGGGAAGTGAAACCACATACTATAATTCCGCCCTG
AAAAGCCGGCTGACAATCATTAAGGACAACAGTAAATCACAGGTGTTCCTGAAGAT
GAATTCCCTGCAGACAGACGATACTGCAATCTACTATTGCGCCAAACACTACTATTA
CGGCGGGAGCTATGCTATGGATTACTGGGGGCAGGGAACCTCCGTCACAGTGTCTA
GTGTTAACTCAGGCGGCGGCGGCAGCGGAGGCGGAGGCAGTGCCAAGCCTACAACC
ACCCCCGCCCCTAGACCTCCTACACCTGCCCCTACCATCGCTAGCCAGCCACTCTCTC
TCAGACCTGAAGCCTGTAGACCCGCCGCTGGCGGCGCCGTGCACACCAGAGGCCTG
GACTTCGCGCCTCGGAAGATCGAGGTGATGTACCCCCCGCCTTACCTGGACAACGAG
AAGTCTAATGGAACAATCATCCACGTGAAGGGAAAACACCTGTGTCCTTCTCCTCTG
TTCCCTGGCCCCAGCAAGCCTTTCTGGGTCCTGGTGGTGGTCGGCGGCGTGCTGGCG
TGCTACAGCCTGCTCGTGACCGTGGCCTTCATCATTTTCTGGGTGCGGAGCAAACGG
AGCAGACTGCTGCATAGCGATTACATGAACATGACCCCAAGAAGACCTGGGCCAAC
CAGAAAGCACTACCAGCCTTACGCCCCCCCCAGAGATTTCGCCGCTTATAGAAGCCG
GGTGAAGTTCTCCAGATCCGCCGACGCCCCTGCTTACCAACAGGGCCAGAACCAGCT
GTATAACGAGCTGAATCTGGGCAGAAGAGAGGAGTACGACGTGCTGGATAAGCGGA
GAGGAAGAGACCCTGAGATGGGCGGCAAGCCCCGGCGGAAGAACCCTCAGGAGGG
CCTGTATAACGAGCTGCAAAAAGATAAGATGGCCGAGGCTTATAGCGAGATCGGAA
TGAAGGGCGAGCGGCGGAGGGGCAAAGGCCACGACGGCCTGTACCAGGGACTGAG
CACAGCCACAAAGGACACCTACGACGCTCTGCACATGCAGGCCCTGCCTCCTAGATA
ATGTACAAGTAAAGCGGCCGGCC
CD69Cyt-TM-GrzB(ADKG)-TNFa-Myc-GrzB(ADKG)-a-CD19sc-Stalk-CD28-CD3z
(SEQ ID NO: 384)
MSSENCFVAENSSLHPESGQENDATSPHFSTRHEGSFQVPVLCAVMNVVFITILIIALIALS
VGQYNGGSGPVRRYSRQGGSGGGLEADKGKLEYDSGGSGSRVFTLEDFVGDWRQTAG
YNLDQVLEQGGVSSLFQNLGVSVTPIQRIVLSGENGLKIDIHVIIPYEGLSGDQMGQIEKIF
KVVYPVDDHHFKVILHYGTLVIDGVTPNMIDYFGRPYEGIAVFDGKKITVTGTLWNGNK
IIDERLINPDGSLLFRVTINGVTGWRLCERILAEQKLISEEDLLEGGSGLEADKGKLEYDG
GSGGGSGLEPRGGSGVNPDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKH
YYYGGSYAMDYWGQGTSVTVSSVNSGGGGSGGGGSAKPTTTPAPRPPTPAPTIASQPLS
LRPEACRPAAGGAVHTRGLDFAPRKIEVMYPPPYLDNEKSNGTIIHVKGKHLCPSPLFPG
PSKPFWVLVVVGGVLACYSLLVTVAFIIFWVRSKRSRLLHSDYMNMTPRRPGPTRKHYQ
PYAPPRDFAAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDTYD
ALHMQALPPR

Which is encoded by cDNA:

(SEQ ID NO: 363)
GCCAGTCCTCCGATTGACTGAGTCGCCCGGATCCCGCCACCATGAGCTCT
GAGAACTGCTTCGTGGCCGAGAACAGCAGCCTCCACCCCGAGAGTGGTCA
GGAGAACGACGCCACCAGCCCCCACTTCAGCACAAGACACGAGGGCTCAT
TCCAGGTGCCTGTGCTGTGCGCCGTGATGAACGTGGTGTTCATCACCATC
CTGATCATCGCCCTGATTGCCCTTTCTGTGGGCCAATACAATGGCGGCTC
CGGTCCTGTGCGCAGATACTCTAGACAGGGCGGCTCTGGCGGAGGCCTGG
AGGCCGACAAGGGCAAGCTGGAGTACGATAGCGGCGGCTCCGGCTCTAGA
GTGTTCACCCTCGAAGATTTCGTGGGCGACTGGAGACAGACCGCCGGCTA
CAACCTGGACCAGGTGCTGGAACAGGGCGGCGTGAGCTCTCTGTTTCAGA
ATCTGGGGGTTAGCGTGACACCAATCCAGAGGATCGTGCTGTCCGGCGAG
AACGGGCTGAAGATCGACATCCACGTGATCATTCCATACGAGGGCCTGTC
TGGCGATCAGATGGGCCAGATCGAGAAGATCTTCAAGGTGGTGTACCCTG
TAGACGACCACCATTTTAAGGTGATCCTGCACTACGGCACCCTGGTCATC
GACGGCGTGACACCCAACATGATCGACTACTTCGGCAGACCATATGAGGG
TATTGCCGTGTTTGATGGAAAGAAGATCACAGTCACCGGCACCCTGTGGA
ACGGCAACAAGATTATCGATGAACGGCTGATCAACCCCGACGGCTCTCTG
CTGTTTAGAGTGACCATCAATGGCGTGACAGGCTGGAGACTGTGCGAGAG
AATCCTGGCTGAGCAGAAACTGATCAGCGAGGAAGACCTGCTCGAGGGCG
GTAGCGGCCTGGAGGCCGACAAGGGCAAGCTGGAGTACGATGGCGGGAGC
GGAGGCGGCAGTGGCCTCGAGCCTAGGGGCGGCAGCGGAGTTAACCCCGA
TATTCAGATGACTCAGACTACCAGCTCCCTGAGCGCATCCCTGGGCGACC
GAGTGACAATCAGTTGCCGAGCCAGCCAGGACATCAGCAAGTATCTGAAC
TGGTACCAGCAGAAGCCTGACGGAACCGTCAAACTGCTGATCTATCACAC
ATCCCGCCTGCATTCTGGCGTGCCAAGTCGGTTCTCTGGAAGTGGCTCAG
GGACCGACTACTCACTGACAATCAGCAACCTGGAGCAGGAAGATATTGCC
ACCTATTTCTGCCAGCAGGGCAATACTCTGCCTTACACCTTTGGCGGGGG
AACTAAGCTGGAGATTACCGGCGGAGGAGGCAGCGGAGGAGGAGGGTCCG
GAGGCGGGGGATCTGAGGTGAAACTGCAGGAAAGCGGACCTGGCCTGGTC
GCTCCAAGCCAGTCCCTGTCTGTGACTTGTACCGTCAGCGGAGTGTCCCT
GCCCGATTACGGCGTCTCTTGGATCAGACAGCCCCCTAGAAAGGGCCTGG
AGTGGCTGGGCGTGATCTGGGGAAGTGAAACCACATACTATAATTCCGCC
CTGAAAAGCCGGCTGACAATCATTAAGGACAACAGTAAATCACAGGTGTT
CCTGAAGATGAATTCCCTGCAGACAGACGATACTGCAATCTACTATTGCG
CCAAACACTACTATTACGGCGGGAGCTATGCTATGGATTACTGGGGGCAG
GGAACCTCCGTCACAGTGTCTAGTGTTAACTCAGGCGGCGGCGGCAGCGG
AGGCGGAGGCAGTGCCAAGCCTACAACCACCCCCGCCCCTAGACCTCCTA
CACCTGCCCCTACCATCGCTAGCCAGCCACTCTCTCTCAGACCTGAAGCC
TGTAGACCCGCCGCTGGCGGCGCCGTGCACACCAGAGGCCTGGACTTCGC
GCCTCGGAAGATCGAGGTGATGTACCCCCCGCCTTACCTGGACAACGAGA
AGTCTAATGGAACAATCATCCACGTGAAGGGAAAACACCTGTGTCCTTCT
CCTCTGTTCCCTGGCCCCAGCAAGCCTTTCTGGGTCCTGGTGGTGGTCGG
CGGCGTGCTGGCGTGCTACAGCCTGCTCGTGACCGTGGCCTTCATCATTT
TCTGGGTGCGGAGCAAACGGAGCAGACTGCTGCATAGCGATTACATGAAC
ATGACCCCAAGAAGACCTGGGCCAACCAGAAAGCACTACCAGCCTTACGC
CCCCCCCAGAGATTTCGCCGCTTATAGAAGCCGGGTGAAGTTCTCCAGAT
CCGCCGACGCCCCTGCTTACCAACAGGGCCAGAACCAGCTGTATAACGAG
CTGAATCTGGGCAGAAGAGAGGAGTACGACGTGCTGGATAAGCGGAGAGG
AAGAGACCCTGAGATGGGCGGCAAGCCCCGGCGGAAGAACCCTCAGGAGG
GCCTGTATAACGAGCTGCAAAAAGATAAGATGGCCGAGGCTTATAGCGAG
ATCGGAATGAAGGGCGAGCGGCGGAGGGGCAAAGGCCACGACGGCCTGTA
CCAGGGACTGAGCACAGCCACAAAGGACACCTACGACGCTCTGCACATGC
AGGCCCTGCCTCCTAGATAATGTACAAGTAAAGCGGCCGGCC

### Example 19: Creation of Lentiviral vectors

The transfer vector containing the gene of interest was transfected into 293T cells along with the lentiviral packaging vectors (pMDLg/pRRE and pRSV-Rev), and an envelope vector (phCMV-VSV-G) and the viral supernatant was harvested.

### Small-Scale Production of VSV-G Pseudotyped Lentiviral Vectors

This method describes the production of VSV-G pseudotyped lentiviral vectors in 6-well plates using a calcium phosphate transfection kit. The amount of virus supernatant per well was 4 ml, and the virus concentration depended on the vector used.

TABLE 6
Materials

| Material | Company | Catalog no |
|---|---|---|
| 293FT cells | Invitrogen | R700-07 |
| DMEM (high glucose) | Invitrogen | 11965-092 |
| FBS | Invitrogen | 16000-044 |
| PBS | Invitrogen | 10010-023 |
| MEM non-essential amino | Sigma-Aldrich | M7145 |
| acid solution (100X) | | |
| L-glutamine (200 mM) | Sigma-Aldrich | G7513 |
| Sodium pyruvate (100 mM) | Sigma-Aldrich | S8636 |
| HEPES solution (1M) | Sigma-Aldrich | H3537 |
| Poly-D-Lysine coated | BD Biocoat | 354550 |
| 150 mm Dish | | |
| TrypLE Express stable | Invitrogen | 12604-013 |
| trypsin replacement | | |
| Calcium phosphate | Sigma-Aldrich | CAPHOS-1KT |
| transfection kit | | |
| Chloroquine | Sigma-Aldrich | C6628 |
| 0.45 μm Millex HV filters | Millipore | SLHV 033RS |

### General Materials:

T75 flasks, cell culture pipettes, micropipettes and tips, 1.5 ml microcentrifuge tubes, 5 ml syringes, Trypan Blue, Virkon, and M-ytdes.

### Plasmids

Vector: depending on the choice
Gag-pol plasmid: pMDLg/pRRE
Rev plasmid: pRSV-Rev
Envelope plasmid: phCMV-VSV-G

### Procedure

### Maintaining IEK293FT Cells

Maintain cells by splitting them every other day 1:5-1:4 and keep them in a T75 or T150 flask. After thawing the cells, culture them for at least 3 passages before using them for virus production so that they recover and start exponential growth. Using cells with high passage numbers is not recommended, as this negatively affects virus production.

### Day 1: Plating & Transfection

Plate 500.000 cells/well in complete growth medium.
Pipette off the medium from the flask.
Wash the cells with 10 ml of room temperature PBS
Add 1 ml (T75) or 2 ml (T150) of TrypLE Express and incubate at 37° C. for 5 minutes
After incubation, add 10-20 ml of the complete medium into the flask, resuspend thoroughly and dissociate all the clumps by pipetting up and down several times
Count the cells using Trypan Blue and make a cell suspension of 250.000 cells/ml
Put 2 ml of this suspension into a well (Poly-L-Lysine coated 6-well plate). Prepare one well for each vector.
Put the plate in the incubator for at least 7-8 hours

After incubation, transfect the cells. Confirm that the cells have been attached, and a confluence of optimally 80% is obtained. If the cells are viable and at an appropriate density, put the plate back in the incubator and prepare the transfection mix. Abort the transfection if confluency is below 60%.
Prepare 1 ml of complete growth medium with the addition of Chloroquine at a final concentration of 25 M. Put it into the incubator for pre-warming. It is important that the components of the calcium phosphate precipitation kit are brought to room temperature before starting the transfection.

Prepare the plasmid mixture into a microcentrifuge tube as follows (4 μg DNA in total):
2 g of LeGO-iG2 vector (including transgene)
1 g of pMDLg/pRRE (Gag/Pol)
0.75 g of pRSV-REV (Rev)
0.25 g of phCMV-VSV-G (Envelope)
Mix the plasmids, and adjust the volume up to 54 μl with ddH₂O
Add 6 μl of 2.5 M CaCl₂ solution to the DNA mixture
Into a separate microtube, put 60 μl of 2×HeBS buffer. Add the CaCl₂/DNA mixture and vortex
Let this mixture sit at room temperature for 15 minutes. Do not allow it to sit for more than 30 minutes, as this might decrease the transfection efficiency.
During these 15 minutes, take your dish out, discard the medium and add 1 ml prewarmed full-growth medium containing 25 M Chloroquine.
After the 15-minute incubation, add the 120 μl mixture into the well dropwise, while gently swirling the dish in a circular motion.
Close the lid of the dish and put it back into the incubator for 10-12 hours.

### Day 2: Medium Change

10-12 hours post-transfection:
Aspirate the medium containing the transfection mix and chloroquine from the wells and discard.
Add 2 ml of complete growth medium per well. Ensure the medium is prewarmed at least to room temperature, preferably 37° C. Put the cells into the incubator.

### Day 3: Collection of Supernatant 1

24 hours post medium change:
Check the cells under a UV microscope for GFP expression. The transfection efficiency should be above 90%.
Prepare a 0.45 m filter, a 5 ml syringe, a 5 ml microtube and a 1.5 tube per well.
Harvest the medium from the dish using the 5 ml syringe. Apply the filter and filter the supernatant into the 5 ml microtube. Be gentle while filtering, do not create air bubbles, and do not apply extreme force. When you are done, drop the syringe and filter into the Virkon solution.
Take a 100 μl aliquot from the filtered supernatant into the 1.5 ml microcentrifuge tube (virus titration). Aliquot the rest of the supernatant as you wish and freeze at −80° C. for long-term storage.
Add 2 ml of full-growth medium per dish. Ensure the medium is prewarmed at least to room temperature, preferably 37° C. Put the cells into the incubator.

### Day 4: Collection of Supernatant 2

48 hours post medium change:
Collect virus supernatants in the same manner as the previous day.
Discard the plates.

### Generation of Cell Lines

The lentiviral particles harvested in the procedure mentioned above were transduced into K562 and KHYG-1 cells, sorted, and expanded. The resulting cells were tested for the expression of the target genes either using the corresponding antibodies or fluorescent-labeled proteins.
1. Prepare medium RPMI 10% FBS=400ul/well
2. Add lentiviral vector and transduction medium.
3. Mark a 24-well plate with the date, name, cell type and what virus you use for transduction.
4. Set the temperature of the centrifuge to 32° C.
5. Count the cells using trypan blue and make a cell suspension with 106 cells/ml in medium+10% FBS.
6. Distribute 250 μl of 50,000 cells to 24 well plate.
7. Take the required protamine sulfate stock for a final concentration of 8 μg/ml.
8. Avoid repeated freeze/thaw of the stock.
9. Add the medium according to the calculations.
10. Keep the virus on dry ice until usage. Thaw the required amount of virus quickly.
11. Mix the virus carefully so that contact with air is minimal.
12. Add the calculated amount of virus to each well.
13. Pipette protamine sulfate (8 μg/ml) and IL-2 (100 IU/ml) to each well in the case of KHYG-1 cells.
14. Mix the cells carefully by pipetting up and down.
15. Centrifuge the plate with 1000×g for 1 hr at 32° C. without a break.
16. Take out the plate and incubate between 4 hr and overnight (depending on your construct and virus titer; should be tested) in the incubator.
17. At the end of the incubation, centrifuge the plate again with 1000×g for 1 hr at 32° C.
18. Remove 80% of the medium carefully from all the wells and fill with 500 ul fresh pre-warmed medium with serum.
19. Put the plate back into the incubator.

Day 1 & 2: Check the cells under the microscope and search for colonies.

Day 3: Analyze the cells with flow cytometry.

### HEK293 NF-κB Luciferase Assay Procedure

1. The day before the assay, harvest NF-κB reporter (Luc)-HEK293 cells and seed cells at a density of 20,000 cells per well into a 96-well microplate in 100 μl of assay medium (DMEM+10% FBS+1% P/S). Incubate cells at 37 C with 5% CO₂ overnight for approximately 24 hours.
2. The next day, aspirate the medium on 96 wells microplate and add 100 L of the supernatant from GAGE expressing cells alone or co-cultured with the target cells into corresponding well and incubate at 37° C. with 5% CO₂ for 6 hours.
3. 6 hours after, aspirate the medium from the cells and add 100 L of 0.1% Triton buffer to each sample well.
4. Rock the plate slowly several times to ensure complete coverage of the cells with Lysis Buffer.
5. Incubate for 10 minutes at room temperature to allow lysis to occur.
6. Transfer 30 μL of lysate to a new white opaque 96-well plate for the Luciferase assay system and set the GLOMAX machine automatically to add 50 μL of D-Luciferine or nanoGLOW assay system (Promega) per well and measure luminescence using a luminometer following the manufacturer's instructions. The luciferase activity data presented in the results are normalized to the percentages and MFI of the GAGE expression in the cells.

### Flow Cytometry

The staining and washes were performed in a flow cytometry acquisition buffer. A single-cell suspension of the cells was incubated with a blocking reagent for 10 min on ice and then stained with antibodies and viability staining for 30 min on ice. Samples are analyzed on a Cytoflex flow cytometer (BD Biosciences), and the data are analyzed using FlowJo software (TreeStar, Ashland, OR). For sorting AriaFusion (BD Biosciences) machine was used. Sorted cells were cultured in a medium with antibiotics for two weeks. Onwards, cells were cultured without antibiotics.

### Example 20

Experiments were performed relating to the following Caspase 3 sequences and constructs:
CD69Cyt-TM-Cas3(DEVD)-TNFa-Myc-Cas3(DEVD)-a-CD19sc-Stalk-CD28-CD3z
Cas3 (DEVDG) sequences are given as BOLD_UNDERLINED_CAPITALIZED

### DNA Sequence

(SEQ ID NO: 386)
ATGAGCTCTGAGAACTGCTTCGTGGCCGAGAACAGCAGCCTCCACCCCGA
GAGTGGTCAGGAGAACGACGCCACCAGCCCCCACTTCAGCACAAGACACG
AGGGCTCATTCCAGGTGCCTGTGCTGTGCGCCGTGATGAACGTGGTGTTC
ATCACCATCCTGATCATCGCCCTGATTGCCCTTTCTGTGGGCCAATACAA
TGGCGGCTCCGGTCCTGTGCGCAGATACTCTAGACAGGGCGGCTCTGGCG
GAGGCGATGAAGTGGACGGCAGCGGCGGCTCCGGCAGCAGCAGCTCTAGA
GTGTTCACCCTCGAAGATTTCGTGGGCGACTGGAGACAGACCGCCGGCTA
CAACCTGGACCAGGTGCTGGAACAGGGCGGCGTGAGCTCTCTGTTTCAGA
ATCTGGGGGTTAGCGTGACACCAATCCAGAGGATCGTGCTGTCCGGCGAG
AACGGGCTGAAGATCGACATCCACGTGATCATTCCATACGAGGGCCTGTC
TGGCGATCAGATGGGCCAGATCGAGAAGATCTTCAAGGTGGTGTACCCTG
TAGACGACCACCATTTTAAGGTGATCCTGCACTACGGCACCCTGGTCATC
GACGGCGTGACACCCAACATGATCGACTACTTCGGCAGACCATATGAGGG
TTATCGATGAACGGCTGATCAACCCCGACGGCTCTCTGCTGTTTAGAGTG
TATTGCCGTGTTTGATGGAAAGAAGATCACAGTCACCGGCACCCTGTGGA
ACGGCAACAAGAACCATCAATGGCGTGACAGGCTGGAGACTGTGCGAGAG
AATCCTGGCTGAGCAGAAACTGATCAGCGAGGAAGACCTGCTCGAGGGCG
GTAGCGGCGATGAAGTGGACGGCGGCGGGAGCGGAGGCGGCAGTGGCCTC
GAGCCTAGGGGCGGCAGCGGAGTTAACCCCGATATTCAGATGACTCAGAC
TACCAGCTCCCTGAGCGCATCCCTGGGCGACCGAGTGACAATCAGTTGCC
GAGCCAGCCAGGACATCAGCAAGTATCTGAACTGGTACCAGCAGAAGCCT
GACGGAACCGTCAAACTGCTGATCTATCACACATCCCGCCTGCATTCTGG
CGTGCCAAGTCGGTTCTCTGGAAGTGGCTCAGGGACCGACTACTCACTGA
CAATCAGCAACCTGGAGCAGGAAGATATTGCCACCTATTTCTGCCAGCAG
GGCAATACTCTGCCTTACACCTTTGGCGGGGGAACTAAGCTGGAGATTAC
CGGCGGAGGAGGCAGCGGAGGAGGAGGGTCCGGAGGCGGGGGATCTGAGG
TGAAACTGCAGGAAAGCGGACCTGGCCTGGTCGCTCCAAGCCAGTCCCTG
TCTGTGACTTGTACCGTCAGCGGAGTGTCCCTGCCCGATTACGGCGTCTC
TTGGATCAGACAGCCCCCTAGAAAGGGCCTGGAGTGGCTGGGCGTGATCT
GGGGAAGTGAAACCACATACTATAATTCCGCCCTGAAAAGCCGGCTGACA
ATCATTAAGGACAACAGTAAATCACAGGTGTTCCTGAAGATGAATTCCCT
GCAGACAGACGATACTGCAATCTACTATTGCGCCAAACACTACTATTACG
GCGGGAGCTATGCTATGGATTACTGGGGGCAGGGAACCTCCGTCACAGTG
TCTAGTGTTAACTCAGGCGGCGGCGGCAGCGGAGGCGGAGGCAGTGCCAA
GCCTACAACCACCCCCGCCCCTAGACCTCCTACACCTGCCCCTACCATCG
CTAGCCAGCCACTCTCTCTCAGACCTGAAGCCTGTAGACCCGCCGCTGGC
GGCGCCGTGCACACCAGAGGCCTGGACTTCGCGCCTCGGAAGATCGAGGT
GATGTACCCCCCGCCTTACCTGGACAACGAGAAGTCTAATGGAACAATCA
TCCACGTGAAGGGAAAACACCTGTGTCCTTCTCCTCTGTTCCCTGGCCCC
AGCAAGCCTTTCTGGGTCCTGGTGGTGGTCGGCGGCGTGCTGGCGTGCTA
CAGCCTGCTCGTGACCGTGGCCTTCATCATTTTCTGGGTGCGGAGCAAAC
GGAGCAGACTGCTGCATAGCGATTACATGAACATGACCCCAAGAAGACCT
GGGCCAACCAGAAAGCACTACCAGCCTTACGCCCCCCCCAGAGATTTCGC
CGCTTATAGAAGCCGGGTGAAGTTCTCCAGATCCGCCGACGCCCCTGCTT
ACCAACAGGGCCAGAACCAGCTGTATAACGAGCTGAATCTGGGCAGAAGA
GAGGAGTACGACGTGCTGGATAAGCGGAGAGGAAGAGACCCTGAGATGGG
CGGCAAGCCCCGGCGGAAGAACCCTCAGGAGGGCCTGTATAACGAGCTGC
AAAAAGATAAGATGGCCGAGGCTTATAGCGAGATCGGAATGAAGGGCGAG
CGGCGGAGGGGCAAAGGCCACGACGGCCTGTACCAGGGACTGAGCACAGC
CACAAAGGACACCTACGACGCTCTGCACATGCAGGCCCTGCCTCCTAGAt
aa

CD69Cyt-TM-Cas3(DEVD)-TNFa-Myc-Cas3(DEVD)-a-CD19sc-Stalk-CD28-CD3z

### Protein Sequence

(SEQ ID NO: 387)
MSSENCFVAENSSLHPESGQENDATSPHFSTRHEGSFQVPVLCAVMNVVF
ITILIIALIALSVGQYNGGSGPVRRYSRQGGSGGGDEVDGSGGSGSSSSR
VFTLEDFVGDWRQTAGYNLDQVLEQGGVSSLFQNLGVSVTPIQRIVLSGE
NGLKIDIHVIIPYEGLSGDQMGQIEKIFKVVYPVDDHHFKVILHYGTLVI
DGVTPNMIDYFGRPYEGIAVFDGKKITVTGTLWNGNKIIDERLINPDGSL
LFRVTINGVTGWRLCERILAEQKLISEEDLLEGGSGDEVDGGGSGGGSGL
EPRGGSGVNPDIQMTQTTSSLSASLGDRVTISCRASQDISKYLNWYQQKP
DGTVKLLIYHTSRLHSGVPSRFSGSGSGTDYSLTISNLEQEDIATYFCQQ
GNTLPYTFGGGTKLEITGGGGSGGGGSGGGGSEVKLQESGPGLVAPSQSL
SVTCTVSGVSLPDYGVSWIRQPPRKGLEWLGVIWGSETTYYNSALKSRLT
IIKDNSKSQVFLKMNSLQTDDTAIYYCAKHYYYGGSYAMDYWGQGTSVTV
SSVNSGGGGSGGGGSAKPTTTPAPRPPTPAPTIASQPLSLRPEACRPAAG
GAVHTRGLDFAPRKIEVMYPPPYLDNEKSNGTIIHVKGKHLCPSPLFPGP
SKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR

CD69Cyt-TM-Cas3(EAALVDMVNDG)-TNFa-Myc-Cas3(EAALVDMVNDG)-a-CD19sc-Stalk-CD28-CD3z

Cas3 (EAALVDMVNDG (SEQ ID NO: 388)) sequences are given as BOLD_UNDERLINED_CAPITALIZED

### DNA Sequence

(SEQ ID NO: 389)
ATGAGCTCTGAGAACTGCTTCGTGGCCGAGAACAGCAGCCTCCACCCCGA
GAGTGGTCAGGAGAACGACGCCACCAGCCCCCACTTCAGCACAAGACACG
AGGGCTCATTCCAGGTGCCTGTGCTGTGCGCCGTGATGAACGTGGTGTTC
ATCACCATCCTGATCATCGCCCTGATTGCCCTTTCTGTGGGCCAATACAA
TGGCGGCTCCGGTCCTGTGCGCAGATACTCTAGAGGCGGCTCTGGCGGAG
GCGAGGCCGCCCTGGTGGATATGGTGAACGACGGCAGCGGCGGCTCCGGC
GGATCTAGAGTGTTCACCCTCGAAGATTTCGTGGGCGACTGGAGACAGAC
CGCCGGCTACAACCTGGACCAGGTGCTGGAACAGGGCGGCGTGAGCTCTC
TGTTTCAGAATCTGGGGGTTAGCGTGACACCAATCCAGAGGATCGTGCTG
TCCGGCGAGAACGGGCTGAAGATCGACATCCACGTGATCATTCCATACGA
GGGCCTGTCTGGCGATCAGATGGGCCAGATCGAGAAGATCTTCAAGGTGG
TGTACCCTGTAGACGACCACCATTTTAAGGTGATCCTGCACTACGGCACC
CTGGTCATCGACGGCGTGACACCCAACATGATCGACTACTTCGGCAGACC
ATATGAGGGTATTGCCGTGTTTGATGGAAAGAAGATCACAGTCACCGGCA
CCCTGTGGAACGGCAACAAGATTATCGATGAACGGCTGATCAACCCCGAC
GGCTCTCTGCTGTTTAGAGTGACCATCAATGGCGTGACAGGCTGGAGACT
GTGCGAGAGAATCCTGGCTGAGCAGAAACTGATCAGCGAGGAAGACCTGC
TCGAGGGCGGCTCTGGCGGAGGCGAGGCCGCCCTGGTGGATATGGTGAAC
GACGGCAGCGGCGGCTCCGGCGGACTCGAGCCTAGGGGCGGCAGCGGAGT
TAACCCCGATATTCAGATGACTCAGACTACCAGCTCCCTGAGCGCATCCC
TGGGCGACCGAGTGACAATCAGTTGCCGAGCCAGCCAGGACATCAGCAAG
TATCTGAACTGGTACCAGCAGAAGCCTGACGGAACCGTCAAACTGCTGAT
CTATCACACATCCCGCCTGCATTCTGGCGTGCCAAGTCGGTTCTCTGGAA
GTGGCTCAGGGACCGACTACTCACTGACAATCAGCAACCTGGAGCAGGAA
GATATTGCCACCTATTTCTGCCAGCAGGGCAATACTCTGCCTTACACCTT
TGGCGGGGGAACTAAGCTGGAGATTACCGGCGGAGGAGGCAGCGGAGGAG
GAGGGTCCGGAGGCGGGGGATCTGAGGTGAAACTGCAGGAAAGCGGACCT
GGCCTGGTCGCTCCAAGCCAGTCCCTGTCTGTGACTTGTACCGTCAGCGG
AGTGTCCCTGCCCGATTACGGCGTCTCTTGGATCAGACAGCCCCCTAGAA
AGGGCCTGGAGTGGCTGGGCGTGATCTGGGGAAGTGAAACCACATACTAT
AATTCCGCCCTGAAAAGCCGGCTGACAATCATTAAGGACAACAGTAAATC
ACAGGTGTTCCTGAAGATGAATTCCCTGCAGACAGACGATACTGCAATCT
ACTATTGCGCCAAACACTACTATTACGGCGGGAGCTATGCTATGGATTAC
TGGGGGCAGGGAACCTCCGTCACAGTGTCTAGTGTTAACTCAGGCGGCGG
CGGCAGCGGAGGCGGAGGCAGTGCCAAGCCTACAACCACCCCCGCCCCTA
GACCTCCTACACCTGCCCCTACCATCGCTAGCCAGCCACTCTCTCTCAGA
CCTGAAGCCTGTAGACCCGCCGCTGGCGGCGCCGTGCACACCAGAGGCCT
GGACTTCGCGCCTCGGAAGATCGAGGTGATGTACCCCCCGCCTTACCTGG
ACAACGAGAAGTCTAATGGAACAATCATCCACGTGAAGGGAAAACACCTG
TGTCCTTCTCCTCTGTTCCCTGGCCCCAGCAAGCCTTTCTGGGTCCTGGT
GGTGGTCGGCGGCGTGCTGGCGTGCTACAGCCTGCTCGTGACCGTGGCCT
TCATCATTTTCTGGGTGCGGAGCAAACGGAGCAGACTGCTGCATAGCGAT
TACATGAACATGACCCCAAGAAGACCTGGGCCAACCAGAAAGCACTACCA
GCCTTACGCCCCCCCCAGAGATTTCGCCGCTTATAGAAGCCGGGTGAAGT
TCTCCAGATCCGCCGACGCCCCTGCTTACCAACAGGGCCAGAACCAGCTG
TATAACGAGCTGAATCTGGGCAGAAGAGAGGAGTACGACGTGCTGGATAA
GCGGAGAGGAAGAGACCCTGAGATGGGCGGCAAGCCCCGGCGGAAGAACC
CTCAGGAGGGCCTGTATAACGAGCTGCAAAAAGATAAGATGGCCGAGGCT
TATAGCGAGATCGGAATGAAGGGCGAGCGGCGGAGGGGCAAAGGCCACGA
CGGCCTGTACCAGGGACTGAGCACAGCCACAAAGGACACCTACGACGCTC
TGCACATGCAGGCCCTGCCTCCTAGAtaa

CD69Cyt-TM-Cas3(EAALVDMVNDG)-TNFa-Myc-Cas3(EAALVDMVNDG)-a-CD19sc-Stalk-CD28-CD3z

### Protein Sequence

(SEQ ID NO: 390)
MSSENCFVAENSSLHPESGQENDATSPHFSTRHEGSFQVPVLCAVMNVVF
ITILIIALIALSVGQYNGGSGPVRRYSRGGSGGGEAALVDMVNDGSGGSG
GSRVFTLEDFVGDWRQTAGYNLDQVLEQGGVSSLFQNLGVSVTPIQRIVL
SGENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINPD
GSLLFRVTINGVTGWRLCERILAEQKLISEEDLLEGGSGGGEAALVDMVN
DGSGGSGGLEPRGGSGVNPDIQMTQTTSSLSASLGDRVTISCRASQDISK
YLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYY
NSALKSRLTIIKDNSKSQVFLKMNSLQTDDTAIYYCAKHYYYGGSYAMDY
WGQGTSVTVSSVNSGGGGSGGGGSAKPTTTPAPRPPTPAPTIASQPLSLR
PEACRPAAGGAVHTRGLDFAPRKIEVMYPPPYLDNEKSNGTIIHVKGKHL
CPSPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR

CD69Cyt-TM-Cas3(EDYGRDSGPP)-TNFa-Myc-Cas3(EDYGRDSGPP)-a-CD19sc-Stalk-CD28-CD3z
Cas3 (EDYGRDSGPP (SEO ID NO: 391)) sequences are given as BOLD_UNDERLINED_CAPITALIZED

### DNA Sequence

(SEQ ID NO: 392)
ATGAGCTCTGAGAACTGCTTCGTGGCCGAGAACAGCAGCCTCCACCCCGA
GAGTGGTCAGGAGAACGACGCCACCAGCCCCCACTTCAGCACAAGACACG
AGGGCTCATTCCAGGTGCCTGTGCTGTGCGCCGTGATGAACGTGGTGTTC
ATCACCATCCTGATCATCGCCCTGATTGCCCTTTCTGTGGGCCAATACAA
TGGCGGCTCCGGTCCTGTGCGCAGATACTCTAGAGGCGGCTCTGGCGGAG
GCGAGGACTACGGCAGAGATAGCGGCCCTCCCAGCGGCGGCTCCGGCAGC
AGCAGCTCTAGAGTGTTCACCCTCGAAGATTTCGTGGGCGACTGGAGACA
ACAGGGCGGCGTGAGCTCTCTGTTTCAGAATCTGGGGGTTAGCGTGACAC
GACCGCCGGCTACAACCTGGACCAGGTGCTGGACAATCCAGAGGATCGTG
CTGTCCGGCGAGAACGGGCTGAAGATCGACATCCACGTGATCATTCCATA
CGAGGGCCTGTCTGGCGATCAGATGGGCCAGATCGAGAAGATCTTCAAGG
TGGTGTACCCTGTAGACGACCACCATTTTAAGGTGATCCTGCACTACGGC
ACCCTGGTCATCGACGGCGTGACACCCAACATGATCGACTACTTCGGCAG
ACCATATGAGGGTATTGCCGTGTTTGATGGAAAGAAGATCACAGTCACCG
GCACCCTGTGGAACGGCAACAAGATTATCGATGAACGGCTGATCAACCCC
GACGGCTCTCTGCTGTTTAGAGTGACCATCAATGGCGTGACAGGCTGGAG
ACTGTGCGAGAGAATCCTGGCTGAGCAGAAACTGATCAGCGAGGAAGACC
TGCTCGAGGGCGGCTCTGGCGGAGGCGAGGACTACGGCAGAGATAGCGGC
CCTCCCAGCGGCGGCTCCGGCAGCAGCAGCCTCGAGCCTAGGGGCGGCAG
CGGAGTTAACCCCGATATTCAGATGACTCAGACTACCAGCTCCCTGAGCG
CATCCCTGGGCGACCGAGTGACAATCAGTTGCCGAGCCAGCCAGGACATC
AGCAAGTATCTGAACTGGTACCAGCAGAAGCCTGACGGAACCGTCAAACT
GCTGATCTATCACACATCCCGCCTGCATTCTGGCGTGCCAAGTCGGTTCT
CTGGAAGTGGCTCAGGGACCGACTACTCACTGACAATCAGCAACCTGGAG
CAGGAAGATATTGCCACCTATTTCTGCCAGCAGGGCAATACTCTGCCTTA
CACCTTTGGCGGGGGAACTAAGCTGGAGATTACCGGCGGAGGAGGCAGCG
GAGGAGGAGGGTCCGGAGGCGGGGGATCTGAGGTGAAACTGCAGGAAAGC
GGACCTGGCCTGGTCGCTCCAAGCCAGTCCCTGTCTGTGACTTGTACCGT
CAGCGGAGTGTCCCTGCCCGATTACGGCGTCTCTTGGATCAGACAGCCCC
CTAGAAAGGGCCTGGAGTGGCTGGGCGTGATCTGGGGAAGTGAAACCACA
TACTATAATTCCGCCCTGAAAAGCCGGCTGACAATCATTAAGGACAACAG
TAAATCACAGGTGTTCCTGAAGATGAATTCCCTGCAGACAGACGATACTG
CAATCTACTATTGCGCCAAACACTACTATTACGGCGGGAGCTATGCTATG
GATTACTGGGGGCAGGGAACCTCCGTCACAGTGTCTAGTGTTAACTCAGG
CGGCGGCGGCAGCGGAGGCGGAGGCAGTGCCAAGCCTACAACCACCCCCG
CCCCTAGACCTCCTACACCTGCCCCTACCATCGCTAGCCAGCCACTCTCT
CTCAGACCTGAAGCCTGTAGACCCGCCGCTGGCGGCGCCGTGCACACCAG
AGGCCTGGACTTCGCGCCTCGGAAGATCGAGGTGATGTACCCCCCGCCTT
ACCTGGACAACGAGAAGTCTAATGGAACAATCATCCACGTGAAGGGAAAA
CACCTGTGTCCTTCTCCTCTGTTCCCTGGCCCCAGCAAGCCTTTCTGGGT
CCTGGTGGTGGTCGGCGGCGTGCTGGCGTGCTACAGCCTGCTCGTGACCG
TGGCCTTCATCATTTTCTGGGTGCGGAGCAAACGGAGCAGACTGCTGCAT
AACCAGAAAGCACTACCAGCCTTACGCCCCCCCCAGAGATTTCGCCGCTT
AGCGATTACATGAACATGACCCCAAGAAGACCTGGGCCATAGAAGCCGGG
TGAAGTTCTCCAGATCCGCCGACGCCCCTGCTTACCAACAGGGCCAGAAC
CAGCTGTATAACGAGCTGAATCTGGGCAGAAGAGAGGAGTACGACGTGCT
GGATAAGCGGAGAGGAAGAGACCCTGAGATGGGCGGCAAGCCCCGGCGGA
AGAACCCTCAGGAGGGCCTGTATAACGAGCTGCAAAAAGATAAGATGGCC
GAGGCTTATAGCGAGATCGGAATGAAGGGCGAGCGGCGGAGGGGCAAAGG
CCACGACGGCCTGTACCAGGGACTGAGCACAGCCACAAAGGACACCTACG
ACGCTCTGCACATGCAGGCCCTGCCTCCTAGAtaa

CD69Cyt-TM-Cas3(EDYGRDSGPP)-TNFa-Myc-Cas3(EDYGRDSGPP)-a-CD19sc-Stalk-CD28-CD3z

### Protein Sequence

(SEQ ID NO: 393)
MSSENCFVAENSSLHPESGQENDATSPHFSTRHEGSFQVPVLCAVMNVVF
ITILIIALIALSVGQYNGGSGPVRRYSRGGSGGGEDYGRDSGPPSGGSGS
SSSRVFTLEDFVGDWRQTAGYNLDQVLEQGGVSSLFQNLGVSVTPIQRIV
LSGENGLKIDIHVIIPYEGLSGDQMGQIEKIFKVVYPVDDHHFKVILHYG
TLVIDGVTPNMIDYFGRPYEGIAVFDGKKITVTGTLWNGNKIIDERLINP
DGSLLFRVTINGVTGWRLCERILAEQKLISEEDLLEGGSGGGEDYGRDSG
PPSGGSGSSSLEPRGGSGVNPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSVNSGGGGSGGGGSAKPTTTPAPRPPTPAPTIASQPLS
LRPEACRPAAGGAVHTRGLDFAPRKIEVMYPPPYLDNEKSNGTIIHVKGK
HLCPSPLFPGPSKPFWVLVVVGGVLACYSLLVTVAFIIFWVRSKRSRLLH
SDYMNMTPRRPGPTRKHYQPYAPPRDFAAYRSRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR

CD69Cyt-TM-Cas3(TPSEPDSGQGPPQ)-TNFa-Myc-Cas3(TPSEPDSGQGPPQ)-a-CD19sc-Stalk-CD28-CD3z
Cas3 (TPSEPDSGQGPPQ (SEQ ID NO: 394)) sequences are given as BOLD_UNDERLINED_CAPITALIZED

### DNA Sequence

(SEQ ID NO: 395)
ATGAGCTCTGAGAACTGCTTCGTGGCCGAGAACAGCAGCCTCCACCCCGA
GAGTGGTCAGGAGAACGACGCCACCAGCCCCCACTTCAGCACAAGACACG
AGGGCTCATTCCAGGTGCCTGTGCTGTGCGCCGTGATGAACGTGGTGTTC
ATCACCATCCTGATCATCGCCCTGATTGCCCTTTCTGTGGGCCAATACAA
TGGCGGCTCCGGTCCTGTGCGCAGATACTCTAGAGGCGGCTCTGGCGGAG
GCACCCCTAGCGAGCCCGGATCTGGCAGAGGCCCTCCACAGAGCGGCGGC
TCCGGCGGATCTAGAGTGTTCACCCTCGAAGATTTCGTGGGCGACTGGAG
ACAGACCGCCGGCTACAACCTGGACCAGGTGCTGGAACAGGGCGGCGTGA
GCTCTCTGTTTCAGAATCTGGGGGTTAGCGTGACACCAATCCAGAGGATC
GTGCTGTCCGGCGAGAACGGGCTGAAGATCGACATCCACGTGATCATTCC
ATACGAGGGCCTGTCTGGCGATCAGATGGGCCAGATCGAGAAGATCTTCA
AGGTGGTGTACCCTGTAGACGACCACCATTTTAAGGTGATCCTGCACTAC
GGCACCCTGGTCATCGACGGCGTGACACCCAACATGATCGACTACTTCGG
CAGACCATATGAGGGTATTGCCGTGTTTGATGGAAAGAAGATCACAGTCA
CCGGCACCCTGTGGAACGGCAACAAGATTATCGATGAACGGCTGATCAAC
CCCGACGGCTCTCTGCTGTTTAGAGTGACCATCAATGGCGTGACAGGCTG
GAGACTGTGCGAGAGAATCCTGGCTGAGCAGAAACTGATCAGCGAGGAAG
ACCTGCTCGAGGGCGGCTCTGGCGGAGGCACCCCTAGCGAGCCCGGATCT
GGCAGAGGCCCTCCACAGAGCGGCGGCTCCGGCGGACTCGAGCCTAGGGG
CGGCAGCGGAGTTAACCCCGATATTCAGATGACTCAGACTACCAGCTCCC
TGAGCGCATCCCTGGGCGACCGAGTGACAATCAGTTGCCGAGCCAGCCAG
GACATCAGCAAGTATCTGAACTGGTACCAGCAGAAGCCTGACGGAACCGT
CAAACTGCTGATCTATCACACATCCCGCCTGCATTCTGGCGTGCCAAGTC
GGTTCTCTGGAAGTGGCTCAGGGACCGACTACTCACTGACAATCAGCAAC
CTGGAGCAGGAAGATATTGCCACCTATTTCTGCCAGCAGGGCAATACTCT
GCCTTACACCTTTGGCGGGGGAACTAAGCTGGAGATTACCGGCGGAGGAG
GCAGCGGAGGAGGAGGGTCCGGAGGCGGGGGATCTGAGGTGAAACTGCAG
GAAAGCGGACCTGGCCTGGTCGCTCCAAGCCAGTCCCTGTCTGTGACTTG
TACCGTCAGCGGAGTGTCCCTGCCCGATTACGGCGTCTCTTGGATCAGAC
AGCCCCCTAGAAAGGGCCTGGAGTGGCTGGGCGTGATCTGGGGAAGTGAA
ACCACATACTATAATTCCGCCCTGAAAAGCCGGCTGACAATCATTAAGGA
CAACAGTAAATCACAGGTGTTCCTGAAGATGAATTCCCTGCAGACAGACG
ATACTGCAATCTACTATTGCGCCAAACACTACTATTACGGCGGGAGCTAT
GCTATGGATTACTGGGGGCAGGGAACCTCCGTCACAGTGTCTAGTGTTAA
CTCAGGCGGCGGCGGCAGCGGAGGCGGAGGCAGTGCCAAGCCTACAACCA
CCCCCGCCCCTAGACCTCCTACACCTGCCCCTACCATCGCTAGCCAGCCA
CTCTCTCTCAGACCTGAAGCCTGTAGACCCGCCGCTGGCGGCGCCGTGCA
CACCAGAGGCCTGGACTTCGCGCCTCGGAAGATCGAGGTGATGTACCCCC
CGCCTTACCTGGACAACGAGAAGTCTAATGGAACAATCATCCACGTGAAG
GGAAAACACCTGTGTCCTTCTCCTCTGTTCCCTGGCCCCAGCAAGCCTTT
CTGGGTCCTGGTGGTGGTCGGCGGCGTGCTGGCGTGCTACAGCCTGCTCG
TGACCGTGGCCTTCATCATTTTCTGGGTGCGGAGCAAACGGAGCAGACTG
CTGCATAGCGATTACATGAACATGACCCCAAGAAGACCTGGGCCAACCAG
AAAGCACTACCAGCCTTACGCCCCCCCCAGAGATTTCGCCGCTTATAGAA
GCCGGGTGAAGTTCTCCAGATCCGCCGACGCCCCTGCTTACCAACAGGGC
CAGAACCAGCTGTATAACGAGCTGAATCTGGGCAGAAGAGAGGAGTACGA
CGTGCTGGATAAGCGGAGAGGAAGAGACCCTGAGATGGGCGGCAAGCCCC
GGCGGAAGAACCCTCAGGAGGGCCTGTATAACGAGCTGCAAAAAGATAAG
ATGGCCGAGGCTTATAGCGAGATCGGAATGAAGGGCGAGCGGCGGAGGGG
CAAAGGCCACGACGGCCTGTACCAGGGACTGAGCACAGCCACAAAGGACA
CCTACGACGCTCTGCACATGCAGGCCCTGCCTCCTAGAtaa

CD69Cyt-TM-Cas3(TPSEPDSGQGPPQ)-TNFa-Myc-Cas3(TPSEPDSGQGPPQ)-a-CD19sc-Stalk-CD28-CD3z
Cas3 (TPSEPDSGQGPPQ (SEQ ID NO: 394)) sequences are given as BOLD_UNDERLINED_CAPITALIZED

### Protein Sequence

(SEQ ID NO: 396)
MSSENCFVAENSSLHPESGQENDATSPHFSTRHEGSFQVPVLCAVMNVVF
ITILIIALIALSVGQYNGGSGPVRRYSRGGSGGGTPSEPGSGRGPPQSGG
SGGSRVFTLEDFVGDWRQTAGYNLDQVLEQGGVSSLFQNLGVSVTPIQRI
VLSGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERLIN
PDGSLLFRVTINGVTGWRLCERILAEQKLISEEDLLEGGSGGGTPSEPGS
GRGPPQSGGSGGLEPRGGSGVNPDIQMTQTTSSLSASLGDRVTISCRASQ
DISKYLNWYQQKPDGTVKLLIYHTSRLHSGVPSRFSGSGSGTDYSLTISN
LEQEDIATYFCQQGNTLPYTFGGGTKLEITGGGGSGGGGSGGGGSEVKLQ
ESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSY
AMDYWGQGTSVTVSSVNSGGGGSGGGGSAKPTTTPAPRPPTPAPTIASQP
LSLRPEACRPAAGGAVHTRGLDFAPRKIEVMYPPPYLDNEKSNGTIIHVK
GKHLCPSPLFPGPSKPFWVLVVVGGVLACYSLLVTVAFIIFWVRSKRSRL
LHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSTATKDTYDALHMQALPPR

We claim:

1. A peptide delivery system for delivering a peptide payload to a target cell or target locus, the peptide delivery system comprising:
a cell, extracellular vesicle, or lipid-containing particle into which an exogenous polypeptide has been anchored, which exogenous polypeptide comprises:
a first and a second transmembrane domain connected by a loop region, which loop region comprises a first cleavage site, a peptide payload, and a second cleavage site;
and optionally, a targeting moiety for targeting the peptide delivery system to the target cell or the target locus.

2. The peptide delivery system of claim 1 wherein the first and/or second cleavage sites comprise proteolytic cleavage substrates, preferably sheddase cleavage sites MMP cleavage sites, GrzB cleavage sites, and Casp3 cleavage sites.

3. The peptide delivery system of claim 2, which is configured to release the peptide payload upon cleavage by a protease, preferably aby a sheddase, which may be endogenous or exogenous (e.g., included as a component of the peptide delivery system).

4. The peptide delivery system of claim 3, wherein the sheddase is present at the target locus and/or released by the target cell.

5. The peptide delivery system of claim 3 or 4, wherein the sheddase is a full-time sheddase selected from ADAM proteases (metalloproteases), BACE proteases, serine-protease Granzyme-B and Site-1 proteases.

6. The peptide delivery system of claim 5, wherein the ADAM proteases include membrane-anchored type 1 proteases such as ADAM8, ADAM9, ADAM10, ADAM12, ADAM15, ADAM17, ADAM19, ADAMthr20, ADAM21, ADAM28, ADAM30, and ADAM33.

7. The peptide delivery system of claim 3 or 4, wherein the sheddase is a part-time sheddase selected from Meprin B, MT-MMPs (Membrane Type Matrix Metalloproteases), Pro-protein convertases, transmembrane serine proteases, Matrix Metalloproteases, Legumain and Cathepsin S and L.

8. The peptide delivery system of claim 7, wherein the MT-MMPs are membrane-anchored type 1 or GPI-anchored and include MT1-MMP, MT2-MMP, MT3-MMP, MT4-MMP, MT5-MMP, and MT6-MMP.

9. The peptide delivery system of claim 7, wherein the Pro-protein convertases are selected from PCSK1/3, KCSK2, Furin, PCSK4, PSCK5/6, PACE4, PCSK7, and PCSK9.

10. The peptide delivery system of claim 7, wherein the transmembrane serine proteases are selected from membrane-anchored type II proteases including Matriptase, Matriptase-2, Matriptase-3, Polymerase-1, Corin, Hepsin, TMPRSS2, TMPRSS3, TMPRSS4, MSPL, Spinesin, Enteropeptidase, HAT, DESCL1, TMPRSS11A, HAT-like 4 and HAT-like 5.

11. The peptide delivery system of claim 7, wherein the Matrix Metalloproteases are selected from soluble proteases including MMPP1, MMP2, MMP3, MMP4, MMP5, MMP6, MMP7, MMP8, MMP9, MMP10, MMPP11, MMP12, MMPP13, MMPP14, MMPP15, MMP16, MMPP17, MMP18, MMP19, MMP20, MMP21, MMP21, MMP 23A-B, MMP24, MMP25, MMP26, MMP27 and MMP28.

12. The peptide delivery system of claim 11, wherein the Matrix Metalloproteases are selected from MMP2, MMP9 and/or MMP25.

13. The peptide delivery system of claim 12, wherein the MMP2 is selected from SEQ ID NOS: 2-6.

14. The peptide delivery system of claim 12, wherein the MMP9 is selected from SEQ ID NOS: 7-8.

15. The peptide delivery system of claim 12, wherein the MMP25 is selected from SEQ ID NOS: 9-17, 18 or 155, 19-29.

16. The peptide delivery system of any of the preceding claims, wherein the transmembrane domain comprises a transmembrane domain from one or more of the following proteins: CD63, CD9, CD81, CD28, CD4, CD8, CD34, CD69, CD19, CD20, Integrins, TNFSF14, TNR1A, Aquaporins, NOTCH, NgR1, NRG1, GPi anchor, EGFR, or Rhodopsin.

17. The peptide delivery system of claim 1, which further comprises an intracellular or extracellular domain selected from or derived from one or more of the following proteins: OX-40, 4-1BB/CD137, CD2, CD7, CD27, CD30, B7-1 (CD80), B7-2 (CD86), PDL-1, Programmed Death-I (PD-1), Inducible T cell Co-Stimulator (ICOS), lymphocyte Function-associated Antigen-I (LFA-1 (CDI la/CD 18), CD3 gamma, CD3 delta, CD3 epsilon, CD247, CD276 (B7-H3), LIGHT, (TNFSF14), NKG2C, lg alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), The costimulatory ligand (PD-L2, 4-1 BBL, OX40L, Inducible Co-Stimulatory Ligand (ICOS-L), Inter-Cellular Adhesion Molecule (ICAM, CD30L, CD70, CD83, HLA-G, MICA, Ml CB, HVEM, lymphotoxin b receptor, 3/TR6, ILT3, ILT4), activating NK cell receptors, BTLA, a Toll ligand receptor, ICAM-1, B7-H3, CDS, ICAM-1, GITR, BAFFR, LIGHT, HVEM (LIGHTR), KIRDS2, NKp80 (KLRFI), NKp44, NKp30, NKp46, CD19, CD8 alpha, CD8 beta, IL-2R beta, IL-2R gamma, IL-7R alpha, ITGA4, VLAl, CD49a, ITGA4, CD49D, ITGA6, VLA-6, CD49f, ITGAD, CDl Id, ITGAE, CD103, ITGAL, CDl la, LFA-1, IT GAM, CDl lb, ITGAX, CDl le, ITGBI, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD 160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

18. The peptide delivery system of claim 1, which comprises a targeting moiety and the targeting moiety binds to BCMA, MUC16 (also known as CA125), EGFR, EGFRvIII, MUCI, Flt-3, WT-1, CD38, CD70, CD90, CD133, MHC-WTI, TSPANI0, MHC-PRAME, MHC-NY-ESOI, HER2 (ERBB2), CA-IX (Carbonic anhydrase IX), LIVI, ADAMI0, CHRNA2, LeY, NKG2D, CSI, CD44v6, CD24, LGR5, ALDH, ALDH1, CD54, Sca1, CD271, CD123, CD36, CD109, CD110, CD71-negative, CCA, ABCG2, Claudin-18.2 (Claudin-18A2, or Claudinl8 isoform 2), PSCA, DLL3 (Delta-like protein 3, Drosophila Delta homolog 3, Delta3), Mud 7 (Mucinl 7, Muc3, Muc3), FAP alpha (Fibroblast Activation Protein alpha), Ly6G6D (Lymphocyte antigen 6 complex locus protein G6d, c6orf23, G6D, MEGTI, NG25), PSMA, MSLN, or RNF43 (E3 ubiquitin-protein ligase RNF43, RING finger protein 43). BAFF, C242 antigen, disialoganglioside (GD2), 4-IBB, 5T4, CD22, CD221, CD23 (IgE receptor), CD28, CD30 (TNFRSF8), CD33, CD4, CD40, CD44v6, CD44 CD51, CD52, CD56, CD74, CEA, CNT0888, CTLA-4, DR5, EpCAM, FAP, fibronectin extradomain-B, folate receptor 1, GD3 ganglioside, glycoprotein 75, GPNMB, HER2/neu, HGF, human scatter factor receptor kinase, IGF-1 receptor, IGF-I, IgGI, LI-CAM, integrin α5β1, integrin αvβ, legumain, MORAb-009, MS4A1, MUC1, mucin CanAg, C-MET, CCR4, CD152, CD10, CD19, CD20, CD200, N-glycolylneuraminic acid, NPC-IC, PDGF-R a, PDL192, phosphatidylserine, tumor antigen CTAA16.88, VEGF-A, VEGFR-1, VEGFR2, vimentin, RANKL, RON, ROR1, SCH900105, SDC1, SLAMF7, TAG-72, tenascin C, TGF-β, TRAIL-R1, TRAIL-R2, folic acid receptor, transferrin receptors, or any combination thereof.

19. The peptide delivery system of claim 1, wherein the second transmembrane domain is connected to an intracellular region comprising an intracellular domain from one or more of 41BB, ICOS, and CD3ζ.

20. The peptide delivery system of claim 19, wherein phosphorylation by a kinase within the intracellular domain of 41BB, ICOS, or CD3ζ activates GrzB and allows GrzB to be transported out of the cell and cleave the the first and/or second cleavage site.

21. The peptide delivery system of claim 1, which comprises a third and fourth transmembrane domain connected by a second loop region, wherein the third transmembrane domain is connected to an intracellular region comprising an intracellular domain from 41BB, ICOS, or CD3z.

22. The peptide delivery system of claim 21, wherein phosphorylation by a kinases within the intracellular domain of 41BB, ICOS or CD3ζ activates the cell to release endogenous GrzB allowing the GrzB to be transported out of the cell and cleave GrzB cleavage present in the first or second loop region thereby releasing the peptide payload.

23. The peptide delivery system of claim 22, wherein the second transmembrane domain is from a NOTCH protein having a cleavage site configured to release a functional domain to which the NOTCH protein transmembrane domain is connected in the intracellular milieu.

24. The peptide delivery system of any of the preceding claims, wherein the payload comprises: an antibody, an fragment of an antibody, a VHH, a cytokine or chemokine consisting a fraction or derivative of a sulfated xylan, which is an antagonist of a ligand selected from the list comprising IL-1a, IL-1B, IL-2, IL-3, IL-4, IL-5, IL-6, IL-7, IL-8, IL-9, IL-10, IL-11, IL-12, IL-3, IL-14, IL-15 or IL-17 family of cytokines including IL-25, interferon, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, PDGF, SCF, TGFa, TGFB, TNFa, TNFB, TPO, VEGF, GH, NGF, NT3, NT4, NT5, NT6, NT7, Oncostatin M (OSM), insulin, MCP-1, MCP-2, MCP-3, MCP-4, MCP-5, a member of the MIP-1 family including MIP-la, MIP-2, eotaxin (eotaxin-1, -2 or -3), PBP (platelet basic protein), SDF-1, PBSF, PF4, RANTES, elastase; an enzyme of the cathepsin family, a cell adhesion molecules such as PECAM-1, a soluble or cell- or virus-bound receptor, cytokine-induced neutrophil chemoattractant (KC), TNF-a and IFN-g), and other soluble mediators of inflammation, such as reactive oxygen species and nitric oxide, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, thrombin, urokinase, a vaccine or any combination thereof.

25. The peptide delivery system of any of the preceding claims, further comprising a linker between the first transmembrane domain and the first cleavage site.

26. The peptide delivery system of claim 25, wherein the linker is 2-128 amino acids in length or 4-20 amino acids in length.

27. The peptide delivery system of claim 25, wherein the linker is selected from the linkers set forth in Table 4.

28. The peptide delivery system of claim 1, which is configured to be sorted specifically into extracellular vesicles.

29. The peptide delivery system of claim 1, configured to release two or more peptide payloads intracellularly or extracellularly.

30. The peptide delivery system of claim 1, which comprises two or more sheddase cleavage sites, and each cleavage site can be the same or different from any of the other cleavage sites.

31. The peptide delivery system of claim 1, which comprises an intracellular domain and the intracellular domain comprises a stalk domain of CD45 phosphatase.

32. The peptide delivery system of claim 1, comprising a single domain antibody or single chain of an antibody which binds the self-associated antigen (SAA) to bring the SAA in proximity, e.g., in cis or in trans.

33. The peptide delivery system of claim 1 or 11, which comprises a single domain antibody or single chain of an antibody which binds the self-associated antigen (SAA), and which further comprises a CD45 phosphatase-binding moiety or an NKG2A-binding moiety, which activates an inhibitory signal in the target cell.

34. The peptide delivery system of claim 1, wherein the peptide payload is any peptide or protein that serves a function within the human body, including hormones, cytokines, chemokines, pro-drugs, and antibodies in various formats, such as chimeric antigen receptors (CARs), bi-specific killer cell engager (BIKE), tri-specific killer cell engager (TRIKE), etc.

35. The peptide delivery system of claim 1, wherein the peptide delivery system is selected from: CD63-MMP2-a-CD16-a-CD33-Myc-MMP9-CD63-GPi (SEQ ID NO: 124), CD63-MMP2-a-CD16-a-CD33-Myc-MMP9-CytCD63-EV Motif (SEQ ID NO: 155), CD63-MMP2-a-CD16-a-CD33-Myc-MMP9-CytCD63 (SEQ ID NO: 36 or 188), TNFSF14-a-CD16-a-CD33-Myc-TNR1A-ADAM17-a-CD38-41BB (SEQ ID NO: 54 or 366 or 367), CD63-GrzB-a-CD16-a-CD33-Myc-GrzB-a-CD38-CD28-CD3z (SEQ ID NO: 71 or 252), CD63-MMP9-2L-TNFa-Myc-MMP9-CD63-Cyt (SEQ ID NO: 88 or 295), CD63-MMP9-2L-TNFa-Myc-MMP9-CD63-D-Cyt (Lacks EVs sorting motif) (SEQ ID NO: 103 or 311), CD63-DL1-MMP2-2L-TNFa-Myc-MMP2-CD63-Cyt (SEQ ID NO: 104 or 368), CD63-MMP9-2L-TNFa-Myc-MMP9-aCD19CAR (SEQ ID NO: 105 or 369), CD63-MMP2-2L-TNFa-Myc-MMP2-CD63-D-Cyt (SEQ ID NO: 106) or 370 or 371, CD63-MMP2-2L-TNFa-Myc-MMP2-aCD19CAR (SEQ ID NO: 107 or 372 o4 373), CD69cyt-TMII-TNFa-Myctag-CD28TM-cyt (SEQ ID NO: 119 or 370), CD69cyt-TMII-MMP9-TNFa-Myc-MMP9-CD28TM-cyt (SEQ ID NO: 120 or 377), CD69Cyt-TM-GrzB-TNFa-Myc-GrzB-a-CD19sc-Stalk-CD28-CD3z (SEQ ID NO: 107 or 372 or 373), CD69Cyt-TM-GrzB-nLuc-Myc-GrzB-CD28-CD3z (Delta sc-GAGE B) (SEQ ID NO: 108 or 374), CD69Cyt-TM-nLuc-Myc-a-CD19sc-Stalk-CD28-CD3z (Delta GrzB, GAGE A) (SEQ ID NO: 109 or 375), CD69Cyt-TM-GrzB (Original)-nLuc-Myc-GrzB(Original)-a-CD19sc-Stalk-CD28-CD3z (SEQ ID NO: 358 or 382), CD69Cyt-TM-GrzB(IEPD)-nLuc-Myc-GrzB(IEPD)-a-CD19sc-Stalk-CD28-CD3z (SEQ ID NO: 110 or 383), CD69Cyt-TM-GrzB(SASA)-nLuc-Myc-, GrzB(SASA)-a-CD19sc-Stalk-CD28-CD3z (SEQ ID NO: 111 or 376), or CD69Cyt-TM-GrzB(ADKG)-nLuc-Myc-GrzB(ADKG)-a-CD19sc-Stalk-CD28-CD3z (SEQ ID NO: 112 or 384).

36. The peptide delivery system of any of the preceding claims, which comprises a cell into which the exogenous polypeptide has been anchored.

37. The peptide delivery system of claim 36, wherein the cell is selected from a Hematopoietic Stem Cells (HSC), an induced Pluripotent Stem Cell (iPSC) and their derived cell products, an Adoptive T cell, a Dendritic cell (DC), Natural killer (NK) cells, or any therapeutic immune or non-immune cells.

38. An adoptive cell or engineered immune cell comprising the peptide payload of claim 1, wherein the immune cells are autologous or allogeneic.

39. The peptide delivery system of claim 1, wherein the exogenous polypeptide is introduced into the cell individually via a CRISPR system, or in the form of viral or nonviral vectors, or one or more mRNAs.

40. The peptide delivery system of claim 39, wherein the exogenous polypeptide is delivered using CPPs, micelles, liposomes, nanoparticles, dendrimers, nanotubes, electroporation, viral transduction, nucleofection, transfection, cell fusion, or microinjection.

41. A genetic construct encoding the exogenous polypeptide of claim 1.

42. A method of treating a patient in need thereof comprising administering the peptide delivery system of any one of claims 1-37 to the patient.

43. A method of delivering a peptide to a target locus in a patient in need thereof, the method comprising: administering to the patient a peptide delivery system comprising a lipid-comprising vescicle (e.g., cell, extracellular vesicle, lipid nanoparticle) into which an exogenous polypeptide has been anchored, the exogenous polypeptide comprising:
a peptide payload;
a first and a second transmembrane region;
the first transmembrane region comprising a transmembrane domain connected to at least one sheddase or other proteolytic cleavage site;
the second transmembrane region comprising a transmembrane domain connected to at least one sheddase or other proteolytic cleavage site; and
optionally, a targeting moiety for targeting the exogenous polypeptide to a target locus, wherein when the lipid-comprising vescicle arrives at the target locus, a sheddase and/or other protease cleaves the cleavage sites of the first and second transmembrane regions releasing the peptide payload.

44. The method of claim 43, wherein the cleaveage sites are derived from proteolytic cleavage substrates naturally present in Glycosylphosphatidylinositol (GPi) anchored proteins, membrane proteins with a single, two, three, four, or more transmembrane domains, intracellular domains, or extracellular domains in the extracellular matrix or in neighboring cells.

45. The method of claim 43, wherein the sheddases are selected from full- or part-time sheddases.

46. The method of claim 45, wherein the full-time sheddases are selected from ADAM proteases, BACE proteases, serine-protease Granzyme-B, and Site-1 proteases.

47. The method of claim 46, wherein the ADAM proteases are selected from ADAM8, ADAM9, ADAM10, ADAM12, ADAM15, ADAM17, ADAM19, ADAM20, ADAM21, ADAM28, ADAM30, and ADAM33.

48. The method of claim 45, wherein part-time sheddases are selected from: Meprin B, MT-MMPs (Membrane Type Matrix Metalloproteases), Pro-protein convertases, transmembrane serine proteases, Matrix Metalloproteases, Legumain and Cathepsin S and L, wherein the Meprin B is a membrane-anchored type 1 metalloprotease, and wherein the MT-MMPs are membrane-anchored type 1 or GPI-anchored, and include MT1-MMP, MT2-MMP, MT3-MMP, MT4-MMP, MT5-MMP, and MT6-MMP.

49. The method of claim 48, wherein the Pro-protein convertases are selected from PCSK1/3, KCSK2, Furin, PCSK4, PSCK5/6, PACE4, PCSK7, and PCSK9.

50. The method of claim 48, wherein the transmembrane serine proteases are selected from membrane-anchored type II proteases, including, for example, Matriptase, Matriptase-2, Matriptase-3, Polymerase-1, Corin, Hepsin, TMPRSS2, TMPRSS3, TMPRSS4, MSPL, Spinesin, Enteropeptidase, HAT, DESCL1, TMPRSS11A, HAT-like 4 and HAT-like 5.

51. The method of claim 48, wherein the Matrix Metalloproteases are soluble proteases and include one or more of MMP1, MMP2, MMP3, MMP4, MMP5, MMP6, MMP7, MMP8, MMP9, MMP10, MMP11, MMPP12, MMPP13, MMP14, MMP15, MMP16, MMPP17, MMP18, MMP19, MMP20, MMP21, MMP21, MMP23A-B, MMP24, MMP25, MMP26, MMP27, and MMP28.

52. The method of claim 51, wherein the Matrix Metalloproteases are selected from MMP2, MMP9, and/or MMP25.

53. The method of claim 52, wherein the MMP2 is selected from SEQ ID NOS: 2-6.

54. The method of claim 52, wherein the MMP9 is selected from SEQ ID NOS: 7-8.

55. The method of claim 52, wherein the MMP25 is selected from SEQ ID NOS: 9-17, 18 or 155, 19-29.

56. The method of claim 43, wherein the transmembrane domain of the first and/or second transmembrane region comprises a transmembrane domain derived from one or more of CD63, CD9, CD81, CD28, CD4, CD8, CD34, CD69, CD19, CD20, Integrins, TNFSF14, TNR1A, Aquaporins, NOTCH, NgR1, NRG1, GPi anchor, EGFR, and Rhodopsin.

57. The method of claim 43, wherein cleavage at one or more of the cleavage sites occurs by endogenous sheddases.

58. The method of claim 43, wherein peptide delivery system further comprises a sheddase, and cleavage occurs at one or more of the cleavage sites by the peptide delivery system sheddase.

59. The method of claim 43, wherein an intracellular or extracellular domain of the exogenous polypeptide is selected from an intracellular or extracellular domain from one or more of the following: OX-40, 4-1BB/CD137, CD2, CD7, CD27, CD30, B7-1 (CD80), B7-2 (CD86), PDL-1, Programmed Death-I (PD-1), Inducible T cell Co-Stimulator (ICOS), lymphocyte Function-associated Antigen-I (LFA-1 (CDI la/CD 18), CD3 gamma, CD3 delta, CD3 epsilon, CD247, CD276 (B7-H3), LIGHT, (TNFSF14), NKG2C, lg alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), The costimulatory ligand (PD-L2, 4-1 BBL, OX40L, Inducible Co-Stimulatory Ligand (ICOS-L), Inter-Cellular Adhesion Molecule (ICAM, CD30L, CD70, CD83, HLA-G, MICA, Ml CB, HVEM, lymphotoxin b receptor, 3/TR6, ILT3, ILT4), activating NK cell receptors, BTLA, a Toll ligand receptor, ICAM-1, B7-H3, CDS, ICAM-1, GITR, BAFFR, LIGHT, HVEM (LIGHTR), KIRDS2, NKp80 (KLRFI), NKp44, NKp30, NKp46, CD19, CD8 alpha, CD8 beta, IL-2R beta, IL-2R gamma, IL-7R alpha, ITGA4, VLAl, CD49a, ITGA4, CD49D, ITGA6, VLA-6, CD49f, ITGAD, CDl Id, ITGAE, CD103, ITGAL, CDl la, LFA-1, IT GAM, CDl lb, ITGAX, CDl le, ITGB1, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAMI, CRT AM, Ly9 (CD229), CD 160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

60. The method of claim 43, wherein the targeting moiety binds BCMA, MUC16 (also known as CA125), EGFR, EGFRvIII, MUCI, Flt-3, WT-1, CD38, CD70, CD90, CD133, MHC-WTI, TSPANI0, MHC-PRAME, MHC-NY-ESOI, HER2 (ERBB2), CA-IX (Carbonic anhydrase IX), LIVI, ADAMI0, CHRNA2, LeY, NKG2D, CSI, CD44v6, CD24, LGR5, ALDH, ALDH1, CD54, Sca1, CD271, CD123, CD36, CD109, CD110, CD71-negative, CCA, ABCG2, Claudin-18.2 (Claudin-18A2, or Claudinl8 isoform 2), PSCA, DLL3 (Delta-like protein 3, Drosophila Delta homolog 3, Delta3), Mud 7 (Mucinl 7, Muc3, Muc3), FAP alpha (Fibroblast Activation Protein alpha), Ly6G6D (Lymphocyte antigen 6 complex locus protein G6d, c6orf23, G6D, MEGTI, NG25), PSMA, MSLN, or RNF43 (E3 ubiquitin-protein ligase RNF43, RING finger protein 43). BAFF, C242 antigen, disialoganglioside (GD2), 4-IBB, 5T4, CD22, CD221, CD23 (IgE receptor), CD28, CD30 (TNFRSF8), CD33, CD4, CD40, CD44v6, CD44 CD51, CD52, CD56, CD74, CEA, CNT0888, CTLA-4, DR5, EpCAM, FAP, fibronectin extradomain-B, folate receptor 1, GD3 ganglioside, glycoprotein 75, GPNMB, HER2/neu, HGF, human scatter factor receptor kinase, IGF-1 receptor, IGF-I, IgGI, LI-CAM, integrin α5β1, integrin αvβ, legumain, MORAb-009, MS4A1, MUC1, mucin CanAg, C-MET, CCR4, CD152, CD10, CD19, CD20, CD200, N-glycolylneuraminic acid, NPC-IC, PDGF-R a, PDL192, phosphatidylserine, tumor antigen CTAA16.88, VEGF-A, VEGFR-1, VEGFR2, vimentin, RANKL, RON, ROR1, SCH900105, SDC1, SLAMF7, TAG-72, tenascin C, TGF-β, TRAIL-R1, TRAIL-R2, folic acid receptor, transferrin receptors, and any combination thereof.

61. The method of claim 43, wherein the first and/or second transmembrane region comprises (i) an intracellular region comprising an intracellular region sequence from 41BB, ICOS, or CD3ζ, and (ii) a GrzB cleavage site; and wherein the action of kinases on the intracellular region sequence from 41BB, ICOS, or CD3ζ induces the release of the GrzB allowing it to be transported out of the cell and cleave GrzB cleavage sites, thereby releasing the peptide payload, the peptide payload optionally comprising a bispecific antibody.

62. The method of claim 61, wherein the second transmembrane region comprises a transmembrane domain from a NOTCH protein and a cleavage site inside the cell membrane and cleavage at the cleavage site inside the cell membrane releases a functional domain in the intracellular milieu.

63. The method of claim 43, wherein the peptide payload comprises: a cytokine or chemokine consisting a fraction or derivative of a sulfated xylan, which is an antagonist of a ligand selected from the list comprising IL-la, IL-1B, IL-2, IL-3, IL-4, IL-5, IL-6, IL-7, IL-8, IL-9, IL-10, IL-11, IL-12, IL-3, IL-14, IL-15 or IL-17 family of cytokines including IL-25, interferon, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, PDGF, SCF, TGFa, TGFB, TNFa, TNFB, TPO, VEGF, GH, NGF, NT3, NT4, NT5, NT6, NT7, Oncostatin M (OSM), insulin, MCP-1, MCP-2, MCP-3, MCP-4, MCP-5, a member of the MIP-1 family including MIP-la, MIP-2, eotaxin (eotaxin-1, -2 or -3), PBP (platelet basic protein), SDF-1, PBSF, PF4, RANTES, elastase; an enzyme of the cathepsin family, a cell adhesion molecules such as PECAM-1, a soluble or cell- or virus-bound receptor, cytokine-induced neutrophil chemoattractant (KC), TNF-α and IFN-g), and other soluble mediators of inflammation, such as reactive oxygen species and nitric oxide, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, thrombin, urokinase, a vaccine or any combination thereof.

64. The method of claim 43, wherein the exogenous polypeptide further comprises at least one linker, which at least one linker is optionally positioned between the cleavage site present in the first transmembrane region and a first binding moiety (e.g., anti-CD3 VHH domain), between a cleavage site present in the second transmembrane region and a second binding moiety (e.g., anti-TAA VHH domain), and/or between the first and second binding moieties.

65. The method of claim 64, wherein the linker is 2-128 amino acids in length, or preferably 4-20 amino acids in length.

66. The method of claim 64, wherein the linker is selected from Table 4.

67. The method of claim 43, wherein the exogenous polypeptide can also be configure for sorting specifically into extracellular vesicles (EVs).

68. The method of claim 43, wherein the peptide payload comprises two or more bispecific engagers, and the bispecific engagers are released intracellularly or extracellularly.

69. The method of claim 43, wherein peptide delivery system comprises sheddase cleavage sites, and each cleavage site can be the same or different from any of the other cleavage sites.

70. The method of claim 43, wherein the peptide delivery system comprises and intracellular domain, and the intracellular domain comprises a stalk domain of CD45 phosphatase.

71. The method of claim 43, wherein the peptide delivery system comprising a single domain antibody or single chain of an antibody which binds the self-associated antigen (SAA) to bring the SAA in proximity, e.g., in cis or in trans.

72. The method of claim 43, which comprises a single domain antibody or single chain of an antibody which binds the self-associated antigen (SAA), and which further comprises a CD45 phosphatase- or an NKG2A-binding moiety, which activates an inhibitory signal in the target cell.

73. The method of claim 43, wherein the peptide payload is any peptide that serves a function within the body is a potential therapeutic, including hormones, cytokines, chemokines, pro-drugs, and antibodies in various formats, such as chimeric antigen receptors (CARs), bi-specific killer cell engager (BIKE), tri-specific killer cell engager (TRIKE), etc.

74. The method of claim 43, wherein the peptide delivery system is selected from: CD63-MMP2-a-CD16-a-CD33-Myc-MMP9-CD63-GPi (SEQ ID NO: 124), CD63-MMP2-a-CD16-a-CD33-Myc-MMP9-CytCD63-EV Motif (SEQ ID NO: 155), CD63-MMP2-a-CD16-a-CD33-Myc-MMP9-CytCD63 (SEQ ID NO: 36 or 188), TNFSF14-a-CD16-a-CD33-Myc-TNR1A-ADAM17-a-CD38-41BB (SEQ ID NO: 54 or 366 or 367), CD63-GrzB-a-CD16-a-CD33-Myc-GrzB-a-CD38-CD28-CD3z (SEQ ID NO: 71 or 252), CD63-MMP9-2L-TNFa-Myc-MMP9-CD63-Cyt (SEQ ID NO: 88 or 295), CD63-MMP9-2L-TNFa-Myc-MMP9-CD63-D-Cyt (Lacks EVs sorting motif) (SEQ ID NO: 103 or 311), CD63-DL1-MMP2-2L-TNFa-Myc-MMP2-CD63-Cyt (SEQ ID NO: 104 or 368), CD63-MMP9-2L-TNFa-Myc-MMP9-aCD19CAR (SEQ ID NO: 105 or 369), CD63-MMP2-2L-TNFa-Myc-MMP2-CD63-D-Cyt (SEQ ID NO: 106 or 370 or 371), CD63-MMP2-2L-TNFa-Myc-MMP2-aCD19CAR (SEQ ID NO: 107 or 372 or 373), CD69cyt-TMII-TNFa-Myctag-CD28TM-cyt (SEQ ID NO: 119 or 370), CD69cyt-TMII-MMP9-TNFa-Myc-MMP9-CD28TM-cyt (SEQ ID NO: 120 or 377), CD69Cyt-TM-GrzB-TNFa-Myc-GrzB-a-CD19sc-Stalk-CD28-CD3z (SEQ ID NO: 107 or 372 or 373), CD69Cyt-TM-GrzB-nLuc-Myc-GrzB-CD28-CD3z (Delta sc-GAGE B) (SEQ ID NO: 108 or 374), CD69Cyt-TM-nLuc-Myc-a-CD19sc-Stalk-CD28-CD3z (Delta GrzB, GAGE A) (SEQ ID NO: 109 or 375), CD69Cyt-TM-GrzB, (Original)-nLuc-Myc-GrzB(Original)-a-CD19sc-Stalk-CD28-CD3z (SEQ ID NO: 358 or 382), CD69Cyt-TM-GrzB(IEPD)-nLuc-Myc-GrzB(IEPD)-a-CD19sc-Stalk-CD28-CD3z (SEQ ID NO: 110 or 383), CD69Cyt-TM-GrzB(SASA)-nLuc-Myc-, GrzB(SASA)-a-CD19sc-Stalk-CD28-CD3z (SEQ ID NO: 111 or 376), or CD69Cyt-TM-GrzB(ADKG)-nLuc-Myc-GrzB(ADKG)-a-CD19sc-Stalk-CD28-CD3z (SEQ ID NO: 112 or 384).

75. The method of claim 43, wherein the lipid-comprising vesicle comprises a cell, and the cell is selected from a Hematopoietic Stem Cells (HSC), induced Pluripotent Stem Cell (iPSC) and a derived cell product, an Adoptive T cell, a Dendritic cell (DC), a Natural killer (NK) cell, or any therapeutic immune cell or non-immune cell.

76. The method of claim 75, wherein the immune cell is autologous or allogeneic.

77. The method of claim 43, wherein the peptide delivery system is introduced into a cell individually in the form of viral or nonviral vectors, mRNAs, peptides, proteins, antibodies, nanobodies, oligonucleotides, or extracellular vesicles (EVs).

78. The method of claim 43, wherein the peptide delivery system is delivered using CPPs, micelle, liposomes, nanoparticles, dendrimer, nanotube, electroporation, viral transduction, nucleofection, transfection, cell fusion or microinjection.
